(19) United States
(12) Patent Application Publication
(10) Pub. No.: US 2020/0308162 A1
(43) Pub. Date: Oct. 1, 2020

(54) DRUGS AND COMPOSITIONS FOR OCULAR DELIVERY

(71) Applicant: Graybug Vision, Inc., Redwood City, CA (US)

(72) Inventors: Jeffrey L. Cleland, San Carlos, CA (US); Ming Yang, Lutherville-Timonium, MD (US); John G. Bauman, El Sobrane, CA (US); Nu Hoang, Annapolis, MD (US); Jane Chisholm, Baltimore, MD (US)

(73) Assignee: Graybug Vision, Inc., Redwood City, CA (US)

(21) Appl. No.: 16/899,422

(22) Filed: Jun. 11, 2020

Related U.S. Application Data
(63) Continuation of application No. PCT/US2018/065843, filed on Dec. 14, 2018.
(60) Provisional application No. 62/663,134, filed on Apr. 26, 2018.
(60) Provisional application No. 62/598,943, filed on Dec. 14, 2017.

(51) Int. Cl. C07D 417/04

(52) U.S. Cl. CPC C07D 417/04; A61K 9/0019

(57) ABSTRACT

New prodrugs of therapeutically active compounds, including oligomeric prodrugs of ethacrynic acid, and compositions to treat medical disorders, for example glaucoma, a disorder or abnormality related to an increase in intraocular pressure (IOP), a disorder requiring neuroprotection, age-related macular degeneration, or diabetic retinopathy. Also a method for the controlled administration of timolol to a patient in need thereof, such as a human, comprising administering a prodrug of timolol in a microparticle in vivo, wherein the timolol prodrug containing microparticle exhibits in vitro drug release kinetics in an aqueous solution at a pH between 6-8 at body temperature of a substantially consistent release of at least 60% timolol itself by molar ratio to the prodrug of timolol or an intermediate metabolite thereof over at least 100 days.

### CROSS-REFERENCE TO RELATED APPLICATIONS

This application is a continuation of International Patent Application No. PCT/US2018/065843, filed in the U.S. Receiving Office on Dec. 14, 2018, which claims the benefit of provisional U.S. Application No. 62/598,943 filed Dec. 14, 2017 and provisional U.S. Application No. 62/663,134 filed Apr. 26, 2018. The entirety of these applications are hereby incorporated by reference for all purposes.

### BACKGROUND

The eye is a complex organ with unique anatomy and physiology. The structure of the eye can be divided into two parts, the anterior and posterior. The cornea, conjunctiva, aqueous humor, iris, ciliary body and lens are in the anterior portion. The posterior portion includes the sclera, choroid, retinal pigment epithelium, neural retina, optic nerve and vitreous humor. The most prevalent diseases affecting the posterior segment of the eye are dry and wet age-related macular degeneration (AMD) and diabetic retinopathy. The most important diseases affecting the anterior segment include glaucoma, allergic conjunctivitis, anterior uveitis and cataracts. Glaucoma, which damages the eye's optic nerve, is a leading cause of vision loss and blindness. A major risk factor for glaucoma is intraocular pressure (IOP) and therefore lowering IOP is currently one approach to treat the disease. Examples of drugs for the treatment of IOP and glaucoma are the loop diuretic ethacrynic acid (ECA) and β-blockers such as Timolol.

To address issues of ocular delivery, a large number of types of delivery systems have been devised. Such include conventional (solution, suspension, emulsion, ointment, inserts and gels); vesicular (liposomes, exosomes, niosomes, discomes and pharmacosomes); advanced materials (scleral plugs, gene delivery, siRNA and stem cells); and, controlled release systems (implants, hydrogels, dendrimers, iontophoresis, collagen shields, polymeric solutions, therapeutic contact lenses, cyclodextrin carriers, microneedles and microemulsions and particulates (microparticles and nanoparticles)).

Typical routes of drug delivery to the eye are topical, systemic, subconjunctival, intravitreal, punctal, intrasceral, transscleral, anterior or posterior sub-Tenon's, suprachoroidal, choroidal, subchoroidal, and subretinal.

Topical drops are the most widely used non-invasive routes of drug administration to treat anterior ocular diseases due to their non-invasiveness and convenience. While topical eye drops of ECA were effective in decreasing IOP in rabbit and monkey eyes, ECA administration also led to corneal edema and moderate diffuse superficial corneal erosion, especially at higher doses (Tingey, D. P. et al. Arch Ophthalmol. 1992; 110: 699-702). ECA ointment to four glaucomatous monkey eyes led to mild eyelid edema, conjunctival hyperemia, and discharge at the highest concentration of 2.5% ECA (Wang, R F. et al. Arch Ophthalmol. 1994; 112: 390-394). Topical administration is currently limited by the adverse side effects observed at the dose required for efficacy. Other barriers to effective topical delivery include tear turnover, nasolacrimal drainage, reflex blinking, and the barrier of the mucosal membrane. It is considered that less than 5% of topically applied dosages reach the deeper ocular tissue.

The patient may be required to instill topical drops up to four times a day. Indeed, certain patients, including corneal transplant recipients, require therapeutic doses of medications to be continuously maintained in the corneal tissues and some patients are required to endure lengthy and arduous dosing regimens that often involve up to hourly application. Each repeat dosing not only requires a further investment of a patient's time, but also increases the chance of irritation and non-compliance.

Drug delivery to the posterior area of the eye usually requires a different mode of administration from topical drops, and is typically achieved via an intravitreal injection, periocular injection or systemic administration. Systemic administration is not preferred given the ratio of volume of the eye to the entire body and thus unnecessary potential systemic toxicity. Therefore, intravitreal injections are currently the most common form of drug administration for posterior disorders. However, intravitreal injections are also associated with risk due to the common side effect of inflammation to the eye caused by administration of foreign material to this sensitive area, endophthalmitis, hemorrhage, retinal detachment and poor patient compliance.

Transscleral delivery with periocular administration is seen as an alternative to intravitreal injections, however, ocular barriers such as the sclera, choroid, retinal pigment epithelium, lymphatic flow and general blood flow compromise efficacy.

To treat ocular diseases, and in particular disease of the posterior chamber, the drug must be delivered in an amount and for a duration to achieve efficacy. This seemingly straightforward goal, which is difficult to achieve in practice with any drug, is especially challenging when administering ECA due to the unfavorable lipophilicity of the compound's anionic form. Because ECA is a carboxylic acid with a pka of approximately 2.8, ECA exists as the anionic form at physiological pH, making it difficult to penetrate the cornea.

Additional examples of common drug classes used for ocular disorders include prostaglandins, carbonic anhydrase inhibitors, receptor tyrosine kinase inhibitors (RTKIs), beta-blockers, alpha-adrenergic agonists, parasympathomimetics, epinephrine, and hyperosmotic agents.

Although a number of prostaglandin carboxylic acids are effective in treating eye disorders, for example, by lowering IOP, their hydrophilic nature can lead to rapid clearance from the surface of the eye before effective therapy can be achieved. As a result, prostaglandins are dosed in the form of selected esters to allow entry to the eye and a “prolonged” residence. When in the eye, native esterase enzymes cleave the prostaglandin ester to release the active species. Despite this innovation, current drop administered prostaglandins, for example, latanoprost, bimatoprost, and travoprost, still require daily or several times daily dosing regimens and may cause irritation or hyperemia to the eye in some patients. In addition, nearly half of all glaucoma patients on prostaglandin therapy require a second agent for control of IOP (Physician Drug and Diagnosis Audit (PDDA) from Verispan, L.L.C. January-June, 2003).

Carbonic anhydrase inhibitors (CAIs) are used as an alternative and sometimes in conjunction with prostaglandins to treat eye disorders. Unfortunately, compliancy issues can occur as these medications also require daily or dosing up to four times a day, and may also cause irritation or hyperemia to the eye in some patients.

Another potential avenue for the treatment of ocular disorders involves protecting neurons directly. Preliminary data on receptor tyrosine kinase inhibitors (RTKIs) and dual leucine zipper kinase inhibitors (DLKIs) suggests that instead of treating increasing ocular pressure, molecules such as Sunitinib and Crizotinib can prevent the associated nerve damage. Unfortunately, Sunitinib has had observed hepatotoxicity in both clinical trials and post-marketing clinical use.

References that describe treatments of ocular disorders and the synthesis of compounds related to treating ocular disorders include U.S. Pat. No. 8,058,467 assigned to Nicox S.A., titled “Prostaglandin derivatives”; WO2009/035565 assigned to Qlt Plug Delivery Inc titled “Prostaglandin analogues for implant devices and methods”; U.S. Pat. No. 5,446,041 assigned to Allergan Inc. titled “Intraocular pressure reducing 11-acyl prostaglandins”; DE2263393 assigned to Upjohn Co. titled “9-O-Acylated prostaglandins F2a”; U.S. Pat. No. 5,292,754 assigned to Shionogi & Co. patent publication titled “Treatment for hypertension or glaucoma in eyes”; EP1329453 assigned to Ragactive titled “Method for obtaining 4-(n-alkylamine)-5, 6-dihydro-4h-thieno-(2,3-b)-thiopyran-2-sulfonamide-7, 7-dioxides and intermediate products”; GB844946 assigned to American Cyanamid Co. titled “2-(N-Substituted)acylamino-1,3,4-thiadiazole-5-sulfonamides”; WO 1998/07044 titled “Timolol Derivatives”; and U.S. 2017-0080092 titled “Compounds and Compositions for the Treatment of Ocular Disorders” assigned to Graybug Vision, Inc.

Other publications include “The modelling and kinetic investigation of the lipase-catalyzed acetylation of stereoisomeric prostaglandins” (Vallikivi, I., et al.; J. Mol. Catal. B: Enzym. 2005, 35(1-3): 62-69); “Lipase-catalyzed acylation of prostanoids” (Parve, O. et al. Bioorg. Med. Chem. Lett. 1999, 9(13): 1853-1858); and, “New prostaglandin (PGF) derivatives from the soft coral Lobophyton depressum” (Carmely, S., et al. Tetrahedron Lett. 1980, 21(9): 875-878).

Publications that describe ECA and ECA analogs for the treatment of ocular disorders include “Effects of topical Ethacrynic acid adducts on intraocular pressure in rabbits and monkeys” (Tingey, D. P. et al. Arch Ophthalmol. 1992; 110: 699-702); “The effect of intracamerally injected Ethacrynic acid on intraocular pressure in patients with glaucoma” (Melamed, S. et al. Am J Ophthalmol 1992, 113:508-512); “Effects of Topical Ethacrynic acid Ointment vs. Timolol on Intraocular Pressure in Glaucomatous Monkey Eyes” (Arch Ophthalmol. 1994; 112: 390-394); “On the Acylation of Hydroxy- and Mercaptocarboxylic Acid Esters Using the Carbodiimide/Acylation Catalyst Method” Rao, N. H.; Roth, H. J. Arch. Pharm. 1989; 322:523-530); “Controlled release of Ethacrynic acid from poly(lactide-co-glycolide) films for glaucoma treatment” (Wang et al. Biomaterials 2004; 25: 4279-4285); and, “Novel antiglaucoma prodrugs and codrugs of Ethacrynic acid” (Cynkowsak G. et al. Bioorganic & Medicinal Chemistry Letters 2005; 15: 3524-3527).

Patent applications that describe ECA prodrugs include WO2006/047466 assigned to Duke University titled “Ophthalmological Drugs”; U.S. Pat. No. 5,565,434 assigned to the University of Iowa Research Foundation titled “Hexose and Pentose Prodrugs of Ethacrynic acid”; WO 2016/118506 titled “Compositions for the Sustained Release of Anti-Glaucoma Agents to control Intraocular Pressure” assigned to the Johns Hopkins University; U.S. Pat. No. 4,661,515 titled “Compounds having Angiotensin Converting Enzyme Inhibitory Activity and Diuretic Activity” assigned to USV Pharmaceutical Corporation; and, CN 103610669 titled “Bis-(p-alkoxy benzene acrylketone) like glutathione-S-transferase potential inhibitor”.

Patent applications that describe derivatives of prostaglandins include U.S. Pat. No. 5,767,154 assigned to Allergan titled “5-tran-prostaglandins of the F series and their use as ocular hypotensives”, EP0667160A2 assigned to Alcon Laboratories titled “Use of certain prostaglandin analogues to treat glaucoma and ocular hypertension”; EP667160 titled “Use of certain prostaglandin analogues to treat glaucoma and ocular hypertension”; EP0850926A2 assigned to Asahi glass company and Santen Pharmaceutical Co., titled “Difluoroprostaglandin derivatives and their use”; JP2000080075 assigned to Asahi Glass Co., titled “Preparation of 15-deoxy-15,15-difluoroprostaglandins as selective and chemically-stable drugs”; JP11255740 titled “Preparation of 15-deoxy-15-monofluoroprostaglandin derivatives”; JP10087607 titled “Preparation of fluorine-containing prostaglandins as agents for inducing labor and controlling animal sexual cycle”; WO9812175 titled “Preparation of fluorinated prostaglandin derivatives for treatment of glaucoma”; JP10259179 assigned to Santen Pharmaceutical Co. titled “Preparation of multi-substituted aryloxy-group containing prostaglandins and their use”; and, EP850926 titled “Preparation of difluoroprostaglandin derivatives and their use for treatment of an eye disease”.

Johns Hopkins University has filed a number of patents claiming formulations for ocular injections including WO2013/138343 titled “Controlled Release Formulations for the Delivery of HIF-1 Inhibitors”, WO2013/138346 titled “Non-linear Multiblock Copolymer-drug Conjugates for the Delivery of Active Agents”, WO2011/106702 titled “Sustained Delivery of Therapeutic Agents to an Eye Compartment”, WO2016/025215 titled “Glucorticoid-loaded Nanoparticles for Prevention of Corneal Allograft Rejection and Neovascularization”, WO2016/100392 titled “Sunitinib Formulations and Methods for Use Thereof in Treatment of Ocular Disorders”, WO2016/100380 titled “Sunitinib Formulation and Methods for Use Thereof in Treatment of Glaucoma”, WO2016/118506 titled “Compositions for the Sustained Release of Anti-Glaucoma Agents to Control Intraocular Pressure”, WO2013/166385 titled “Nanocrystals, Compositions, and Methods that Aid Particle Transport in Mucus”, WO2005/072710 titled “Drug and Gene Carrier Particles that Rapidly move Through Mucus Barriers,” WO2008/030557 titled “Compositions and Methods for Enhancing Transport through Mucus”, WO2012/061703 titled “Compositions and Methods Relating to Reduced Mucoadhesion,” WO2012/039979 titled “Large Nanoparticles that Penetrate Tissue,” WO2012/109363 titled “Mucus Penetrating Gene Carriers”, WO2013/090804 titled “Biodegradable Stealth Nanoparticles Prepared by a Novel Self-Assembly Emulsification Method,” WO2013/110028 titled “Nanoparticles Formulations with Enhanced Mucosal Penetration”, and WO2013/166498 titled “Lipid-based Drug Carriers for Rapid Penetration through Mucus Linings”.

GrayBug Vision, Inc. discloses prodrugs for the treatment of ocular therapy in US 2018-0110864, granted U.S. Pat. Nos. 9,808,531, 9,956,302, 10,098,965, 10,117,950, and 10,111,964 and PCT applications WO2017/053638 and WO2018/175922. Aggregating microparticles for ocular therapy are described in US 2017-0135960, US 2018-0326078, WO2017/083779, and WO2018/209155.

U.S. Patent application 2010/227865 titled “Oligomer-Beta Blocker Conjugates” describes beta-blocker mono prodrugs.

The object of this invention is to provide additional compounds, compositions and methods to treat ocular disorders, including intraocular pressure (IOP).

### SUMMARY

The present invention provides new prodrugs, including oligomeric prodrugs of ethacrynic acid and Timolol, and compositions thereof of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII:

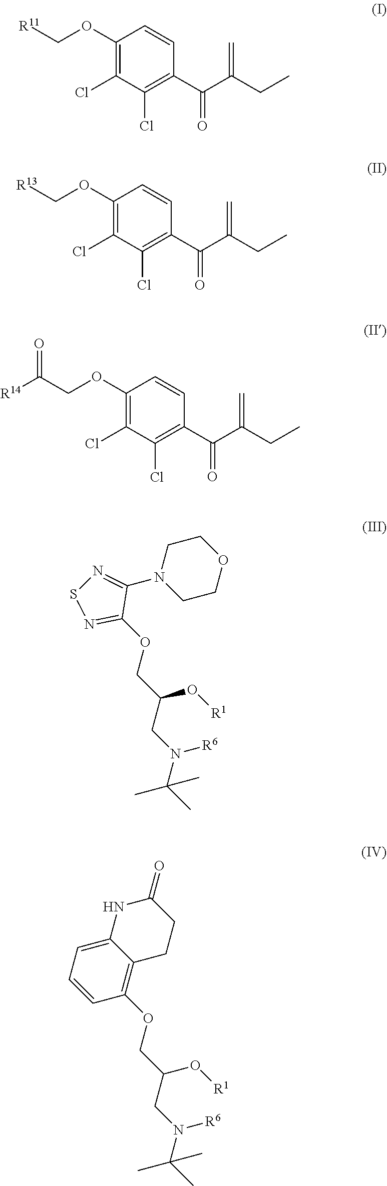

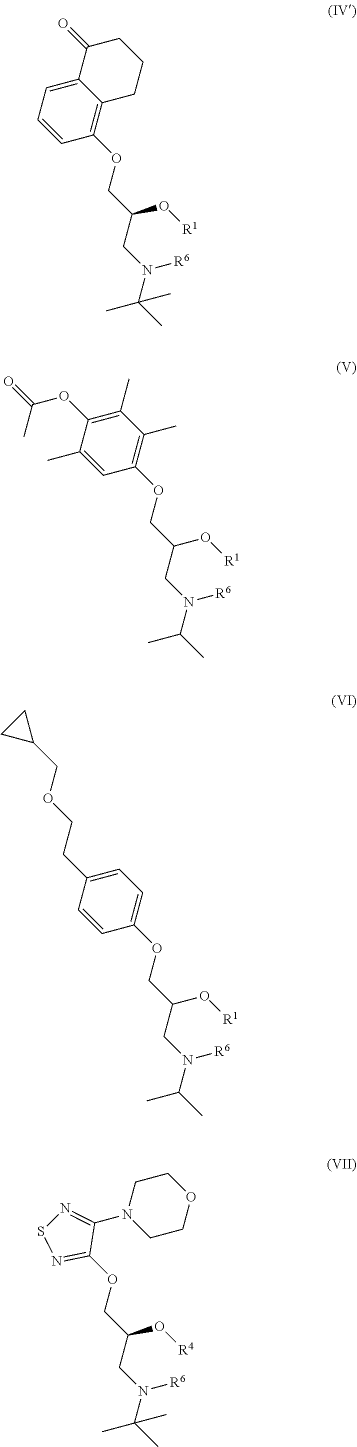

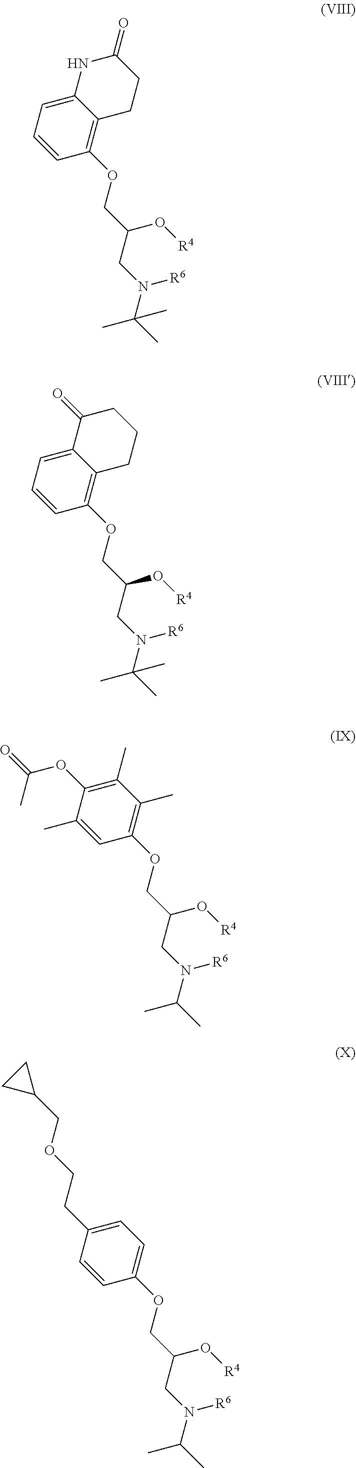

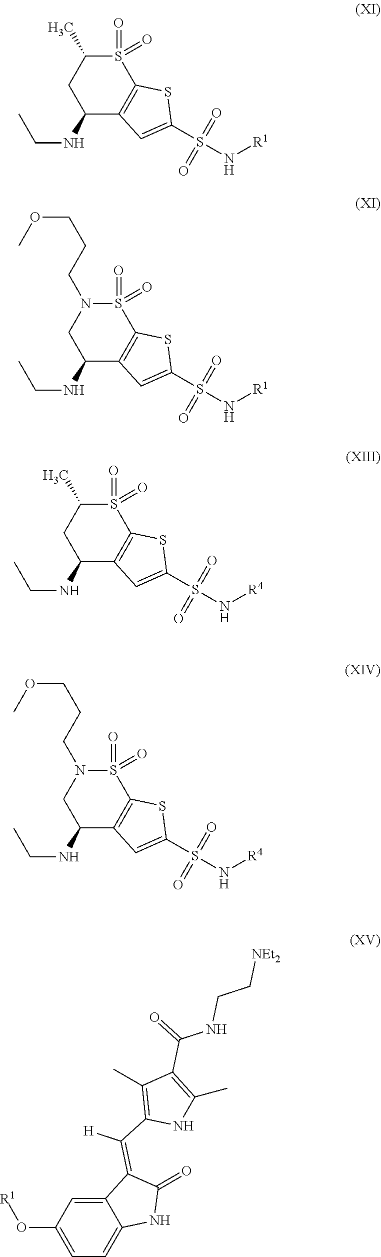

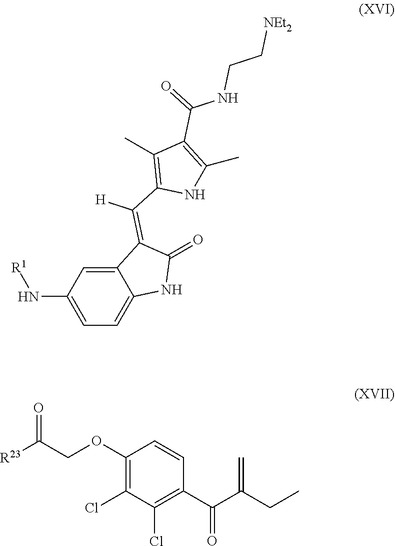

In one embodiment, the invention is a method for delivering an active prodrug Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII to the eye that includes presenting it as discussed herein in a controlled delivery, for example a microparticle or a nanoparticle, that allows for sustained delivery. In one embodiment, the sustained release of the active agent lowers intraocular pressure (IOP). In one embodiment, ethacrynic acid is linked to a hydrophobic polymer that allows for the release of ethacrynic acid for ocular delivery. As discussed in Example 9, ethacrynic acid linked to PLA allowed for release of ethacrynic acid in vitro. Ethacrynic acid linked to two PLA units (2) degraded faster to parent ethacrynic acid compared to ethacrynic acid linked to four PLA units (1) (FIG. 1 and FIG. 2, Example 9), but ethacrynic acid linked to six PLA units (25) degraded slightly slower than ethacrynic acid linked to four PLA units (1) (FIG. 1 and FIG. 3). This highlights the advantage of a PLA chain length of four or six over a chain length of two. Additionally, ECA-PLA(n=6) (25) microparticles have a more linear release profile than ECA-PLA(n=4) (1) microparticles (FIG. 11). ECA-PLA(n=6) (25) maintains linearity within the 3-6 month ideal release window for the duration of release, an important factor to ensure consistent daily exposure of the drug at the site of action.

In embodiments of the invention, at least one of the active therapeutic agents delivered in modified form is selected from the loop diuretic ethacrynic acid, a tyrosine kinase inhibitor, a carbonic anhydrase inhibitor, and a beta blocker. Non-limiting examples of active therapeutic agents include ethacrynic acid, Sunitinib or a derivatized version of Sunitinib (for example, with a hydroxyl, amino, thio, carboxy, keto or other functional group instead of fluoro that can be used to covalently connect the hydrophobic moiety), Brinzolamide, Dorzolamide, Timolol, Levobunolol, Carteolol, Metipranolol, and Betaxolol.

The compounds of the invention can be used for the controlled administration of active compounds to the eye, over a period of at least two, three, four, five or six months or more in a manner that maintains at least a concentration in the eye that is effective for the disorder to be treated.

In some embodiments, the prodrug is provided in a microparticle, microcapsule, vesicle, reservoir, or nanoparticle. In one embodiment, the drug is administered in a polymeric formulation that provides a controlled release that is linear. In another embodiment, the release is not linear; however, even the lowest concentration of release over the designated time period is at or above a therapeutically effective dose. In one embodiment, this is achieved by formulating a hydrophobic prodrug of the invention in a polymeric delivery material such as a polymer or copolymer that includes at least moieties of lactic acid, glycolic acid, propylene oxide or ethylene oxide. In a particular embodiment, the polymeric delivery system includes PLGA, PLA or PGA with or without covalently attached or admixed polyethylene glycol. For example, the hydrophobic drug may be delivered in a mixture of PLGA and PLGA-PEG, PEG, PLA, or PLA-PEG. The hydrophobic drug may be delivered in a mixture of PLA and PLGA-PEG, PEG, PLGA, or PLA-PEG. In another embodiment, the polymer includes a polyethylene oxide (PEO) or polypropylene oxide (PPO).

Another disclosed invention is a method for the controlled administration of Timolol to a patient in need thereof, comprising administering a prodrug of Timolol in a microparticle in vivo, wherein the Timolol prodrug containing microparticle exhibits in vitro drug release kinetics in an aqueous solution at a pH between 6-8 at body temperature of a substantially consistent release of at least 60% Timolol itself by molar ratio to the prodrug of Timolol or an intermediate metabolite thereof (i.e., a breakdown product of the prodrug of Timolol on the way to the parent Timolol) over at least 100 days. In certain embodiments, the aqueous solution is a buffered solution, for example, a phosphate buffered solution. In other embodiments, there can be a substantially consistent release of at least 70%, 75%, 80%, 85% or 90% or more of the parent Timolol itself by molar ratio to the prodrug of Timolol or an intermediate metabolite thereof over at least 100, 110 or even 120 or more days. The term “total drug” as used herein refers to the Timolol prodrug and intermediate metabolites together which ultimately break down to the parent Timolol. This can occur when the prodrug of Timolol has multiple labile bonds that can be metabolically or hydrolytically cleaved, such as ester and/or amide bonds. Examples of Timolol prodrugs are those, for example, with glycolic acid and/or lactic acid moieties. In some embodiments, the prodrug of Timolol is a Timolol-N-glycolic acid-containing prodrug, a Timolol-O-glycolic acid-containing prodrug, Timolol-N,O-bis-glycolic acid-containing prodrug, Timolol-N,O-bis-glycolic acid-O-acetyl, Timolol-N,O-bis-glycolic acid-O-(PLA)₄-acetyl, or for example wherein the prodrug is an ester-containing prodrug or an amide-containing prodrug.

It has been surprisingly discovered that selected Timolol prodrug microparticles as described herein exhibit substantially linear release rates over at least 2, 3 or 4 months in vitro where the correlation between parent drug release and total drug (i.e., Timolol prodrug and intermediate metabolic breakdown products of the prodrug on the way to the parent Timolol) release is high. In other words, the microparticle with Timolol prodrug is capable of consistently delivering a high molar percentage of the active compound, Timolol, which is advantageous for therapy.

In a non-limiting embodiment, as discussed in Example 14 and shown in FIG. 22, Compound 50 is an example of a Timolol prodrug with such properties. This is unexpected because other Timolol prodrugs (for example shown in Example 14) with similar chemical structures do not exhibit substantially linear kinetics in vitro with at least 2, 3 or 4 month release in combination with a high degree of consistent parent Timolol release. For example, Compound 51, which only differs from Compound 50 in that Compound 51 has two PLA unit on the polymeric branches, while Compound 50 has four, does not exhibit substantially linear 4-month release in vitro. Compound 51 also does not exhibit kinetics where the correlation between total drug release and parent Timolol is high (FIG. 24).

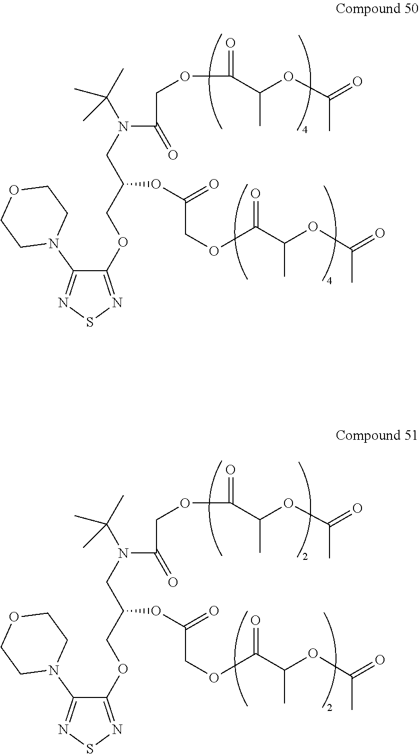

In certain embodiments, the drug or prodrug is delivered in a microparticle or nanoparticle that is a blend of two polymers, for example (i) a PLGA polymer or PLA polymer as described herein and (ii) a PLGA-PEG or PLA-PEG copolymer. In another embodiment, the microparticle or nanoparticle is a blend of three polymers, such as, for example, (i) a PLGA polymer; (ii) a PLA polymer; and, (iii) a copolymer of PLGA-PEG or PLA-PEG. In an additional embodiment, the microparticle or nanoparticle is a blend of (i) a PLA polymer; (ii) a PLGA polymer; (iii) a PLGA polymer that has a different ratio of lactide and glycolide monomers than the PLGA in (ii); and, (iv) a PLGA-PEG or PLA-PEG copolymer. Any ratio of lactide and glycolide in the PLGA can be used that achieves the desired therapeutic effect. In certain illustrative non-limiting embodiments, the ratio of PLA to PLGA by weight in a polymer blend as described is 77/22, 69/30, 49/50, 54/45, 59/40, 64/35, 69/30, 74/25, 79/20, 84/15, 89/10, 94/5, or 99/1. In certain embodiments, the prodrug is Compound 50.

In certain embodiments, a blend of three polymers that has (i) PLA, (ii) PLGA, and (iii) PLGA with a different ratio of lactide and glycolide monomers than PLGA in (ii) wherein the ratio by weight is 74/20/5 by weight, 69/20/10 by weight, 69/25/5 by weight, or 64/20/15 by weight. In certain embodiments, the PLGA in (ii) has a ratio of lactide to glycolide of 85/15, 75/25, or 50/50. In certain embodiments the PLGA in (iii) has a ratio of lactide to glycolide of 85/15, 75/25, or 50/50.

In certain aspects, the drug or prodrug may be delivered in a blend of PLGA or PLA and PEG-PLGA, including but not limited to (i) PLGA+approximately by weight 1% PEG-PLGA or (ii) PLA+approximately by weight 1% PEG-PLGA. In certain aspects, the drug may be delivered in a blend of (iii) PLGA/PLA+approximately by weight 1% PEG-PLGA. In certain embodiments, the blend of PLA, PLGA, or PLA/PGA with PLGA-PEG contains approximately from about 0.5% to about 10% by weight of a PEG-PLGA, from about 0.5% to about 5% by weight of a PEG-PLGA, from about 0.5% to about 4% by weight of a PEG-PLGA, from about 0.5% to about 3% by weight of a PEG-PLGA, from about 1.0% to about 3.0% by weight of a PEG-PLGA, from about 0.1% to about 10% of a PEG-PLGA, from about 0.1% to about 5% of a PEG-PLGA, from about 0.1% to about 1% PEG-PLGA, or from about 0.1% to about 2% PEG-PLGA.

In certain non-limiting embodiments, the ratio by weight percent of PLGA to PEG-PLGA in a two polymer blend as described is about 40/1, 45/1, 50/1, 55/1, 60/1, 65/1, 70/1, 75/1, 80/1, 85/1, 90/1, 95/1, 96/1, 97/1, 98/1, 99/1. The PLGA can be acid or ester capped. In non-limiting aspects, the drug can be delivered in a two polymer blend of PLGA75:25 4A+approximately 1% PEG-PLGA50:50; PLGA85:15 5A+approximately 1% PEG-PLGA5050; PLGA75:25 6E+approximately 1% PEG-PLGA50:50; or, PLGA50:50 2A+approximately 1% PEG-PLGA50:50.

In certain non-limiting embodiments, the ratio by weight percent of PLA/PLGA-PEG in a polymer blend as described is about 40/1, 45/1, 50/1, 55/1, 60/1, 65/1, 70/1, 75/1, 80/1, 85/1, 90/1, 95/1, 96/1, 97/1, 98/1, 99/1. The PLA can be acid capped or ester capped. In certain aspects, the PLA is PLA 4.5A. In non-limiting aspects, the drug is delivered in a blend of PLA 4.5A+1% PEG-PLGA.

The PEG segment of the PEG-PLGA may have, for example, in non-limiting embodiments, a molecular weight of at least about 1 kDa, 2 kDa, 3 kDa, 4 kDa, 5 kDa, 6 kDa, 7 kDa, 8 kDa, 9 kDa, or 10 kDa, and typically not greater than 10 kDa, 15 kDa, 20 kDa, or 50 kDa, or in some embodiments, 6 kDa, 7 kDa, 8 kDa, or 9 kDa. In certain embodiment, the PEG segment of the PEG-PLGA has a molecular weight between about 3 kDa and about 7 kDa or between about 2 kDa and about 7 kDa. Non-limiting examples of the PLGA segment of the PEG-PLGA is PLGA50:50, PLGA75:25, or PLGA85:15. In one embodiment, the PEG-PLGA segment is PEG (5 kDa)-PLGA50:50.

When the drug or prodrug is delivered in a blend of PLGA+PEG-PLGA, any ratio of lactide and glycolide in the PLGA or the PLGA-PEG can be used that achieves the desired therapeutic effect. Non-limiting illustrative embodiments of the ratio of lactide/glycolide in the PLGA or PLGA-PEG are about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In one embodiment, the PLGA is a block co-polymer, for example, diblock, triblock, multiblock, or star-shaped block. In one embodiment, the PLGA is a random co-polymer. In certain aspects, the PLGA is PLGA75:25 4A; PLGA85:15 5A; PLGA75:25 6E; or, PLGA50:50 2A.

The decreased rate of release of the active material to the ocular compartment may result in decreased inflammation, which has been a significant side effect of ocular therapy to date.

It is also important that the decreased rate of release of the drug or prodrug while maintaining efficacy over an extended time of up to 2, 3, 4, 5 or 6 months be achieved using a particle that is small enough for administration through a needle without causing significant damage or discomfort to the eye and not to give the illusion to the patient of black spots floating in the eye. This typically means the controlled release particle should be less than approximately 300, 250, 200, 150, 100, 50, 45, 40, 35, or 30 μm, such as less than approximately 30, 29, 28, 27, 26, 25, 24, 23, 22 21, or 20 μm. In one aspect, the particles do not agglomerate in vivo to form larger particles, but instead in general maintain their administered size and decrease in size over time.

The hydrophobicity of the conjugated drug or prodrug can be measured using a partition coefficient (P; such as Log P in octanol/water), or distribution coefficient (D; such as Log D in octanol/water) according to methods well known to those of skill in the art. Log P is typically used for compounds that are substantially un-ionized in water and Log D is typically used to evaluate compounds that ionize in water. In certain embodiments, the conjugated derivatized drug has a Log P or Log D of greater than approximately 2.5, 3, 3.5, 4, 4.5, 5, 5.5 or 6. In other embodiments, the conjugated derivatized drug has a Log P or Log D which is at least approximately 1, 1.5, 2, 2.5, 3, 3.5 or 4 Log P or Log D units, respectively, higher than the parent hydrophilic drug.

This invention includes an active compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII or a pharmaceutically acceptable salt or composition thereof. In one embodiment, an active compound or its salt or composition, as described herein, is used to treat a medical disorder which is glaucoma, a disorder mediated by carbonic anhydrase, a disorder mediated by a Rho-associated kinase, a disorder mediated by a tyrosine kinase inhibitor, a disorder mediated by a dual leucine zipper kinase, a disorder mediated by VEGF, a disorder mediated by an α2 adrenergic receptor, a disorder or abnormality related to an increase in intraocular pressure (IOP), a disorder mediated by nitric oxide synthase (NOS), or a disorder requiring neuroprotection such as to regenerate/repair optic nerves. In another embodiment more generally, the disorder treated is allergic conjunctivitis, anterior uveitis, cataracts, dry or wet age-related macular degeneration (AMD), neovascular age-related macular degeneration, geographic atrophy, or diabetic retinopathy. In one embodiment, an active compound or its salt or composition, as described herein, is used to decrease IOP. In one embodiment, an active compound or its salt or composition is used to treat optic nerve damage associated with IOP.

Compounds of Formula I are single agent prodrugs of ethacrynic acid.

In alternative embodiments, compound of Formula I are pharmaceutically acceptable salts of hydrophobic prodrugs of ethacrynic acid.

In embodiments, compounds of Formula II and Formula II′ are pharmaceutically acceptable salts of prodrug conjugates of ethacrynic acid and Brimonidine allowing release of both compounds in the eye. In one embodiment both compounds are released concurrently.

In alternative embodiments, compounds of Formula II and Formula II′ are prodrug conjugates of a carbonic anhydrase inhibitor and ethacrynic acid allowing release of both compounds in the eye. In one embodiment both compounds are released concurrently.

In alternative embodiments, compounds of Formula II and Formula II′ are prodrug conjugates of a dual leucine zipper kinase inhibitor and ethacrynic acid allowing release of both compounds in the eye. In one embodiment both compounds are released concurrently.

In alternative embodiments, compounds of Formula II and Formula II′ are prodrug conjugates of ethacrynic acid and a Sunitinib derivative allowing release of both compounds in the eye. In one embodiment both compounds are released concurrently.

In alternative embodiments, compounds of Formula II and Formula II′ are single agent prodrug conjugates of ethacrynic acid and a prostaglandin derivative allowing release of both compounds in the eye. In one embodiment both compounds are released concurrently.

In alternative embodiments, compounds of Formula II and Formula II′ are single agent prodrug conjugates of a ROCK inhibitor and ethacrynic acid allowing release of both compounds in the eye. In one embodiment both compounds are released concurrently.

In alternative embodiments, compounds of Formula II and Formula II′ are single agent prodrug conjugates of Timolol and ethacrynic acid allowing release of both compounds in the eye.

In one embodiment both compounds are released concurrently.

Compounds of Formula III are single agent hydrophobic prodrugs of the beta-blocker Timolol.

In alternative embodiments, compound of Formula III are pharmaceutically acceptable salts of hydrophobic prodrugs of the beta-blocker Timolol.

Compounds of Formula IV are single agent hydrophobic prodrugs of the beta-blocker Carteolol.

In alternative embodiments, Compound of Formula IV′ are single agent hydrophobic prodrugs of the beta-blocker Levobunolol.

In alternative embodiments, compound of Formula IV or Formula IV′ are pharmaceutically acceptable salts of hydrophobic prodrugs of the beta-blocker Carteolol or Levobunolol, respectively.

Compounds of Formula V are single agent hydrophobic prodrugs of the beta-blocker Metipranolol.

In alternative embodiments, compound of Formula V are pharmaceutically acceptable salts of hydrophobic prodrugs of the beta-blocker Metipranolol.

Compounds of Formula VI are single agent hydrophobic prodrugs of the beta-blocker Betaxolol.

In alternative embodiments, compound of Formula VI are pharmaceutically acceptable salts of hydrophobic prodrugs of the beta-blocker Betaxolol.

In embodiments, compounds of Formula VII, Formula VIII, Formula VIII′, Formula IX, and Formula X are prodrug conjugates of a carbonic anhydrase inhibitor and a beta-blocker allowing release of both compounds in the eye. In one embodiment both compounds are released concurrently.

In alternative embodiments, compounds of Formula VII, Formula VIII, Formula VIII′, Formula IX, and Formula X are prodrug conjugates of a dual leucine zipper kinase inhibitor and a beta-blocker allowing release of both compounds in the eye. In one embodiment both compounds are released concurrently.

In alternative embodiments, compounds of Formula VII, Formula VIII, Formula VIII′, Formula IX, and Formula X are prodrug conjugates of a beta-blocker and a Sunitinib derivative allowing release of both compounds in the eye. In one embodiment both compounds are released concurrently.

In alternative embodiments, compounds of Formula VII, Formula VIII, Formula VIII′, Formula IX, and Formula X are single agent prodrug conjugates of a beta-blocker and a prostaglandin derivative allowing release of both compounds in the eye. In one embodiment both compounds are released concurrently.

In alternative embodiments, compounds of Formula VII, Formula VIII, Formula VIII′, Formula IX, and Formula X are single agent prodrug conjugates of a ROCK inhibitor and a beta-blocker allowing release of both compounds in the eye. In one embodiment both compounds are released concurrently.

In alternative embodiments, compounds of Formula VII, Formula VIII, Formula VIII′, Formula IX, and Formula X are single agent prodrug conjugates of ethacrynic acid and a beta-blocker allowing release of both compounds in the eye. In one embodiment both compounds are released concurrently.

Compounds of Formula XI are single agent hydrophobic prodrugs of the carbonic anhydrase inhibitor Dorzolamide.

In alternative embodiments, compound of Formula XI are pharmaceutically acceptable salts of hydrophobic prodrugs of the carbonic anhydrase inhibitor Dorzolamide.

Compounds of Formula XII are single agent hydrophobic prodrugs of the carbonic anhydrase inhibitor Brinzolamide.

In alternative embodiments, compound of Formula XII are pharmaceutically acceptable salts of hydrophobic prodrugs of the carbonic anhydrase inhibitor Brinzolamide.

In embodiments, compounds of Formula XIII and Formula XIV are prodrug conjugates of a carbonic anhydrase inhibitor and a beta-blocker allowing release of both compounds in the eye. In one embodiment both compounds are released concurrently.

In alternative embodiments, compounds of Formula XIII and Formula XIV are prodrug conjugates of a dual leucine zipper kinase inhibitor and a carbonic anhydrase inhibitor allowing release of both compounds in the eye. In one embodiment both compounds are released concurrently.

In alternative embodiments, compounds of Formula XIII and Formula XIV are prodrug conjugates of a carbonic anhydrase inhibitor and a Sunitinib derivative allowing release of both compounds in the eye. In one embodiment both compounds are released concurrently.

In alternative embodiments, compounds of Formula XIII and Formula XIV are single agent prodrug conjugates of a carbonic anhydrase inhibitor and a prostaglandin derivative allowing release of both compounds in the eye. In one embodiment both compounds are released concurrently.

In alternative embodiments, compounds of Formula XIII and Formula XIV are single agent prodrug conjugates of a ROCK inhibitor and a carbonic anhydrase inhibitor allowing release of both compounds in the eye. In one embodiment both compounds are released concurrently.

In alternative embodiments, compounds of Formula XIII and Formula XIV are single agent prodrug conjugates of ethacrynic acid and a carbonic anhydrase inhibitor allowing release of both compounds in the eye. In one embodiment both compounds are released concurrently.

Compounds of Formula XV and Formula XVI are single agent hydrophobic prodrugs of the tyrosine kinase inhibitor Sunitinib.

In alternative embodiments, compound of Formula XV and Formula XVI are pharmaceutically acceptable salts of hydrophobic prodrugs of the tyrosine kinase inhibitor Sunitinib.

Compounds of Formula XVII are single agent prodrugs of ethacrynic acid allowing release of two units of ethacrynic acid in the eye. In one embodiment both compounds are released concurrently.

These compounds can be used to treat an ocular disorder in a host, for example a human, in need thereof. In one embodiment, a method for the treatment of such a disorder is provided that includes the administration of an effective amount of a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIV, Formula XV, Formula XVI, or Formula XVII, or a pharmaceutically acceptable salt or composition thereof, optionally in a pharmaceutically acceptable carrier, including a polymeric carrier, as described in more detail below.

Another embodiment is provided that includes the administration of an effective amount of an active compound or a pharmaceutically acceptable salt thereof, optionally in a pharmaceutically acceptable carrier, including a polymeric carrier, to a host to treat an ocular or other disorder that can benefit from topical or local delivery. The therapy can be delivery to the anterior or posterior chamber of the eye. In specific aspects, the active compound is administered to treat a disorder of the cornea, conjunctiva, aqueous humor, iris, ciliary body, lens sclera, choroid, retinal pigment epithelium, neural retina, optic nerve or vitreous humor.

Any of the compounds described herein (Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII) can be administered to the eye in a composition as described further herein in any desired form of administration, including via intravitreal, intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, and tear duct injections, or through a mucus, mucin, or a mucosal barrier, in an immediate or controlled release fashion.

In any of the Formulas described herein (Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII) if the stereochemistry of a chiral carbon is not specifically designated in the Formula it is intended that the carbon can be used as an R enantiomer, an S enantiomer, or a mixture of enantiomers including a racemic mixture. In Formula II, Formula II′, Formula III, Formula VII, Formula XV, and Formula XVI, Timolol has the (S)-stereochemistry as used in commercial Timolol maleate ophthalmic solutions, such as Istalol® and Timoptic®. On both U.S. FDA labels, Timolol maleate is described as a single enantiomer ((−)-1-(tert-butylamino)-3-[(4-morpholino-1,2,5-thiadiazol-3-yl)oxy]-2-propanol maleate) that “possesses an asymmetric carbon atom in its structure and is provided as the levo-isomer.” The (S)-enantiomer has CAS No. 26839-75-8 and the (R)-enantiomer has CAS No. 26839-76-9, but only the (S)-enantiomer is described as “Timolol”. Likewise, compounds presented which are or are analogs of commercial products are provided in their approved stereochemistry for regulatory use, unless otherwise instructed.

In addition, prodrug moieties that have repetitive units of the same or varying monomers, for example including but not limited to an oligomer of polylactic acid, polylactide-coglycolide, or polypropylene oxide, that has a chiral carbon can be used with the chiral carbons all having the same stereochemistry, random stereochemistry (by either monomer or oligomer), racemic (by either monomer or oligomer) or ordered but different stereochemistry such as a block of S enantiomer units followed by a block of R enantiomer units in each oligomeric unit. In some embodiments lactic acid is used in its naturally occurring S enantiomeric form.

In certain embodiments, the conjugated active drug is delivered in a biodegradable microparticle or nanoparticle that has at least approximately 5, 7.5, 10, 12.5, 15, 20, 25 or 30% or more by weight conjugated active drug. In some embodiments, the biodegradable microparticle degrades or provides controlled delivery that lasts over a period of time and in any event at least approximately 2 months, 3 months, 4 months, 5 months or 6 months or more. In some embodiments, the loaded microparticles are administered via subconjunctival or subchoroidal injection.

In certain embodiments, the conjugated active drug is delivered as the pharmaceutically acceptable salt form. Salt forms of a compound will exhibit distinctive solution and solid-state properties compared to their respective free base or free acid form, and for this reason pharmaceutical salts are used in drug formulations to improve aqueous solubility, chemical stability, and physical stability issues. Lipophilic salt forms of compounds, which have enhanced solubility in lipidic vehicles relative to the free acid or free base forms of compounds, are often advantageous in terms of pharmacological properties due in part to their low melting points. Lipophilic salt forms of compounds are used to increase aqueous solubility for oral and parenteral drug delivery, enhance permeation across hydrophobic barriers, and enhance drug loading in lipid-based formulations.

In all of the polymer moieties described in this specification, where the structures are depicted as block copolymers (for example, blocks of “x” followed by blocks of “y”) it is intended that the polymer can alternately be a random or alternating copolymer (for example, “x” and “y”, are either randomly distributed or alternate). Unless stereochemistry is specifically indicated, each individual moiety of each oligomer that has a chiral center can be presented at the chiral carbon in (R) or (S) configuration or a mixture thereof, including a racemic mixture.

Various Formulas below use R groups defined in other Formulas, each of which R group is meant to have the definition as presented in the first Formula that it was presented in unless explicitly changed by context.

In most of the Formulas presented herein, the prodrugs are depicted as one or several active moieties covalently bound to or through a described prodrug moiety(ies) with a defined variable range of each of the active moiety and the prodrug moiety, typically through the use of descriptors x, y, or z. As indicated below, these descriptors can independently have numerical ranges provided below, and in most embodiments, are typically within a smaller range, also as provided below. Each variable is independent such that any of the integers of one variable can be used with any of the integers of the other variable, and each combination is considered separately and independently disclosed, and set out below like this only for space considerations.

For example, x, y, and z can independently be any integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29 or 30). In certain embodiments, x or y or z can independently be 1, 2, 3, 4, 5, 6, 7, 8, 9, 10, 11, or 12 and in certain aspects, 1, 2, 3, 4, 5, or 6. In certain embodiments, x is 1, 2, 3, 4, 5, 6, 7, or 8. In certain embodiments, y is 1, 2, 3, 4, 5, 6, 7, or 8. In certain embodiments, x is 1, 2, 3, 4, 5, or 6. In certain embodiments, y is 1, 2, 3, 4, 5, or 6. In certain embodiments, z is 1, 2, 3, 4, 5, or 6. In certain embodiments, y is 1, 2, or 3. In certain embodiments, x is 1, 2, or 3. In certain embodiments, x is 1, 2, or 3 and y is 1, 2, 3, 4, 5, or 6. In certain embodiments, x is 1, 2, or 3 and y is 1, 2, 3, or 4. In certain embodiments, x is an integer selected from 1, 2, 3, and 4 and y is 1. In certain embodiments, x is an integer selected from 1, 2, 3, and 4 and y is 2. In certain embodiments, x is in integer selected from 1, 2, 3, and 4 and y is 3.

Where x, y, or z is used in connection with a single atom, such as

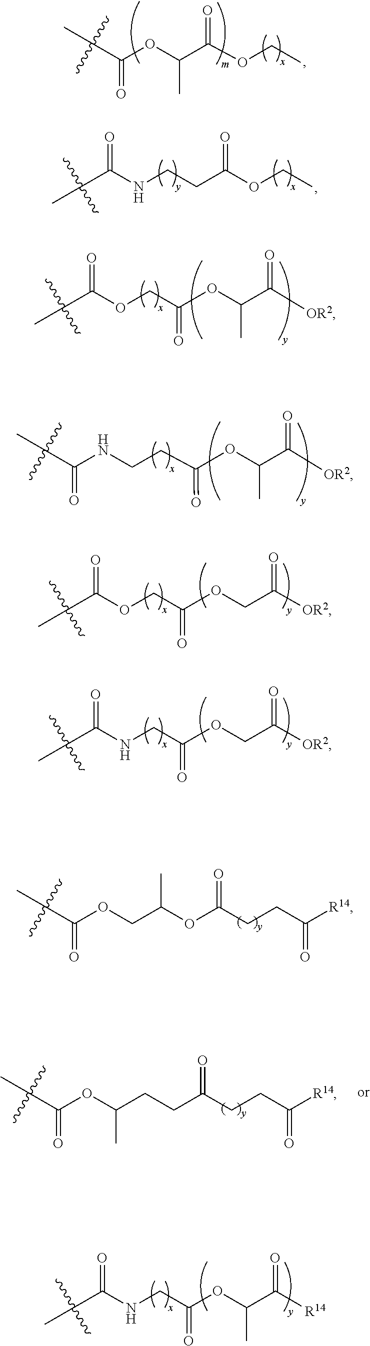

x,y, or z are typically independently 1, 2, 3, 4, 5, 6, 7, 8, 9, 10, 11 or 12, and more typically 1, 2, 3, 4, 5 or 6, and even 1, 2, 3 or 4 or 1 or 2.

Where x, y, or z is used in connection with the monomeric residue in an oligomer, including for example but not limited to:

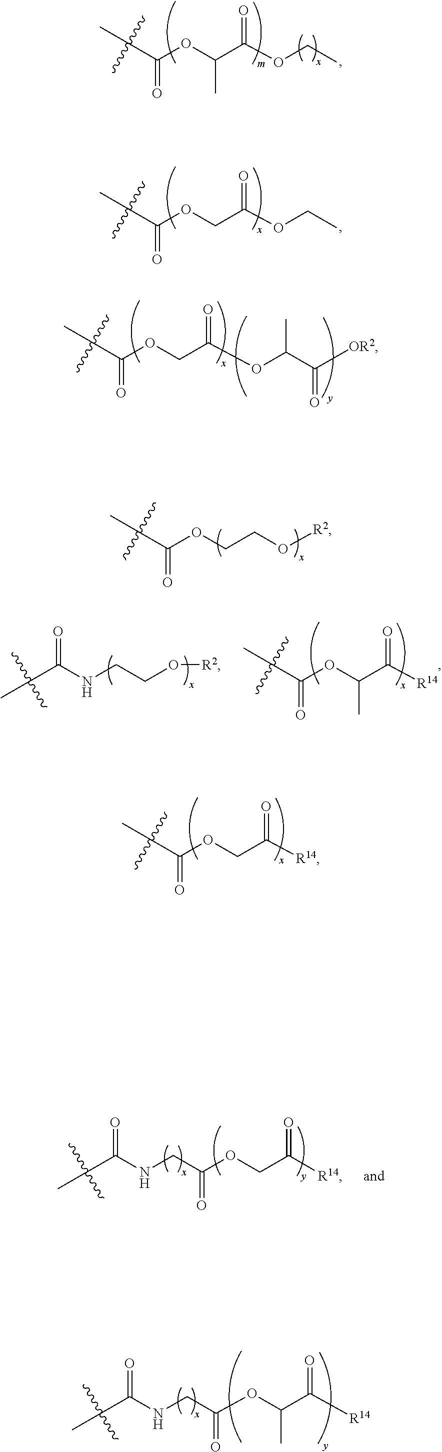

then x, y or z is in some embodiments independently 1, 2, 3, 4, 5, 6, 7 or 8, and even for example, 2, 4 or 6.

The disclosure provides a prodrug of Formula I:

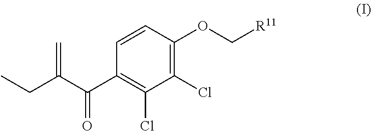

or a pharmaceutically acceptable composition, salt, or isotopic derivative thereof.

R¹¹ is selected from:
(i) —C(O)OC₅-C₃₀alkylR³, —C(O)OC₂-C₃₀alkenylR³, —C(O)OC₂-C₃₀alkynylR³, —C(O)OC₄-C₃₀alkenylalkynylR³, —C(O)OC₅-C₃₀alkyl, —C(O)OC₂-C₃₀alkenyl, —C(O)OC₂-C₃₀alkynyl, and —C(O)OC₄-C₃₀alkenylalkynyl;
(ii) —C(O)O(C₁₋₃₀alkyl with at least one R³ substituent on the alkyl chain), —C(O)O(C₁₋₃₀alkenyl with at least one R³ substituent on the alkenyl chain), and —C(O)O(C₁₋₃₀alkynyl with at least one R³ substituent on the alkynyl chain);
(iii) —C(O)(OCH₂C(O))₁₋₂₀OC₁₋₃₀alkyl, —C(O)(OCH(CH₃)C(O))₁₋₂₀OC₁₋₃₀alkyl, —C(O)(OCH₂C(O))₁₋₁₀OC₁₋₃₀alkyl, —C(O)(OCH(CH₃)C(O))₁₋₁₀OC₁₋₃₀alkyl, —C(O)(OCH₂C(O))₄₋₂₀OC₁₋₃₀alkyl, —C(O)(OCH(CH₃)C(O))₁₋₂₀OC₁₋₃₀alkyl, —C(O)(OCH₂C(O))₁₋₂₀OC₁₋₁₀alkyl, —C(O)(OCH(CH₃)C(O))₁₋₂₀OC₁₋₁₀alkyl, —C(O)(OCH₂C(O))₁₋₂₀OC₄₋₁₀alkyl, —C(O)(OCH(CH₃)C(O))₁₋₂₀OC₄₋₁₀alkyl, —C(O)(OCH₂C(O))₁₋₂₀OH, —C(O)(OCH(CH₃)C(O))₁₋₂₀OH, —C(O)(OCH₂C(O))₁₋₁₀OH, —C(O)(OCH(CH₃)C(O))₁₋₁₀OH, —C(O)(OCH₂C(O))₄₋₂₀OH, —C(O)(OCH(CH₃)C(O))₄₋₂₀OH, —C(O)(OCH₂C(O))₄₋₁₀OH, —C(O)(OCH(CH₃)C(O))₄₋₁₀OH, —C(O)(OCH(CH₃)C(O))₄₋₁₀OC₁₋₁₀alkyl, —C(O)(OCH₂C(O))₄₋₁₀OC₁₋₁₀alkyl, —C(O)(OCH(CH₃)C(O))₁₋₁₀OC₁₋₁₀alkyl, —C(O)(OCH₂C(O))₁₋₁₀OC₁₋₁₀alkyl, —C(O)(OCH(CH₃)C(O))₁₋₁₀OC₄₋₁₀alkyl, —C(O)(OCH₂C(O))₁₋₁₀OC₄₋₁₀alkyl, —C(O)(OCH₂C(O))₁₋₁₀OC₄₋₁₀alkyl, —C(O)(OCH(CH₃)C(O))₁₋₁₀OC₄₋₁₀alkyl, —C(O)(OCH₂C(O))₁₋₁₀OC₄₋₁₀alkyl, —C(O)(OCH(CH₃)C(O))₁₋₁₀OC₄₋₁₀alkyl, —C(O)(OCH₂C(O))₁₋₁₀(OCH(CH₃)C(O))₁₋₁₀OC₁₋₃₀alkyl, —C(O)(OCH₂C(O))₂₋₁₀(OCH(CH₃)C(O))₂₋₁₀OC₁₋₃₀alkyl, —C(O)(OCH₂C(O))₁₋₁₀(OCH(CH₃)C(O))₁₋₁₀OC₁₋₁₂alkyl, —C(O)(OCH₂C(O))₁₋₁₀(OCH(CH₃)C(O))₁₋₁₀OC₄₋₂₂alkyl, —C(O)(OCH(CH₃)C(O))₁₋₁₀(OCH₂C(O))₁₋₁₀OC₁₋₃₀alkyl, —C(O)(OCH(CH₃)C(O))₂₋₁₀(OCH₂C(O))₂₋₁₀OC₁₋₃₀alkyl, —C(O)(OCH(CH₃)C(O))₁₋₁₀(OCH₂C(O))₁₋₁₀OC₁₋₁₂alkyl, and —C(O)(OCH(CH₃)C(O))₁₋₁₀(OCH₂C(O))₁₋₁₀OC₄₋₂₂alkyl;
(iv) polylactic acid, poly(lactic-co-glycolic acid), polyglycolic acid, polyester, polyamide, and other biodegradable polymers, each of which can be capped to complete the terminal valence or to create a terminal ether or ester; and

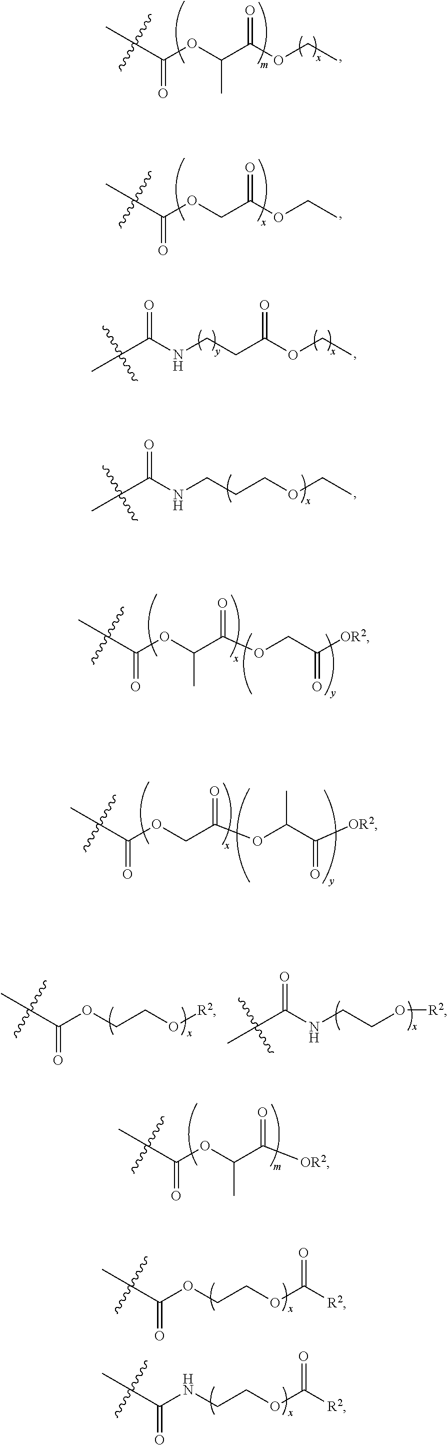

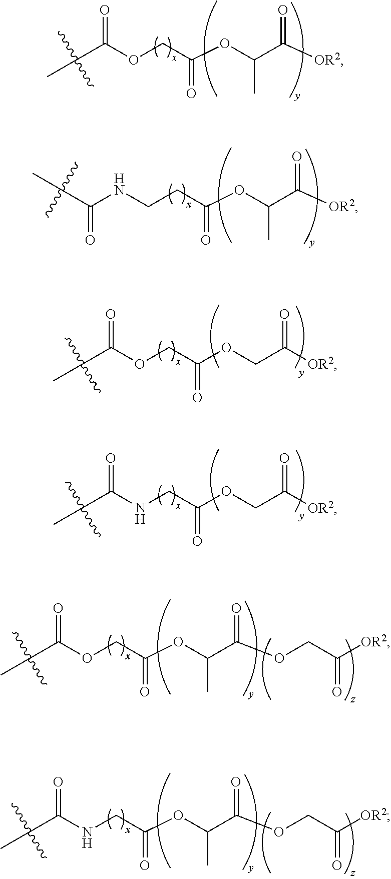

or R¹¹ is selected from

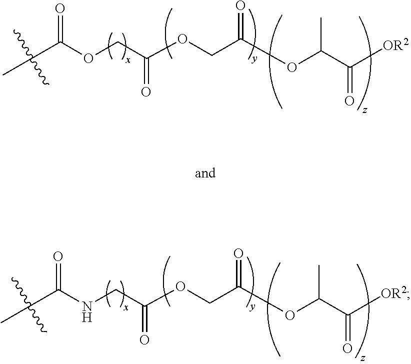

or in an alternative embodiment, R¹¹ is

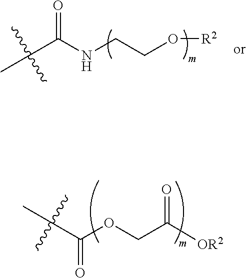

R² is hydrogen, alkyl, alkenyl, alkynyl cycloalkyl, cycloalkylalkyl, heterocyclyl, heterocycloalkyl, aryl, arylalkyl, heteroaryl, or heteroarylalkyl, each of which except hydrogen, may be optionally substituted, for example with halogen, alkyl, aryl, heterocycle or heteroaryl if desired and if the resulting compound is stable and achieves the desired purpose, wherein the group cannot be substituted with itself, for example alkyl would not be substituted with alkyl;

R³ is selected from halogen, hydroxyl, cyano, mercapto, amino, alkoxy, cycloalkyl, cycloalkylalkyl, heterocyclyl, heterocycloalkyl, aryl, arylalkyl, heteroaryl, heteroarylalkyl, aryloxy, —S(O)₂alkyl, —S(O)alkyl, —P(O)(Oalkyl)₂, B(OH)₂, —Si(CH₃)₃, —COOH, —COOalkyl, and —CONH₂, each of which except halogen, cyano, and —Si(CH₃)₃ may be optionally substituted, for example with halogen, alkyl, aryl, heterocycle or heteroaryl if desired and if the resulting compound is stable and achieves the desired purpose, wherein the group cannot be substituted with itself, for example alkyl would not be substituted with alkyl;

m is any integer between 4 and 10 (4, 5, 6, 7, 8, 9, or 10); and

x, y, and z can independently be any integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29 or 30).

In one embodiment, x, y, and z are independently an integer between 1 and 12 (1, 2, 3, 4, 5, 6, 7, 8, 9, 10, 11, or 12).

In one embodiment, x, y, and z are independently an integer between 1 and 10 (1, 2, 3, 4, 5, 6, 7, 8, 9, or 10).

In one embodiment, x, y, and z are independently an integer between 1 and 8 (1, 2, 3, 4, 5, 6, 7, or 8).

In one embodiment, x, y, and z are independently an integer between 1 and 6 (1, 2, 3, 4, 5, or 6).

In one embodiment, x, y, and z are independently an integer between 4 and 10 (4, 5, 6, 7, 8, 9, or 10).

In one embodiment, x is an integer between 1 and 12 (1, 2, 3, 4, 5, 6, 7, 8, 9, 10, 11, or 12) and y is an integer between 1 and 6 (1, 2, 3, 4, 5, or 6).

In one embodiment, y is an integer between 1 and 12 (1, 2, 3, 4, 5, 6, 7, 8, 9, 10, 11, or 12) and x is an integer between 1 and 6 (1, 2, 3, 4, 5, or 6).

In one embodiment, x is an integer between 1 and 6 (1, 2, 3, 4, 5, or 6) and y is an integer between 1 and 3 (1, 2, or 3).

In one embodiment, y is an integer between 1 and 6 (1, 2, 3, 4, 5, or 6) and x is an integer between 1 and 3 (1, 2, or 3).

In an alternative embodiment, x is 2.

In an alternative embodiment, x is 3.

In an alternative embodiment, x is 4.

In an alternative embodiment x is 1 and y is 1.

In an alternative embodiment x is 1 and y is 2.

In an alternative embodiment x is 1 and y is 3.

In an alternative embodiment x is 1 and y is 4.

In an alternative embodiment x is 1 and y is 5.

In an alternative embodiment x is 1 and y is 6.

In an alternative embodiment x is 1 and y is 7.

In an alternative embodiment x is 1 and y is 8.

In an alternative embodiment x is 2 and y is 1.

In an alternative embodiment x is 2 and y is 2.

In an alternative embodiment x is 2 and y is 3.

In an alternative embodiment x is 2 and y is 4.

In an alternative embodiment x is 2 and y is 5.

In an alternative embodiment x is 2 and y is 6.

In an alternative embodiment x is 2 and y is 7.

In an alternative embodiment x is 2 and y is 8.

In certain embodiments, x and y are independently selected from 1, 2, 3, 4, 5, or 6, and z is 1.

In certain embodiments, x and y are independently selected from 1, 2, 3, 4, 5, or 6, and z is 2.

In one embodiment, R¹¹ is

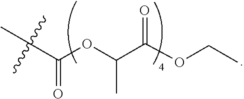

In one embodiment, R¹¹ is

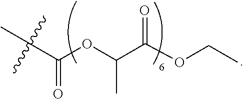

In one embodiment, R¹¹ is

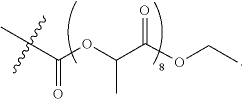

In one embodiment, R¹¹ is —C(O)(OCH(CH₃)C(O))₄₋₂₀OCHCH₃.

In one embodiment, R¹¹ is —C(O)(OCH(CH₃)C(O))₄₋₂₀O(CH₂)₁₀CH₃.

In one embodiment, R¹¹ is —C(O)(OCH(CH₃)C(O))₄₋₂₀O(CH₂)₁₆CH₃.

In one embodiment, R¹¹ is —C(O)(OCH(CH₃)C(O))₄OCH₂CH₃.

In one embodiment, R¹¹ is —C(O)(OCH(CH₃)C(O))₄O(CH₂)₁₀CH₃.

In one embodiment, R¹¹ is —C(O)(OCH(CH₃)C(O))₄OCH₂)₁₆CH₃.

In one embodiment, R¹¹ is —C(O)(OCH(CH₃)C(O))₆COCH₂CH₃.

In one embodiment, R¹¹ is —C(O)(OCH(CH₃)C(O))₆O(CH₂)₁₀CH₃.

In one embodiment, R¹¹ is —C(O)(OCH(CH₃)C(O))₆O(CH₂)₁₆CH₃.

In one embodiment, R¹¹ is —C(O)(OCH(CH₃)C(O))OOCH₂CH₃.

In one embodiment, R¹¹ is —C(O)(OCH(CH₃)C(O))₈O(CH₂)₁₀CH₃.

In one embodiment, R¹¹ is —C(O)(OCH(CH₃)C(O))₈O(CH₂)₁₆CH₃.

In an alternative embodiment, R¹¹ is —C(O)(OCH(CH₃)C(O))₄₋₂₀O(CH₂)₉₋₁₇CH₃.

In an alternative embodiment, R¹¹ is —C(O)(OCH(CH₃)C(O))₄₋₂₀O(CH₂)₁₁₋₁₇CH₃.

In an alternative embodiment, R¹¹ is —C(O)(OCH(CH₃)C(O))₄₋₂₀O(CH₂)₁₃₋₁₇CH₃.

In an alternative embodiment, R¹¹ is —C(O)(OCH(CH₃)C(O))₄₋₂₀O(CH₂)₁₅₋₁₇CH₃.

In an alternative embodiment, R¹¹ is —C(O)(OCH(CH₃)C(O))₄₋₂₀O(CH₂)ₙCH₃.

In an alternative embodiment, R¹¹ is —C(O)(OCH(CH₃)C(O))₄₋₂₀O(CH₂)₁₇CH₃.

In an alternative embodiment, R¹¹ is —C(O)(OCH₂C(O))₁₋₂(OCH(CH₃)C(O))₄₋₂₀OCH₂CH₃.

In an alternative embodiment, R¹¹ is

—C(O)(OCH₂C(O))₁₋₂(OCH(CH₃)C(O))₄₋₂₀O(CH₂)₁₁CH₃.

In an alternative embodiment, R¹¹ is

—C(O)(OCH₂C(O))₁₋₂(OCH(CH₃)C(O))₄₋₂₀O(CH₂)₁₇CH₃.

In an alternative embodiment, R¹¹ is

—C(O)(OCH₂C(O))₁₋₂(OCH(CH₃)C(O))₄₋₂₀O(CH₂)₉₋₁₇CH₃.

In an alternative embodiment, R¹¹ is

—C(O)(OCH₂C(O))₁₋₂(OCH(CH₃)C(O))₄₋₂₀O(CH₂)₁₁₋₁₇CH₃.

In an alternative embodiment, R¹¹ is

—C(O)(OCH₂C(O))₁₋₂(OCH(CH₃)C(O))₄₋₂₀O(CH₂)₁₃₋₁₇CH₃.

In an alternative embodiment, R¹¹ is

—C(O)(O)(OCH₂C(O))₁₋₂(OCH(CH₃)C(O))₄₋₂₀O(CH₂)₈₋₁₇CH₃.

In one embodiment, C₁₋₃₀alkyl as used in the definition of R¹¹ is C₁₋₂₈, C₁₋₂₆, C₁₋₂₄, C₁₋₂₂, C₁₋₂₀, C₁₋₁₈, C₁₋₁₆, C₁₋₁₄, C₁₋₁₂, C₁₋₁₀, C₁₋₈, C₁₋₆, or C₁₋₄.

In an alternative embodiment, C₁₋₃₀alkyl as used in the definition of R¹¹ is C₁₀₋₃₀, C₁₂₋₃₀, C₁₄₋₃₀, C₁₆₋₃₀, C₁₈₋₃₀, C₂₀₋₃₀, or C₂₅₋₃₀.

In an alternative embodiment, C₅₋₃₀alkyl as used in the definition of R¹¹ is C₁₀₋₃₀, C₁₂₋₃₀, C₁₄₋₃₀, C₁₆₋₃₀, C₁₈₋₃₀, C₂₀₋₃₀, or C₂₅₋₃₀.

In an alternative embodiment, R¹¹ is selected from —C(O)OC₁₀-C₃₀alkylR³, —C(O)OC₁₀-C₃₀alkyl, and —C(O)O(C₁₀₋₃₀alkyl with at least one R³ substituent on the alkyl chain).

The disclosure also provides a prodrug of Formula II or Formula II′:

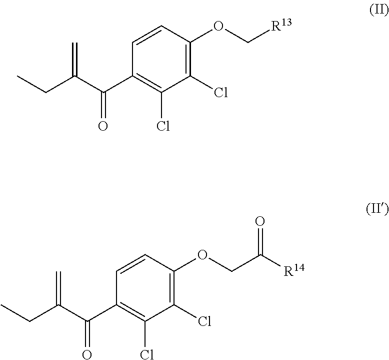

or a pharmaceutically acceptable composition, salt, or isotopic derivative thereof.

R¹³ is selected from:

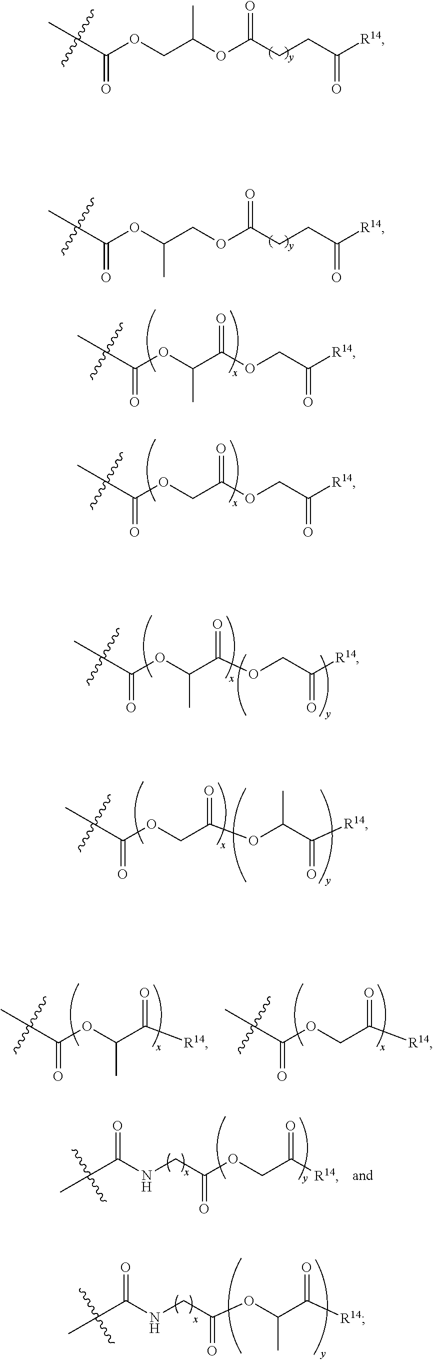

or

R¹³ is

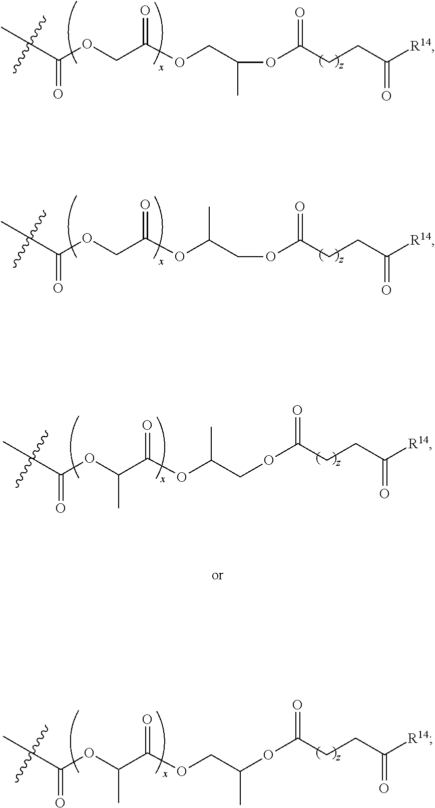

R¹⁴ is selected from

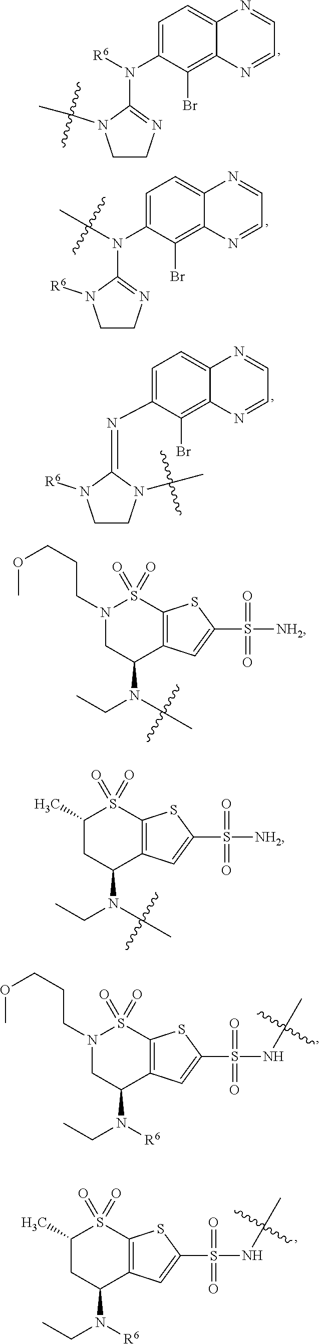

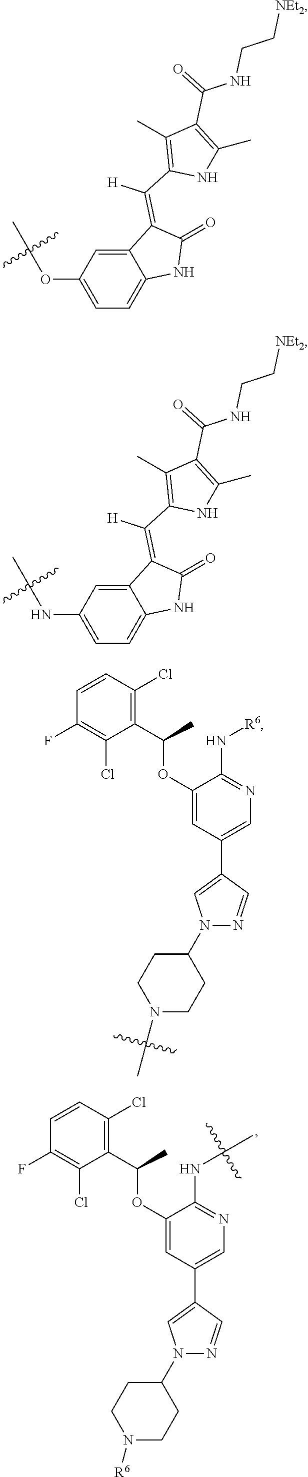

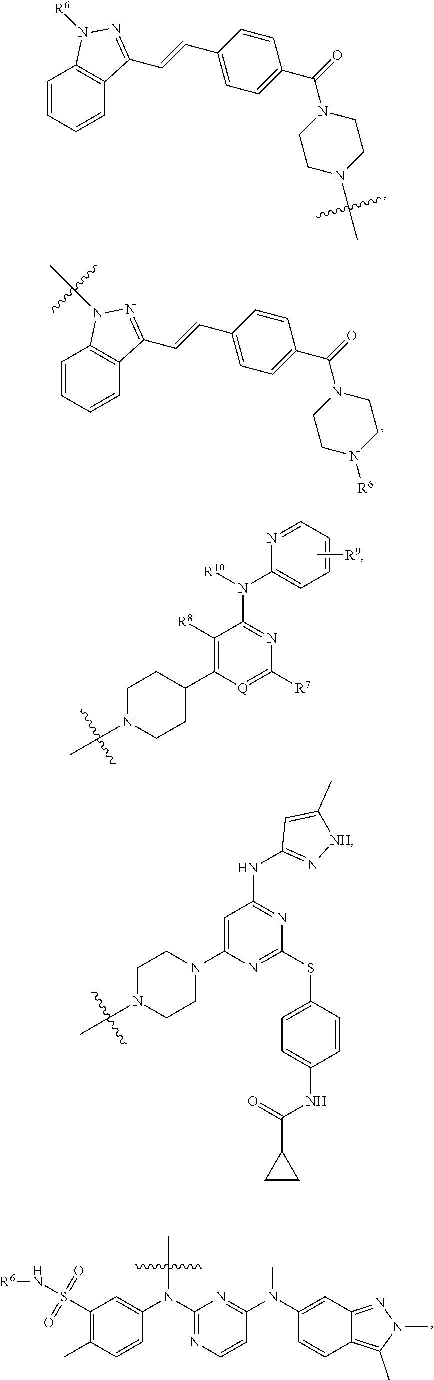

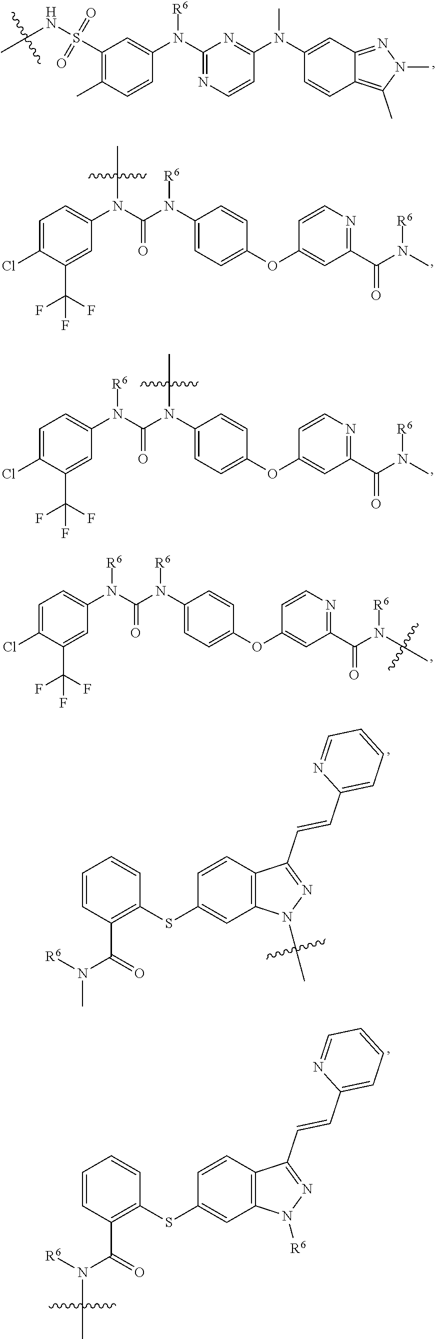

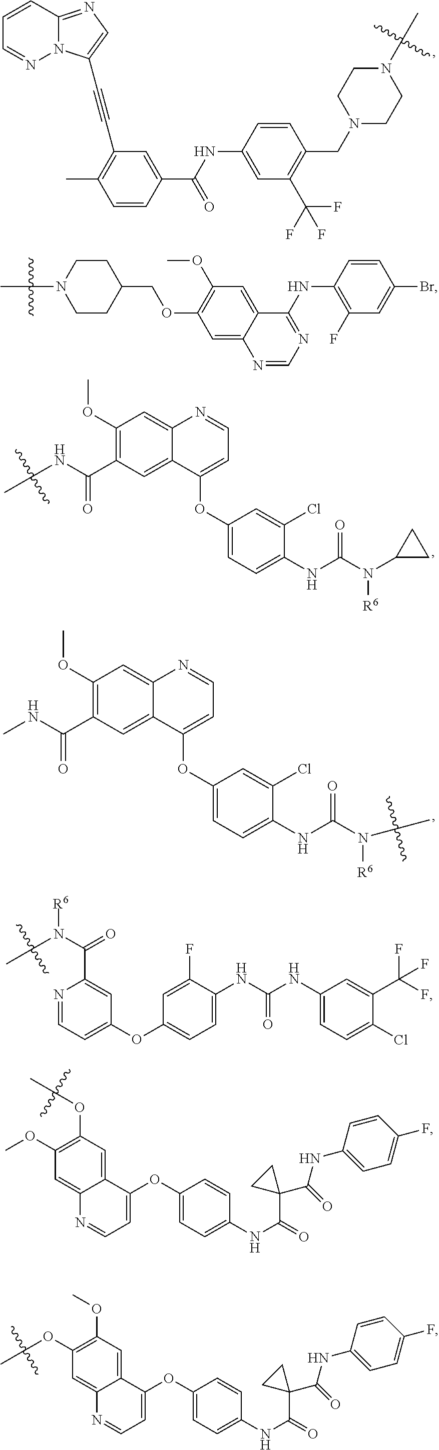

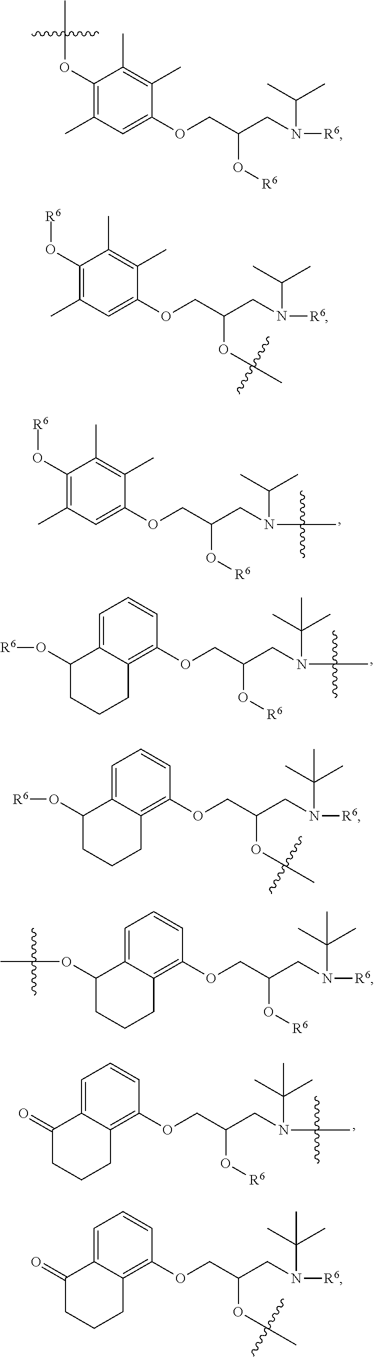

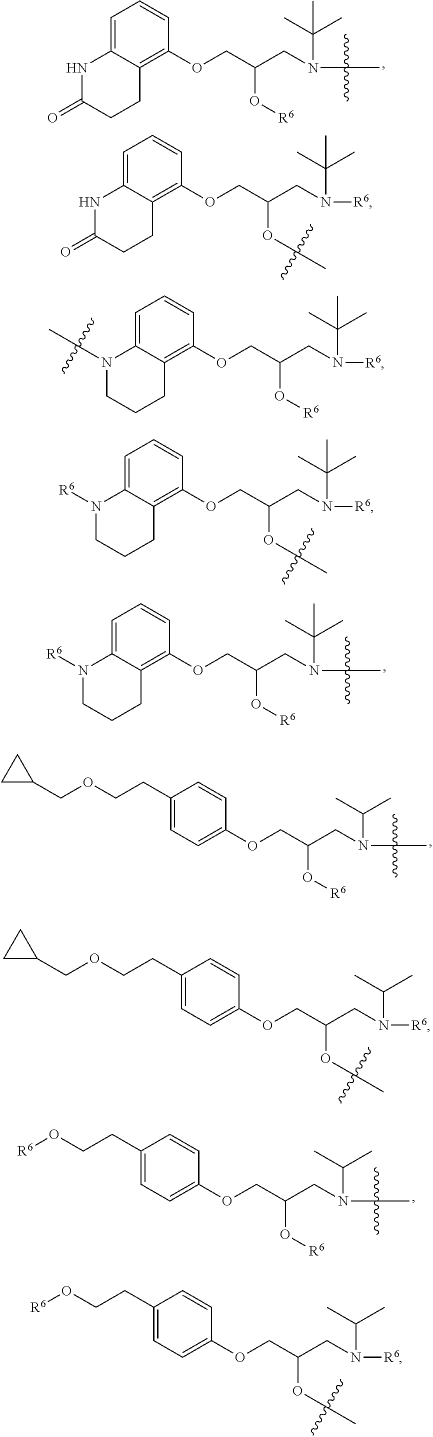

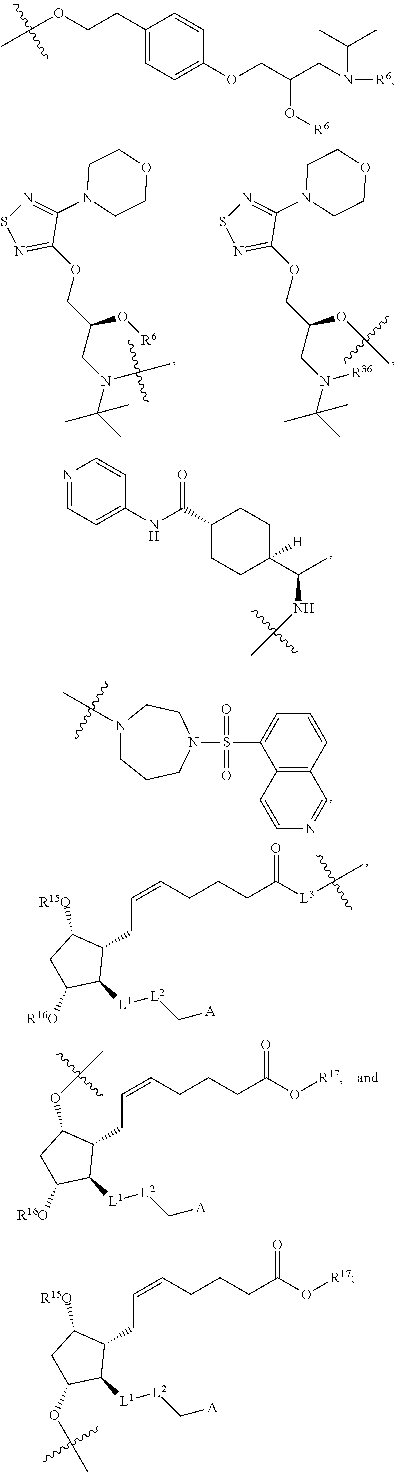

L³ is selected from: bond, —OC₁-C₃₀alkyl-O—, —NHC₁-C₃₀alkyl-O—, N(alkyl)C₁-C₃₀alkyl-O—, —NHC₁-C₃₀alkyl-NH—, N(alkyl)C₁-C₃₀alkyl-NH—, —NHC₁-C₃₀alkyl-N(alkyl)-, —N(alkyl)C₁-C₃₀alkyl-N-(alkyl)-, —OC₁-C₃₀alkenyl-O—, —NHC₁-C₃₀alkenyl-O—, N(alkyl)C₁-C₃₀alkenyl-O—, —NHC₁-C₃₀alkenyl-NH—, N(alkyl)C₁-C₃₀alkenyl-NH—, —NHC₁-C₃₀alkenyl-N(alkyl)-, —N(alkyl)C₁-C₃₀alkenyl-N-(alkyl)-, —OC₁-C₃₀alkynyl-O—, —NHC₁-C₃₀alkynyl-O—, N(alkyl)C₁-C₃₀alkynyl-O—, —NHC₁-C₃₀alkynyl-NH—, N(alkyl)C₁-C₃₀alkynyl-NH—, —NHC₁-C₃₀alkynyl-N(alkyl)-, and —N(alkyl)C₁-C₃₀alkynyl-N-(alkyl)-;

R⁶ is independently selected at each occurrence from C(O)A and hydrogen or in an alternative embodiment, R⁶ is R³⁶;

R⁷, R⁸, and R⁹ are independently selected from: hydrogen, halogen, hydroxyl, cyano, mercapto, nitro, amino, aryl, alkyl, alkoxy, alkenyl, alkynyl cycloalkyl, cycloalkylalkyl, heterocyclyl, heterocycloalkyl, aryl, arylalkyl, heteroaryl, heteroarylalkyl, aryloxy, —S(O)₂alkyl, —S(O)alkyl, —P(O)(Oalkyl)₂, B(OH)₂, —Si(CH₃)₃, —COOH, —COOalkyl, —CONH₂,

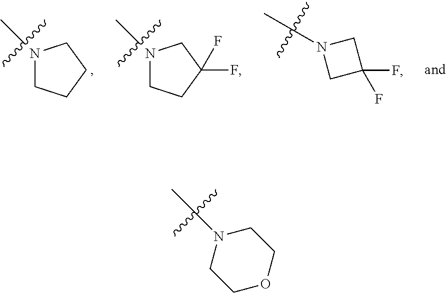

each of which except halogen, nitro, and cyano, may be optionally substituted, for example with halogen, alkyl, aryl, heterocycle or heteroaryl;

R¹⁰ is selected from H, C(O)A, —C₀-C₁₀alkylR³, —C₂-C₁₀alkenylR³, —C₂-C₁₀alkynylR³, —C₂-C₁₀alkenyl, and —C₂-C₁₀alkynyl;

R¹⁵ and R¹⁶ are independently selected from: —C(O)R¹⁸, C(O)A, and hydrogen, each of which except hydrogen can be optionally substituted with R³;

R¹⁷ is selected from:
(i) polyethylene glycol, polypropylene glycol, polypropylene oxide, polylactic acid, and poly(lactic-co-glycolic acid), polyglycolic acid, or a polyester, a polyamide, or other biodegradable polymers, wherein a terminal hydroxy or carboxy group can be substituted to create an ether or ester, respectively;
(ii) —C₁₀-C₃₀alkylR³, —C₁₀-C₃₀alkenylR³, —C₁₀-C₃₀alkynylR³, —C₁₀-C₃₀alkenylalkynylR³, —C₁₀-C₃₀alkyl, —C₁₀-C₃₀alkenyl, —C₁₀-C₃₀alkynyl, —C₁₀-C₃₀alkenylalkynyl;
(iii) an unsaturated fatty acid residue including but not limited the carbon fragment taken from linoleic acid (—(CH₂)₈(CH)₂CH₂(CH)₂(CH₂)₄CH₃)), docosahexaenoic acid (—(CH₂)₃(CHCHCH₂)₆CH₃)), eicosapentaenoic acid (—(CH₂)₄(CHCHCH₂)₅CH₃)), alpha-linolenic acid (—(CH₂)₈(CHCHCH₂)₃CH₃)) stearidonic acid, y-linolenic acid, arachidonic acid, docosatetraenoic acid, palmitoleic acid, vaccenic acid, paullinic acid, oleic acid, elaidic acid, gondoic acid, uric acid, nervonic acid or mead acid; and
(iv) alkyl, cycloalkyl, cycloalkylalkyl, heterocycle, heterocycloalkyl, arylalkyl, heteroarylalkyl;

R¹⁸ is selected from:
(i) —C₁₀-C₃₀alkylR³, —C₁₀-C₃₀alkenylR³, —C₁₀-C₃₀alkynylR³, —C₁₀-C₃₀alkenylalkynylR³, —C₁₀-C₃₀alkyl, —C₁₀-C₃₀alkenyl, —C₁₀-C₃₀alkynyl, —C₁₀-C₃₀alkenylalkynyl; and
(ii) an unsaturated fatty acid residue including but not limited to the carbon chains from linoleic acid (—(CH₂)₈(CH)₂CH₂(CH)₂(CH₂)₄CH₃)), docosahexaenoic acid (—(CH₂)₃(CHCHCH₂)₆CH₃)), eicosapentaenoic acid (—(CH₂)₄(CHCHCH₂)₅CH₃)), alpha-linolenic acid (—(CH₂)₈(CHCHCH₂)₃CH₃)), stearidonic acid, y-linolenic acid, arachidonic acid, docosatetraenoic acid, palmitoleic acid, vaccenic acid, paullinic acid, oleic acid, elaidic acid, gondoic acid, uric acid, nervonic acid and mead acid, and wherein, if desired, each of which can be substituted with R³;

R³⁶ is selected from C(O)A,

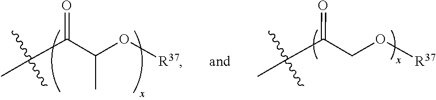

or in an alternative embodiment, R³⁶ is selected from

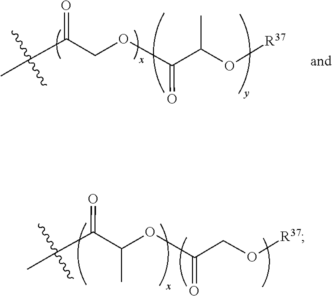

R³⁷ is selected from hydrogen, —C(O)A, —C(O)alkyl, aryl, alkyl, cycloalkyl, cycloalkylalkyl, heterocyclyl, heterocycloalkyl, arylalkyl, heteroaryl, and heteroarylalkyl;

L¹ is selected from:

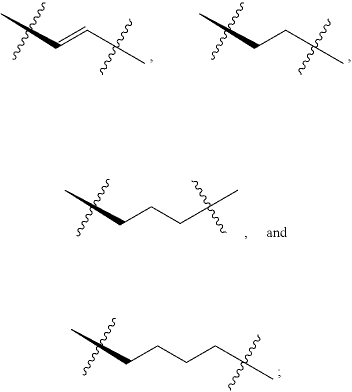

L² is selected from:

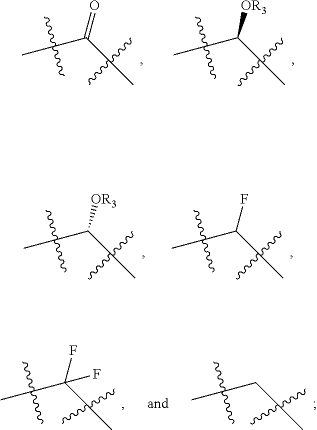

A is selected from H, alkyl, cycloalkyl, cycloalkylalkyl, heterocycle, heterocycloalkyl, aryl, arylalkyl, heteroaryl, heteroarylalkyl, aryloxy, and alkyloxy wherein each group can be optionally substituted with another desired substituent group which is pharmaceutically acceptable and sufficiently stable under the conditions of use, for example selected from R³; and

R³, x and y are defined above.

In one embodiment of Formula II, R¹³ is

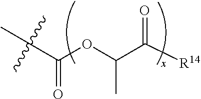

and x is 4.

In one embodiment of Formula II or Formula II′, R¹⁴ is selected from

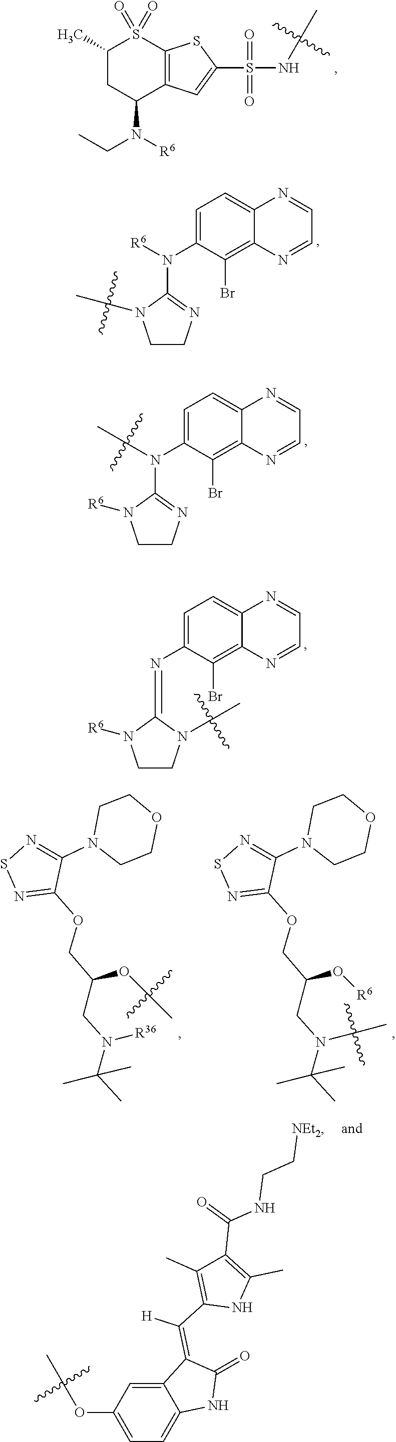

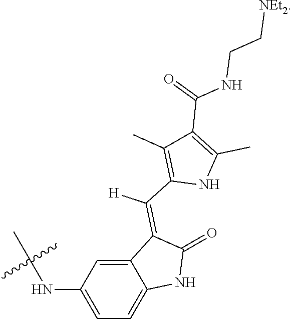

In an alternative embodiment, R¹⁴ is

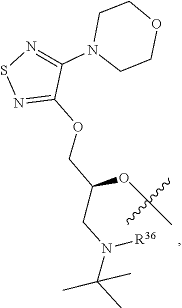

and R³⁶ is selected from

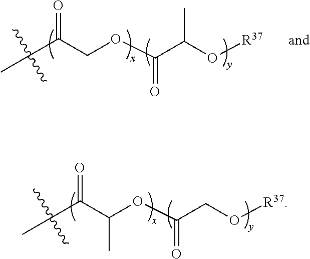

In an alternative embodiment, R¹⁴ is

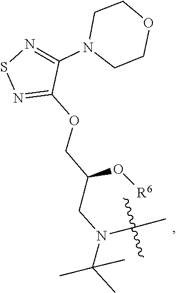

R⁶ is R³⁶ and R³⁶ is selected from

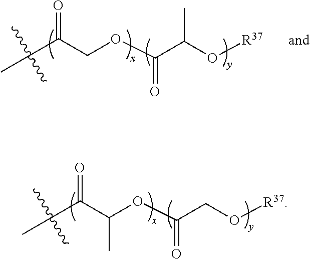

In one embodiment, x and y are independently an integer between 1 and 12 (1, 2, 3, 4, 5, 6, 7, 8, 9, 10, 11, or 12).

In one embodiment, x and y are independently an integer between 1 and 10 (1, 2, 3, 4, 5, 6, 7, 8, 9, or 10).

In one embodiment, x and y are independently an integer between 1 and 8 (1, 2, 3, 4, 5, 6, 7, or 8).

In one embodiment, x and y are independently an integer between 1 and 6 (1, 2, 3, 4, 5, or 6).

In one embodiment, x and y are independently an integer between 4 and 10 (4, 5, 6, 7, 8, 9, or 10).

In one embodiment, x is an integer between 1 and 12 (1, 2, 3, 4, 5, 6, 7, 8, 9, 10, 11, or 12) and y is an integer between 1 and 6 (1, 2, 3, 4, 5, or 6).

In one embodiment, y is an integer between 1 and 12 (1, 2, 3, 4, 5, 6, 7, 8, 9, 10, 11, or 12) and x is an integer between 1 and 6 (1, 2, 3, 4, 5, or 6).

In one embodiment, x is an integer between 1 and 6 (1, 2, 3, 4, 5, or 6) and y is an integer between 1 and 3 (1, 2, or 3).

In one embodiment, y is an integer between 1 and 6 (1, 2, 3, 4, 5, or 6) and x is an integer between 1 and 3 (1, 2, or 3).

In an alternative embodiment x is 1 and y is 1.

In an alternative embodiment x is 1 and y is 2.

In an alternative embodiment x is 1 and y is 3.

In an alternative embodiment x is 1 and y is 4.

In an alternative embodiment x is 1 and y is 5.

In an alternative embodiment x is 1 and y is 6.

In an alternative embodiment x is 1 and y is 7.

In an alternative embodiment x is 1 and y is 8.

In an alternative embodiment x is 2 and y is 1.

In an alternative embodiment x is 2 and y is 2.

In an alternative embodiment x is 2 and y is 3.

In an alternative embodiment x is 2 and y is 4.

In an alternative embodiment x is 2 and y is 5.

In an alternative embodiment x is 2 and y is 6.

In an alternative embodiment x is 2 and y is 7.

In an alternative embodiment x is 2 and y is 8.

The disclosure provides a prodrug of Formula III, Formula IV, Formula IV′, Formula V, and Formula VI:

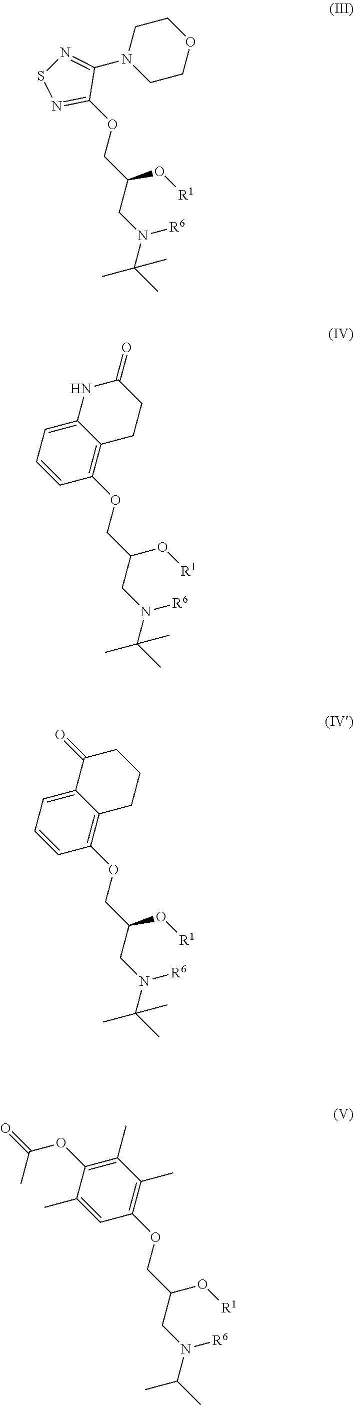

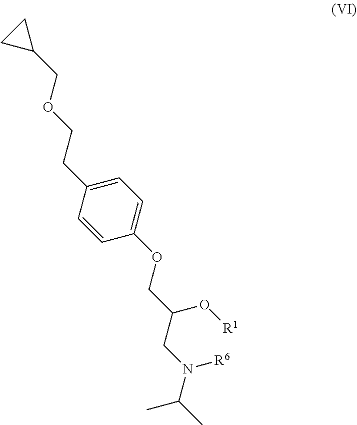

or a pharmaceutically acceptable composition, salt, or isotopic derivative thereof.
R¹ is selected from

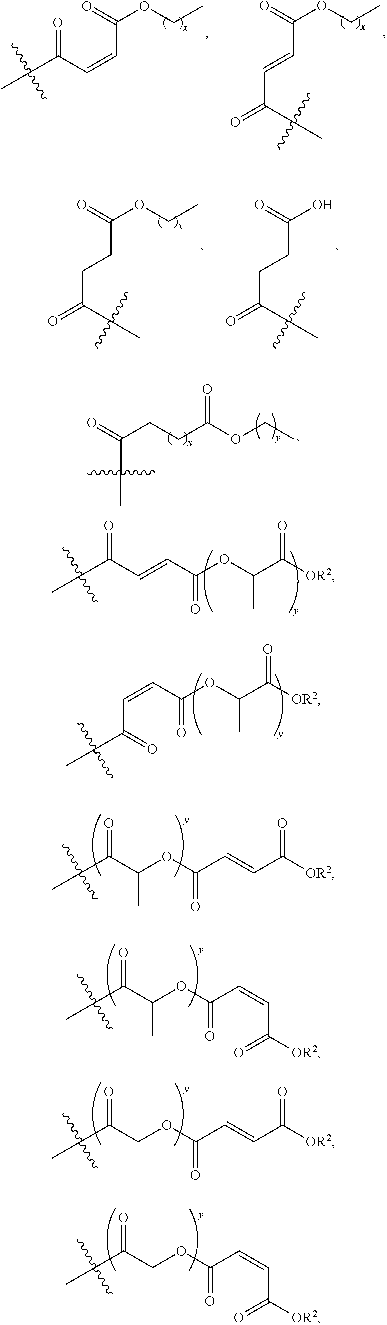

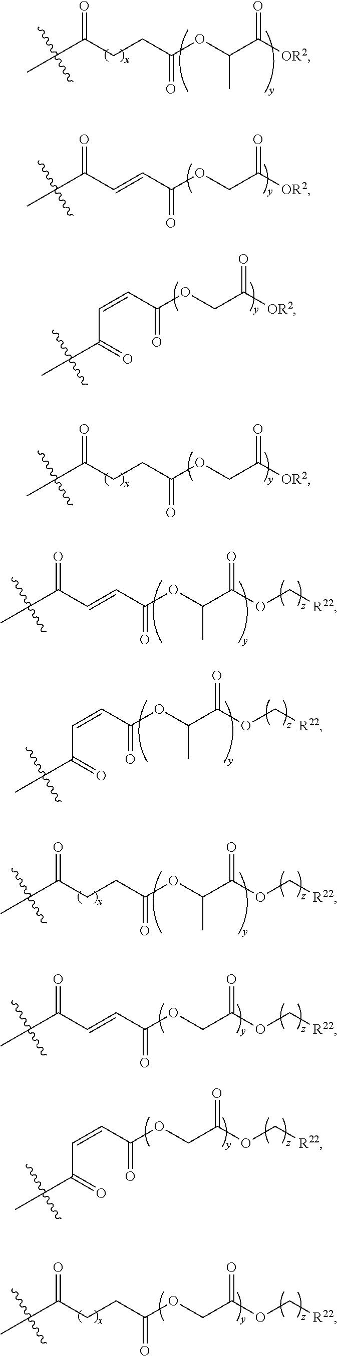

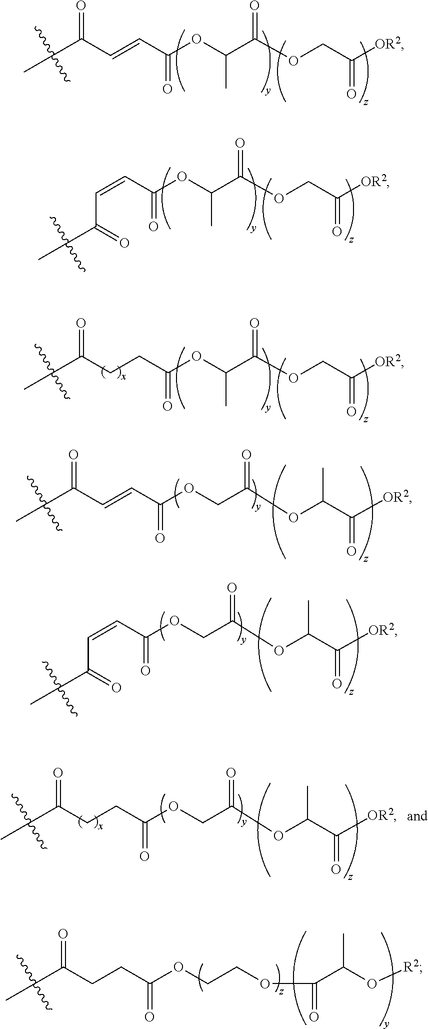

or

R² is hydrogen, alkyl, alkenyl, alkynyl cycloalkyl, cycloalkylalkyl, heterocyclyl, heterocycloalkyl, aryl, arylalkyl, heteroaryl, or heteroarylalkyl;

R²² is hydrogen, hydroxy, amino, A, alkyl, alkoxy, alkenyl, alkynyl cycloalkyl, cycloalkylalkyl, heterocyclyl, heterocycloalkyl, aryl, arylalkyl, heteroaryl, heteroarylalkyl, aryloxy, or stearoyl;

A is selected from H, alkyl, cycloalkyl, cycloalkylalkyl, heterocycle, heterocycloalkyl, aryl, arylalkyl, heteroaryl, heteroarylalkyl, aryloxy, and alkyloxy wherein each group can be optionally substituted with another desired substituent group which is pharmaceutically acceptable and sufficiently stable under the conditions of use, for example selected from R³;

R³ is selected from halogen, hydroxyl, cyano, mercapto, amino, alkoxy, cycloalkyl, cycloalkylalkyl, heterocyclyl, heterocycloalkyl, aryl, arylalkyl, heteroaryl, heteroarylalkyl, aryloxy, —S(O)₂alkyl, —S(O)alkyl, —P(O)(Oalkyl)₂, B(OH)₂, —Si(CH₃)₃, —COOH, —COOalkyl, and —CONH₂, each of which except halogen, cyano, and —Si(CH₃)₃ may be optionally substituted, for example with halogen, alkyl, aryl, heterocycle or heteroaryl if desired and if the resulting compound is stable and achieves the desired purpose, wherein the group cannot be substituted with itself, for example alkyl would not be substituted with alkyl;

R⁶, x, y, and z are defined above.

In one embodiment, R¹ is

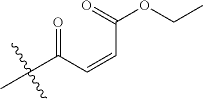 [SMILES: *C(=O)/C=C\C(=O)OCC]

and R⁶ is hydrogen.

In one embodiment, R¹ is

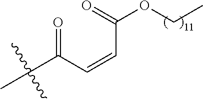

and R⁶ is hydrogen.

In one embodiment, R¹ is

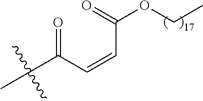

and R⁶ is hydrogen.

In one embodiment, R¹ is

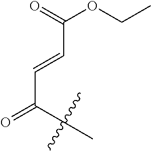 [SMILES: *C(=O)/C=C/C(=O)OCC]

and R⁶ is hydrogen.

In one embodiment, R¹ is

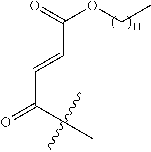

and R⁶ is hydrogen.

In one embodiment, R¹ is

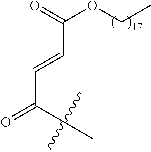

and R⁶ is hydrogen.

In one embodiment, R¹ is

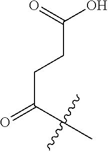 [SMILES: *C(=O)CCC(=O)O]

and R⁶ is hydrogen.

In one embodiment, R¹ is

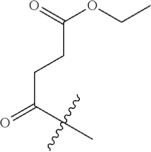 [SMILES: *C(=O)CCC(=O)OCC]

and R⁶ is hydrogen.

In one embodiment, R¹ is

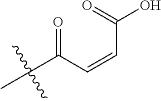 [SMILES: *C(=O)/C=C\C(=O)O]

and R⁶ is hydrogen.

In one embodiment, R¹ is

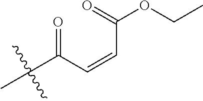 [SMILES: *C(=O)/C=C\C(=O)OCC]

and R⁶ is hydrogen.

In an alternative embodiment, R¹ is selected from

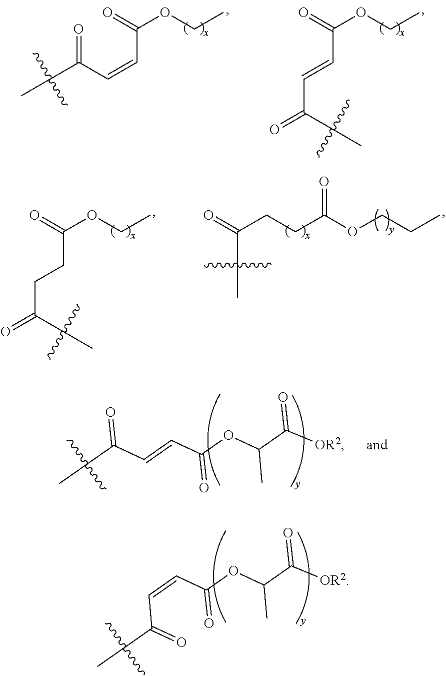

In one embodiment, a compound of Formula III is the pharmaceutically acceptable HCl salt.

In one embodiment, a compound of Formula III is the pharmaceutically acceptable maleic salt.

The disclosure also provides a prodrug of Formula VII, Formula VIII, Formula VIII′, Formula IX, or Formula X:

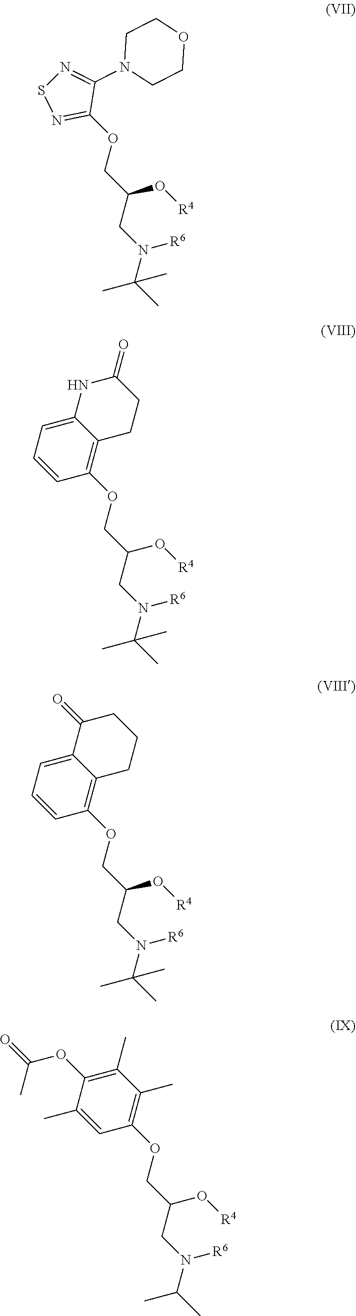

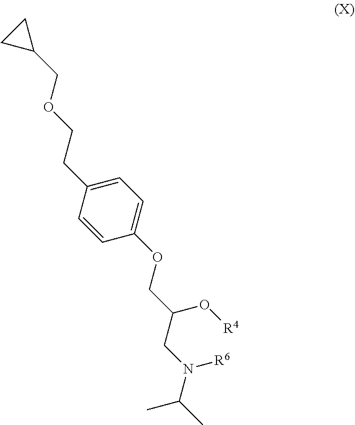

or a pharmaceutically acceptable composition, salt, or isotopic derivative thereof.

R⁴ is selected from

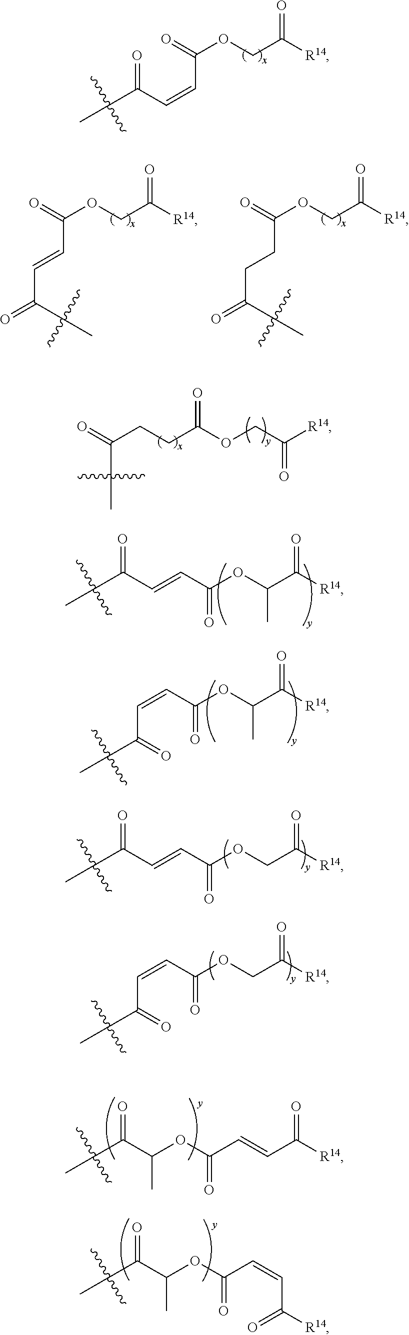

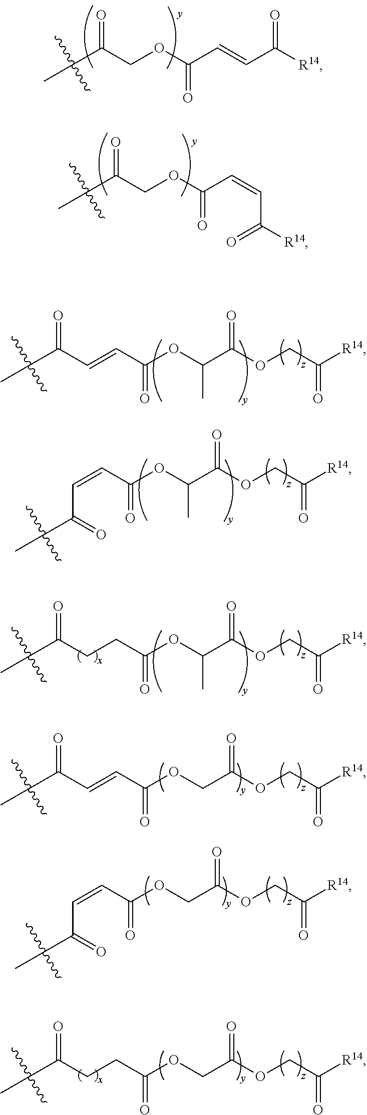

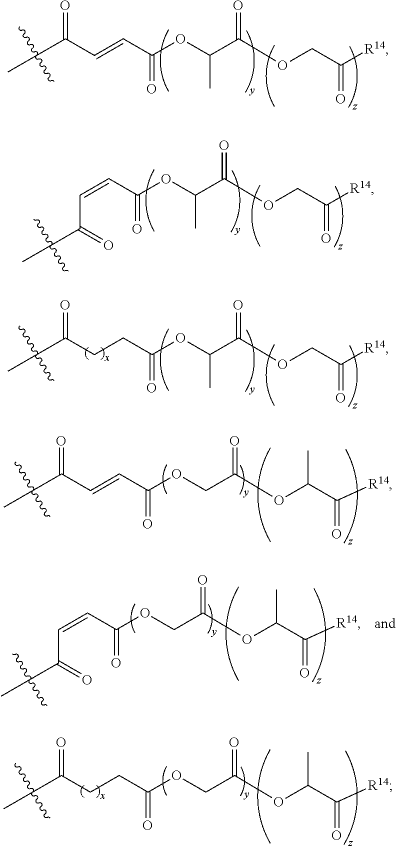

R¹⁴, x, y, and z are defined above.

In one embodiment, a compound of Formula VII is the pharmaceutically acceptable HCl salt.

In one embodiment, a compound of Formula VII is the pharmaceutically acceptable maleic salt.

This disclosure provides a prodrug of Formula XI and Formula XII

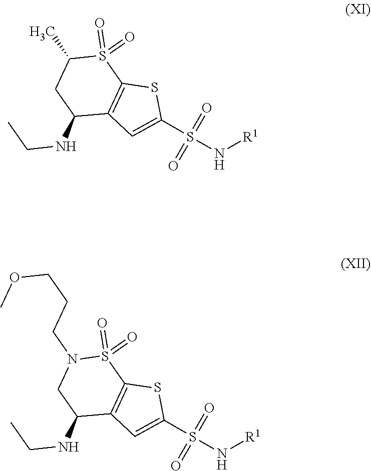

or a pharmaceutically acceptable composition, salt, or isotopic derivative thereof.

R¹ is defined above.

In one embodiment, R¹ is

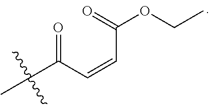 [SMILES: *C(=O)/C=C\C(=O)OCC]

In one embodiment, R¹ is

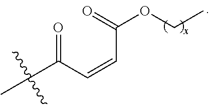

In one embodiment, R¹ is

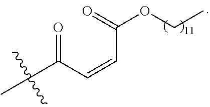

In one embodiment, R¹ is

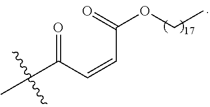

In one embodiment, R¹ is

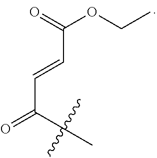 [SMILES: *C(=O)/C=C/C(=O)OCC]

In one embodiment, R¹ is

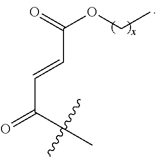

In one embodiment, R¹ is

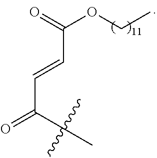

In one embodiment, R¹ is

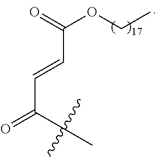

This disclosure provides a prodrug of Formula XIII and XIV

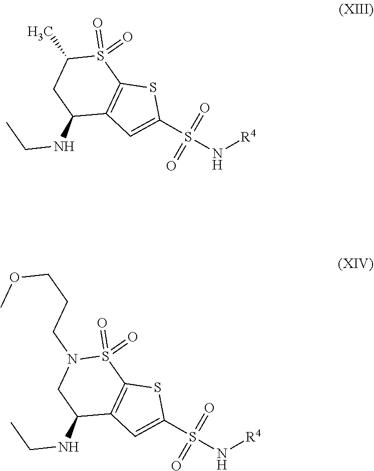

or a pharmaceutically acceptable composition, salt, or isotopic derivative thereof;

wherein R⁴ is defined above.

This disclosure provides a prodrug of Formula XV and Formula XVI

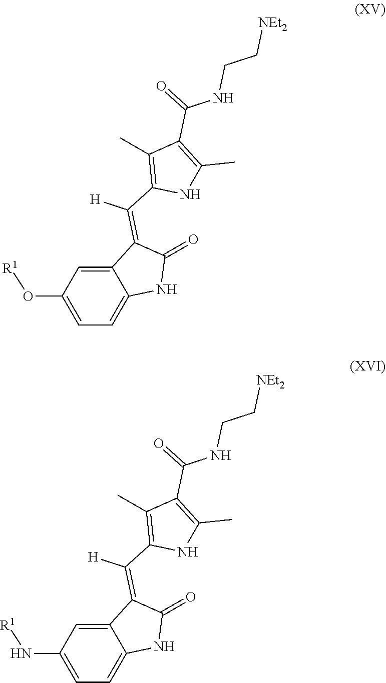

or a pharmaceutically acceptable composition, salt, or isotopic derivative thereof;

wherein R¹ is defined above.

In one embodiment, R¹ is

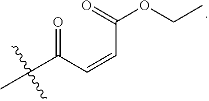 [SMILES: *C(=O)/C=C\C(=O)OCC]

In one embodiment, R¹ is

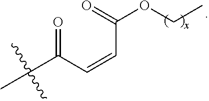

In one embodiment, R¹ is

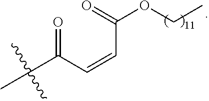

In one embodiment, R¹ is

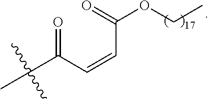

In one embodiment, R¹ is

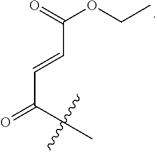 [SMILES: *C(=O)/C=C/C(=O)OCC]

In one embodiment, R¹ is

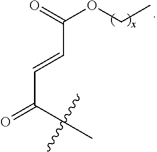

In one embodiment, R¹ is

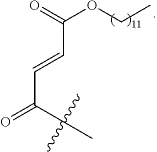

In one embodiment, R¹ is

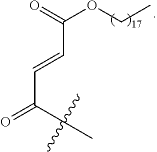

The disclosure also provides a prodrug of Formula XVII:

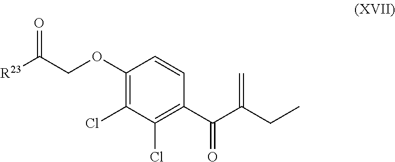

or a pharmaceutically acceptable composition, salt, or isotopic derivative thereof.

wherein:

R²³ is selected from:

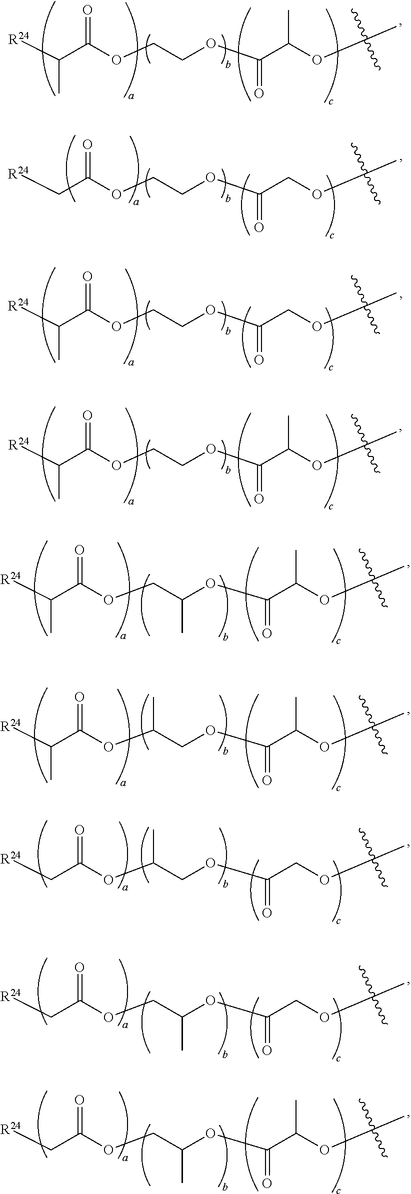

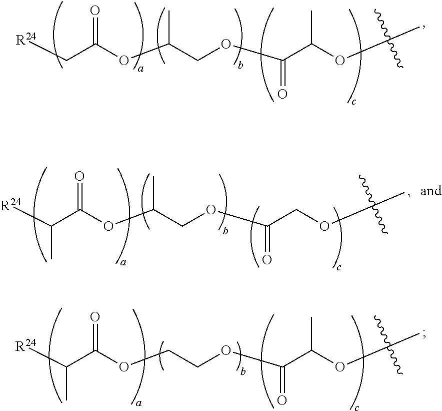

R²⁴ is

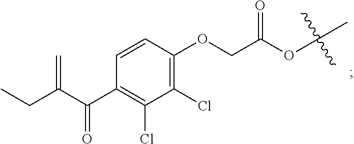

a, b, and c are independently an integer selected from 0 to 30 (0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) wherein a and c cannot both be 0.

The polymer moieties described in Formula XVII above are depicted as block copolymers (for example, blocks of “a” followed by blocks of “b” followed by blocks of “c”), but it is intended that the polymer can be a random or alternating copolymer (for example, “a” “b” and “c” are either randomly distributed or alternate).

In one embodiment, a, b, and c are independently selected from an integer between 1 and 12 (1, 2, 3, 14, 5, 6, 7, 8, 9, 10, 11, or 12).

In an alternative embodiment, a, b, and c are independently selected from an integer between 1 and 8 (1, 2, 3, 4, 5, 6, 7, or 8).

In an alternative embodiment, a, b, and c are independently selected from an integer between 1 and 6 (1, 2, 3, 4, 5, or 6).

In an alternative embodiment, a, b, and c are independently selected from an integer between 1 and 3 (1, 2, or 3).

In an alternative embodiment, a and c are independently selected from an integer between 1 and 6 (1, 2, 3, 4, 5, or 6) and b is 1.

In an alternative embodiment, a and c are independently selected from an integer between 1 and 3 (1, 2, or 3) and b is 1.

In an alternative embodiment, a and c are independently selected from an integer between 1 and 12 (1, 2, 3, 4, 5, 6, 7, 8, 9, 10, 11, or 12) and b is selected from an integer between 1 and 6 (1, 2, 3, 4, 5, or 6).

In an alternative embodiment, a and c are independently selected from an integer between 1 and 6 (1, 2, 3, 4, 5, or 6) and b is selected from an integer between 1 and 3 (1, 2, or 3).

In an alternative embodiment, a and c independently selected from an integer between 1, 2, 3, and 4 and b is 1.

In an alternative embodiment, a and c are 2 and b is 1.

In an alternative embodiment, a and c are 3 and b is 1.

In an alternative embodiment, a and c are 4 and b is 1.

In an alternative embodiment, the prodrug is Compound 52, Compound 53, Compound 55, or Compound 56:

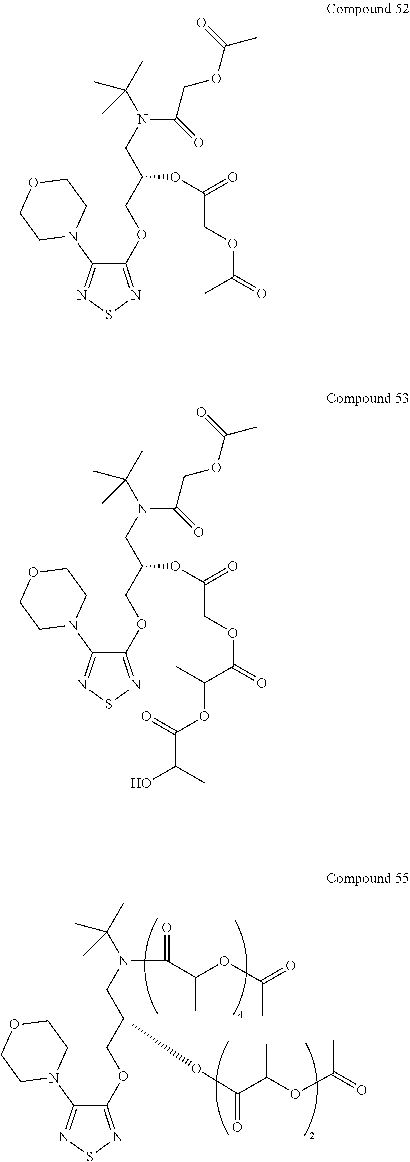

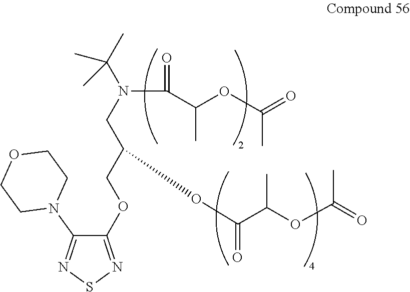

Pharmaceutical compositions comprising a compound or salt of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII together with a pharmaceutically acceptable carrier are also disclosed.

Pharmaceutical compositions comprising a compound or salt of Compound 52, Compound 53, Compound 55, or Compound 56 together with a pharmaceutically acceptable carrier are also disclosed.

Methods of treating or preventing ocular disorders, including glaucoma, a disorder mediated by carbonic anhydrase, a disorder mediated by a Rho-associated kinase, a disorder mediated by a dual leucine zipper kinase, a disorder mediated by an α2 adrenergic receptor, a disorder mediated a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), geographic atrophy, or diabetic retinopathy are disclosed comprising administering a therapeutically effective amount of a compound or salt or Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII to a host, including a human, in need of such treatment.

In another embodiment, an effective amount of a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, or Formula XIV is provided to decrease intraocular pressure (IOP) caused by glaucoma. In an alternative embodiment, the compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII can be used to decrease intraocular pressure (IOP), regardless of whether it is associated with glaucoma.

In one embodiment, the disorder is associated with an increase in intraocular pressure (IOP) caused by potential or previously poor patient compliance to glaucoma treatment. In yet another embodiment, the disorder is associated with potential or poor neuroprotection through neuronal nitric oxide synthase (NOS). The active compound or its salt or prodrug provided herein may thus dampen or inhibit glaucoma in a host, by administration of an effective amount in a suitable manner to a host, typically a human, in need thereof.

Methods for the treatment of a disorder associated with glaucoma, increased intraocular pressure (IOP), and optic nerve damage caused by either high intraocular pressure (IOP) or neuronal nitric oxide synthase (NOS) are provided that includes the administration of an effective amount of a compound Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII or a pharmaceutically acceptable salt thereof, optionally in a pharmaceutically acceptable carrier are also disclosed.

Methods for the treatment of a disorder associated with age-related macular degeneration (AMD) and geographic atrophy are provided that includes the administration of an effective amount of a compound Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII or a pharmaceutically acceptable salt thereof, optionally in a pharmaceutically acceptable carrier are also disclosed. In one embodiment, the age-related macular degeneration is wet age-related macular degeneration. In one embodiment, the age-related macular degeneration is neovascular age-related macular degeneration.

Methods for treatment of a disorder mediated by a carbonic anhydrase are provided to treat a patient in need thereof wherein a prodrug of a carbonic anhydrase inhibitor as described herein is provided.

Methods for treatment of a disorder mediated by a Rho-associated kinase are provided to treat a patient in need thereof wherein a prodrug of a Rho-associated kinase inhibitor as described herein is provided.

Methods for treatment of a disorder mediated by a beta-blocker are provided to treat a patient in need thereof wherein a prodrug of a beta blocker as described herein is provided.

Methods for treatment of a disorder mediated by a dual leucine zipper kinase are provided to treat a patient in need thereof wherein a prodrug of a dual leucine zipper kinase inhibitor as described herein is provided.

Methods for treatment of a disorder mediated by a cu adrenergic are provided to treat a patient in need thereof also disclosed wherein a prodrug of a cu adrenergic agonist as described herein is provided.

The present invention includes at least the following features:
(a) a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII as described herein, or a pharmaceutically acceptable salt or prodrug thereof (each of which and all subgenuses and species thereof are considered individually and specifically described);
(b) a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII as described herein, or a pharmaceutically acceptable salt or prodrug thereof, for use in treating or preventing an ocular disorder as further described herein;
(c) a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII as described herein, or a pharmaceutically acceptable salt or prodrug thereof for use in treating or preventing disorders related to an ocular disorder such as glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), geographic atrophy or diabetic retinopathy;
(d) use of a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII or a pharmaceutically acceptable salt or prodrug thereof in the manufacture of a medicament for use in treating or preventing glaucoma and disorders involving increased intraocular pressure (IOP) or nerve damage related to either IOP or nitric oxide synthase (NOS) and other disorders described further herein;
(e) use of a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII or a pharmaceutically acceptable salt or prodrug thereof in the manufacture of a medicament for use in treating or preventing age-related macular degeneration (AMD) and other disorders described further herein;
(f) a process for manufacturing a medicament intended for the therapeutic use for treating or preventing glaucoma and disorders involving nerve damage related to both (IOP) and nitric oxide synthase (NOS) and other disorders described further herein characterized in that a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII as described herein is used in the manufacture;
(g) a pharmaceutical formulation comprising an effective host-treating amount of the a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII or a pharmaceutically acceptable salt or prodrug thereof together with a pharmaceutically acceptable carrier or diluent;
(h) a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII as described herein in substantially pure form, (e.g., at least 90 or 95%);
(i) processes for the manufacture of a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII or a pharmaceutically acceptable salt or prodrug thereof; and
(j) processes for the preparation of therapeutic products including drug delivery agents that contain an effective amount a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII as described herein.
(k) A method for the controlled administration of timolol to a patient in need thereof, comprising administering a prodrug of timolol in a microparticle in vivo, wherein the timolol prodrug containing microparticle exhibits in vitro drug release kinetics in an aqueous solution at a pH between 6-8 at body temperature of a substantially consistent release of at least 60% timolol itself by molar ratio to the prodrug of timolol or an intermediate metabolite thereof over at least 100 days.

### BRIEF DESCRIPTION OF THE FIGURES

FIG. 1 illustrates the stability of ethacrynic acid-PLA (n=4) ethyl ester (1) at 37° C. over 6 days. As discussed in Example 9, as the ester linkages are hydrolyzed, parent ethacrynic acid is generated at a linear rate. Parent ethacrynic acid is denoted by 0′ and 1′-4′ denotes uncapped ethacrynic-acid conjugated to 1-4 PLA repeat units. The x-axis is time measured in days and the y-axis is the area under the curve measured in intensity.

FIG. 2 illustrates the stability of ethacrynic acid-PLA (n=2) ethyl ester (2) at 37° C. over 6 days. As discussed in Example 9, as the ester linkages are hydrolyzed, parent ethacrynic acid is generated at a linear rate. The x-axis is time measured in days and the y-axis is the area under the curve measured in intensity.

FIG. 3 illustrates the stability of ethacrynic acid-PLA(n=6)-ethyl ester (25) at 37° C. over 6 days as discussed in Example 9. Parent ethacrynic acid is denoted by 0′ denotes and 1′-5′ denotes uncapped ethacrynic-acid conjugated to 1-5 PLA repeat units. The x-axis is time measured in days and the y-axis is the area under the curve measured in intensity.

FIG. 4 illustrates that the rate of drug release increased with increased drug loading (DL) for ECA-PLA(n=6) (25) as described in Example 10. The x-axis is time measured in days and the y-axis is the normalized cumulative drug release measured in percent.

FIG. 5 illustrates the drug release kinetics of Timolol-O-ethyl fumurate (17) from microparticles of different polymer blends: (i) PLA/PGA, (ii) PLGA/PLGA where the PLGA polymers have different ratios of lactide to glycolide, (iii) PLA/PLGA/PLGA where the PLGA polymer have different ratios of lactide to glycolide, (iv) PLA, and (v) PLGA (Example 11). All blends also contain 1% PEG-PLGA. The x-axis is time measured in days and the y-axis is the normalized cumulative drug release measured in percent.

FIG. 6 illustrates the drug release kinetics of ethacrynic acid-PLA (n=4) ethyl ester (1) from microparticles composed of PLA/PLGA blends, PLGA blends, as discussed in Example 11. The drug was released over a period of approximately 66 days. The x-axis is time measured in days and the y-axis is the cumulative drug release measured in percent.

FIG. 7 illustrates the drug release kinetics of Timolol-O-laurylfumurate maleate (12) and Timolol-O-stearylfumurate-maleate (13) compared to bis-prodrugs of Timolol (Timolol-succinic acid-Timolol-maleate, Timolol-glutaric acid-Timolol-maleate, and Timolol-fumurate-Timolol-maleate) from 77/22 (PLA 4.5A/PLGA8515 5A) blended microparticles as discussed in Example 11. The x-axis is time measured in days and the y-axis is the cumulative drug release measured in percent.

FIG. 8 illustrates the drug release kinetics of ethacrynic acid-PLA(n=4)-ethyl ester (2) from microparticles of different polymer blends: (i) PLA/PGA, (ii) PLGA/PLGA where the PLGA polymers have different ratios of lactide to glycolide, (iii) PLA/PLGA/PLGA where the PLGA polymer have different ratios of lactide to glycolide, (iv) PLA, and (v) PLGA (Example 11). All blends also contain 1% PEG-PLGA. The x-axis is time measured in days and the y-axis is the cumulative drug release measured in percent.

FIG. 9 illustrates the drug release kinetics of ethacrynic acid-PLA(n=6)-ethyl ester (25) from microparticles of different polymer blends: (i) PLA/PGA, (ii) PLGA/PLGA where the PLGA polymers have different ratios of lactide to glycolide, (iii) PLA/PLGA/PLGA where the PLGA polymer have different ratios of lactide to glycolide, (iv) PLA, and (v) PLGA (Example 11). All blends also contain 1% PEG-PLGA. The x-axis is time measured in days and the y-axis is the cumulative drug release measured in percent.

FIG. 10 illustrates the in vitro release profile of a formulation of ECA-PLA(n=6) (25) prepared in 1% PVA in PBS vs 1% PVA in water as discussed in Example 11. The x-axis is time measured in days and the y-axis is the cumulative drug release measured in percent.

FIG. 11 is a comparison of the in vitro release profiles of formulations of ECA-PLA(n=4) (1) and ECA-PLA(n=6) (25) microparticles as discussed in Example 11. The x-axis is time measured in days and the y-axis is the cumulative drug release measured in percent.

FIG. 12 is the synthesis of Compound 1, an ethacrynic acid mono-prodrug.

FIG. 13 is the synthesis of Compound 9, a Timolol mono-prodrug.

FIG. 14 is the synthesis of Compound 11, a Timolol mono-prodrug.

FIG. 15 is the synthesis of Compound 23, a bis-prodrug of Sunitinib and ethacrynic acid. Compound 24 is synthesized via the same reaction.

FIG. 16 is an image of the representative particle morphology of a microparticle encapsulating a bis-prodrug of Timolol as described in Example 14.

FIG. 17 is a graph of drug release from polymeric microparticles encapsulating Compound 54 (batch 54-2 from Table 11) as described in Example 14. The total drug release is compared to parent Timolol release. The total release includes Compound 54, all known intermediates and parent Timolol. Parent release refers to only the release rate corresponding to the parent Timolol compound signal. The dashed lines represent the projected release over 3 months and 6 months. The x-axis represents the time measured in days and the y-axis represents release measured in percent.

FIG. 18 is a graph of drug release from polymeric microparticles encapsulating Compound 54 (batch 54-1 from Table 11) as described in Example 14. The total drug release is compared to parent Timolol release. The total release includes Compound 54, all known intermediates and parent Timolol. Parent release refers to only the release rate corresponding to the parent Timolol compound signal. The dashed lines represent the projected release over 3 months and 6 months. The x-axis represents the time measured in days and the y-axis represents release measured in percent.

FIG. 19 is a graph of drug release from polymeric microparticles encapsulating Compound 55 (batches 55-1, 55-2, and 55-3 from Table 11) as described in Example 14. The total drug release is compared to parent Timolol release for each batch. The total release includes Compound 55, all known intermediates and parent Timolol. Parent release refers to only the release rate corresponding to the parent Timolol compound signal. The dashed lines represent the projected release over 3 months and 6 months. The x-axis represents the time measured in days and the y-axis represents release measured in percent.

FIG. 20 is a graph of drug release from polymeric microparticles encapsulating Compound 56 (batches 56-1, 56-2, and 56-3 from Table 11) as described in Example 14. The total drug release is compared to parent Timolol release for each batch. The total release includes Compound 56, all known intermediates and parent Timolol. Parent release refers to only the release rate corresponding to the parent Timolol compound signal. The dashed lines represent the projected release over 3 months and 6 months. The x-axis represents the time measured in days and the y-axis represents release measured in percent.

FIG. 21 is a graph of drug release from polymeric microparticles encapsulating Compound 52 (batches 52-1 from Table 11) as described in Example 14. The total drug release is compared to parent Timolol release. The total release includes Compound 52, all known intermediates and parent Timolol. Parent release refers to only the release rate corresponding to the parent Timolol compound signal. The dashed lines represent the projected release over 3 months and 6 months. The x-axis represents the time measured in days and the y-axis represents release measured in percent.

FIG. 22 is a graph of drug release from polymeric microparticles encapsulating Compound 50 (batches 50-1 from Table 11) as described in Example 14. The total drug release is compared to parent Timolol release. The total release includes Compound 50, all known intermediates and parent Timolol. Parent release refers to only the release rate corresponding to the parent Timolol compound signal. The dashed lines represent the projected release over 3 months and 6 months. The x-axis represents the time measured in days and the y-axis represents release measured in percent. As discussed in Example 14, Compound 50 exhibited linear drug release and the correlation between total drug release and parent drug release was high. The x-axis represents the time measured in days and the y-axis represents release measured in percent.

FIG. 23 is a graph of the drug release from polymeric microparticles encapsulating Compound 50 (batches 50-A and 50-B from Table 12) as described in Example 14. For each batch, the total drug release and the parent drug release is shown. The total release includes Compound 50, all known intermediates and parent Timolol. Parent release refers to only the release rate corresponding to the parent Timolol compound signal. The dashed lines represent the projected release over 3 months and 6 months. As discussed in Example 14, Compound 50 exhibited linear drug release and the correlation between total drug release and parent drug release was high. The x-axis represents the time measured in days and the y-axis represents release measured in percent.

FIG. 24 is a graph of the drug release from polymeric microparticles encapsulating Compound 51 (batch 51-A from Table 12) as described in Example 14. The total drug release is compared to parent Timolol release. The total release includes Compound 51, all known intermediates and parent Timolol. Parent release refers to only the release rate corresponding to the parent Timolol compound signal. The dashed lines represent the projected release over 3 months and 6 months. As discussed in Example 14, Compound 51 did not exhibit linear drug release. The x-axis represents the time measured in days and the y-axis represents release measured in percent.

FIG. 25 is a graph of the drug release from polymeric microparticles encapsulating Compound 53 (batches 53-A and 53-B from Table 12) as described in Example 14. For the batches, the total drug release and the parent drug release is shown. Total release includes Compound 53, all known intermediates and parent Timolol. Parent release refers to only the release rate corresponding to the parent Timolol compound signal. The dashed lines represent the projected release over 3 months and 6 months. As discussed in Example 14, Compound 53 did not exhibit linear drug release. The x-axis represents the time measured in days and the y-axis represents release measured in percent.

FIG. 26 is a measure of the stability of Compound 50 in PBS as measured via HPLC. Compound 50 (prodrug with a retention time of 6.773 minutes) and the other breakdown products, including parent Timolol are labeled with their respective retention times as measured in minutes. As discussed in Example 15, Compound 50 breaks down over the course of 5 days into the breakdown products and parent Timolol. The x-axis is time measured in days and the y-axis is intensity measured as area under the curve (AUC).

FIG. 27A is a measure of the stability of Compound 51 in 100% serum as measured via HPLC. Compound 51 (prodrug with a retention time of 6.555 minutes) and the other breakdown products, including parent Timolol are labeled with their respective retention times measured in minutes. As discussed in Example 15, Compound 51 breaks down over the course of 5 days into the breakdown products and parent Timolol. The x-axis is time measured in days and the y-axis is intensity measured as area under the curve (AUC).

FIG. 27B is a measure of the stability of Compound 51 in 50% serum and 50% PBS as measured via HPLC. Compound 51 (prodrug with a retention time of 6.555 minutes) and the other breakdown products, including parent Timolol are labeled with their respective retention times measured in minutes. As discussed in Example 15, Compound 51 breaks down over the course of 5 days into the breakdown products and parent Timolol. The x-axis is time measured in days and the y-axis is intensity measured as area under the curve (AUC).

FIG. 27C is a measure of the stability of Compound 51 in 100% PBS as measured via HPLC. Compound 51 (prodrug with a retention time of 6.553 minutes) and the other breakdown products, including parent Timolol are labeled with their respective retention times measured in minutes. As discussed in Example 15, Compound 51 breaks down over the course of 5 days into the breakdown products and parent Timolol. The x-axis is time measured in days and the y-axis is intensity measured as area under the curve (AUC).

FIG. 28 is a measure of the stability of Compound 52 in 100% PBS as measured via HPLC. Compound 52 (prodrug with a retention time of 6.102 minutes) and the other breakdown products, including parent Timolol are labeled with their respective retention times measured in minutes. As discussed in Example 15, Compound 52 breaks down over the course of 15 days into the breakdown products and parent Timolol. The x-axis is time measured in days and the y-axis is intensity measured as area under the curve (AUC).

FIG. 29 is a measure of the stability of Compound 53 in 100% PBS as measured via HPLC. Compound 53 (prodrug with a retention time of 5.972 minutes) and the other breakdown products, including parent Timolol are labeled with their respective retention times measured in minutes. As discussed in Example 15, Compound 53 breaks down over the course of 8 days into the breakdown products and parent Timolol. The x-axis is time measured in days and the y-axis is intensity measured as area under the curve (AUC).

### DETAILED DESCRIPTION

### I. Terminology

The presently disclosed subject matter may be embodied in many different forms and should not be construed as limited to the embodiments set forth herein. Indeed, many modifications and other embodiments of the presently disclosed subject matter will come to mind for one skilled in the art to which the presently disclosed subject matter pertains having the benefit of the teachings presented in the descriptions included herein. Therefore, it is to be understood that the presently disclosed subject matter is not to be limited to the specific embodiments disclosed and that modifications and other embodiments are intended to be included within the scope of the disclosed subject matter.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Compounds are described using standard nomenclature. Unless defined otherwise, all technical and scientific terms used herein have the same meaning as is commonly understood by one of skill in the art to which this invention belongs.

The compounds in any of the Formulas described herein include enantiomers, mixtures of enantiomers, diastereomers, cis/trans isomers, tautomers, racemates and other isomers, such as rotamers, as if each is specifically described.

The compounds in any of the Formulas may be prepared by chiral or asymmetric synthesis from a suitable optically pure precursor or obtained from a racemate or mixture of enantiomers or diastereomers by any conventional technique, for example, by chromatographic resolution using a chiral column, TLC or by the preparation of diastereoisomers, separation thereof and regeneration of the desired enantiomer or diastereomer. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, N Y, 1962); and S. H. Wilen Tables of Resolving Agents and Optical Resolutions 268 (E. L. Eliel ed., Univ. of Notre Dame Press, Notre Dame, Ind., 1972, Stereochemistry of Organic Compounds, Ernest L. Eliel, Samuel H. Wilen and Lewis N. Manda (1994 John Wiley & Sons, Inc.), and Stereoselective Synthesis A Practical Approach, Mihály Nógrádi (1995 VCH Publishers, Inc., NY, NY).

The terms “a” and “an” do not denote a limitation of quantity, but rather denote the presence of at least one of the referenced item. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and are independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

The present invention includes compounds of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, or Formula XIV and the use of compounds with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorus, fluorine, and chlorine, such as ²H, ³H, ¹¹C, ¹³C, ¹⁴C, ¹⁵N, ¹⁸F, ³¹P, ³²P, 3S, ³⁶Cl, ¹²⁵I respectively. The invention includes isotopically modified compounds of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII. Isotopically labeled compounds of this invention and prodrugs thereof can generally be prepared by carrying out the procedures disclosed in the schemes or in the examples and preparations described below by substituting an isotopically labeled reagent for a non-isotopically labeled reagent.

By way of general example and without limitation, isotopes of hydrogen, for example, deuterium (²H) and tritium (³H) may be used anywhere in described structures that achieves the desired result. Alternatively or in addition, isotopes of carbon, e.g., ¹³C and ¹⁴C, may be used. In one embodiment, the isotopic substitution is deuterium for hydrogen at one or more locations on the molecule to improve the performance of the drug, for example, the pharmacodynamics, pharmacokinetics, biodistribution, half-life, stability, AUC, Tₘₐₓ, Cₘₐₓ, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete.

Partial deuterium substitution means that at least one hydrogen is substituted with deuterium. In certain embodiments, the isotope is 90, 95 or 99% or more enriched at any location of interest. In one embodiment deuterium is 90, 95 or 99% enriched at a desired location.

In one embodiment, the substitution of a hydrogen atom for a deuterium atom can be provided in any of A, L¹, L², or L³. In one embodiment, the substitution of a hydrogen atom for a deuterium atom occurs within an R group selected from any of R¹, R², R³, R⁴, R⁶, R⁷, R⁸, R⁹, R¹⁰, R¹¹, R¹², R¹³, R¹⁴, R¹⁵, R¹⁶, R¹⁷, R¹⁸, R²³, R²⁴, R¹²¹, R¹²², R¹³⁴, R¹³⁵, R¹⁴¹, R³⁰¹, R³³³, R³³⁴, R³³⁵, R³⁵⁰. For example, when any of R groups are, or contain for example through substitution, methyl, ethyl, or methoxy, the alkyl residue may be deuterated (in non-limiting embodiments, CD₃, CH₂CD₃, CD₂CD₃, CDH₂, CD₂H, CD₃, CHDCH₂D, CH₂CD₃, CHDCHD₂, OCDH₂, OCD₂H, or OCD₃ etc.

The compound of the present invention may form a solvate with a solvent (including water). Therefore, in one embodiment, the invention includes a solvated form of the active compound. The term “solvate” refers to a molecular complex of a compound of the present invention (including salts thereof) with one or more solvent molecules. Examples of solvents are water, ethanol, dimethyl sulfoxide, acetone and other common organic solvents. The term “hydrate” refers to a molecular complex comprising a compound of the invention and water.

Pharmaceutically acceptable solvates in accordance with the invention include those wherein the solvent may be isotopically substituted, e.g. D₂O, d₆-acetone, d₆-DMSO. A solvate can be in a liquid or solid form.

A dash (“—”) is defined by context and can in addition to its literary meaning indicate a point of attachment for a substituent. For example, —(C═O)NH₂ is attached through carbon of the keto (C═O) group. A dash (“—”) can also indicate a bond within a chemical structure. For example —C(O)—NH₂ is attached through carbon of the keto group which is bound to an amino group (NH₂).

An equal sign (“=”) is defined by context and can in addition to its literary meaning indicate a point of attachment for a substituent wherein the attachment is through a double bond. For example, ═CH₂ represents a fragment that is doubly bonded to the parent structure and consists of one carbon with two hydrogens bonded in a terminal fashion. ═CHCH₃ on the other hand represents a fragment that is doubly bonded to the parent structure and consists of two carbons. In the above example it should be noted that the stereoisomer is not delineated and that both the cis and trans isomer are independently represented by the group.

The term “substituted”, as used herein, means that any one or more hydrogens on the designated atom or group is replaced with a moiety selected from the indicated group, provided that the designated atom's normal valence is not exceeded. For example, when the substituent is oxo (i.e., ═O), then in one embodiment, two hydrogens on the atom are replaced. When an oxo group replaces two hydrogens in an aromatic moiety, the corresponding partially unsaturated ring replaces the aromatic ring. For example a pyridyl group substituted by oxo is a pyridone. Combinations of substituents and/or variables are permissible only if such combinations result in stable compounds or useful synthetic intermediates.

A stable compound or stable structure refers to a compound with a long enough residence time to either be used as a synthetic intermediate or as a therapeutic agent, as relevant in context.

“Alkyl” is a straight chain saturated aliphatic hydrocarbon group. In certain embodiments, the alkyl is C₁-C₂, C₁-C₃, C₁-C₆, or C₁-C₃₀ (i.e., the alkyl chain can be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbons in length).

The specified ranges as used herein indicate an alkyl group with length of each member of the range described as an independent species. For example, the term C₁-C₆ alkyl as used herein indicates a straight alkyl group having from 1, 2, 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Alkyls can be further substituted with alkyl to make branched alkyls. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane and 2,3-dimethylbutane. In one embodiment, the alkyl group is optionally substituted as described above.

“Alkenyl” is a straight chain aliphatic hydrocarbon group having one or more carbon-carbon double bonds each of which is independently either cis or trans that may occur at a stable point along the chain. In one embodiment, the double bond in a long chain similar to a fatty acid has the stereochemistry as commonly found in nature. Non-limiting examples are C₂-C₃₀alkenyl, C₁₀-C₃₀alkenyl (i.e., having 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbons), and C₂-C₄alkenyl. The specified ranges as used herein indicate an alkenyl group having each member of the range described as an independent species, as described above for the alkyl moiety. Examples of alkenyl include, but are not limited to, ethenyl and propenyl. Alkenyls can be further substituted with alkyl to make branched alkenyls. In one embodiment, the alkenyl group is optionally substituted as described above.

“Alkynyl” is a straight chain aliphatic hydrocarbon group having one or more carbon-carbon triple bonds that may occur at any stable point along the chain, for example, C₂-C₈alkynyl or C₁₀-C₃₀alkynyl (i.e., having 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbons). The specified ranges as used herein indicate an alkynyl group having each member of the range described as an independent species, as described above for the alkyl moiety. Alkynyls can be further substituted with alkyl to make branched alkynyls. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. In one embodiment, the alkynyl group is optionally substituted as described above.

“Alkylene” is a bivalent saturated hydrocarbon. Alkylenes, for example, can be a 1 to 8 carbon moiety, 1 to 6 carbon moiety, or an indicated number of carbon atoms, for example C₁-C₄alkylene, C₁-C₃alkylene, or C₁-C₂alkylene.

“Alkenylene” is a bivalent hydrocarbon having at least one carbon-carbon double bond. Alkenylenes, for example, can be a 2 to 8 carbon moiety, 2 to 6 carbon moiety, or an indicated number of carbon atoms, for example C₂-C₄alkenylene.

“Alkynylene” is a bivalent hydrocarbon having at least one carbon-carbon triple bond. Alkynylenes, for example, can be a 2 to 8 carbon moiety, 2 to 6 carbon moiety, or an indicated number of carbon atoms, for example C₂-C₄alkynylene.

“Alkenylalkynyl” in one embodiment is a bivalent hydrocarbon having at least one carbon-carbon double bond and at least one carbon-carbon triple bond. It will be recognized to one skilled in the art that the bivalent hydrocarbon will not result in hypervalency, for example, hydrocarbons that include —C═C≡C—C or —C≡C≡C—C, and must be stable. Alkenylalkynyls, for example, can be a 4 to 8 carbon moiety, 4 to 6 carbon moiety, or an indicated number of carbon atoms, for example C₄-C₆alkenylalkynyls.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a “thioalkyl” group is an alkyl group as defined above with the indicated number of carbon atoms covalently bound through a sulfur bridge (—S—). In one embodiment, the alkoxy group is optionally substituted as described above.

“Alkenyloxy” is an alkenyl group as defined covalently bound to the group it substitutes by an oxygen bridge (—O—).

“Amide” or “carboxamide” is —C(O)NR^{a}R^{b} wherein R^{a} and R^{b} are each independently selected from hydrogen, alkyl, for example, C₁-C₆alkyl, alkenyl, for example, C₂-C₆alkenyl, alkynyl, for example, C₂-C₆alkynyl, —C₀-C₄alkyl(C₃-C₇cycloalkyl), —C₀-C₄alkyl(C₃-C₇heterocycloalkyl), —C₀-C₄alkyl(aryl), and —C₀-C₄alkyl(heteroaryl); or together with the nitrogen to which they are bonded, R^{a} and R^{b} can form a C₃-C₇heterocyclic ring. In one embodiment, the R^{a} and R^{b} groups are each independently optionally substituted as described above.

“Carbocyclic group”, “carbocyclic ring”, or “cycloalkyl” is a saturated or partially unsaturated (i.e., not aromatic) group containing all carbon ring atoms. A carbocyclic group typically contains 1 ring of 3 to 7 carbon atoms or 2 fused rings each containing 3 to 7 carbon atoms. Cycloalkyl substituents may be pendant from a substituted nitrogen or carbon atom, or a substituted carbon atom that may have two substituents can have a cycloalkyl group, which is attached as a spiro group. Examples of carbocyclic rings include cyclohexenyl, cyclohexyl, cyclopentenyl, cyclopentyl, cyclobutenyl, cyclobutyl and cyclopropyl rings. In one embodiment, the carbocyclic ring is optionally substituted as described above. In one embodiment, the cycloalkyl is a partially unsaturated (i.e., not aromatic) group containing all carbon ring atoms. In another embodiment, the cycloalkyl is a saturated group containing all carbon ring atoms. In another embodiment, a carbocyclic ring comprises a caged carbocyclic group. In one embodiment, a carbocyclic ring comprises a bridged carbocyclic group. An example of a caged carbocyclic group is adamantane. An example of a bridged carbocyclic group includes bicyclo[2.2.1]heptane (norbornane). In one embodiment, the caged carbocyclic group is optionally substituted as described above. In one embodiment, the bridged carbocyclic group is optionally substituted as described above.

“Hydroxyalkyl” is an alkyl group as previously described, substituted with at least one hydroxyl substituent.

“Halo” or “halogen” indicates independently any of fluoro, chloro, bromo, and iodo.

“Aryl” indicates aromatic groups containing only carbon in the aromatic ring or rings. In one embodiment, the aryl groups contain 1 to 3 separate or fused rings and is 6 to about 14 or 18 ring atoms, without heteroatoms as ring members. When indicated, such aryl groups may be further substituted with carbon or non-carbon atoms or groups. Such substitution may include fusion to a 4 to 7-membered saturated cyclic group that optionally contains 1 or 2 heteroatoms independently chosen from N, O, B, and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described above. In one embodiment, aryl groups include, for example, dihydroindole, dihydrobenzofuran, isoindoline-1-one and indolin-2-one that can be optionally substituted.

The term “heterocycle,” or “heterocyclic ring” as used herein refers to a saturated or a partially unsaturated (i.e., having one or more double and/or triple bonds within the ring without aromaticity) carbocyclic radical of 3 to about 12, and more typically 3, 5, 6, 7 to 10 ring atoms in which at least one ring atom is a heteroatom selected from nitrogen, oxygen, phosphorus, silicon, boron and sulfur, the remaining ring atoms being C, where one or more ring atoms is optionally substituted independently with one or more substituents described above. A heterocycle may be a monocycle having 3 to 7 ring members (2 to 6 carbon atoms and 1 to 4 heteroatoms selected from N, O, P, and S) or a bicycle having 5 to 10 ring members (4 to 9 carbon atoms and 1 to 6 heteroatoms selected from N, O, P, and S), for example: a bicyclo [4,5], [5,5], [5,6], or [6,6] system. In one embodiment, the only heteroatom is nitrogen. In one embodiment, the only heteroatom is oxygen. In one embodiment, the only heteroatom is sulfur. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. Spiro moieties are also included within the scope of this definition. Examples of a heterocyclic group wherein 1 or 2 ring carbon atoms are substituted with oxo (═O) moieties are pyrimidinonyl and 1,1-dioxo-thiomorpholinyl. The heterocycle groups herein are optionally substituted independently with one or more substituents described herein.

“Heteroaryl” refers to a stable monocyclic, bicyclic, or multicyclic aromatic ring which contains from 1 to 3, or in some embodiments from 1, 2, or 3 heteroatoms selected from N, O, S, B or P with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In one embodiment, the only heteroatom is nitrogen. In one embodiment, the only heteroatom is oxygen. In one embodiment, the only heteroatom is sulfur. Monocyclic heteroaryl groups typically have from 5, 6, or 7 ring atoms. In some embodiments bicyclic heteroaryl groups are 8- to 10-membered heteroaryl groups, that is, groups containing 8 or 10 ring atoms in which one 5, 6, or 7 member aromatic ring is fused to a second aromatic or non-aromatic ring. When the total number of S and O atoms in the heteroaryl group exceeds 1, these heteroatoms are not adjacent to one another. In one embodiment, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl.

“Heterocycloalkyl” is a saturated ring group. It may have, for example, 1, 2, 3, or 4 heteroatoms independently chosen from N, S, and O, with remaining ring atoms being carbon. In a typical embodiment, nitrogen is the heteroatom. Monocyclic heterocycloalkyl groups typically have from 3 to about 8 ring atoms or from 4 to 6 ring atoms. Examples of heterocycloalkyl groups include morpholinyl, piperazinyl, piperidinyl, and pyrrolinyl.

The term “esterase” refers to an enzyme that catalyzes the hydrolysis of an ester. As used herein, the esterase can catalyze the hydrolysis of prostaglandins described herein. In certain instances, the esterase includes an enzyme that can catalyze the hydrolysis of amide bonds of prostaglandins.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

A “pharmaceutical composition” is a composition comprising at least one active agent, such as a compound or salt of Formula I, Formula II, Formula II′, Formula III, Formula IV, Formula IV′, Formula V, Formula VI, Formula VII, Formula VIII, Formula VIII′, Formula IX, or Formula X, and at least one other substance, such as a pharmaceutically acceptable carrier. “Pharmaceutical combinations” are combinations of at least two active agents which may be combined in a single dosage form or provided together in separate dosage forms with instructions that the active agents are to be used together to treat any disorder described herein.

A “pharmaceutically acceptable salt” includes a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salt can be prepared by reacting free acid forms of these compounds with a stoichiometric amount of the appropriate base (such as Na, Ca, Mg, or K hydroxide, carbonate, bicarbonate, or the like), or by reacting a free base form of the compound with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

Additional non-limiting examples of salts include 1-hydroxy-2-naphthoic acid, 2,2-dichloroacetic acid, 2-oxoglutaric acid, 4-acetamidobenzoic acid, 4-aminosalicylic acid, adipic acid, aspartic acid, benzenesulfonic acid, camphoric acid, camphor-10-sulfonic acid, capric acid, caproic acid, caprylic acid, carbonic acid, cinnamic acid, cyclamic acid, dodecylsulfuric acid, ethane-1,2-disulfonic acid, ethanesulfonic acid, formic acid, galactaric acid, gentisic acid, glucoheptonic acid, gluconic acid, glucuronic acid, glutaric acid, glycerophosphoric acid, hippuric acid, isobutyric acid, lactobionic acid, lauric acid, malonic acid, mandelic acid, naphthalene-1,5-disulfonic acid, naphthalene-2-sulfonic acid, nicotinic acid, nitric acid, oleic acid, palmitic acid, pyroglutamic acid, sebacic acid, thiocyanic acid, and undecylenic acid. Lists of additional suitable salts may be found, e.g., in Remington's Pharmaceutical Sciences, 17th ed., Mack Publishing Company, Easton, Pa., p. 1418 (1985).

The term “carrier” refers to a diluent, excipient, or vehicle with which an active compound is provided.

A “patient” or “host” or “subject” is typically a human, however, may be more generally a mammal. In an alternative embodiment it can refer to for example, a cow, sheep, goat, horses, dog, cat, rabbit, rat, mice, fish, bird and the like.

A “prodrug” as used herein, means a compound which when administered to a host in vivo is converted into a parent drug. As used herein, the term “parent drug” means the active form of the compounds that renders the biological effect to treat any of the disorders described herein, or to control or improve the underlying cause or symptoms associated with any physiological or pathological disorder described herein in a host, typically a human. Prodrugs can be used to achieve any desired effect, including to enhance properties of the parent drug or to improve the pharmaceutic or pharmacokinetic properties of the parent. Prodrug strategies exist which provide choices in modulating the conditions for in vivo generation of the parent drug, all of which are deemed included herein. Non-limiting examples of prodrug strategies include covalent attachment of removable groups, or removable portions of groups, for example, but not limited to acylation, phosphorylation, phosphonylation, phosphoramidate derivatives, amidation, reduction, oxidation, esterification, alkylation, other carboxy derivatives, sulfoxy or sulfone derivatives, carbonylation or anhydride, among others. In certain aspects of the present invention, at least one hydrophobic group is covalently bound to the parent drug to slow release of the parent drug in vivo.

A “therapeutically effective amount” of a pharmaceutical composition/combination of this invention means an amount effective, when administered to a patient, to provide a therapeutic benefit such as an amelioration of symptoms of the selected disorder, typically an ocular disorder In certain aspects, the disorder is glaucoma, a disorder mediated by carbonic anhydrase, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD) or diabetic retinopathy.

“y-linolenic acid” is gamma-linolenic acid.

The term “polymer” as used herein includes oligomers.

### II. Detailed Description of the Active Compounds

In certain embodiments, compounds for ocular delivery are provided that are lipophilic monoprodrugs of, for example, Ethacrynic acid, Timolol, Metipranolol, Levobunolol, Carteolol, or Betaxolol, covalently linked to a biodegradable oligomer, as described in more detail herein.

In various embodiments, two biologically active compounds are covalently linked (optionally with a biodegradable linker(s), for example, that includes a linking ester, amide, etc. bond as exemplified throughout this specification in detail, e.g., -“ ”linked to”--) for ocular combination therapy. In some embodiments, the bis-prodrug is in a biodegradable polymeric delivery system, such as a biodegradable microparticle or nanoparticle, for controlled delivery. In one embodiment, ethacrynic acid is covalently linked to a β-blocker (for example, Timolol, Metipranolol, Levobunolol, Carteolol or Betaxolol). In another embodiment, ethacrynic acid is covalently linked to a carbonic anhydrase inhibitor (for example, Brinzolamide or Dorzolamide). In another embodiment, ethacrynic acid is covalently linked to an α-agonist (for example, brimonidine or apraclonidine). In another embodiment, ethacrynic acid is covalently linked to a Rho associated kinase inhibitor (for example Y-27637, AMA0076, AR-13324, RKI-1447, RKI-1313, Wf536, CID 5056270, K-115 or fasudil). In another embodiment, ethacrynic acid is covalently linked to a neuroprotectant DLK inhibitor (for example, Sunitinib, SR8165 axitinib, bosutinib, neratinib, Crizotinib, Tozasertib, lestautinib, foretinib or TAE-684). This invention includes the specific combination of each of the named actives with each other named active in the bis-prodrug, as if each combination were individually described (and is only written like this for efficiency of space).

In yet another embodiment, a β-blocker (for example, Timolol, Metipranolol, Levobunolol, Carteolol or Betaxolol) is covalently linked to a carbonic anhydrase inhibitor (for example, Brinzolamide or Dorzolamide). In another embodiment, a β-blocker (for example, Timolol, Metipranolol, Levobunolol, Carteolol or Betaxolol) is covalently linked to an α-agonist (for example Brimonidine or apraclonidine). In another embodiment, a β-blocker (for example, Timolol, Metipranolol, Levobunolol, Carteolol or Betaxolol) is covalently linked to a Rho associated kinase inhibitor (for example Y-27637, AMA0076, AR-13324, RKI-1447, RKI-1313, Wf536, CID 5056270, K-115 or fasudil). In another embodiment, a β-blocker (for example, Timolol, Metipranolol, Levobunolol, Carteolol or Betaxolol) is covalently linked to a neuroprotectant DLK inhibitor (for example, Sunitinib, SR8165 Axitinib, Bosutinib, Neratinib, Crizotinib, Tozasertib, Lestautinib, Foretinib or TAE-684). In alternative embodiments, a ROCK inhibitor can be selected for these embodiments selected from those disclosed in Pireddu, et. al., Pyridylthiazole-based urease as inhibitors of Rho associated protein kinases (ROCK 1 and 2), Med. Chem. Comm. 2012, 3, 699; Patel, et al., Identification of novel ROCK inhibitors with anti-migratory and anti-invasive activities, Oncogene (2014) 33, 550-555; Patel, et al, RKI-1447 is a potent inhibitor of the Rho-Associated ROCK Kinase with anti-Invasive and Antitumor Activities in Breast Cancer, Cancer Research, online Jul. 30, 2012, 5025-5033). See also U.S. Pat. Nos. 9,221,808 and 9,409,868, herein incorporated in their entirety by reference. Again, this invention includes the specific combination of each of the named actives with each other named active in the bis-prodrug, as if each combination were individually (and is only written like this for efficiency of space).

In specific embodiments, Sunitinib is covalently linked to one of the β-blockers named above. In another embodiment, Sunitinib is covalently linked to ethacrynic acid. Again, this invention includes the specific combination of each of the named actives with each other named active in the bis-prodrug, as if each combination were individually (and is only written like this for efficiency of space).

In other various embodiments, the biologically active compound as described herein for ocular therapy is covalently linked (optionally with a biodegradable linker(s) that include a linking ester, amide, etc. bond as exemplified throughout this specification in detail) to a second same biologically active compound, to create a biodegradable dimer for ocular combination therapy.

The dimer is more lipophilic and thus will enhance the controlled delivery of the active compound over time, in particular in a polymeric delivery system, for example, when administered in a hydrophilic intravitreal fluid of the eye. Biologically active compounds that can be dimerized with a biodegradable linker for use in a biodegradable polymeric composition include, but are not limited to, ethacrynic acid, Timolol, Metipranolol, Levobunolol, Carteolol, or Betaxolol. Methods to dimerize these compounds with a biodegradable linker are exemplified throughout this specification.

According to the present invention, compounds of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, or Formula XIV are provided:

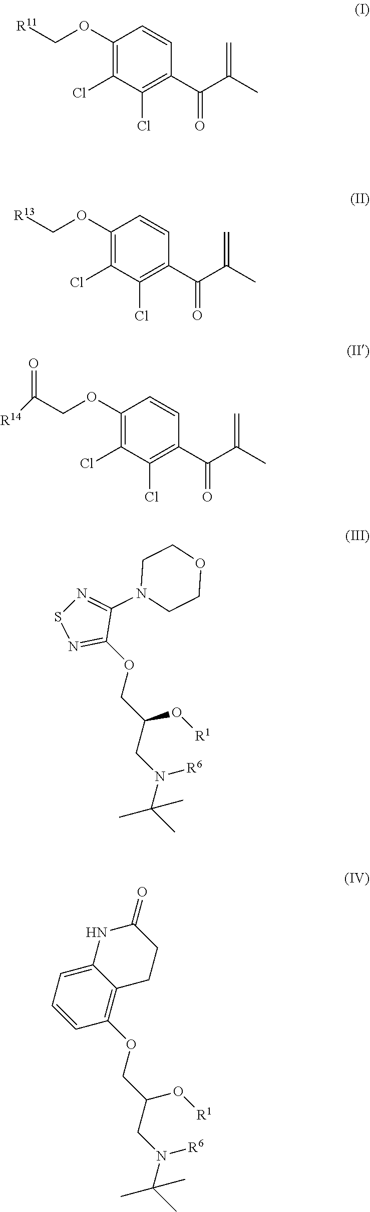

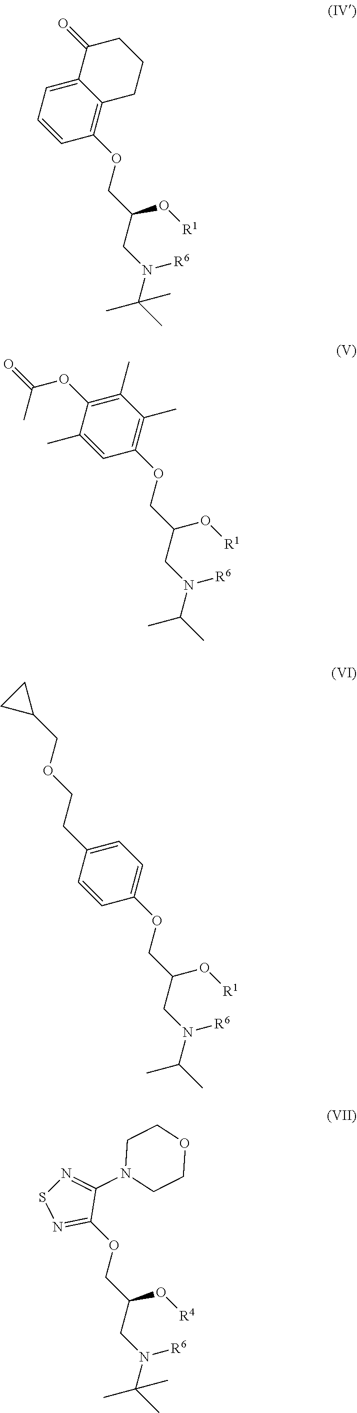

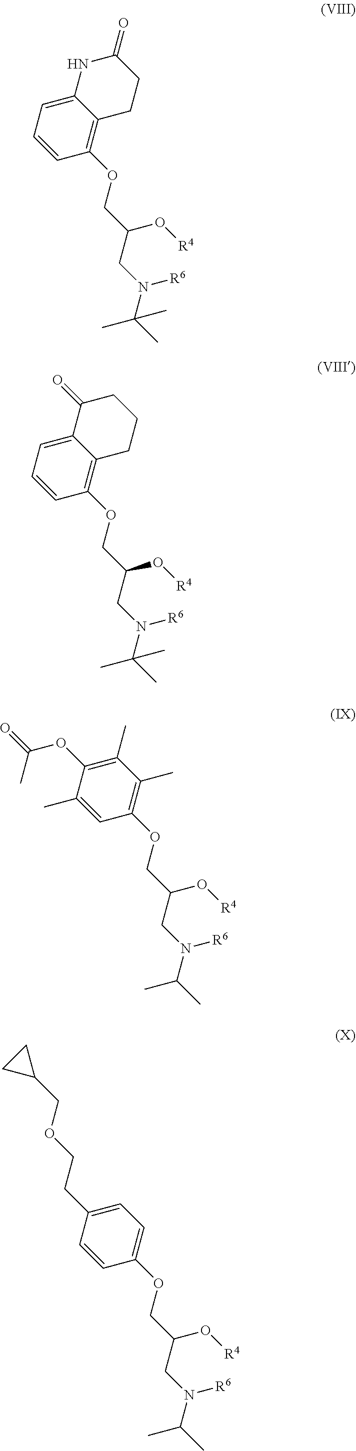

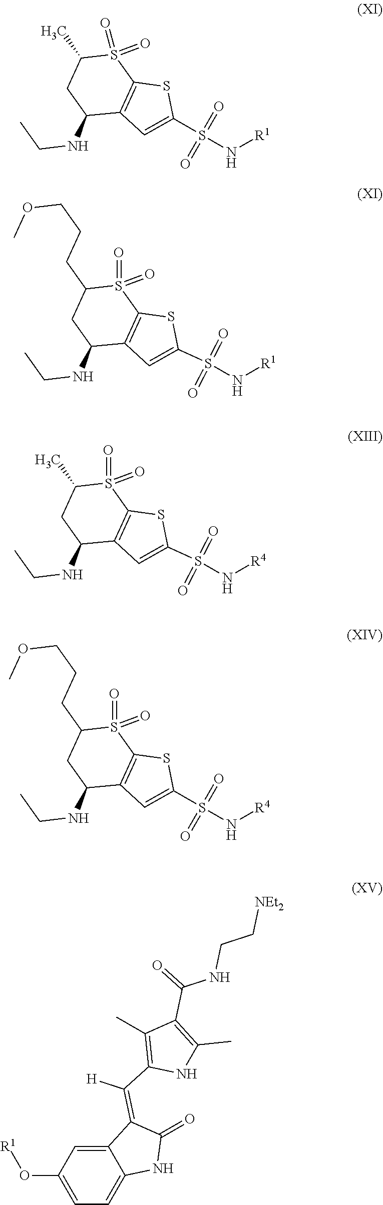

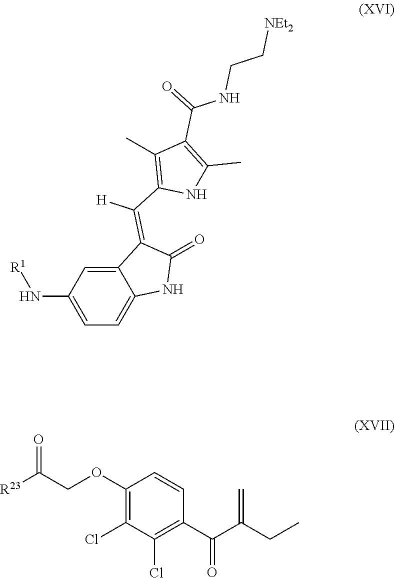

as well as the pharmaceutically acceptable salts and compositions thereof. Formula I can be considered ethacrynic acid covalently bound to a hydrophobic moiety through an ester or amide linkage that may be metabolized in the eye to afford ethacrynic acid. In one embodiment, a compound of Formula I is ethacrynic acid linked to PLA wherein the PLA is 4 or 6 units long. Formula II and Formula II′ can be considered ethacrynic acid covalently bound to a carbonic anhydrase inhibitor, an α-agonist, a Rho associated kinase inhibitor, DLK inhibitor, or a β-blocker through either a direct bond or a connecting fragment bound to both species that may be metabolized in the eye to afford both active species. Formula III can be considered Timolol covalently bound to a hydrophobic moiety through an ester linkage that may be metabolized in the eye to afford Timolol. Formula IV can be considered Carteolol covalently bound to a hydrophobic moiety through an ester linkage that may be metabolized in the eye to afford Carteolol. Formula IV′ can be considered Levobunolol covalently bound to a hydrophobic moiety through an ester linkage that may be metabolized in the eye to afford Levobunolol. Formula V can be considered Metipranolol covalently bound to a hydrophobic moiety through an ester linkage that may be metabolized in the eye to afford Metipranolol. Formula VI can be considered Betaxolol covalently bound to a hydrophobic moiety through an ester linkage that may be metabolized in the eye to afford Betaxolol. Formula VII can be considered Timolol covalently bound to a carbonic anhydrase inhibitor, an α-agonist, a Rho associated kinase inhibitor, DLK inhibitor, a β-blocker, or ethacrynic acid through either a direct bond or a connecting fragment bound to both species that may be metabolized in the eye to afford both active species. Formula VIII can be considered Carteolol covalently bound to a carbonic anhydrase inhibitor, an α-agonist, a Rho associated kinase inhibitor, DLK inhibitor, a β-blocker, or ethacrynic acid through either a direct bond or a connecting fragment bound to both species that may be metabolized in the eye to afford both active species. Formula VII′ can be considered Levobunolol covalently bound to a carbonic anhydrase inhibitor, an α-agonist, a Rho associated kinase inhibitor, DLK inhibitor, a β-blocker, or ethacrynic acid through either a direct bond or a connecting fragment bound to both species that may be metabolized in the eye to afford both active species. Formula IX can be considered Metipranolol covalently bound to a carbonic anhydrase inhibitor, an α-agonist, a Rho associated kinase inhibitor, DLK inhibitor, a β-blocker, or ethacrynic acid through either a direct bond or a connecting fragment bound to both species that may be metabolized in the eye to afford both active species.

Formula X can be considered Betaxolol covalently bound to a carbonic anhydrase inhibitor, an α-agonist, a Rho associated kinase inhibitor, DLK inhibitor, a β-blocker, or ethacrynic acid through either a direct bond or a connecting fragment bound to both species that may be metabolized in the eye to afford both active species. Formula XI can be considered Dorzolamide covalently bound to a hydrophobic moiety through an ester or amide linkage that may be metabolized in the eye to afford Dorzolamide. Formula XII can be considered Brinzolamide covalently bound to a hydrophobic moiety through an ester or amide linkage that may be metabolized in the eye to afford Brinzolamide. Formula XIII can be considered Dorzolamide covalently bound to a carbonic anhydrase inhibitor, an α-agonist, a Rho associated kinase inhibitor, DLK inhibitor, a β-blocker, or ethacrynic acid through either a direct bond or a connecting fragment bound to both species that may be metabolized in the eye to afford both active species. Formula XIV can be considered Brinzolamide covalently bound to a carbonic anhydrase inhibitor, an α-agonist, a Rho associated kinase inhibitor, DLK inhibitor, a β-blocker, or ethacrynic acid through either a direct bond or a connecting fragment bound to both species that may be metabolized in the eye to afford both active species. Formula XV and Formula XVI can be considered Sunitinib covalently bound to a hydrophobic moiety through an ester or amide linkage that may be metabolized in the eye to afford Sunitinib. Formula XVII can be considered ethacrynic acid covalently bound to ethacrynic acid through a connecting fragment bound to both species that may be metabolized in the eye to afford both active species. In one embodiment, the compound is a treatment for glaucoma, and therefore can be used as an effective amount to treat a host in need of glaucoma treatment. In another embodiment, the compound acts through a mechanism other than those associated with glaucoma to treat a disorder described herein in a host, typically a human.

The compounds, as described herein, may include, for example, prodrugs, which are hydrolysable to form the diuretic ethacrynic acid. Thus, when a compound of Formula I, Formula II, Formula II′, or Formula XVII is administered to a mammalian subject, typically a human, the ester or amide modification may be cleaved to release ethacrynic acid.

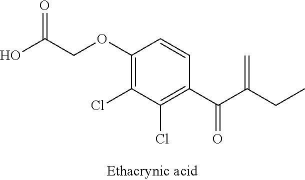 [SMILES: C=C(CC)C(=O)c1ccc(OCC(=O)O)c(Cl)c1Cl]

The compounds, as described herein, may include, for example, prodrugs, which are hydrolysable to release the active β-blocker. Thus when a compound of Formula III, Formula IV, Formula IV′, Formula V, Formula VI, Formula VII, Formula VIII, Formula VIII′, Formula IX, Formula X, or Formula XVII is administered to a mammalian subject, typically a human, the ester bond may be cleaved to release Timolol, Levobunolol, Carteolol, Metipranolol, and Betaxolol.

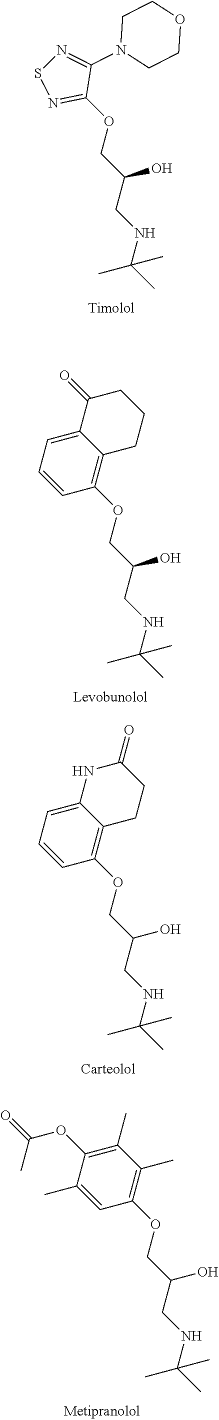 [SMILES: CC(C)(C)NC[C@H](O)COc1nsnc1N1CCOCC1 ; CC(C)(C)NC[C@H](O)COc1cccc2c1CCCC2=O ; CC(C)(C)NCC(O)COc1cccc2c1CCC(=O)N2 ; CC(=O)Oc1c(C)cc(OCC(O)CNC(C)(C)C)c(C)c1C]

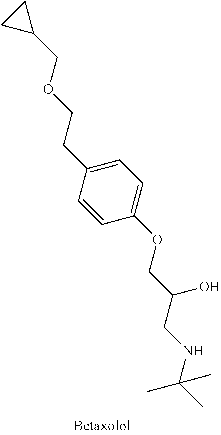 [SMILES: CC(C)(C)NCC(O)COc1ccc(CCOCC2CC2)cc1]

The compounds, as described herein, may include, for example, prodrugs, which are hydrolysable to form the active carboxylic acid compound. Thus, when a compound of Formula II, Formula II′, Formula VII, Formula VIII, Formula VIII′, Formula IX, Formula X, Formula XIII, or Formula XIV is administered to a mammalian subject, typically a human, the amide or ester modifications may be cleaved to release the parent free acid compound:

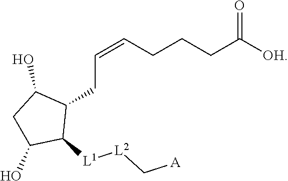

The compounds, as described herein, may include, for example, prodrugs, which are hydrolysable to form the active imidazole compound. Thus when a compound of Formula II, Formula II′, Formula VII, Formula VIII, Formula VIII′, Formula IX, Formula X, Formula XIII, or Formula XIV is administered to a mammalian subject, typically a human, the amide modifications may be cleaved to release Brimonidine.

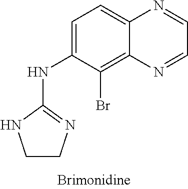 [SMILES: Brc1c(NC2=NCCN2)ccc2nccnc12]

The compounds, as described herein, may include, for example, prodrugs, which are hydrolysable to form the active sulfonamide compound. Thus when a compound of Formula II, Formula II′, Formula VII, Formula VIII, Formula VIII′, Formula IX, Formula X, Formula XIII, or Formula XIV is administered to a mammalian subject, typically a human, the amide modifications may be cleaved to release Brinzolamide, Dorzolamide, Acetazolamide, or Methazolamide.

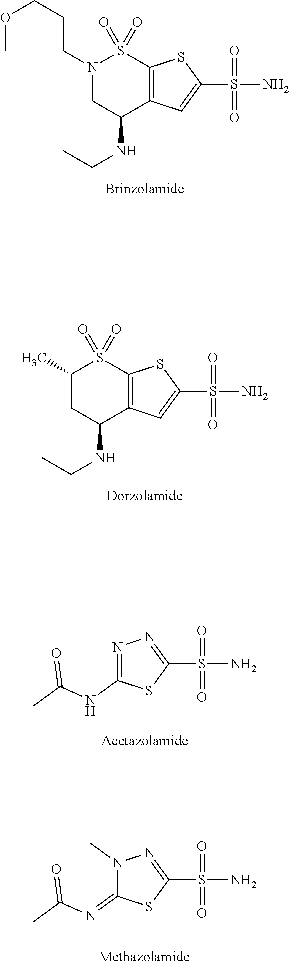 [SMILES: CCN[C@H]1CN(CCCOC)S(=O)(=O)c2sc(S(N)(=O)=O)cc21 ; CCN[C@H]1C[C@H](C)S(=O)(=O)c2sc(S(N)(=O)=O)cc21 ; CC(=O)Nc1nnc(S(N)(=O)=O)s1 ; CC(=O)/N=c1/sc(S(N)(=O)=O)nn1C]

The compounds, as described herein, may include, for example, prodrugs, which are hydrolysable to form the active Sunitinib derivative and an active carboxylic acid or an active sulfonamide compound. Thus when a compound of Formula II, Formula II′, Formula VII, Formula VIII, Formula VIII′, Formula IX, Formula X, Formula XIII, Formula XIV, Formula XV, or Formula XVI is administered to a mammalian subject, typically a human, the prodrug may be cleaved to release the parent Sunitinib derivative. The active Sunitinib derivative is a phenol compound that has been demonstrated in the literature to be an active RTKI (Kuchar, M., et al. (2012). “Radioiodinated Sunitinib as a potential radiotracer for imaging angiogenesis-radiosynthesis and first radiopharmacological evaluation of 5-[125I]Iodo-Sunitinib.” Bioorg Med Chem Lett 22(8): 2850-2855. Formulations of Sunitinib for the treatment of ocular disorders and glaucoma have been described in WO2016/100392 and WO2016/100380, respectively.

The compounds, as described herein, may include, for example, prodrugs, which are hydrolysable to release the active DLK inhibitor. Thus when a compound of Formula II, Formula II′, Formula VII, Formula VIII, Formula VIII′, Formula IX, Formula X, Formula XIII, or Formula XIV is administered to a mammalian subject, typically a human, the amide bond may be cleaved to release Crizotinib, KW-2449, a piperidino DLK inhibitor, or a Tozasertib derivative respectively.

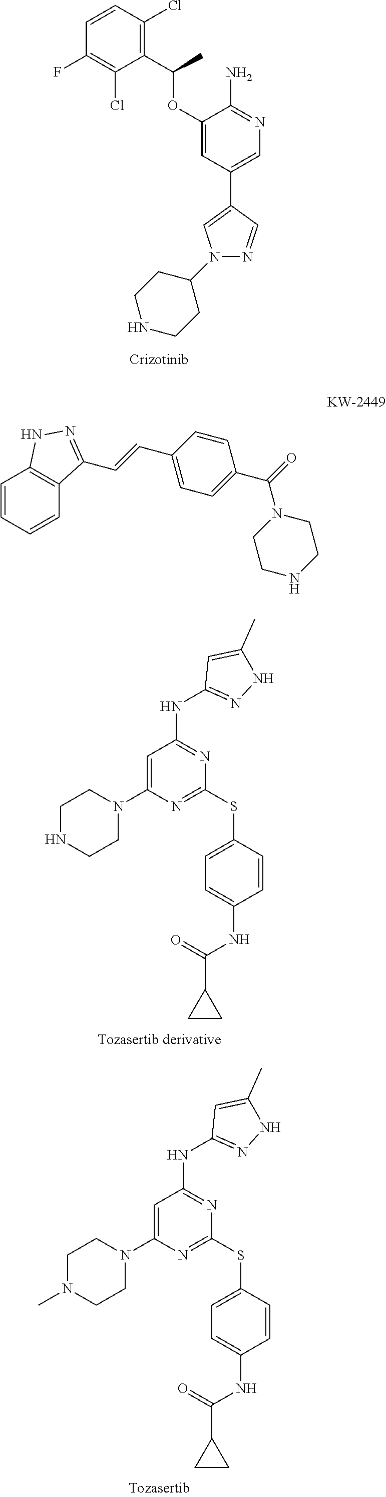 [SMILES: C[C@@H](Oc1cc(-c2cnn(C3CCNCC3)c2)cnc1N)c1c(Cl)ccc(F)c1Cl ; O=C(c1ccc(/C=C/c2n[nH]c3ccccc23)cc1)N1CCNCC1 ; Cc1cc(Nc2cc(N3CCNCC3)nc(Sc3ccc(NC(=O)C4CC4)cc3)n2)n[nH]1 ; Cc1cc(Nc2cc(N3CCN(C)CC3)nc(Sc3ccc(NC(=O)C4CC4)cc3)n2)n[nH]1]

The amides and esters of commercial prostaglandins are believed to act as prodrugs in the eye, in that the ester or amide form, is hydrolyzed by an endogenous ocular enzyme, releasing the parent compound as a free acid which is the active pharmacologic agent. However, this also releases a potentially toxic and potentially irritating small aliphatic alcohol, for example, isobutanol into the eye. While effective in reducing intraocular pressure, most drugs currently in use, including latanoprost, bimatoprost, travoprost, may cause a significant level of eye irritation in some patients.

In addition to the foregoing, the isopropyl esters of prostaglandins, for example, latanoprost and fluprostenol, are highly viscous, glassy oils, which can be difficult to handle and to formulate into ophthalmic solutions. In addition, these compounds can be prone to the retention of potentially toxic process solvents. The higher alkyl esters or amides of prostaglandins can be easier to handle and may not release as irritating of an alcohol or amine upon hydrolysis.

In addition to the irritation caused by the prostaglandins themselves, and particularly the naturally-occurring and synthetic prostaglandins of the type presently on the market, the preservatives typically used in ophthalmic solutions are known to potentially irritate a percentage of the population. Thus, despite the fact that the prostaglandins represent an important class of potent therapeutic agents for the treatment of glaucoma, the unwanted side effects of these drugs, particularly ocular irritation and inflammation, may limit patient use and can be related to patient withdrawal from the use of these drugs. The higher alkyl esters and amides of prostaglandins as disclosed herein, can be less irritating to patients yet therapeutically effective.

Another disclosed invention is a method for the controlled administration of Timolol to a patient in need thereof, comprising administering a prodrug of Timolol in a microparticle in vivo or in vitro, wherein the Timolol prodrug containing microparticle exhibits in vitro drug release kinetics in an aqueous solution at a pH between 6-8 at body temperature of a substantially consistent release of at least 60% Timolol itself by molar ratio to the prodrug of Timolol or an intermediate metabolite thereof (i.e., a breakdown product of the prodrug of Timolol on the way to the parent Timolol) over at least 100 days. In certain embodiments, the aqueous solution is a buffered solution, for example, a phosphate buffered solution. In other embodiments, there can be a substantially consistent release of at least 70%, 75%, 80%, 85% or 90% or more of the parent Timolol itself by molar ratio to the prodrug of Timolol or an intermediate metabolite thereof over at least 100, 110 or even 120 or more days. The term “total drug” as used herein refers to the Timolol prodrug and intermediate metabolites together which ultimately break down to the parent Timolol. This can occur when the prodrug of Timolol has multiple labile bonds that can be metabolically or hydrolytically cleaved, such as ester and/or amide bonds. Examples of Timolol prodrugs are those, for example, with glycolic acid and/or lactic acid moieties. In some embodiments, the prodrug of Timolol is a Timolol-N-glycolic acid-containing prodrug, a Timolol-O-glycolic acid-containing prodrug, Timolol-N,O-bis-glycolic acid-containing prodrug, Timolol-N,O-bis-glycolic acid-O-acetyl, Timolol-N,O-bis-glycolic acid-O-(PLA)₄₋acetyl, or for example wherein the prodrug is an ester-containing prodrug or an amide-containing prodrug.

It has been surprisingly discovered that selected Timolol prodrug microparticles as described herein exhibit substantially linear release rates over at least 2, 3 or 4 months in vitro where the correlation between parent drug release and total drug (i.e., Timolol prodrug and intermediate metabolic breakdown products of the prodrug on the way to the parent Timolol) release is high. In other words, the microparticle with Timolol prodrug is capable of consistently delivering a high molar percentage of the active compound, Timolol, which is advantageous for therapy.

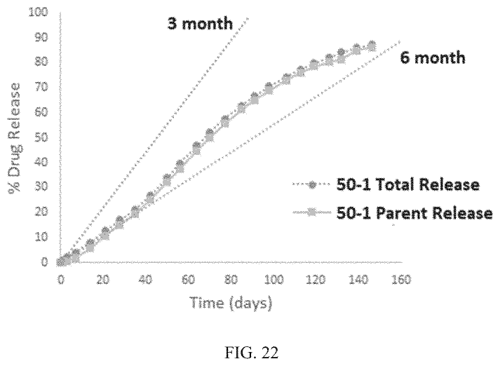

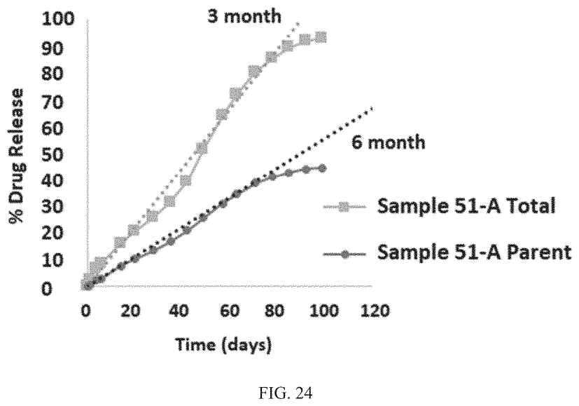

In a non-limiting embodiment, as discussed in Example 14 and shown in FIG. 22, Compound 50 is an example of a Timolol prodrug with such properties. This is unexpected because other Timolol prodrugs (for example shown in Example 14) with similar chemical structures do not exhibit substantially linear kinetics at least 2, 3 or 4 month release in combination with a high degree of consistent parent Timolol release. For example, Compound 51, which only differs from Compound 50 in that Compound 51 has two PLA unit on the polymeric branches, while Compound 50 has four, does not exhibit linear 4-month release. Compound 51 also does not exhibit kinetics where the correlation between total drug release and parent Timolol is high (FIG. 24).

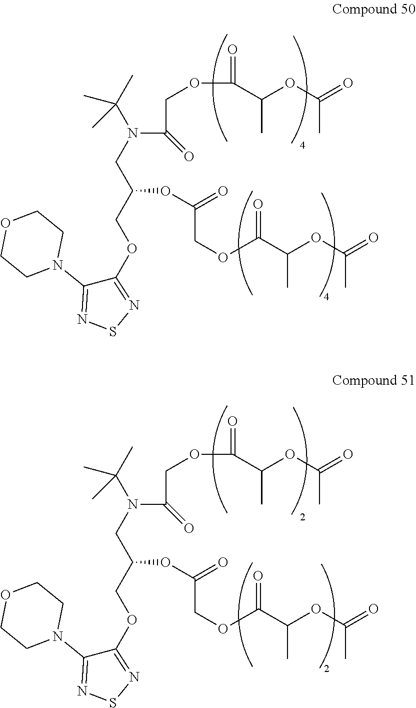

In certain embodiments, the prodrug is delivered in a microparticle or nanoparticle that is a blend of two polymers, for example (i) a PLGA polymer or PLA polymer as described herein and (ii) a PLGA-PEG or PLA-PEG copolymer. In another embodiment, the microparticle or nanoparticle is a blend of three polymers, such as, for example, (i) a PLGA polymer; (ii) a PLA polymer; and, (iii) a copolymer of PLGA-PEG or PLA-PEG. In an additional embodiment, the microparticle or nanoparticle is a blend of (i) a PLA polymer; (ii) a PLGA polymer; (iii) a PLGA polymer that has a different ratio of lactide and glycolide monomers than the PLGA in (ii); and, (iv) a PLGA-PEG or PLA-PEG copolymer. Any ratio of lactide and glycolide in the PLGA can be used that achieves the desired therapeutic effect. In certain illustrative non-limiting embodiments, the ratio of PLA to PLGA by weight in a polymer blend as described is 77/22, 69/30, 49/50, 54/45, 59/40, 64/35, 69/30, 74/25, 79/20, 84/15, 89/10, 94/5, or 99/1. In certain embodiments, the prodrug is Compound 50.

In one embodiment, the in vitro drug release kinetics are measured in an aqueous solution at a pH between 4-10. In one embodiment, the pH is between 4 and 8. In one embodiment, the pH is between 6 and 8, or between about 6 and 7. In one embodiment, the pH is between 8 and 10. In one embodiment, the in vitro release kinetics are measured at body temperature, i.e, between 35° C. and 40° C., for example, about 36, 37, 38 or 39° C. In one embodiment, the in vitro release kinetics are measured at about 37° C. In one embodiment, the aqueous solution is buffered saline. In one embodiment, the aqueous solution is phosphate buffered saline.

In one embodiment, the in vitro release of the parent Timolol and/or the prodrug of Timolol over 100 days from the microparticle under the conditions described herein is substantially linear. In one embodiment, the microparticle exhibits in vitro drug release kinetics of a substantially consistent release of at least 60%, at least 65%, at least 70%, at least 75%, at least 80%, at least 85%, at least 90%, or at least 95% Timolol by molar ratio to the total drug (i.e., the Timolol prodrug and intermediate metabolites together which ultimately break down to the parent Timolol) over at least 100 days. In one embodiment, the microparticle exhibits in vitro drug release kinetics of a substantially consistent release of at least 60% Timolol by molar ratio of total drug over at least 100 days, at least 110 days, at least 120 days, at least 125 days, at least 130 days, at least 135 days, or at least 140 days.

In certain embodiments, the Timolol prodrug is delivered in a microparticle that is a blend of two polymers, for example (i) a PLGA polymer or PLA polymer as described herein and (ii) a PLGA-PEG or PLA-PEG copolymer. In another embodiment, the microparticle or nanoparticle is a blend of three polymers, such as, for example, (i) a PLGA polymer; (ii) a PLA polymer; and, (iii) a copolymer of PLGA-PEG or PLA-PEG. In an additional embodiment, the microparticle or nanoparticle is a blend of (i) a PLA polymer; (ii) a PLGA polymer; (iii) a PLGA polymer that has a different ratio of lactide and glycolide monomers than the PLGA in (ii); and, (iv) a PLGA-PEG or PLA-PEG copolymer. Any ratio of lactide and glycolide in the PLGA can be used that achieves the desired therapeutic effect. In certain illustrative non-limiting embodiments, the ratio of PLA to PLGA by weight in a polymer blend as described is 77/22, 69/30, 49/50, 54/45, 59/40, 64/35, 69/30, 74/25, 79/20, 84/15, 89/10, 94/5, or 99/1. In certain embodiments, a blend of three polymers that has (i) PLA, (ii) PLGA, and (iii) PLGA with a different ratio of lactide and glycolide monomers than PLGA in (ii) wherein the ratio by weight is 74/20/5 by weight, 69/20/10 by weight, 69/25/5 by weight, or 64/20/15 by weight. In certain embodiments, the PLGA in (ii) has a ratio of lactide to glycolide of 85/15, 75/25, or 50/50. In certain embodiments the PLGA in (iii) has a ratio of lactide to glycolide of 85/15, 75/25, or 50/50.

In certain aspects, the Timolol prodrug may be delivered in a blend of PLGA or PLA and PEG-PLGA, including but not limited to (i) PLGA+approximately by weight 1% PEG-PLGA or (ii) PLA+approximately by weight 1% PEG-PLGA. In certain aspects, the Timolol prodrug may be delivered in a blend of (iii) PLGA/PLA+approximately by weight 1% PEG-PLGA. In certain embodiments, the blend of PLA, PLGA, or PLA/PGA with PLGA-PEG contains approximately from about 0.5% to about 10% by weight of a PEG-PLGA, from about 0.5% to about 5% by weight of a PEG-PLGA, from about 0.5% to about 4% by weight of a PEG-PLGA, from about 0.5% to about 3% by weight of a PEG-PLGA, from about 1.0% to about 3.0% by weight of a PEG-PLGA, from about 0.1% to about 10% of a PEG-PLGA, from about 0.1% to about 5% of a PEG-PLGA, from about 0.1% to about 1% PEG-PLGA, or from about 0.1% to about 2% PEG-PLGA.

In certain non-limiting embodiments, the ratio by weight percent of PLGA to PEG-PLGA in a two polymer blend as described is about 40/1, 45/1, 50/1, 55/1, 60/1, 65/1, 70/1, 75/1, 80/1, 85/1, 90/1, 95/1, 96/1, 97/1, 98/1, 99/1. The PLGA can be acid or ester capped. In non-limiting aspects, the Timolol prodrug can be delivered in a two polymer blend of PLGA75:25 4A+approximately 1% PEG-PLGA50:50; PLGA85:15 5A+approximately 1% PEG-PLGA5050; PLGA75:25 6E+approximately 1% PEG-PLGA50:50; or, PLGA50:50 2A+approximately 1% PEG-PLGA50:50.

In certain non-limiting embodiments, the ratio by weight percent of PLA/PLGA-PEG in a polymer blend as described is about 40/1, 45/1, 50/1, 55/1, 60/1, 65/1, 70/1, 75/1, 80/1, 85/1, 90/1, 95/1, 96/1, 97/1, 98/1, 99/1. The PLA can be acid capped or ester capped. In certain aspects, the PLA is PLA 4.5A. In non-limiting aspects, the Timolol prodrug drug is delivered in a blend of PLA 4.5A+1% PEG-PLGA. The PEG segment of the PEG-PLGA may have, for example, in non-limiting embodiments, a molecular weight of at least about 1 kDa, 2 kDa, 3 kDa, 4 kDa, 5 kDa, 6 kDa, 7 kDa, 8 kDa, 9 kDa, or 10 kDa, and typically not greater than 10 kDa, 15 kDa, 20 kDa, or 50 kDa, or in some embodiments, 6 kDa, 7 kDa, 8 kDa, or 9 kDa. In certain embodiment, the PEG segment of the PEG-PLGA has a molecular weight between about 3 kDa and about 7 kDa or between about 2 kDa and about 7 kDa. Non-limiting examples of the PLGA segment of the PEG-PLGA is PLGA50:50, PLGA75:25, or PLGA85:15. In one embodiment, the PEG-PLGA segment is PEG (5 kDa)-PLGA50:50.

When the Timolol prodrug is delivered in a blend of PLGA+PEG-PLGA, any ratio of lactide and glycolide in the PLGA or the PLGA-PEG can be used that achieves the desired therapeutic effect. Non-limiting illustrative embodiments of the ratio of lactide/glycolide in the PLGA or PLGA-PEG are about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In one embodiment, the PLGA is a block co-polymer, for example, diblock, triblock, multiblock, or star-shaped block. In one embodiment, the PLGA is a random co-polymer. In certain aspects, the PLGA is PLGA75:25 4A; PLGA85:15 5A; PLGA75:25 6E; or, PLGA50:50 2A.

In specific embodiments, the polymeric microparticle comprises 64% PLA, 20% PLGA 8515, 15% PLGA and 1% PLGA-PEG. In specific embodiments, the polymeric microparticle comprises 77% PLA, 22% PLGA 8515, and 1% PLGA-PEG. In specific embodiments, the polymeric microparticle comprises 99% PLA and 1% PLGA-PEG.

In one embodiment, the polymeric microparticles have a mean diameter between 10 μm and 60 μm. In one embodiment, the polymeric microparticles have a mean diameter between 20 μm and 50 μm. In one embodiment, the polymeric microparticles have a mean diameter between 30 μm and 40 μm. In one embodiment, the polymeric microparticles have a mean diameter between 25 μm and 35 μm. In one embodiment, the polymeric microparticles have a mean diameter between 20 μm and 40 μm.

In one embodiment, the release rate is assayed at least every 3 days, at least every 5 days, at least every 7 days, or at least every 10 days over the 100 days. In one embodiment, the release rate is assayed every other day. In a preferred embodiment, the release rate is assayed every 7 days.

In one embodiment, the prodrug of Timolol in the polymeric microparticle is Compound 50 or Compound 52:

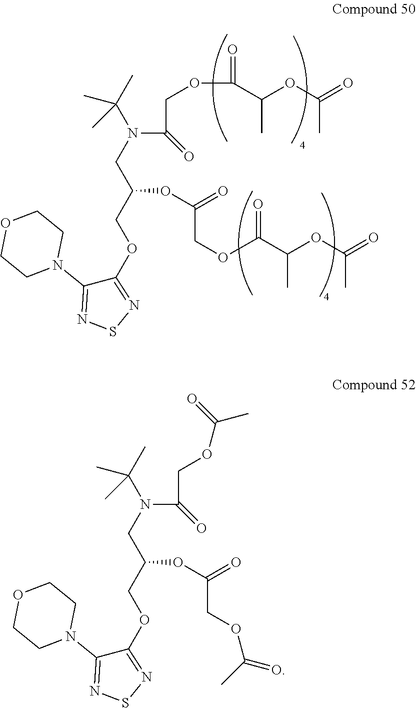

In an alternative embodiment, the prodrug of Timolol in the polymeric microparticle is Compound 51, Compound 53, Compound 54, Compound 55, or Compound 56.

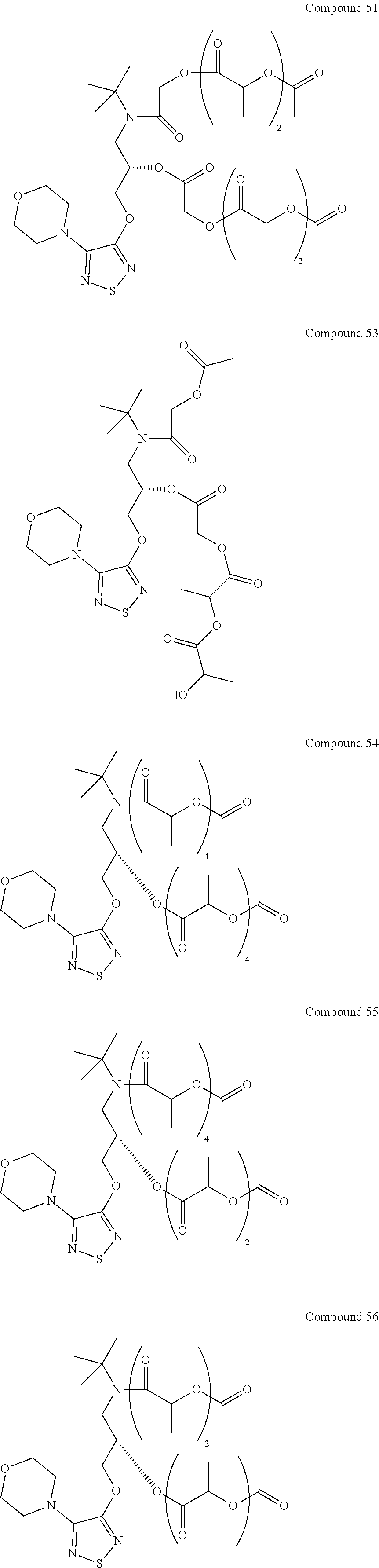

### III. Pharmaceutical Preparations

One embodiment provides compositions including the compounds described herein. In certain embodiments, the composition includes a compound of Formula I, Formula II, Formula II′, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, or Formula XVI in combination with a pharmaceutically acceptable carrier, excipient or diluent. In one embodiment, the composition is a pharmaceutical composition for treating an eye disorder or eye disease. Non-limiting exemplary eye disorder or disease treatable with the composition includes age related macular degeneration, alkaline erosive keratoconjunctivitis, allergic conjunctivitis, allergic keratitis, anterior uveitis, Behcet's disease, blepharitis, blood-aqueous barrier disruption, chorioiditis, chronic uveitis, conjunctivitis, contact lens-induced keratoconjunctivitis, corneal abrasion, corneal trauma, corneal ulcer, crystalline retinopathy, cystoid macular edema, dacryocystitis, diabetic keratophathy, diabetic macular edema, diabetic retinopathy, dry eye disease, dry age-related macular degeneration, geographic atrophy, eosinophilic granuloma, episcleritis, exudative macular edema, Fuchs' Dystrophy, giant cell arteritis, giant papillary conjunctivitis, glaucoma, glaucoma surgery failure, graft rejection, herpes zoster, inflammation after cataract surgery, iridocorneal endothelial syndrome, iritis, keratoconjunctiva sicca, keratoconjunctival inflammatory disease, keratoconus, lattice dystrophy, map-dot-fingerprint dystrophy, necrotic keratitis, neovascular diseases involving the retina, uveal tract or cornea, for example, neovascular glaucoma, corneal neovascularization, neovascularization resulting following a combined vitrectomy and lensectomy, neovascularization of the optic nerve, and neovascularization due to penetration of the eye or contusive ocular injury, neuroparalytic keratitis, non-infectious uveitisocular herpes, ocular lymphoma, ocular rosacea, ophthalmic infections, ophthalmic pemphigoid, optic neuritis, panuveitis, papillitis, pars planitis, persistent macular edema, phacoanaphylaxis, posterior uveitis, post-operative inflammation, proliferative diabetic retinopathy, proliferative sickle cell retinopathy, proliferative vitreoretinopathy, retinal artery occlusion, retinal detachment, retinal vein occlusion, retinitis pigmentosa, retinopathy of prematurity, rubeosis iritis, scleritis, Stevens-Johnson syndrome, sympathetic ophthalmia, temporal arteritis, thyroid associated ophthalmopathy, uveitis, vernal conjunctivitis, vitamin A insufficiency-induced keratomalacia, vitreitis, and wet age-related macular degeneration.

Compounds of Formula I, Formula II, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, or Formula XVI or its salt, can be delivered by any method known for ocular delivery. Methods include but are not limited to conventional (solution, suspension, emulsion, ointment, inserts and gels); vesicular (liposomes, niosomes, discomes and pharmacosomes), particulates (microparticles and nanoparticles), advanced materials (scleral plugs, gene delivery, siRNA and stem cells); and controlled release systems (implants, hydrogels, dendrimers, iontoporesis, collagen shields, polymeric solutions, therapeutic contact lenses, cyclodextrin carriers, microneedles and microemulsions).

In certain aspects, a delivery system is used including but not limited to the following; i) a degradable polymeric composition; ii) a non-degradable polymeric composition; (iii) a gel, including a hydrogel; (iv) a depot; (v) a particle containing a core; vi) a surface-coated particle; vii) a multi-layered polymeric or non-polymeric or mixed polymeric and non-polymeric particle; viii) a polymer blend and/or ix) a particle with a coating on the surface of the particle. The polymers can include, for example, hydrophobic regions. In some embodiments, at least about 30, 40 or 50% of the hydrophobic regions in the coating molecules have a molecular mass of least about 2 kDa.

In some embodiments, at least about 30, 40 or 50% of the hydrophobic regions in the coating molecules have a molecular mass of least about 3 kDa. In some embodiments, at least about 30, 40 or 50% of the hydrophobic regions in the coating molecules have a molecular mass of least about 4 kDa. In some embodiments, at least about 30, 40 or 50% of the hydrophobic regions in the coating molecules have a molecular mass of least about 5 kDa. In certain embodiments, up to 5, 10, 20, 30, 40, 50, 60, 70, 80, 90 or even 95% or more of a copolymer or polymer blend consists of a hydrophobic polymer or polymer segment. In some embodiments, the polymeric material includes up to 2, 3, 4, 5, 6, 7, 8, 9, or 10% or more hydrophilic polymer. In one embodiment, the hydrophobic polymer is a polymer or copolymer of lactic acid or glycolic acid, including PLGA.

In one embodiment, the hydrophilic polymer is polyethylene glycol. In certain embodiments a triblock polymer such as a Pluronic is used. The drug delivery system can be suitable for administration into an eye compartment of a patient, for example by injection into the eye compartment. In some embodiments, the core includes a biocompatible polymer. As used herein, unless the context indicates otherwise, “drug delivery system”, “carrier”, and “particle composition” can all be used interchangeably. In a typical embodiment this delivery system is used for ocular delivery.

The particle in the drug delivery system can be of any desired size that achieves the desired result. The appropriate particle size can vary based on the method of administration, the eye compartment to which the drug delivery system is administered, the therapeutic agent employed and the eye disorder to be treated, as will be appreciated by a person of skill in the art in light of the teachings disclosed herein. For example, in some embodiments the particle has a diameter of at least about 1 nm, or from about 1 nm to about 50 microns. The particle can also have a diameter of, for example, from about 1 nm to about 15, 16, 17, 18, 19, 2, 21, 22, 23, 24, 25, 26, 27, 28, 29 or 30 microns; or from about 10 nm to about less than 30, 35, 40, 45 or 50 microns; or from about 10 nm to about less than 28 microns; from about 1 nm to about 5 microns; less than about 1 nm; from about 1 nm to about 3 microns; or from about 1 nm to about 1000 nm; or from about 25 nm to about 75 nm; or from about 20 nm to less than or about 30 nm; or from about 100 nm to about 300 nm. In some embodiments, the average particle size can be about up to 1 nm, 10 nm, 25 nm, 30 nm, 50 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, or more. In some embodiments, the particle size can be about 100 microns or less, about 50 microns or less, about 30 microns or less, about 10 microns or less, about 6 microns or less, about 5 microns or less, about 3 microns or less, about 1000 nm or less, about 800 nm or less, about 600 nm or less, about 500 nm or less, about 400 nm or less, about 300 nm or less, about 200 nm or less, or about 100 nm or less. In some embodiments, the particle can be a nanoparticle or a microparticle. In some embodiments, the drug delivery system can contain a plurality of sizes particles. The particles can be all nanoparticles, all microparticles, or a combination of nanoparticles and microparticles.

When delivering the active material in a polymeric delivery composition, the active material can be distributed homogeneously, heterogeneously, or in one or more polymeric layers of a multi-layered composition, including in a polymer coated core or a bare uncoated core.

In some embodiments, the drug delivery system includes a particle comprising a core. In some embodiments a compound of Formula I, Formula II, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, or Formula XVI can be present in the core in a suitable amount, e.g., at least about 1% weight (wt), at least about 5% wt, at least about 10% wt, at least about 20% wt, at least about 30% wt, at least about 40% wt, at least about 50% wt, at least about 60% wt, at least about 70% wt, at least about 80% wt, at least about 85% wt, at least about 90% wt, at least about 95% wt, or at least about 99% wt of the core. In one embodiment, the core is formed of 100% wt of the pharmaceutical agent. In some cases, the pharmaceutical agent may be present in the core at less than or equal to about 100% wt, less than or equal to about 90% wt, less than or equal to about 80% wt, less than or equal to about 70% wt, less than or equal to about 60% wt, less than or equal to about 50% wt, less than or equal to about 40% wt, less than or equal to about 30% wt, less than or equal to about 20% wt, less than or equal to about 10% wt, less than or equal to about 5% wt, less than or equal to about 2% wt, or less than or equal to about 1% wt. Combinations of the above-referenced ranges are also possible (e.g., present in an amount of at least about 80% wt and less than or equal to about 100% wt). Other ranges are also possible.

In embodiments in which the core particles comprise relatively high amounts of a pharmaceutical agent (e.g., at least about 50% wt of the core particle), the core particles generally have an increased loading of the pharmaceutical agent compared to particles that are formed by encapsulating agents into polymeric carriers. This is an advantage for drug delivery applications, since higher drug loadings mean that fewer numbers of particles may be needed to achieve a desired effect compared to the use of particles containing polymeric carriers.

In some embodiments, the core is formed of a solid material having a relatively low aqueous solubility (i.e., a solubility in water, optionally with one or more buffers), and/or a relatively low solubility in the solution in which the solid material is being coated with a surface-altering agent. For example, the solid material may have an aqueous solubility (or a solubility in a coating solution) of less than or equal to about 5 mg/mL, less than or equal to about 2 mg/mL, less than or equal to about 1 mg/mL, less than or equal to about 0.5 mg/mL, less than or equal to about 0.1 mg/mL, less than or equal to about 0.05 mg/mL, less than or equal to about 0.01 mg/mL, less than or equal to about 1 μg/mL, less than or equal to about 0.1 μg/mL, less than or equal to about 0.01 μg/mL, less than or equal to about 1 ng/mL, less than or equal to about 0.1 ng/mL, or less than or equal to about 0.01 ng/mL at 25° C. In some embodiments, the solid material may have an aqueous solubility (or a solubility in a coating solution) of at least about 1 pg/mL, at least about 10 pg/mL, at least about 0.1 ng/mL, at least about 1 ng/mL, at least about 10 ng/mL, at least about 0.1 μg/mL, at least about 1 μg/mL, at least about 5 μg/mL, at least about 0.01 mg/mL, at least about 0.05 mg/mL, at least about 0.1 mg/mL, at least about 0.5 mg/mL, at least about 1.0 mg/mL, at least about 2 mg/mL. Combinations of the above-noted ranges are possible (e.g., an aqueous solubility or a solubility in a coating solution of at least about 10 pg/mL and less than or equal to about 1 mg/mL). Other ranges are also possible. The solid material may have these or other ranges of aqueous solubilities at any point throughout the pH range (e.g., from pH 1 to pH 14).

In some embodiments, the core may be formed of a material within one of the ranges of solubilities classified by the U.S. Pharmacopeia Convention: e.g., very soluble: >1,000 mg/mL; freely soluble: 100-1,000 mg/mL; soluble: 33-100 mg/mL; sparingly soluble: 10-33 mg/mL; slightly soluble: 1-10 mg/mL; very slightly soluble: 0.1-1 mg/mL; and practically insoluble: <0.1 mg/mL.

Although a core may be hydrophobic or hydrophilic, in many embodiments described herein, the core is substantially hydrophobic. “Hydrophobic” and “hydrophilic” are given their ordinary meaning in the art and, as will be understood by those skilled in the art, in many instances herein, are relative terms. Relative hydrophobicities and hydrophilicities of materials can be determined by measuring the contact angle of a water droplet on a planar surface of the substance to be measured, e.g., using an instrument such as a contact angle goniometer and a packed powder of the core material.

In some embodiments, the core particles described herein may be produced by nanomilling of a solid material (e.g., a compound of Formula I, Formula II, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, or Formula XVI) in the presence of one or more stabilizers/surface-altering agents. Small particles of a solid material may require the presence of one or more stabilizers/surface-altering agents, particularly on the surface of the particles, in order to stabilize a suspension of particles without agglomeration or aggregation in a liquid solution. In some such embodiments, the stabilizer may act as a surface-altering agent, forming a coating on the particle.

In a wet milling process, milling can be performed in a dispersion (e.g., an aqueous dispersion) containing one or more stabilizers (e.g., a surface-altering agent), a grinding medium, a solid to be milled (e.g., a solid pharmaceutical agent), and a solvent. Any suitable amount of a stabilizer/surface-altering agent can be included in the solvent. In some embodiments, a stabilizer/surface-altering agent may be present in the solvent in an amount of at least about 0.001% (wt or % weight to volume (w:v)), at least about 0.01, at least about 0.1, at least about 0.5, at least about 1, at least about 2, at least about 3, at least about 4, at least about 5, at least about 6, at least about 7, at least about 8, at least about 10, at least about 12, at least about 15, at least about 20, at least about 40, at least about 60, or at least about 80% of the solvent. In some cases, the stabilizer may be present in the solvent in an amount of about 100% (e.g., in an instance where the stabilizer/surface-altering agent is the solvent). In other embodiments, the stabilizer may be present in the solvent in an amount of less than or equal to about 100, less than or equal to about 80, less than or equal to about 60, less than or equal to about 40, less than or equal to about 20, less than or equal to about 15, less than or equal to about 12, less than or equal to about 10, less than or equal to about 8, less than or equal to about 7%, less than or equal to about 6%, less than or equal to about 5%, less than or equal to about 4%, less than or equal to about 3%, less than or equal to about 2%, or less than or equal to about 1% of the solvent. Combinations of the above-referenced ranges are also possible (e.g., an amount of less than or equal to about 5% and at least about 1% of the solvent). Other ranges are also possible. The particular range chosen may influence factors that may affect the ability of the particles to penetrate mucus such as the stability of the coating of the stabilizer/surface-altering agent on the particle surface, the average thickness of the coating of the stabilizer/surface-altering agent on the particles, the orientation of the stabilizer/surface-altering agent on the particles, the density of the stabilizer/surface altering agent on the particles, stabilizer/drug ratio, drug concentration, the size and polydispersity of the particles formed, and the morphology of the particles formed.

The compound of Formula I, Formula II, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, or Formula XVI (or salt thereof) may be present in the solvent in any suitable amount. In some embodiments, the pharmaceutical agent (or salt thereof) is present in an amount of at least about 0.001% (wt % or % weight to volume (w:v)), at least about 0.01%, at least about 0.1%, at least about 0.5%, at least about 1%, at least about 2%, at least about 3%, at least about 4%, at least about 5%, at least about 6%, at least about 7%, at least about 8%, at least about 10%, at least about 12%, at least about 15%, at least about 20%, at least about 40%, at least about 60%, or at least about 80% of the solvent. In some cases, the pharmaceutical agent (or salt thereof) may be present in the solvent in an amount of less than or equal to about 100%, less than or equal to about 90%, less than or equal to about 80%, less than or equal to about 60%, less than or equal to about 40%, less than or equal to about 20%, less than or equal to about 15%, less than or equal to about 12%, less than or equal to about 10%, less than or equal to about 8%, less than or equal to about 7%, less than or equal to about 6%, less than or equal to about 5%, less than or equal to about 4%, less than or equal to about 3%, less than or equal to about 2%, or less than or equal to about 1% of the solvent. Combinations of the above-referenced ranges are also possible (e.g., an amount of less than or equal to about 20% and at least about 1% of the solvent). In some embodiments, the pharmaceutical agent is present in the above ranges but in w:v.

The ratio of stabilizer/surface-altering agent to pharmaceutical agent (or salt thereof) in a solvent may also vary. In some embodiments, the ratio of stabilizer/surface-altering agent to pharmaceutical agent (or salt thereof) may be at least 0.001:1 (weight ratio, molar ratio, or w:v ratio), at least 0.01:1, at least 0.01:1, at least 1:1, at least 2:1, at least 3:1, at least 5:1, at least 10:1, at least 25:1, at least 50:1, at least 100:1, or at least 500:1. In some cases, the ratio of stabilizer/surface-altering agent to pharmaceutical agent (or salt thereof) may be less than or equal to 1000:1 (weight ratio or molar ratio), less than or equal to 500:1, less than or equal to 100:1, less than or equal to 75:1, less than or equal to 50:1, less than or equal to 25:1, less than or equal to 10:1, less than or equal to 5:1, less than or equal to 3:1, less than or equal to 2:1, less than or equal to 1:1, or less than or equal to 0.1:1.

Combinations of the above-referenced ranges are possible (e.g., a ratio of at least 5:1 and less than or equal to 50:1). Other ranges are also possible.

Stabilizers/surface-altering agents may be, for example, polymers or surfactants. Examples of polymers are those suitable for use in coatings, as described in more detail below. Non-limiting examples of surfactants include L-a-phosphatidylcholine (PC), 1,2-dipalmitoylphosphatidycholine (DPPC), oleic acid, sorbitan trioleate, sorbitan mono-oleate, sorbitan monolaurate, polyoxyethylene sorbitan monolaurate, polyoxyethylene sorbitan monooleate, natural lecithin, oleyl polyoxyethylene ether, stearyl polyoxyethylene ether, lauryl polyoxyethylene ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, and sunflower seed oil. Derivatives of the above-noted compounds are also possible. Combinations of the above-noted compounds and others described herein may also be used as surface-altering agents in the inventive particles. As described herein, in some embodiments a surface-altering agent may act as a stabilizer, a surfactant, and/or an emulsifier. In some embodiments, the surface altering agent may aid particle transport in mucus.

It should be appreciated that while in some embodiments the stabilizer used for milling forms a coating on a particle surface, which coating renders particle mucus penetrating, in other embodiments, the stabilizer may be exchanged with one or more other surface-altering agents after the particle has been formed. For example, in one set of methods, a first stabilizer/surface-altering agent may be used during a milling process and may coat a surface of a core particle, and then all or portions of the first stabilizer/surface-altering agent may be exchanged with a second stabilizer/surface-altering agent to coat all or portions of the core particle surface. In some cases, the second stabilizer/surface-altering agent may render the particle mucus penetrating more than the first stabilizer/surface-altering agent. In some embodiments, a core particle having a coating including multiple surface-altering agents may be formed.

In other embodiments, core particles may be formed by a precipitation technique. Precipitation techniques (e.g., microprecipitation techniques, nanoprecipitation techniques) may involve forming a first solution comprising a compound of Formula I, Formula II, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, or Formula XVI and a solvent, wherein the material is substantially soluble in the solvent. The solution may be added to a second solution comprising another solvent in which the material is substantially insoluble, thereby forming a plurality of particles comprising the material. In some cases, one or more surface-altering agents, surfactants, materials, and/or bioactive agents may be present in the first and/or second solutions. A coating may be formed during the process of precipitating the core (e.g., the precipitating and coating steps may be performed substantially simultaneously). In other embodiments, the particles are first formed using a precipitation technique, following by coating of the particles with a surface-altering agent.

In some embodiments, a precipitation technique may be used to form particles (e.g., nanocrystals) of a salt of a compound of Formula I, Formula II, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, or Formula XVI. Generally, a precipitation technique involves dissolving the material to be used as the core in a solvent, which is then added to a miscible anti-solvent with or without excipients to form the core particle. This technique may be useful for preparing particles of pharmaceutical agents that are soluble in aqueous solutions (e.g., agents having a relatively high aqueous solubility). In some embodiments, pharmaceutical agents having one or more charged or ionizable groups can interact with a counter ion (e.g., a cation or an anion) to form a salt complex.

As described herein, in some embodiments, a method of forming a core particle involves choosing a stabilizer that is suitable for both nanomilling and for forming a coating on the particle and rendering the particle mucus penetrating. For example, as described in more detail below, it has been demonstrated that 200-500 nm nanoparticles of a model compound pyrene produced by nanomilling of pyrene in the presence of Pluronic® F127 resulted in particles that can penetrate physiological mucus samples at the same rate as well-established polymer-based MPP.

Interestingly, it was observed that only a handful of stabilizers/surface-altering agents tested fit the criteria of being suitable for both nanomilling and for forming a coating on the particle that renders the particle mucus penetrating, as described in more detail below.

### IV. Description of Polymeric Delivery Materials

The particles of the drug delivery system can include a biocompatible polymer. As used herein, the term “biocompatible polymer” encompasses any polymer than can be administered to a patient without an unacceptable adverse effect to the patient.

Examples of biocompatible polymers include but are not limited to polystyrenes; poly(hydroxy acid); poly(lactic acid); poly(glycolic acid); poly(lactic acid-co-glycolic acid); poly(lactic-co-glycolic acid); poly(lactide); poly(glycolide); poly(lactide-co-glycolide); polyanhydrides; polyorthoesters; polyamides; polycarbonates; polyalkylenes; polyethylenes; polypropylene; polyalkylene glycols; poly(ethylene glycol); polyalkylene oxides; poly(ethylene oxides); polyalkylene terephthalates; poly(ethylene terephthalate); polyvinyl alcohols; polyvinyl ethers; polyvinyl esters; polyvinyl halides; poly(vinyl chloride); polyvinylpyrrolidone; polysiloxanes; poly(vinyl alcohols); poly(vinyl acetate); polyurethanes; co-polymers of polyurethanes; derivativized celluloses; alkyl cellulose; hydroxyalkyl celluloses; cellulose ethers; cellulose esters; nitro celluloses; methyl cellulose; ethyl cellulose; hydroxypropyl cellulose; hydroxy-propyl methyl cellulose; hydroxybutyl methyl cellulose; cellulose acetate; cellulose propionate; cellulose acetate butyrate; cellulose acetate phthalate; carboxylethyl cellulose; cellulose triacetate; cellulose sulfate sodium salt; polymers of acrylic acid; methacrylic acid; copolymers of methacrylic acid; derivatives of methacrylic acid; poly(methyl methacrylate); poly(ethyl methacrylate); poly(butylmethacrylate); poly(isobutyl methacrylate); poly(hexylmethacrylate); poly(isodecyl methacrylate); poly(lauryl methacrylate); poly(phenyl methacrylate); poly(methyl acrylate); poly(isopropyl acrylate); poly(isobutyl acrylate); poly(octadecyl acrylate); poly(butyric acid); poly(valeric acid); poly(lactide-co-caprolactone); copolymers of poly(lactide-co-caprolactone); blends of poly(lactide-co-caprolactone); hydroxyethyl methacrylate (HEMA); copolymers of HEMA with acrylate; copolymers of HEMA with polymethylmethacrylate (PMMA); polyvinylpyrrolidone/vinyl acetate copolymer (PVPNA); acrylate polymers/copolymers; acrylate/carboxyl polymers; acrylate hydroxyl and/or carboxyl copolymers; polycarbonate-urethane polymers; silicone-urethane polymers; epoxy polymers; cellulose nitrates; polytetramethylene ether glycol urethane; polymethylmethacrylate-2-hydroxyethylmethacrylate copolymer; polyethylmethacrylate-2-hydroxyethylmethacrylate copolymer; polypropylmethacrylate-2-hydroxyethylmethacrylate copolymer; polybutylmethacrylate-2-hydroxyethylmethacrylate copolymer; polymethylacrylate-2-hydroxyethylmethacrylate copolymer; polyethylacrylate-2-hydroxyethylmethacrylate copolymer; polypropylacrylate-2-hydroxymethacrylate copolymer; polybutylacrylate-2-hydroxyethylmethacrylate copolymer; copolymermethylvinylether maleicanhydride copolymer; poly (2-hydroxyethyl methacrylate) polymer/copolymer; acrylate carboxyl and/or hydroxy copolymer; olefin acrylic acid copolymer; ethylene acrylic acid copolymer; polyamide polymers/copolymers; polyimide polymers/copolymers; ethylene vinylacetate copolymer; polycarbonate urethane; silicone urethane; polyvinylpyridine copolymers; polyether sulfones; polygalactin, poly-(isobutyl cyanoacrylate), and poly(2-hydroxyethyl-L-glutamine); polydimethyl siloxane; poly(caprolactones); poly(ortho esters); polyamines; polyethers; polyesters; polycarbamates; polyureas; polyimides; polysulfones; polyacetylenes; polyethyeneimines; polyisocyanates; polyacrylates; polymethacrylates; polyacrylonitriles; polyarylates; and combinations, copolymers and/or mixtures of two or more of any of the foregoing. In some cases, the particle includes a hydrophobic material and at least one bioactive agent. In certain embodiments, the hydrophobic material is used instead of a polymer. In other embodiments, the hydrophobic material is used in addition to a polymer.

An active compound as described herein can be physically mixed in the polymeric material, including in an interpenetrating polymer network or can be covalently bound to the polymeric material

Linear, non-linear or linear multiblock polymers or copolymers can be used to form nanoparticles, microparticles, and implants (e.g., rods, discs, wafers, etc.) useful for the delivery to the eye. The polymers can contain one or more hydrophobic polymer segments and one or more hydrophilic polymer segments covalently connected through a linear link or multivalent branch point to form a non-linear multiblock copolymer containing at least three polymeric segments. The polymer can be a conjugate further containing one or more therapeutic, prophylactic, or diagnostic agents covalently attached to the one or more polymer segments. By employing a polymer-drug conjugate, particles can be formed with more controlled drug loading and drug release profiles. In addition, the solubility of the conjugate can be controlled so as to minimize soluble drug concentration and, therefore, toxicity.

The one or more hydrophobic polymer segments, independently, can be any biocompatible hydrophobic polymer or copolymer. In some cases, the one or more hydrophobic polymer segments are also biodegradable. Examples of suitable hydrophobic polymers include polyesters such as polylactic acid, polyglycolic acid, or polycaprolactone, polyanhydrides, such as polysebacic anhydride, and copolymers thereof. In certain embodiments, the hydrophobic polymer is a polyanhydride, such as polysebacic anhydride or a copolymer thereof. The one or more hydrophilic polymer segments can be any hydrophilic, biocompatible, non-toxic polymer or copolymer. The hydrophilic polymer segment can be, for example, a poly(alkylene glycol), a polysaccharide, poly(vinyl alcohol), polypyrrolidone, a polyoxyethylene block copolymer (PLURONIC®) or a copolymers thereof. In preferred embodiments, the one or more hydrophilic polymer segments are, or are composed of, polyethylene glycol (PEG).

WO 2016/100380A1 and WO 2016/100392 A1 describe certain Sunitinib delivery systems, which can also be used in the present invention to deliver the IOP lowering agents provided by the current invention, and as described further herein. For example, a process similar to that used in WO 2016/100380A1 and WO 2016/100392 A1 to prepare a polymeric Sunitinib drug formulation can be utilized: (i) dissolve or disperse the IOP lowering agent or its salt in an organic solvent; (ii) mix the solution/dispersion of step (i) with a polymer solution that has a viscosity of at least about 300 cPs (or perhaps at least about 350, 400, 500, 600, 700 or 800 or more cPs); (iii) mix the drug polymer solution/dispersion of step (ii) with an aqueous solution optionally with a surfactant or emulsifier, to form a solvent-laden encapsulated microparticle; and (iv) isolate the microparticles. Drug loading is also significantly affected by the method of making and the solvent used. For example, S/O/W single emulsion method will yield a higher loading than O/W single emulsion method. In addition, W/O/W double emulsions have been shown to significantly improve drug loading of less hydrophobic salt forms over single O/W emulsions. The ratio of continuous phase to dispersed phase can also significantly alter the encapsulation efficiency and drug loading by modulation of the rate of particle solidification. The rate of polymer solidification with the evaporation of solvent affects the degree of porosity within microparticles.

A large CP:DP ratio results in faster polymer precipitation, less porosity, and higher encapsulation efficiency and drug loading. However, decreasing the rate of evaporation of the solvent during particle preparation can also lead to improvements in drug loading of highly polar compounds. As the organic phase evaporates, highly polar compounds within the organic phase is driven to the surface of the particles resulting in poor encapsulation and drug loading. By decreasing the rate of solvent evaporation by decreasing the temperature or rate of stirring, encapsulation efficiency and % drug loading can be increased for highly polar compounds. These technologies can be used by one of skill in the art to deliver any of the active compounds as described generally in this specification.

U.S. Pat. No. 8,889,193 and PCT/US2011/026321 disclose, for example, a method for treating an eye disorder in a patient in need thereof, comprising administering into the eye, for example, by intravitreal injection into the vitreous chamber of the eye, an effective amount of a drug delivery system which comprises: (i) a microparticle including a core which includes the biodegradable polymer polylactide-co-glycolide; (ii) a coating associated with the core which is non-covalently associated with the microparticle particle; wherein the coating molecule has a hydrophilic region and a hydrophobic region, and wherein the hydrophilic region is polyethylene glycol; and (iii) a therapeutically effective amount of a therapeutic agent, wherein the drug delivery system provides sustained release of the therapeutic agent into the vitreous chamber over a period of time of at least three months; and wherein the vitreous chamber of the eye exhibits at least 10% less inflammation or intraocular pressure than if the particle were uncoated. In certain embodiments, the microparticle can be about 50 or 30 microns or less. The delivery system described in U.S. Pat. No. 8,889,193 and PCT/US2011/026321 can be used to deliver any of the active agents described herein.

In some embodiments, the drug delivery systems contain a particle with a coating on the surface, wherein the coating molecules have hydrophilic regions and, optionally, hydrophobic regions,

The drug delivery system can include a coating. The coating can be disposed on the surface of the particle, for example by bonding, adsorption or by complexation. The coating can also be intermingled or dispersed within the particle as well as disposed on the surface of the particle.

The homogeneous or heterogenous polymer or polymeric coating can be, for example, polyethylene glycol, polyvinyl alcohol (PVA), or similar substances. The coating can be, for example, vitamin E-PEG 1k or vitamin E-PEG 5k or the like. Vitamin E-PEG 5k can help present a dense coating of PEG on the surface of a particle. The coating can also include nonionic surfactants such as those composed of polyalkylene oxide, e.g., polyoxyethylene (PEO), also referred to herein as polyethylene glycol; or polyoxypropylene (PPO), also referred to herein as polypropylene glycol (PPG), and can include a copolymer of more than one alkylene oxide.

The polymer or copolymer can be, for example, a random copolymer, an alternating copolymer, a block copolymer or graft copolymer.

In some embodiments, the coating can include a polyoxyethylene-polyoxypropylene copolymer, e.g., block copolymer of ethylene oxide and propylene oxide. (i.e., poloxamers). Examples of poloxamers suitable for use in the present invention include, for example, poloxamers 188, 237, 338 and 407. These poloxamers are available under the trade name Pluronic® (available from BASF, Mount Olive, N.J.) and correspond to Pluronic® F-68, F-87, F-108 and F-127, respectively. Poloxamer 188 (corresponding to Pluronic® F-68) is a block copolymer with an average molecular mass of about 7,000 to about 10,000 Da, or about 8,000 to about 9,000 Da, or about 8,400 Da. Poloxamer 237 (corresponding to Pluronic® F-87) is a block copolymer with an average molecular mass of about 6,000 to about 9,000 Da, or about 6,500 to about 8,000 Da, or about 7,7000 Da. Poloxamer 338 (corresponding to Pluronic® F-108) is a block copolymer with an average molecular mass of about 12,000 to about 18,000 Da, or about 13,000 to about 15,000 Da, or about 14,600 Da. Poloxamer 407 (corresponding to Pluronic® F-127) is a polyoxyethylene-polyoxypropylene triblock copolymer in a ratio of between about E₁₀₁ P₅₆ E₁₀₁ to about E₁₀₆ P₇₀ E₁₀₆, or about E₁₀₁ P₅₆ E₁₀₁, or about E₁₀₆ P₇₀ E₁₀₆, with an average molecular mass of about 10,000 to about 15,000 Da, or about 12,000 to about 14,000 Da, or about 12,000 to about 13,000 Da, or about 12,600 Da. For example, the NF forms of poloxamers or Pluronic® polymers can be used.

In some embodiments, the polymer can be, for example Pluronic® P103 or Pluronic® P105. Pluronic® P103 is a block copolymer with an average molecular mass of about 3,000 Da to about 6,000 Da, or about 4,000 Da to about 6,000 Da, or about 4,950 Da. Pluronic® P105 is a block copolymer with an average molecular mass of about 5,000 Da to about 8,000 Da, or about 6,000 Da to about 7,000 Da, or about 6,500 Da.

In some embodiments, the polymer can have an average molecular weight of about 9,000 Da or greater, about 10,000 Da or greater, about 11,000 Da or greater or about 12,000 Da or greater.

In exemplary embodiments, the polymer can have an average molecular weight of from about 10,000 to about 15,000 Da, or about 12,000 to about 14,000 Da, or about 12,000 to about 13,000 Da, or about 12,600 Da. In some embodiments, the polymer can be selected from Pluronic® P103, P105, F-68, F-87, F-108 and F-127, from Pluronic® P103, P105, F-87, F-108 and F-127, or from Pluronic® P103, P105, F-108 and F-127, or from Pluronic® P103, P105 and F-127. In some embodiments, the polymer can be Pluronic® F-127. In representative embodiments, the polymer is associated with the particles. For example, the polymer can be covalently attached to the particles. In representative embodiments, the polymer comprises polyethylene glycol, which is covalently attached to a selected polymer, yielding what is commonly referred to as a PEGylated particle.

In some embodiments, a coating is non-covalently associated with a core particle. This association can be held together by any force or mechanism of molecular interaction that permits two substances to remain in substantially the same positions relative to each other, including intermolecular forces, dipole-dipole interactions, van der Waals forces, hydrophobic interactions, electrostatic interactions and the like. In some embodiments, the coating is adsorbed onto the particle. According to representative embodiments, a non-covalently bound coating can be comprised of portions or segments that promote association with the particle, for example by electrostatic or van der Waals forces. In some embodiments, the interaction is between a hydrophobic portion of the coating and the particle. Embodiments include particle coating combinations which, however attached to the particle, present a hydrophilic region, e.g. a PEG rich region, to the environment around the particle coating combination. The particle coating combination can provide both a hydrophilic surface and an uncharged or substantially neutrally-charged surface, which can be biologically inert.

Suitable polymers for use according to the compositions and methods disclosed herein can be made up of molecules having hydrophobic regions as well as hydrophilic regions. Without wishing to be bound by any particular theory, when used as a coating, it is believed that the hydrophobic regions of the molecules are able to form adsorptive interactions with the surface of the particle, and thus maintain a non-covalent association with it, while the hydrophilic regions orient toward the surrounding, frequently aqueous, environment. In some embodiments the hydrophilic regions are characterized in that they avoid or minimize adhesive interactions with substances in the surrounding environment. Suitable hydrophobic regions in a coatings can include, for example, PPO, vitamin E and the like, either alone or in combination with each other or with other substances. Suitable hydrophilic regions in the coatings can include, for example, PEG, heparin, polymers that form hydrogels and the like, alone or in combination with each other or with other substances.

Representative coatings according to the compositions and methods disclosed herein can include molecules having, for example, hydrophobic segments such as PPO segments with molecular weights of at least about 1.8 kDa, or at least about 2 kDa, or at least about 2.4 kDa, or at least about 2.8 kDa, or at least about 3.2 kDa, or at least about 3.6 kDa, or at least about 4.0 kDa, or at least about 4.4 kDa, or at least about 4.8 kDa or at least about 5.2 kDa, or at least 5.6 kDa, or at least 6.0 kDa, or at least 6.4 kDa or more. In some embodiments, the coatings can have PPO segments with molecular weights of from about 1.8 kDa to about 10 kDa, or from about 2 kDa to about 5 kDa, or from about 2.5 kDa to about 4.5 kDa, or from about 2.5 kDa to about 3.5 kDa, or from about 3 kDa to about 5 kDa, or from about 3 kDa to about 6 kDa, or from about 4 kDa to about 6 kDa, or from about 4 kDa to about 7 kDa. In some embodiments, at least about 10%, or at least about 25%, or at least about 50%, or at least about 75%, or at least about 90%, or at least about 95%, or at least about 99% or more of the hydrophobic regions in these coatings have molecular weights within these ranges. In some embodiments, the coatings are biologically inert. Compounds that generate both a hydrophilic surface and an uncharged or substantially neutrally-charged surface can be biologically inert.

Representative coatings according to the compositions and methods disclosed herein can include molecules having, for example, hydrophobic segments such as PEG segments with molecular weights of at least about 1.8 kDa, or at least about 2 kDa, or at least about 2.4 kDa, or at least about 2.8 kDa, or at least about 3.2 kDa, or at least about 3.6 kDa, or at least about 4.0 kDa, or at least about 4.4 kDa, or at least about 4.8 kDa, or at least about 5.2 kDa, or at least 5.6 kDa, or at least 6.0 kDa, or at least 6.4 kDa or more. In some embodiments, the coatings can have PEG segments with molecular weights of from about 1.8 kDa to about 10 kDa, or from about 2 kDa to about 5 kDa, or from about 2.5 kDa to about 4.5 kDa, or from about 2.5 kDa to about 3.5 kDa. In some embodiments, at least about 10%, or at least about 25%, or at least about 50%, or at least about 75%, or at least about 90%, or at least about 95%, or at least about 99% or more of the hydrophobic regions in these coatings have molecular weights within these ranges. In some embodiments, the coatings are biologically inert. Compounds that generate both a hydrophilic surface and an uncharged or substantially neutrally-charged surface can be biologically inert.

Representative coatings according to the compositions and methods disclosed herein can include molecules having, for example, segments such as PLGA segments with molecular weights of at least about 4 kDa, or at least about 8 kDa, or at least about 12 kDa, or at least about 16 kDa, or at least about 20 kDa, or at least about 24 kDa, or at least about 28 kDa, or at least about 32 kDa, or at least about 36 kDa, or at least about 40 kDa, or at least about 44 kDa, of at least about 48 kDa, or at least about 52 kDa, or at least about 56 kDa, or at least about 60 kDa, or at least about 64 kDa, or at least about 68 kDa, or at least about 72 kDa, or at least about 76 kDa, or at least about 80 kDa, or at least about 84 kDa, or at least about 88 kDa or more. In some embodiments, at least about 10%, or at least about 25%, or at least about 50%, or at least about 75%, or at least about 90%, or at least about 95%, or at least about 99% or more of the regions in these coatings have molecular weights within these ranges. In some embodiments, the coatings are biologically inert. Compounds that generate both a hydrophilic surface and an uncharged or substantially neutrally-charged surface can be biologically inert.

In some embodiments, s coating can include, for example, one or more of the following: anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin), mucolytic agents, N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4, dornase alfa, neltenexine, erdosteine, various DNases including rhDNase, agar, agarose, alginic acid, amylopectin, amylose, beta-glucan, callose, carrageenan, cellodextrins, cellulin, cellulose, chitin, chitosan, chrysolaminarin, curdlan, cyclodextrin, dextrin, ficoll, fructan, fucoidan, galactomannan, gellan gum, glucan, glucomannan, glycocalyx, glycogen, hemicellulose, hydroxyethyl starch, kefiran, laminarin, mucilage, glycosaminoglycan, natural gum, paramylon, pectin, polysaccharide peptide, schizophyllan, sialyl lewis x, starch, starch gelatinization, sugammadex, xanthan gum, xyloglucan, L-phosphatidylcholine (PC), 1,2-dipalmitoylphosphatidycholine (DPPC), oleic acid, sorbitan trioleate, sorbitan monooleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, polyoxyethylene (4) lauryl ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, sunflower seed oil, lecithin, oleic acid, sorbitan trioleate, and combinations of two or more of any of the foregoing.

A particle-coating combinations can be made up of any combination of particle and coating substances disclosed or suggested herein. Examples of such combinations include, for example, polystyrene-PEG, or PLGA-Pluronic® F-127.

In one aspect of the present invention, an effective amount of an active compound as described herein is incorporated into a nanoparticle, e.g. for convenience of delivery and/or extended release delivery. The use of materials in nanoscale provides one the ability to modify fundamental physical properties such as solubility, diffusivity, blood circulation half-life, drug release characteristics, and/or immunogenicity. These nanoscale agents may provide more effective and/or more convenient routes of administration, lower therapeutic toxicity, extend the product life cycle, and ultimately reduce health-care costs. As therapeutic delivery systems, nanoparticles can allow targeted delivery and controlled release.

In another aspect of the present invention, the nanoparticle or microparticle is coated with a surface agent that facilitates passage of the particle through mucus. Said nanoparticles and microparticles have a higher concentration of surface agent than has been previously achieved, leading to the unexpected property of extremely fast diffusion through mucus. The present invention further comprises a method of producing said particles. The present invention further comprises methods of using said particles to treat a patient.

A number of companies have developed microparticles for treatment of eye disorders that can be used in conjunction with the present invention. For example, Allergan has disclosed a biodegradable microsphere to deliver a therapeutic agent that is formulated in a high viscosity carrier suitable for intraocular injection or to treat a non-ocular disorder (see U.S. publication 2010/0074957 and U.S. publication 2015/0147406). In one embodiment, the '957 application describes a biocompatible, intraocular drug delivery system that includes a plurality of biodegradable microspheres, a therapeutic agent, and a viscous carrier, wherein the carrier has a viscosity of at least about 10 cps at a shear rate of 0.1/second at 25° C. Allergan has also disclosed a composite drug delivery material that can be injected into the eye of a patient that includes a plurality of microparticles dispersed in a media, wherein the microparticles contain a drug and a biodegradable or bioerodible coating and the media includes the drug dispersed in a depot-forming material, wherein the media composition may gel or solidify on injection into the eye (see WO 2013/112434 A1, claiming priority to Jan. 23, 2012). Allergan states that this invention can be used to provide a depot means to implant a solid sustained drug delivery system into the eye without an incision. In general, the depot on injection transforms to a material that has a viscosity that may be difficult or impossible to administer by injection. In addition, Allergan has disclosed biodegradable microspheres between 40 and 200 μm in diameter, with a mean diameter between 60 and 150 μm that are effectively retained in the anterior chamber of the eye without producing hyperemia, see, US 2014/0294986. The microspheres contain a drug effective for an ocular condition with greater than seven day release following administration to the anterior chamber of the eye. The administration of these large particles is intended to overcome the disadvantages of injecting 1-30 μm particles which are generally poorly tolerated.

In another embodiment any of the above delivery systems can be used to facilitate or enhance delivery through mucus.

Common techniques for preparing particles include, but are not limited to, solvent evaporation, solvent removal, spray drying, phase inversion, coacervation, and low temperature casting. Suitable methods of particle formulation are briefly described below. Pharmaceutically acceptable excipients, including pH modifying agents, disintegrants, preservatives, and antioxidants, can optionally be incorporated into the particles during particle formation.

### Solvent Evaporation

In this method, the drug (or polymer matrix and one or more Drugs) is dissolved in a volatile organic solvent, such as methylene chloride. The organic solution containing the drug is then suspended in an aqueous solution that contains a surface active agent such as poly(vinyl alcohol). The resulting emulsion is stirred until most of the organic solvent evaporated, leaving solid nanoparticles. The resulting nanoparticles are washed with water and dried overnight in a lyophilizer. Nanoparticles with different sizes and morphologies can be obtained by this method.

Drugs which contain labile polymers, such as certain polyanhydrides, may degrade during the fabrication process due to the presence of water. For these polymers, the following two methods, which are performed in completely anhydrous organic solvents, can be used.

### Solvent Removal

Solvent removal can also be used to prepare particles from drugs that are hydrolytically unstable. In this method, the drug (or polymer matrix and one or more Drugs) is dispersed or dissolved in a volatile organic solvent such as methylene chloride. This mixture is then suspended by stirring in an organic oil (such as silicon oil) to form an emulsion. Solid particles form from the emulsion, which can subsequently be isolated from the supernatant. The external morphology of spheres produced with this technique is highly dependent on the identity of the drug.

In one embodiment a compound of the present invention is administered to a patient in need thereof as particles formed by solvent removal. In another embodiment the present invention provides particles formed by solvent removal comprising a compound of the present invention and one or more pharmaceutically acceptable excipients as defined herein. In another embodiment the particles formed by solvent removal comprise a compound of the present invention and an additional therapeutic agent. In a further embodiment the particles formed by solvent removal comprise a compound of the present invention, an additional therapeutic agent, and one or more pharmaceutically acceptable excipients. In another embodiment any of the described particles formed by solvent removal can be formulated into a tablet and then coated to form a coated tablet.

In an alternative embodiment the particles formed by solvent removal are formulated into a tablet but the tablet is uncoated.

### Spray Drying

In this method, the drug (or polymer matrix and one or more Drugs) is dissolved in an organic solvent such as methylene chloride. The solution is pumped through a micronizing nozzle driven by a flow of compressed gas, and the resulting aerosol is suspended in a heated cyclone of air, allowing the solvent to evaporate from the micro droplets, forming particles. Particles ranging between 0.1-10 microns can be obtained using this method.

In one embodiment a compound of the present invention is administered to a patient in need thereof as a spray dried dispersion (SDD). In another embodiment the present invention provides a spray dried dispersion (SDD) comprising a compound of the present invention and one or more pharmaceutically acceptable excipients as defined herein. In another embodiment the SDD comprises a compound of the present invention and an additional therapeutic agent. In a further embodiment the SDD comprises a compound of the present invention, an additional therapeutic agent, and one or more pharmaceutically acceptable excipients. In another embodiment any of the described spray dried dispersions can be coated to form a coated tablet. In an alternative embodiment the spray dried dispersion is formulated into a tablet but is uncoated.

### Phase Inversion

Particles can be formed from drugs using a phase inversion method. In this method, the drug (or polymer matrix and one or more Drugs) is dissolved in a “good” solvent, and the solution is poured into a strong non solvent for the drug to spontaneously produce, under favorable conditions, microparticles or nanoparticles. The method can be used to produce nanoparticles in a wide range of sizes, including, for example, about 100 nanometers to about 10 microns, typically possessing a narrow particle size distribution.

In one embodiment a compound of the present invention is administered to a patient in need thereof as particles formed by phase inversion. In another embodiment the present invention provides particles formed by phase inversion comprising a compound of the present invention and one or more pharmaceutically acceptable excipients as defined herein. In another embodiment the particles formed by phase inversion comprise a compound of the present invention and an additional therapeutic agent. In a further embodiment the particles formed by phase inversion comprise a compound of the present invention, an additional therapeutic agent, and one or more pharmaceutically acceptable excipients. In another embodiment any of the described particles formed by phase inversion can be formulated into a tablet and then coated to form a coated tablet.

In an alternative embodiment the particles formed by phase inversion are formulated into a tablet but the tablet is uncoated.

### Coacervation

Techniques for particle formation using coacervation are known in the art, for example, in GB-B-929 406; GB-B-929 40 1; and U.S. Pat. Nos. 3,266,987, 4,794,000, and 4,460,563. Coacervation involves the separation of a drug (or polymer matrix and one or more Drugs) solution into two immiscible liquid phases. One phase is a dense coacervate phase, which contains a high concentration of the drug, while the second phase contains a low concentration of the drug. Within the dense coacervate phase, the drug forms nanoscale or microscale droplets, which harden into particles. Coacervation may be induced by a temperature change, addition of a non-solvent or addition of a micro-salt (simple coacervation), or by the addition of another polymer thereby forming an interpolymer complex (complex coacervation).

In one embodiment a compound of the present invention is administered to a patient in need thereof as particles formed by coacervation. In another embodiment the present invention provides particles formed by coacervation comprising a compound of the present invention and one or more pharmaceutically acceptable excipients as defined herein. In another embodiment the particles formed by coacervation comprise a compound of the present invention and an additional therapeutic agent. In a further embodiment the particles formed by coacervation comprise a compound of the present invention, an additional therapeutic agent, and one or more pharmaceutically acceptable excipients. In another embodiment any of the described particles formed by coacervation can be formulated into a tablet and then coated to form a coated tablet. In an alternative embodiment the particles formed by coacervation are formulated into a tablet but the tablet is uncoated.

### Low Temperature Casting

Methods for very low temperature casting of controlled release microspheres are described in U.S. Pat. No. 5,019,400 to Gombotz et al. In this method, the drug (or polymer matrix and Sunitinib) is dissolved in a solvent. The mixture is then atomized into a vessel containing a liquid non-solvent at a temperature below the freezing point of the drug solution which freezes the drug droplets. As the droplets and non-solvent for the drug are warmed, the solvent in the droplets thaws and is extracted into the non-solvent, hardening the microspheres.

In one embodiment a compound of the present invention is administered to a patient in need thereof as particles formed by low temperature casting. In another embodiment the present invention provides particles formed by low temperature casting comprising a compound of the present invention and one or more pharmaceutically acceptable excipients as defined herein. In another embodiment the particles formed by low temperature casting comprise a compound of the present invention and an additional therapeutic agent. In a further embodiment the particles formed by low temperature casting comprise a compound of the present invention, an additional therapeutic agent, and one or more pharmaceutically acceptable excipients. In another embodiment any of the described particles formed by low temperature casting can be formulated into a tablet and then coated to form a coated tablet. In an alternative embodiment the particles formed by low temperature casting are formulated into a tablet but the tablet is uncoated.

### V. Controlled Release of Therapeutic Agent

The rate of release of the therapeutic agent can be related to the concentration of therapeutic agent dissolved in polymeric material. In many embodiments, the polymeric composition includes non-therapeutic agents that are selected to provide a desired solubility of the therapeutic agent.

The selection of polymer can be made to provide the desired solubility of the therapeutic agent in the matrix, for example, a hydrogel may promote solubility of hydrophilic material. In some embodiments, functional groups can be added to the polymer to increase the desired solubility of the therapeutic agent in the matrix. In some embodiments, additives may be used to control the release kinetics of therapeutic agent, for example, the additives may be used to control the concentration of therapeutic agent by increasing or decreasing solubility of the therapeutic agent in the polymer so as to control the release kinetics of the therapeutic agent. The solubility may be controlled by including appropriate molecules and/or substances that increase and/or decrease the solubility of the dissolved from of the therapeutic agent to the matrix. The solubility of the therapeutic agent may be related to the hydrophobic and/or hydrophilic properties of the matrix and therapeutic agent. Oils and hydrophobic molecules and can be added to the polymer to increase the solubility of hydrophobic treatment agent in the matrix.

Instead of or in addition to controlling the rate of migration based on the concentration of therapeutic agent dissolved in the matrix, the surface area of the polymeric composition can be controlled to attain the desired rate of drug migration out of the composition. For example, a larger exposed surface area will increase the rate of migration of the active agent to the surface, and a smaller exposed surface area will decrease the rate of migration of the active agent to the surface. The exposed surface area can be increased in any number of ways, for example, by any of castellation of the exposed surface, a porous surface having exposed channels connected with the tear or tear film, indentation of the exposed surface, protrusion of the exposed surface. The exposed surface can be made porous by the addition of salts that dissolve and leave a porous cavity once the salt dissolves. In the present invention, these trends can be used to decrease the release rate of the active material from the polymeric composition by avoiding these paths to quicker release. For example, the surface area can be minimized, or channels avoided.

Further, an implant may be used that includes the ability to release two or more drugs in combination, for example, the structure disclosed in U.S. Pat. No. 4,281,654 (Shell), for example, in the case of glaucoma treatment, it may be desirable to treat a patient with multiple prostaglandins or a prostaglandin and a cholinergic agent or an adrenergic antagonist (beta blocker), for example, Alphagan (Allegan, Irvine, Calif., USA), or a prostaglandin and a carbonic anhydrase inhibitor.

In addition, drug impregnated meshes may be used, for example, those disclosed in U.S. Patent Application Publication No. 2002/0055701 or layering of biostable polymers as described in U.S. Patent Application Publication No. 2005/0129731. Certain polymer processes may be used to incorporate drug into the devices, as described herein, for example, so-called “self-delivering drugs” or Polymer Drugs (Polymerix Corporation, Piscataway, N.J., USA) are designed to degrade only into therapeutically useful compounds and physiologically inert linker molecules, further detailed in U.S. Patent Application Publication No. 2005/0048121 (East), hereby incorporated by reference in its entirety. Such delivery polymers may be employed in the devices, as described herein, to provide a release rate that is equal to the rate of polymer erosion and degradation and is constant throughout the course of therapy. Such delivery polymers may be used as device coatings or in the form of microspheres for a drug depot injectable (for example, a reservoir described herein). A further polymer delivery technology may also be adapted to the devices, as described herein, for example, that described in U.S. Patent Application Publication No. 2004/0170685 (Carpenter), and technologies available from Medivas (San Diego, Calif., USA).

### VI. Process of Preparation of Compounds of Formula I, Formula II, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, Formula XVI, or Formula XVII

### Abbreviations

CAN Acetonitrile
Ac Acetyl
Ac₂O Acetic anhydride
AcOEt, EtOAc ethyl acetate
AcOH Acetic acid
Boc₂O di-tert-butyl dicarbonate
Bu Butyl
CAN Ceric ammonium nitrate
CBz Carboxybenzyl
CDI Carbonyldiimidazole
CH₃OH, MeOH Methanol
CsF Cesium fluoride
Cul Cuprous iodide
DCM, CH₂Cl₂ Dichloromethane
DIEA, DIPEA N,N-diisopropylethylamine
DL Drug loading
DMA
DMAP 4-Dimethylaminopyridine
DMF N,N-dimethylformamide
DMS Dimethyl sulfide
DMSO Dimethylsulfoxide
DPPA Diphenyl phosphoryl azide
EDCI 1-Ethyl-3-(3-dimethylaminopropyl)carbodiimide
Et Ethyl
Et₃N, TEA Triethylamine
EtOAc Ethyl acetate
EtOH Ethanol
HATU 1-[Bis(dimethylamino)methylene]-1H-1,2,3-triazolo[4,5-b]pyridinium3-oxide hexafluorophosphate
HCl Hydrochloric acid
HOBT Hydroxybenzotriazole
iBu, i-Bu, isoBu Isobutyl
iPr, i-Pr, isoPr Isopropyl
iPr₂NEt N,N-diisopropylethylamine
K₂CO₃ Potassium carbonate
K₂CO₃ Potassium carbonate
LiOH Lithium hydroxide
Me Methyl
MeI Methyl iodide
Ms Mesyl
MsCl Mesylchloride
MTBE Methyl tbutylether
Na₂SO₄ Sodium sulfate
NaCl Sodium chloride
NaH Sodium hydride
NaHCO₃Sodium bicarbonate
NBS N-bromo succinimide
NCS N-chloro succinimide
NEt₃ Trimethylamine
NMP N-Methyl-2-pyrrolidone
PCC Pyridinium chlorochromate
Pd (OAc)₂ Palladium acetate
Pd(dppf)Cl₂ [1,1′-Bis(diphenylphosphino) ferrocene]dichloropalladium(II)
Pd(PPh₃)₂Cl₂ Bis(triphenylphosphine)palladium(II) dichloride
Pd(PPh₃)₄ Tetrakis(triphenylphosphine)palladium(0)
Pd/C Palladium on carbon
Pd₂(dba)₃ Tris(dibenzylideneacetone)dipalladium(0)
PMB 4-Methoxybenzyl ether
PPh₃ Triphenylphosphine
Pr Propyl
Py, py Pyridine
RT Room temperature
TBAF Tetra-n-butylammonium fluoride
TBAT Tetrabutylammonium difluorotriphenylsilicate
tBu, t-Bu Tertbutyl
tBuOK Potassium tert-butoxide
TEA Trimethylamine
Tf₂O Trifluoromethanesulfonic anhydride
TFA Trifluoroacetic acid
THF Tetrahydrofuran
TMS Trimethylsilane
TMSBr Bromotrimethylsilane
t_{R} Retention time
Troc 2,2,2-Trichlorethoxycarbonyl chloride
Zn (CN)₂ Zinc cyanide

### General Methods

All nonaqueous reactions were performed under an atmosphere of dry argon or nitrogen gas using anhydrous solvents. The progress of reactions and the purity of target compounds were determined using one of the two liquid chromatography (LC) methods listed below. The structure of starting materials, intermediates, and final products was confirmed by standard analytical techniques, including NMR spectroscopy and mass spectrometry.

The compounds described herein can be prepared by methods known by those skilled in the art. In one non-limiting example the disclosed compounds can be made by the schemes below.

Compounds of the present invention with stereocenters may be drawn without stereochemistry for convenience. One skilled in the art will recognize that pure enantiomers and diastereomers can be prepared by methods known in the art. Examples of methods to obtain optically active materials include at least the following.

i) Physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) Simultaneous crystallization—a technique whereby the individual enantiomers are separately crystallized from a solution of the racemate, possible only if the latter is a conglomerate in the solid state;

iii) Enzymatic resolutions—a technique whereby partial or complete separation of a racemate by virtue of differing rates of reaction for the enantiomers with an enzyme;

iv) Enzymatic asymmetric synthesis—a synthetic technique whereby at least one step of the synthesis uses an enzymatic reaction to obtain an enantiomerically pure or enriched synthetic precursor of the desired enantiomer;

v) Chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) Diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) First- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) Kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) Enantiospecific synthesis from non-racemic precursors—a synthetic technique whereby the desired enantiomer is obtained from non-chiral starting materials and where the stereochemical integrity is not or is only minimally compromised over the course of the synthesis;

x) Chiral liquid chromatography—a technique whereby the enantiomers of a racemate are separated in a liquid mobile phase by virtue of their differing interactions with a stationary phase (including via chiral HPLC). The stationary phase can be made of chiral material or the mobile phase can contain an additional chiral material to provoke the differing interactions;

xi) Chiral gas chromatography—a technique whereby the racemate is volatilized and enantiomers are separated by virtue of their differing interactions in the gaseous mobile phase with a column containing a fixed non-racemic chiral adsorbent phase;

xii) Extraction with chiral solvents—a technique whereby the enantiomers are separated by virtue of preferential dissolution of one enantiomer into a particular chiral solvent;

xiii) Transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

xiv) Simulated moving bed chromatography, is used in one embodiment. A wide variety of chiral stationary phases are commercially available.

### Example 1. Non-Limiting Examples of Compounds of Formula I

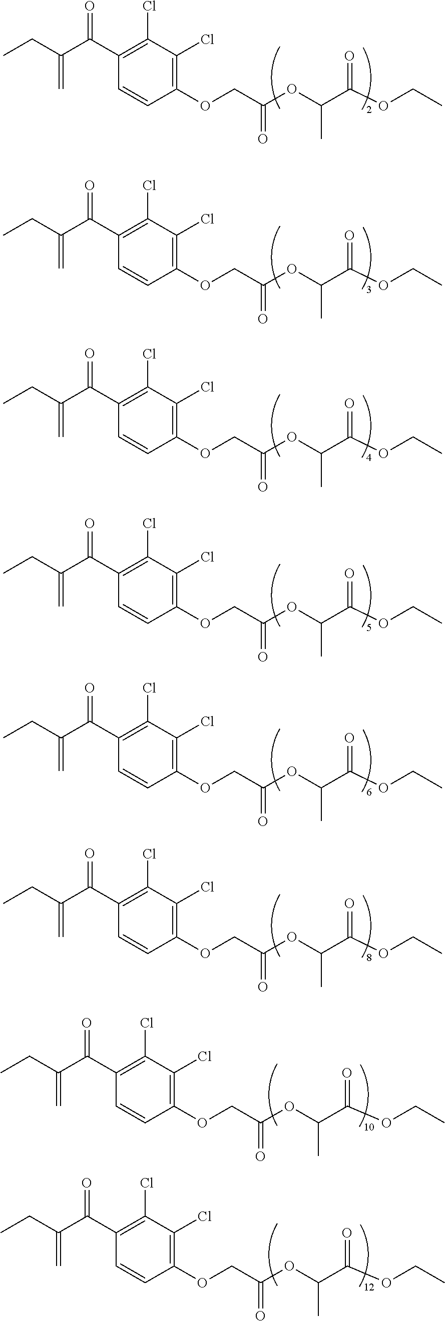

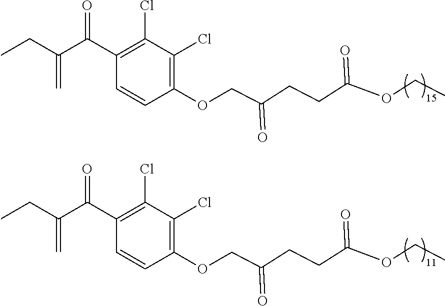

### Example 2A. Non-Limiting Examples of Compounds of Formula II

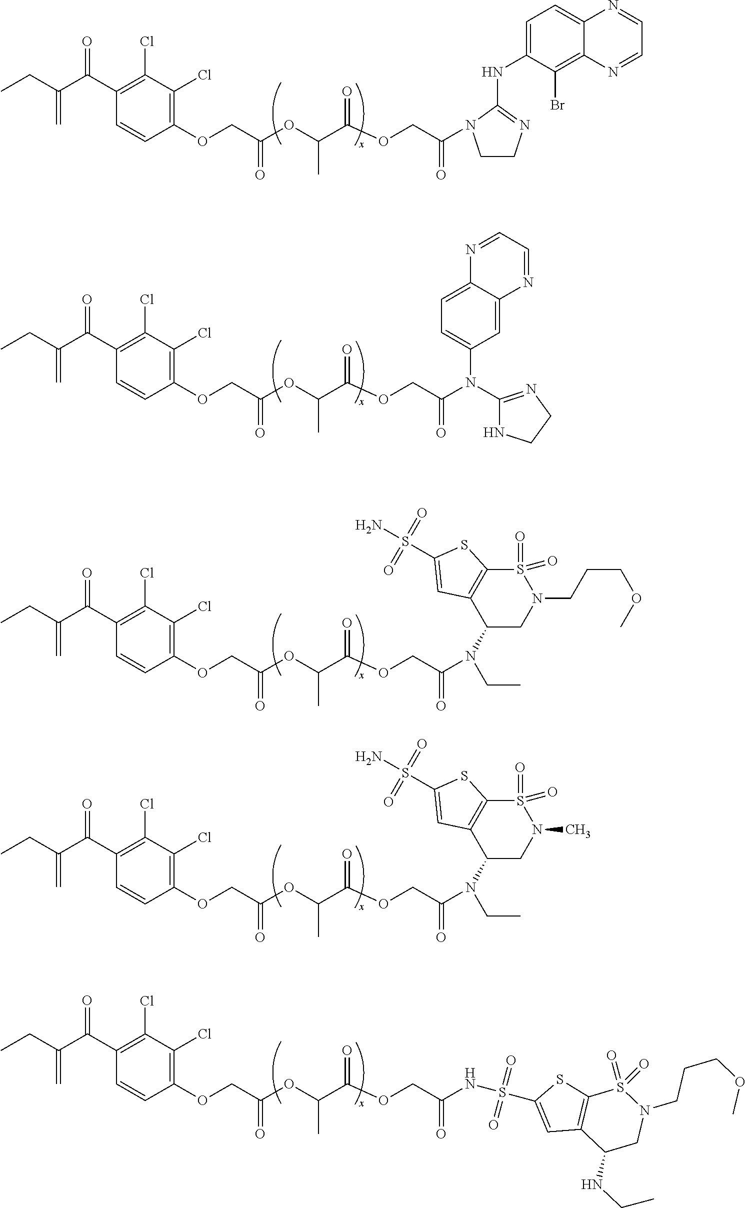

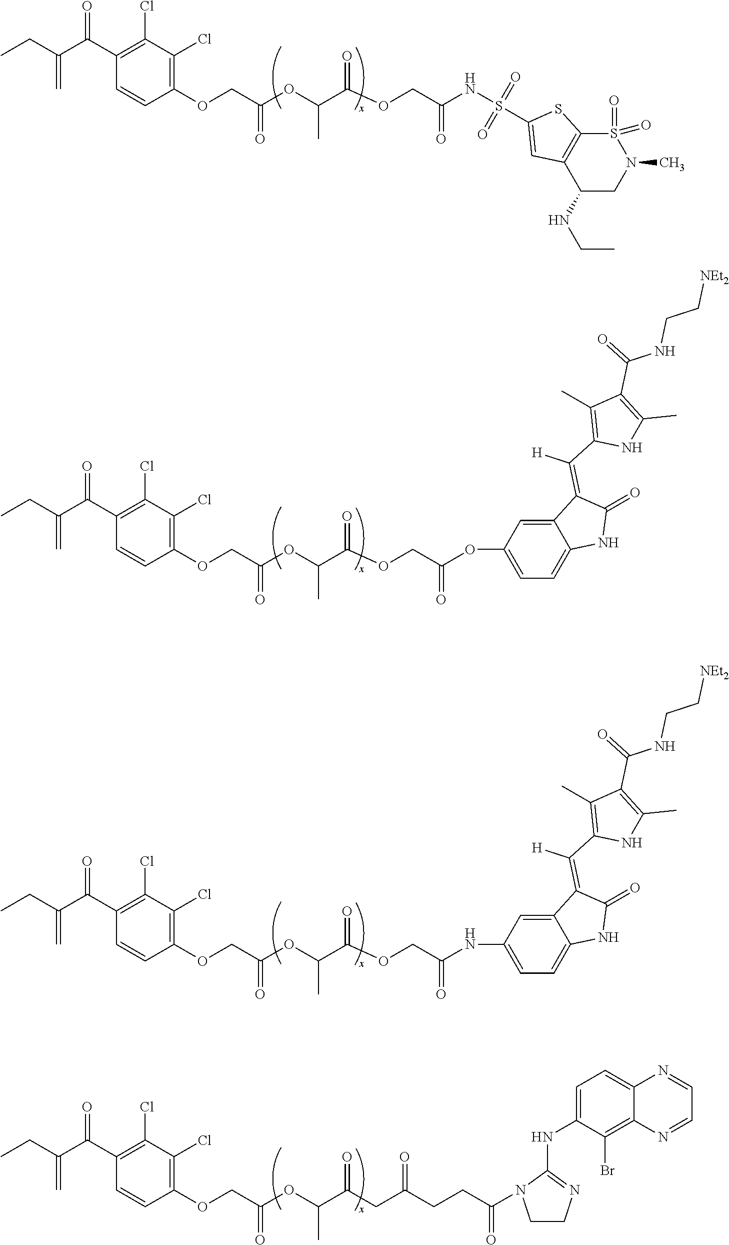

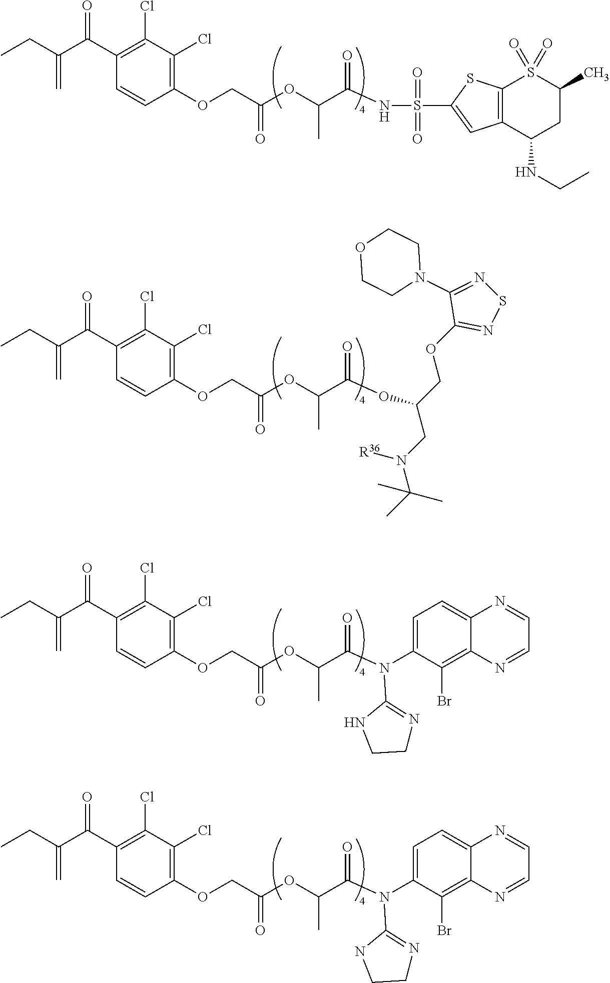

### Example 2B. Non-Limiting Examples of Compounds of Formula II

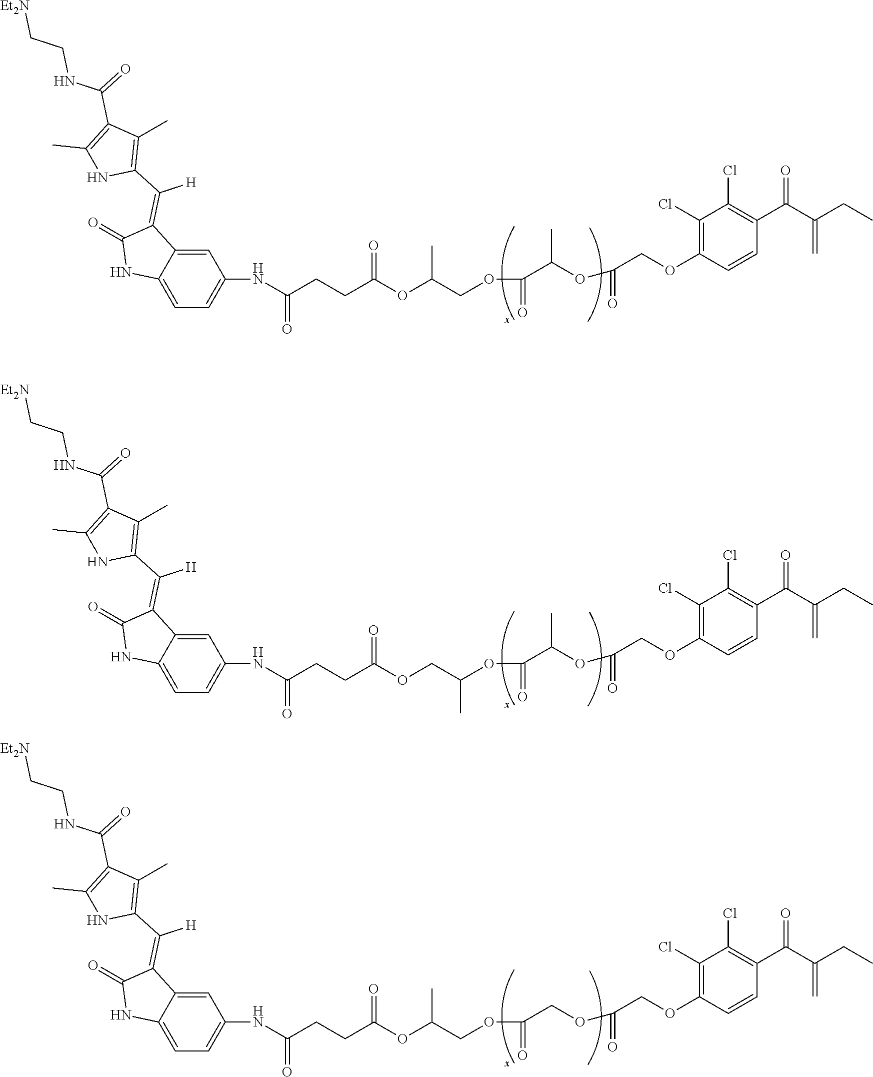

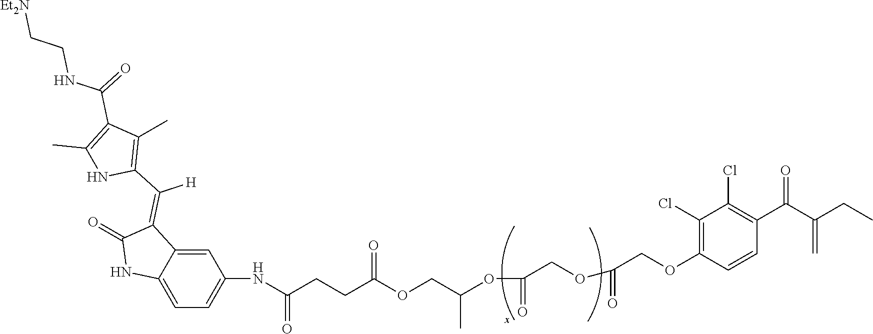

### Example 3. Non-limiting Examples of Compounds of Formula III, Formula IV, Formula IV′, Formula V, Formula VI, Formula X, and Formula XII

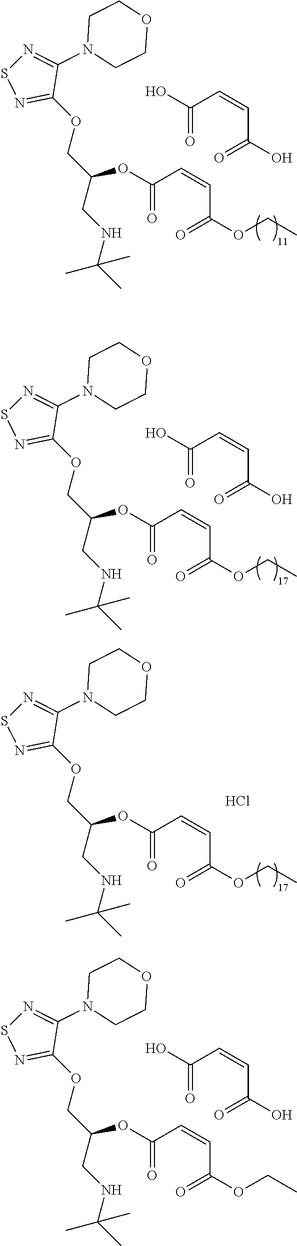

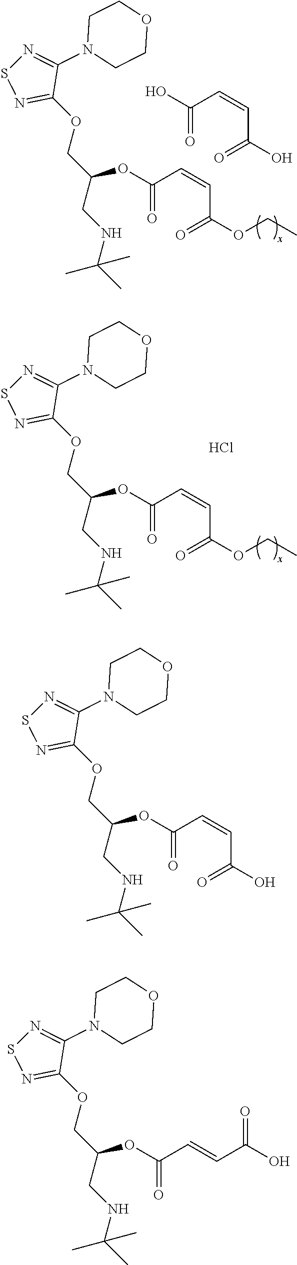

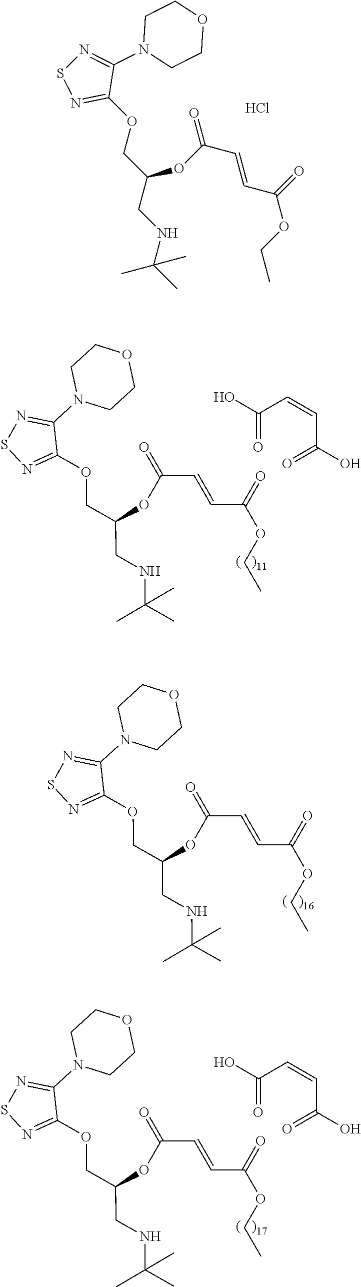

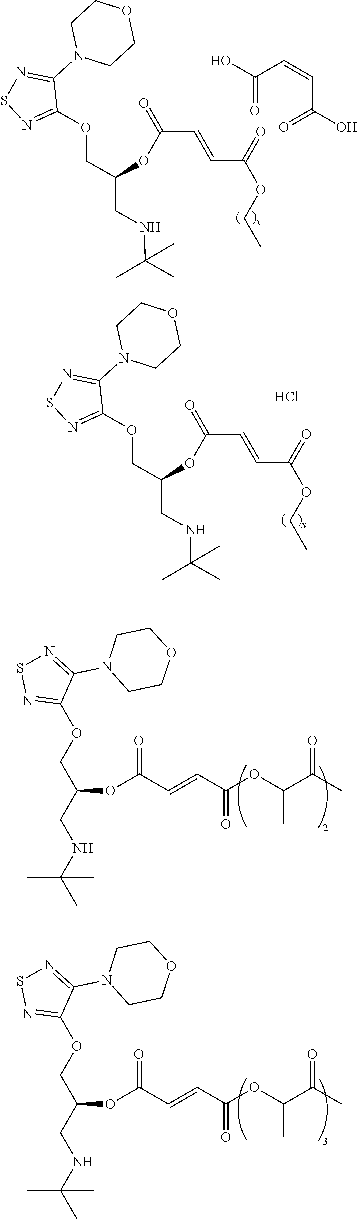

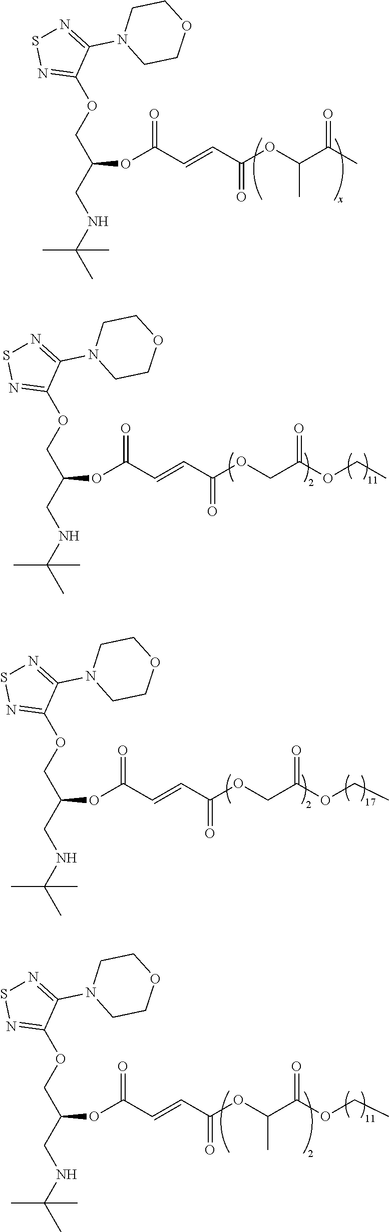

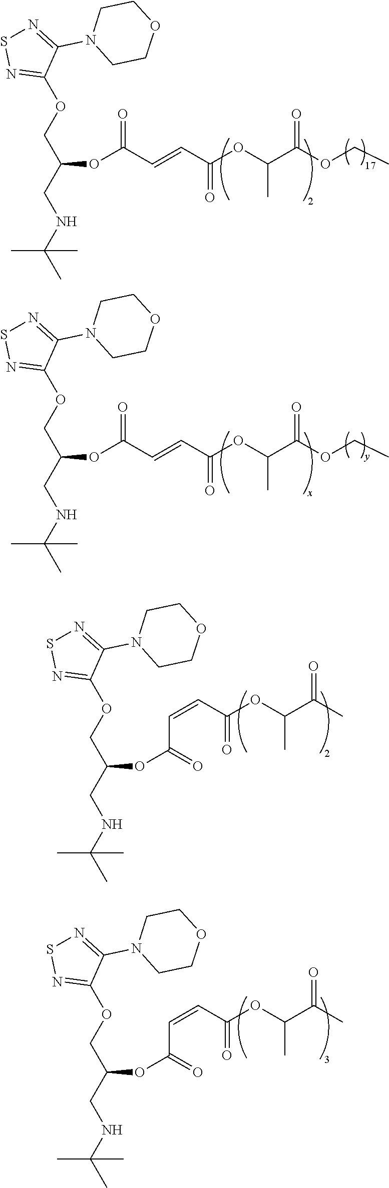

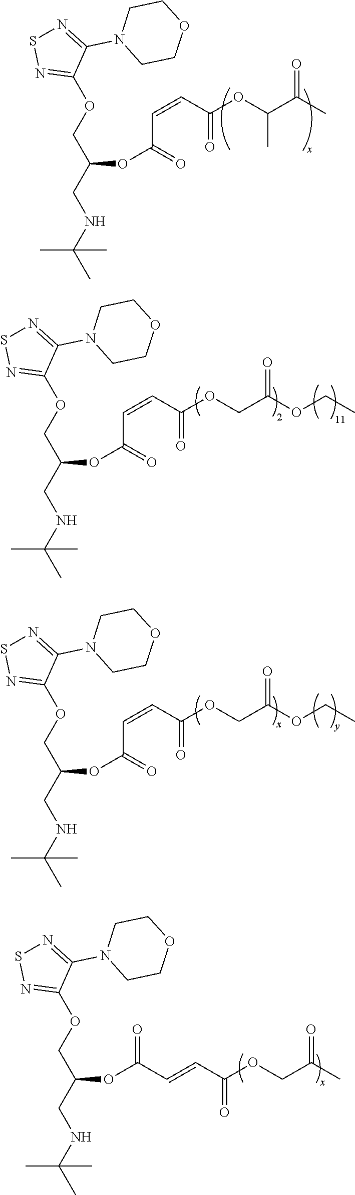

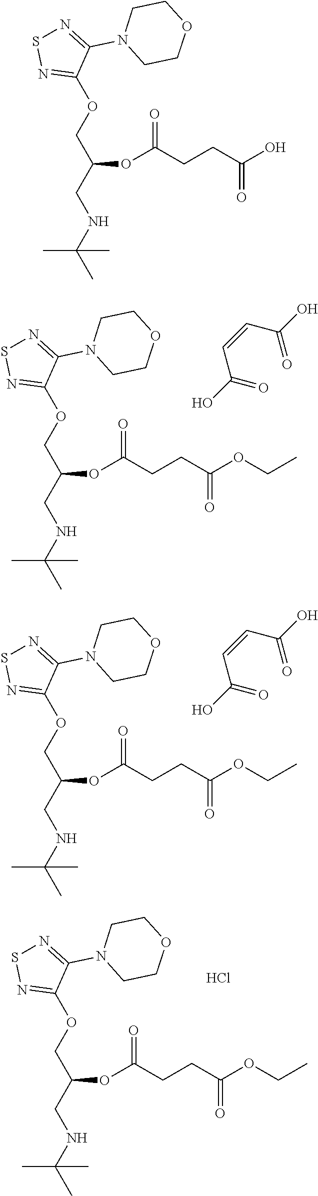

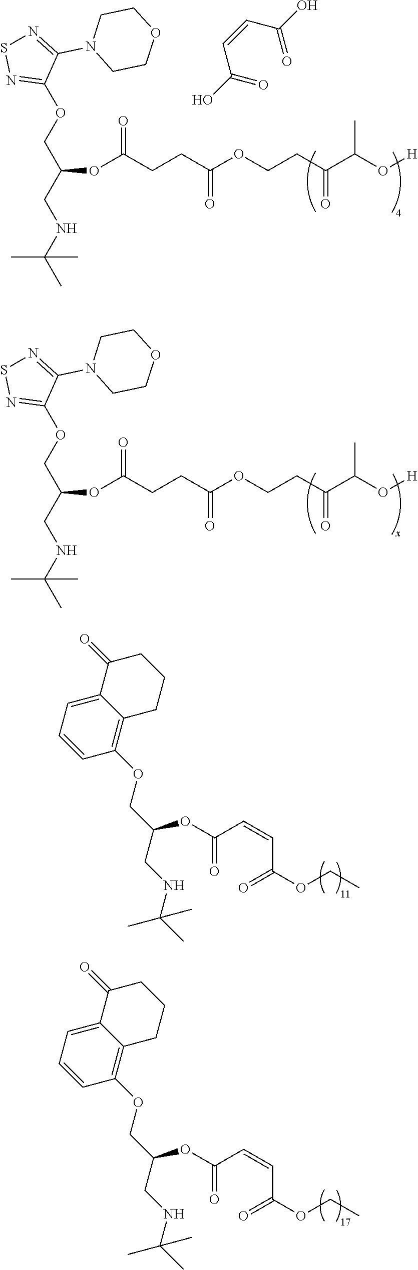

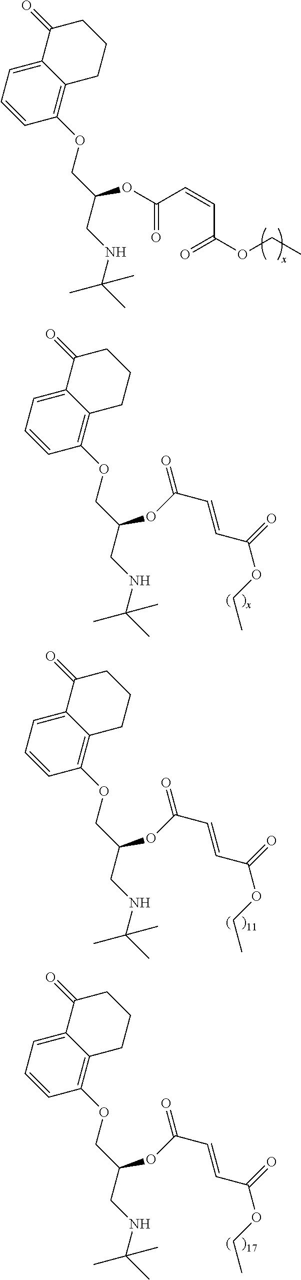

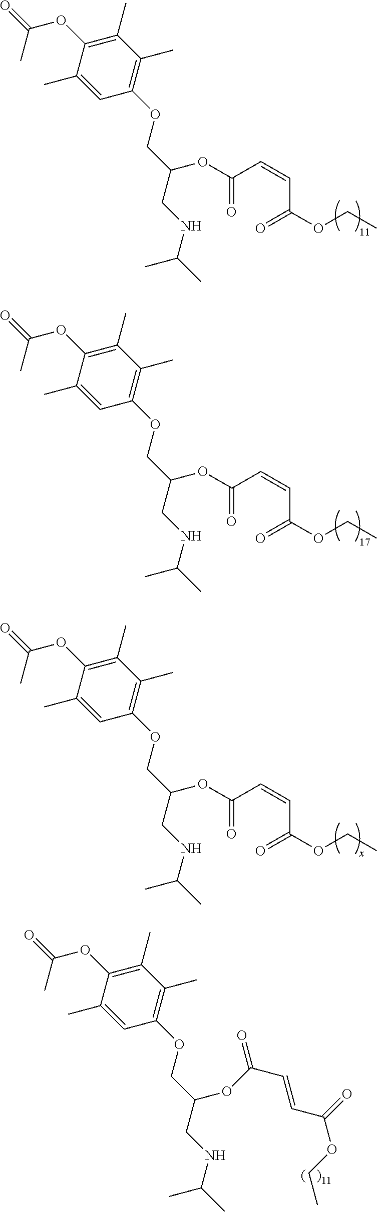

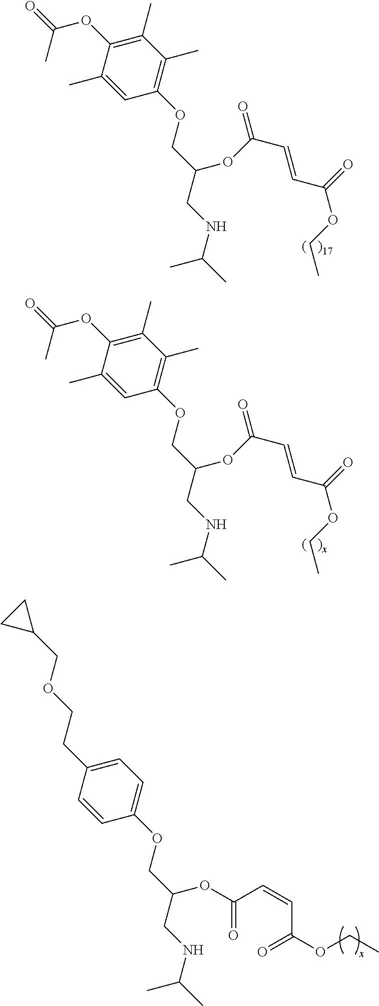

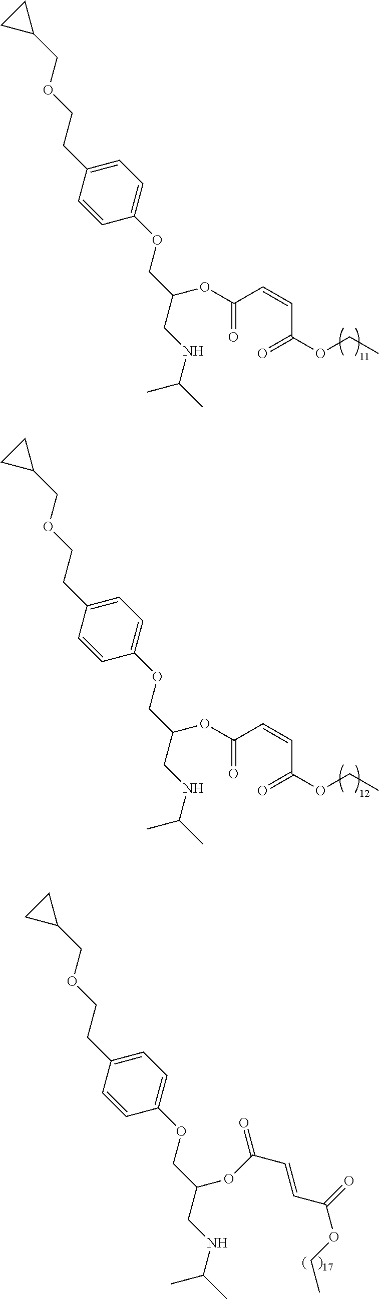

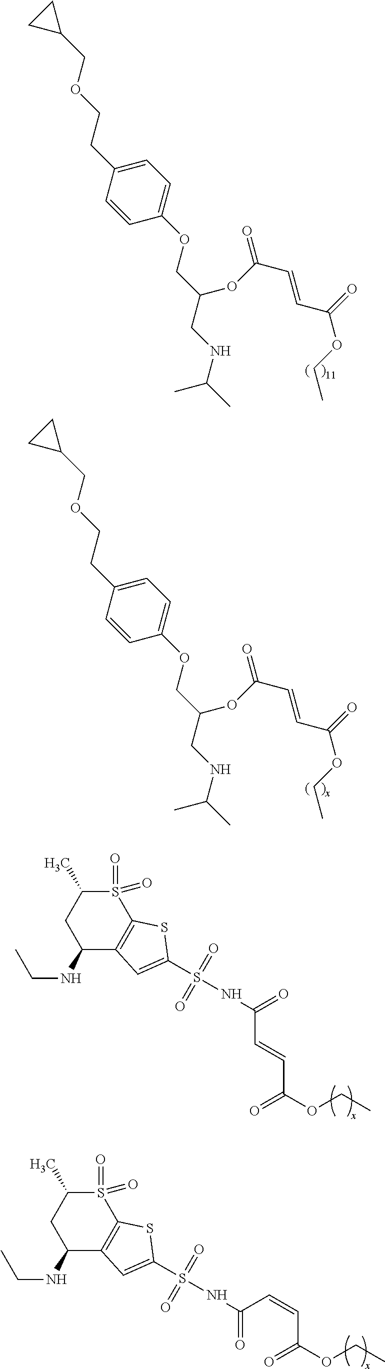

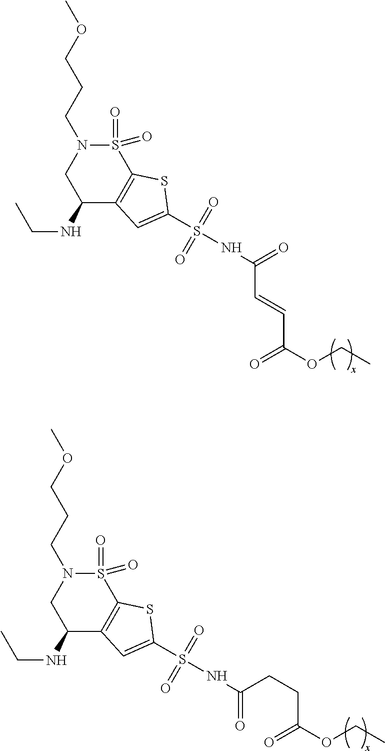

### Example 4. Non-limiting Examples of Compounds of Formula VII, Formula VIII, Formula VIII′, Formula IX, and Formula X

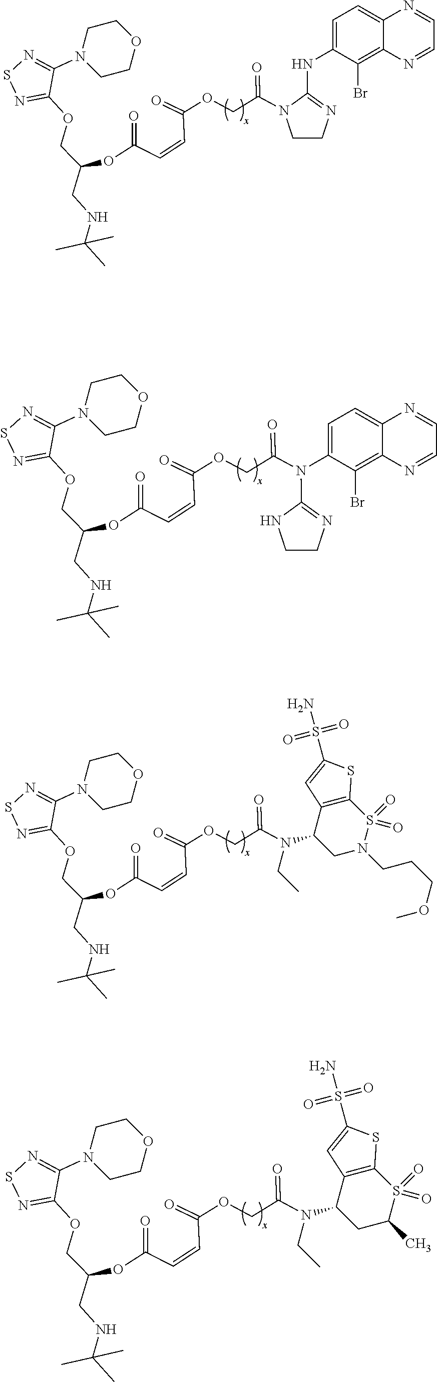

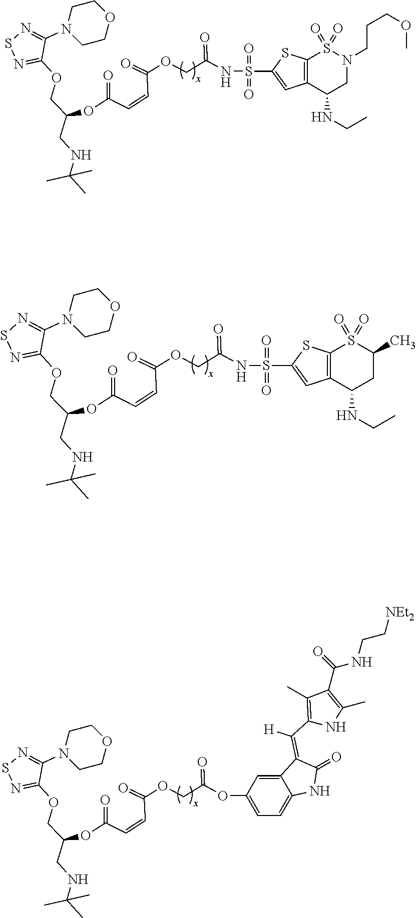

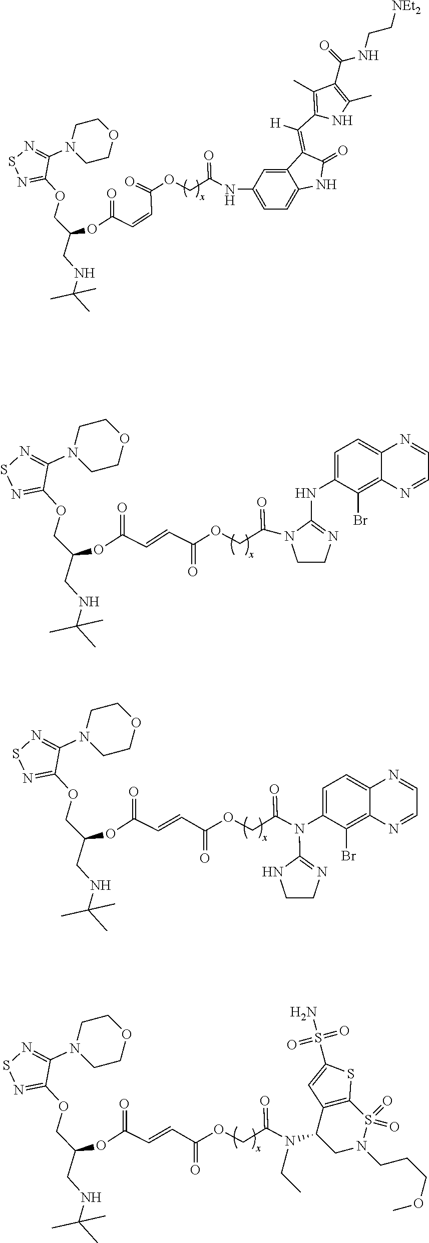

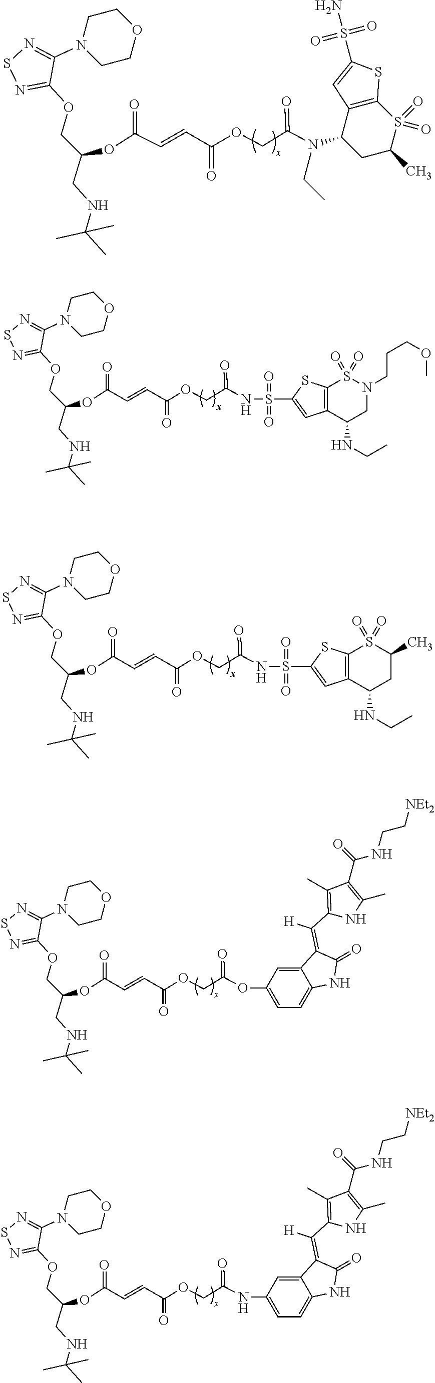

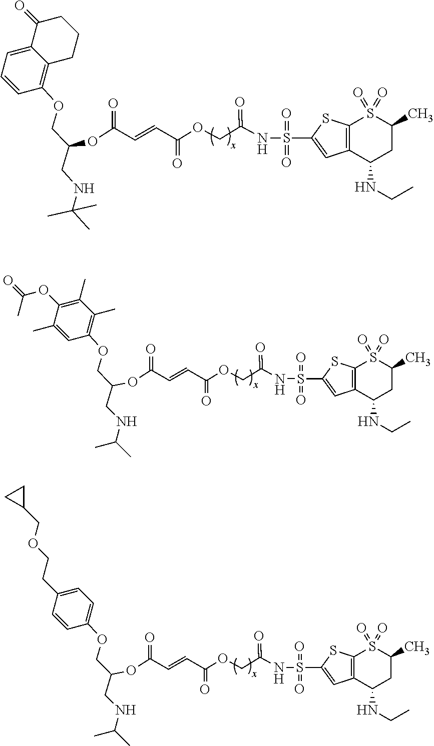

### Embodiments of x and y

In one embodiment x is 1 and y is 1.

In one embodiment x is 1 and y is 2.

In one embodiment x is 1 and y is 3.

In one embodiment x is 1 and y is 4.

In one embodiment x is 1 and y is 5.

In one embodiment x is 1 and y is 6.

In one embodiment x is 1 and y is 7.

In one embodiment x is 1 and y is 8.

In one embodiment x is 2 and y is 1.

In one embodiment x is 2 and y is 2.

In one embodiment x is 2 and y is 3.

In one embodiment x is 2 and y is 4.

In one embodiment x is 2 and y is 5.

In one embodiment x is 2 and y is 6.

In one embodiment x is 2 and y is 7.

In one embodiment x is 2 and y is 8.

In one embodiment x is 3 and y is 1.

In one embodiment x is 3 and y is 2.

In one embodiment x is 3 and y is 3.

In one embodiment x is 3 and y is 4.

In one embodiment x is 3 and y is 5.

In one embodiment x is 3 and y is 6.

In one embodiment x is 3 and y is 7.

In one embodiment x is 3 and y is 8.

In one embodiment x is 4 and y is 1.

In one embodiment x is 4 and y is 2.

In one embodiment x is 4 and y is 3.

In one embodiment x is 4 and y is 4.

In one embodiment x is 4 and y is 5.

In one embodiment x is 4 and y is 6.

In one embodiment x is 4 and y is 7.

In one embodiment x is 4 and y is 8.

In one embodiment x is 5 and y is 1.

In one embodiment x is 5 and y is 2.

In one embodiment x is 5 and y is 3.

In one embodiment x is 5 and y is 4.

In one embodiment x is 5 and y is 5.

In one embodiment x is 5 and y is 6.

In one embodiment x is 5 and y is 7.

In one embodiment x is 5 and y is 8.

In one embodiment x is 6 and y is 1.

In one embodiment x is 6 and y is 2.

In one embodiment x is 6 and y is 3.

In one embodiment x is 6 and y is 4.

In one embodiment x is 6 and y is 5.

In one embodiment x is 6 and y is 6.

In one embodiment x is 6 and y is 7.

In one embodiment x is 6 and y is 8.

In one embodiment x is 7 and y is 1.

In one embodiment x is 7 and y is 2.

In one embodiment x is 7 and y is 3.

In one embodiment x is 7 and y is 4.

In one embodiment x is 7 and y is 5.

In one embodiment x is 7 and y is 6.

In one embodiment x is 7 and y is 7.

In one embodiment x is 7 and y is 8.

In one embodiment x is 8 and y is 1.

In one embodiment x is 8 and y is 2.

In one embodiment x is 8 and y is 3.

In one embodiment x is 8 and y is 4.

In one embodiment x is 8 and y is 5.

In one embodiment x is 8 and y is 6.

In one embodiment x is 8 and y is 7.

In one embodiment x is 8 and y is 8.

### Example 5. Non-Limiting Examples of Compounds of the Present Invention

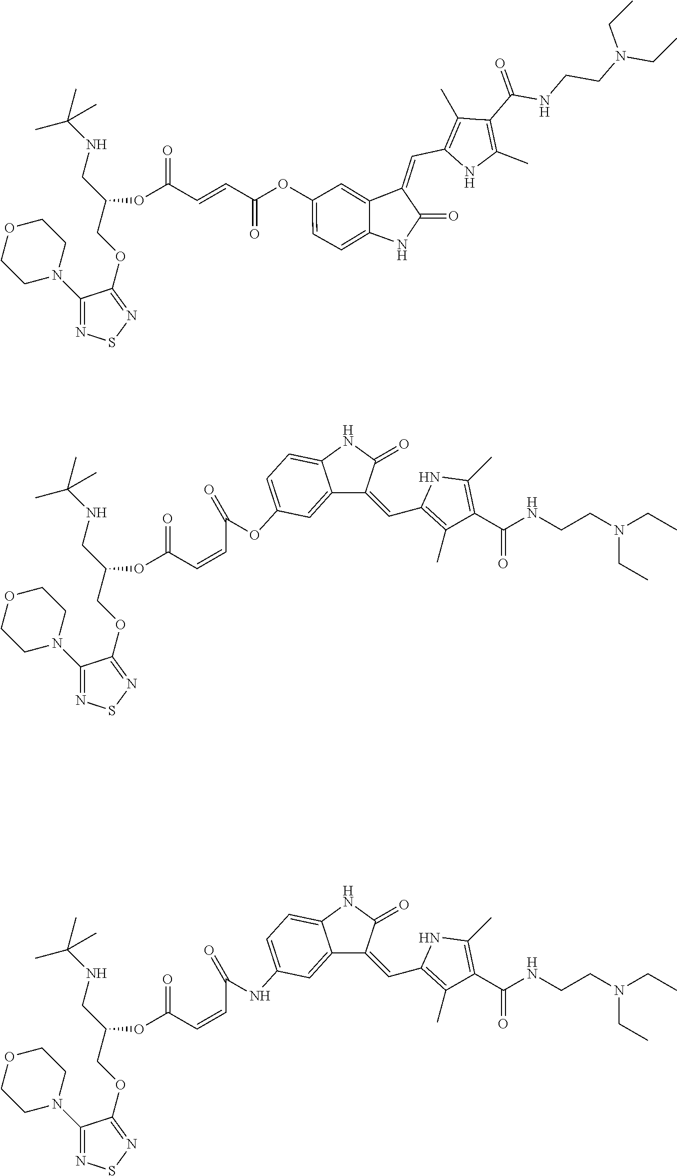 [SMILES: CCN(CC)CCNC(=O)C1=C(C)NC(/C=C2\C(=O)Nc3ccc(OC(=O)/C=C/C(=O)O[C@@H](CNC(C)(C)C)COc4nsnc4N4CCOCC4)cc32)=C(C)C1 ; CCN(CC)CCNC(=O)c1c(C)[nH]c(/C=C2\C(=O)Nc3ccc(OC(=O)/C=C\C(=O)O[C@@H](CNC(C)(C)C)COc4nsnc4N4CCOCC4)cc32)c1C ; CCN(CC)CCNC(=O)c1c(C)[nH]c(/C=C2\C(=O)Nc3ccc(NC(=O)/C=C\C(=O)O[C@@H](CNC(C)(C)C)COc4nsnc4N4CCOCC4)cc32)c1C]

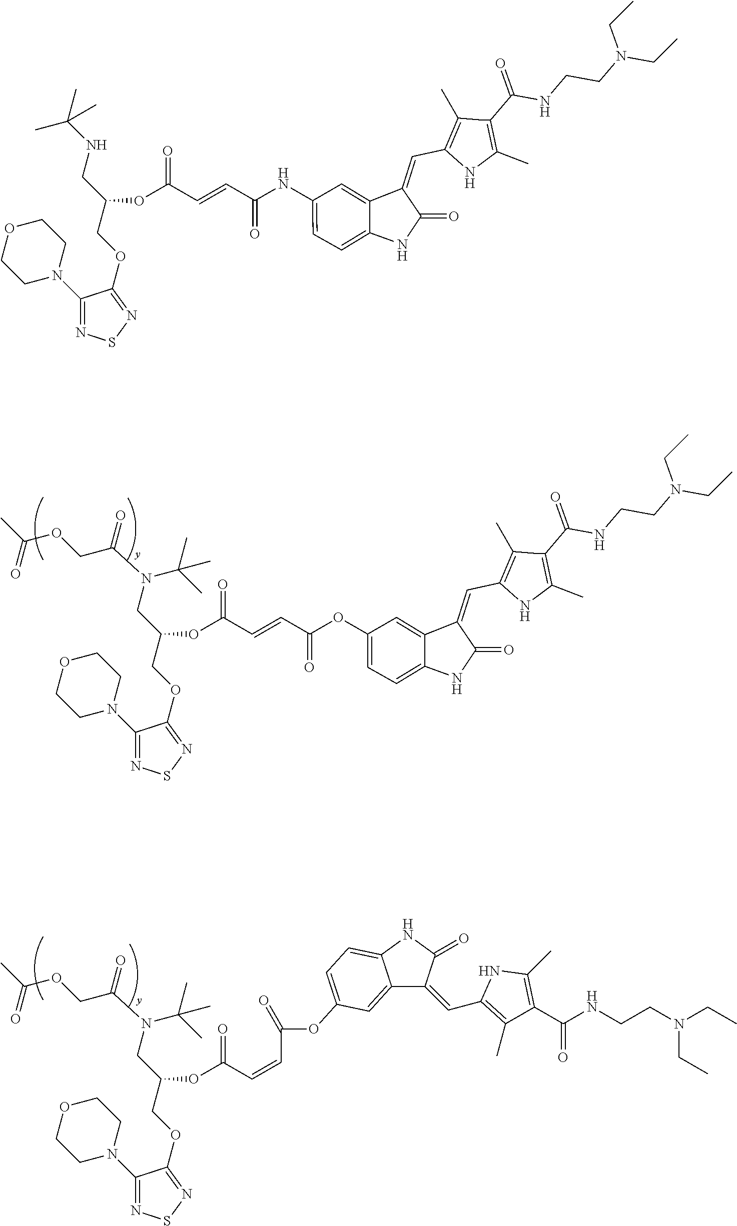

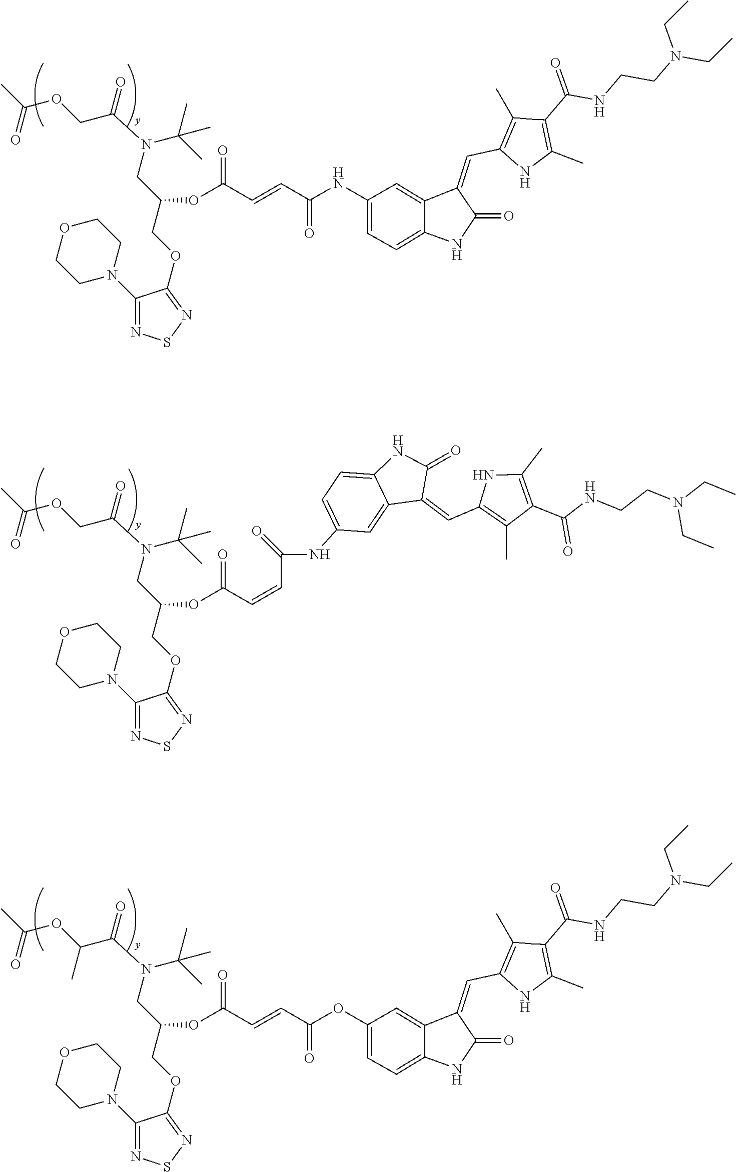

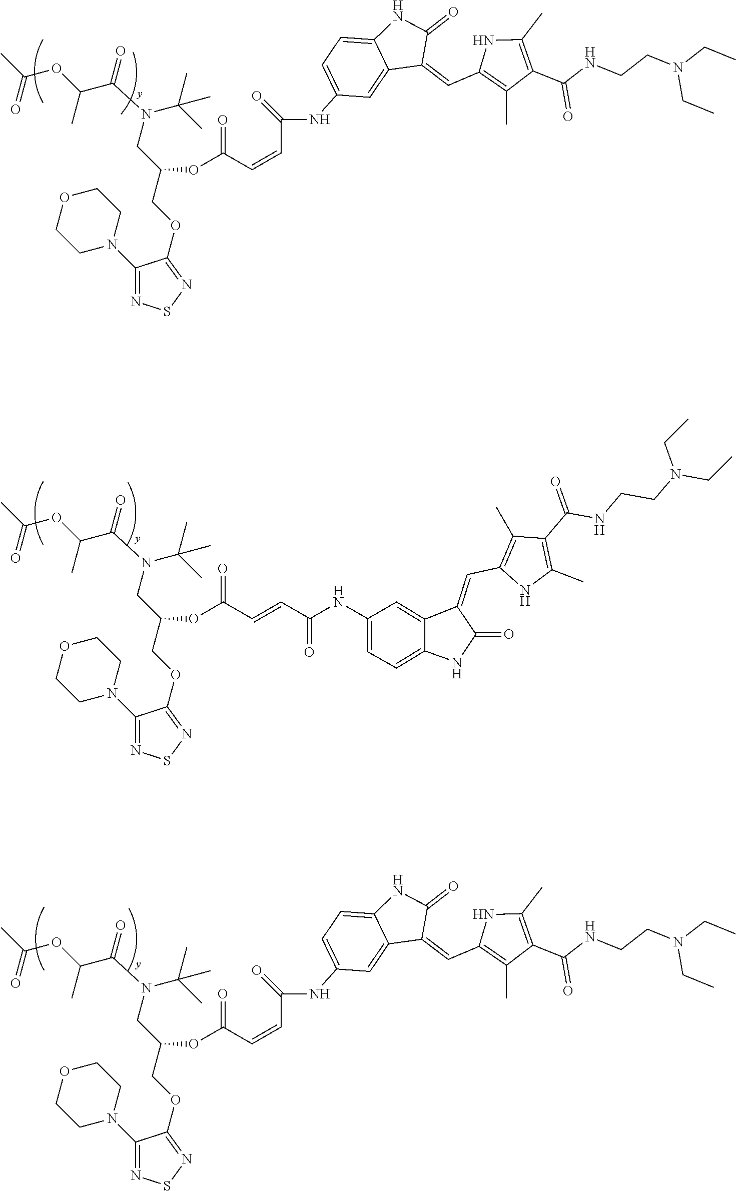

In one embodiment of the above structures,

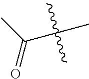 [SMILES: *C(C)=O]

is replaced with

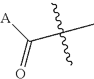

wherein A is defined above.
In another embodiment of the above structures,

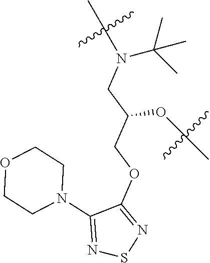 [SMILES: *O[C@H](COc1nsnc1N1CCOCC1)CN(*)C(C)(C)C]

is replaced with

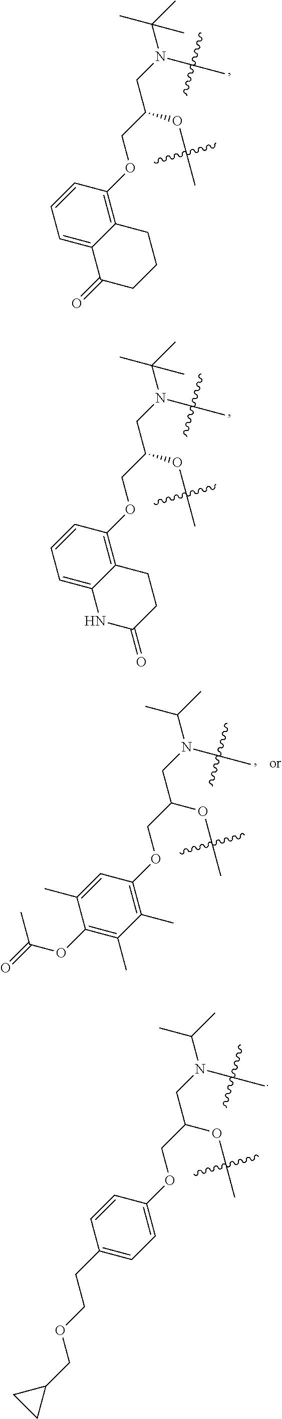

### Example 6. Additional Non-Limiting Examples of Compounds of the Present Invention

Table 1, Table 2, and Table 3 illustrate non-limiting examples of Compounds of Formula I, Formula II, Formula III, Formula IV, Formula IV′, Formula V, Formula VI, Formula VII, Formula VIII, Formula VIII′, Formula IX, Formula X, Formula XI, Formula XII, Formula XIII, Formula XIV, Formula XV, or Formula XVI. Characterization data is provided for select compounds of the present invention presented in Table 1.

TABLE 1
Compounds of the Present Invention

| Compd | |
|---|---|
| No. | Structure |
| 1 | 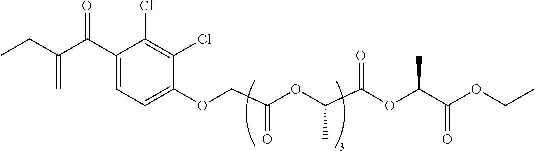 |
| | Characterization: ¹H-NMR (400 MHz, DMSO-d6) δ 7.32 (d, 1H, 8.6 Hz), 7.16 (d, |
| | 1H, 8.6 Hz), 6.07 (s, 1H), 5.56 (s, 1H), 5.28-5.04 (m, 6H), 4.17-4.04 (m, 2H), 2.44- |
| | 2.30 (m, 2H), 1.51-1.38 (m, 12H), 1.18 (t, 3H), 1.08 (t, 3H); MS m/z [M + Na]⁺ 498.4. |
| 2 | 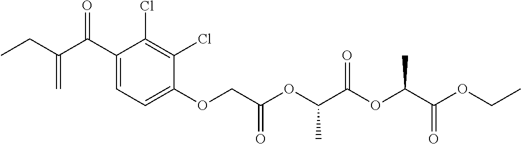 [SMILES: C=C(CC)C(=O)c1ccc(OCC(=O)O[C@@H](C)C(=O)O[C@@H](C)C(=O)OCC)c(Cl)c1Cl] |
| | Characterization: ¹H-NMR (400 MHz, DMSO-d6) δ 7.33 (d, 1H, 8.8 Hz), 7.16 (d, |
| | 1H, 8.8 Hz), 6.07 (s, 1H), 5.56 (s, 1H), 5.26-5.06 (m, 4H), 4.17-4.05 (m, 2H), 2.42- |
| | 2.28 (m, 2H), 1.49 (d, 3H), 1.43 (d, 3H), 1.17 (t, 3H), 1.08 (t, 3H); MS m/z [M + H]⁺ |
| | 475.8, [M + Na]⁺ 492.6. |
| 3 | 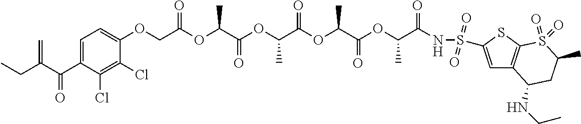 [SMILES: C=C(CC)C(=O)c1ccc(OCC(=O)O[C@@H](C)C(=O)O[C@@H](C)C(=O)O[C@@H](C)C(=O)O[C@@H](C)C(=O)NS(=O)(=O)c2cc3c(s2)S(=O)(=O)[C@@H](C)C[C@@H]3NCC)c(Cl)c1Cl] |
| | Characterization: ¹H-NMR (400 MHz, DMSO-d6) δ 7.46 (bs, 1H), 7.33 (d, 1H, 8.6 |
| | Hz), 7.17 (d, 1H, 8.6 Hz), 6.06 (s, 1H), 5.56 (s, 1H), 5.27-5.13 (m, 4H), 5.09 (q, 1H), |
| | 4.79 (q, 1H), 4.0-3.8 (m, 2H), 2.9-2.5 (bm, 2H), 2.5-2.3 (m, 4H), 1.51-1.43 (m, 9H), |
| | 1.34 (d, 3H), 1.30 (d, 3H), 1.07 (t, 6H); MS m/z [M + H]⁺ 898.4. |
| 4 | 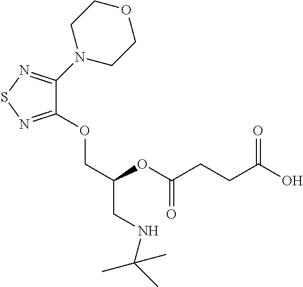 [SMILES: CC(C)(C)NC[C@@H](COc1nsnc1N1CCOCC1)OC(=O)CCC(=O)O] |
| | Characterization: ¹H-NMR (400 MHz, DMSO-d6) δ 5.19 (quintet, 1H), 4.59 (dd, |
| | 1H), 4.47 (dd, 1H), 3.72-3.65 (m, 4H), 3.45-3.28 (m, 4H), 2.82-2.72 (m, 2H), 2.5- |
| | 2.35 (m, 4H), 1.04 (s, 9H). MS m/z [M + H]⁺ 417.7. |
| 5 | 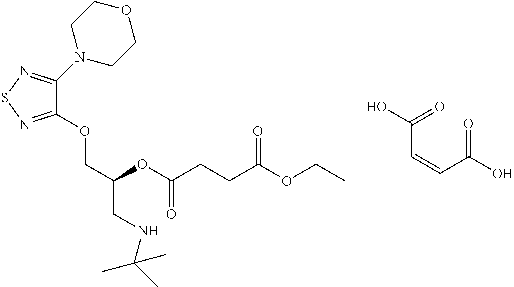 [SMILES: CCOC(=O)CCC(=O)O[C@@H](CNC(C)(C)C)COc1nsnc1N1CCOCC1 ; O=C(O)/C=C\C(=O)O] |
| | Characterization: ¹H-NMR (400 MHz, DMSO-d6) δ 8.55 (bs, 1H), 8.29 (bs, 1H), |
| | 6.01 (s, 2H), 5.45-5.35 (m, 1H), 4.63 (dd, 1H), 4.48 (dd, 1H), 4.03 (q, 2H), 3.73-3.67 |
| | (m, 4H), 3.6-3.4 (m, 5H), 3.28-3.13 (m, 1H), 2.7-2.5 (m, 4H), 1.29 (s, 9H), 1.16, (t, |
| | 3H). MS m/z [M + H]⁺ 445.6. |
| 6 | 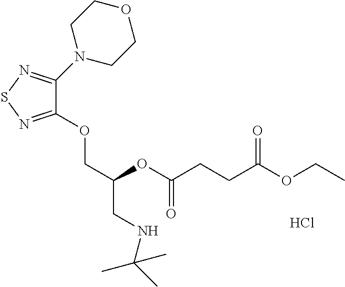 [SMILES: CCOC(=O)CCC(=O)O[C@@H](CNC(C)(C)C)COc1nsnc1N1CCOCC1 ; Cl] |
| | Characterization: ¹H-NMR (400 MHz, DMSO-d6) δ 9.30 (bs, 1H), 8.81 (bs, 1H), |
| | 5.50-5.40 (m, 1H), 4.62 (dd, 1H), 4.50 (dd, 1H), 4.02 (q, 2H), 3.72-3.64 (m, 4H), |
| | 3.49-3.29 (m, 5H), 3.25-3.12 (m, 1H), 2.76-2.55 (m, 4H), 1.32 (s, 9H), 1.15, (t, 3H). |
| | MS m/z [M + H]⁺ 445.7. |
| 7 | 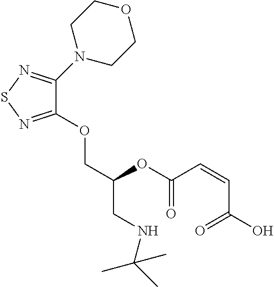 [SMILES: CC(C)(C)NC[C@@H](COc1nsnc1N1CCOCC1)OC(=O)/C=C\C(=O)O] |
| | Characterization: ¹H-NMR (400 MHz, DMSO-d6) δ 6.31(d, 1H), 5.78 (d, 1H), |
| | 5.51-5.41 (m, 1H), 4.64 (dd, 1H), 4.47 (dd, 1H), 3.74-3.66 (m, 4H), 3.5-3.1 (m, 6H), |
| | 1.32 (s, 9H), MS m/z [M + H]⁺ 415.6. |
| 8 | 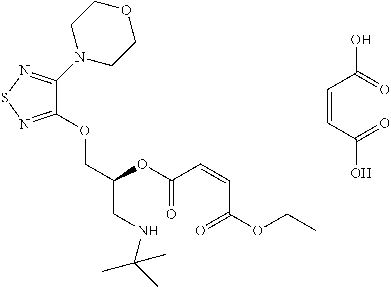 [SMILES: CCOC(=O)/C=C\C(=O)O[C@@H](CNC(C)(C)C)COc1nsnc1N1CCOCC1 ; O=C(O)/C=C\C(=O)O] |
| | Characterization: ¹H-NMR (400 MHz, DMSO-d6) δ 6.31(d, 1H), 5.78 (d, 1H), |
| | 5.51-5.41 (m, 1H), 4.64 (dd, 1H), 4.47 (dd, 1H), 3.74-3.66 (m, 4H), 3.5-3.1 (m, 6H), |
| | 1.32 (s, 9H), MS m/z [M + H]⁺ 415.6. |
| 9 | 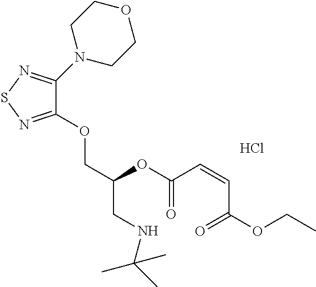 [SMILES: CCOC(=O)/C=C\C(=O)O[C@@H](CNC(C)(C)C)COc1nsnc1N1CCOCC1 ; Cl] |
| | Characterization: ¹H-NMR (400 MHz, DMSO-d6) δ 9.33 (bt, 1H), 8.73 (bt, 1H), |
| | 6.86 (d, 1H), 6.78 (d, 1H), 5.65-5.55 (m, 1H), 4.72 (dd, 1H), 4.53 (dd, 1H), 4.22 (q, |
| | 1H), 3.70-3.64 (m, 4H), 3.45-3.2 (m, 6H), 1.32 (s, 9H), 1.25 (t, 3H). MS m/z [M + H]⁺ |
| | 443.6. |
| 10 | 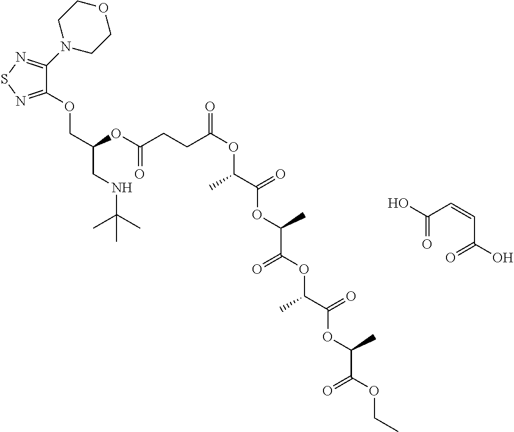 [SMILES: CCOC(=O)[C@H](C)OC(=O)[C@H](C)OC(=O)[C@H](C)OC(=O)[C@H](C)OC(=O)CCC(=O)O[C@@H](CNC(C)(C)C)COc1nsnc1N1CCOCC1 ; O=C(O)/C=C\C(=O)O] |
| 11 | 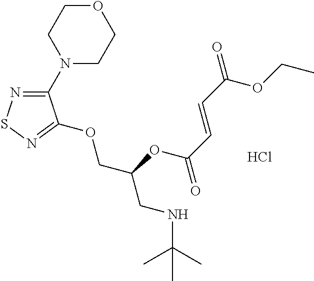 [SMILES: CCOC(=O)/C=C/C(=O)O[C@@H](CNC(C)(C)C)COc1nsnc1N1CCOCC1 ; Cl] |
| | Characterization: ¹H-NMR (400 MHz, DMSO-d6) δ 9.20 (bs, 1H), 8.32 (bs, 1H), |
| | 6.87 (d, 1H), 6.78 (d, 1H), 5.58-5.54 (m, 1H), 4.73 (dd, 1H), 4.53 (dd, 1H), 4.22 (q, |
| | 2H), 3.72-3.64 (m, 4H), 3.5-3.2 (m, 6H), 1.32 (s, 9H), 1.25 (t, 3H). MS m/z [M + H]⁺ |
| | 443.7. |
| 12 | 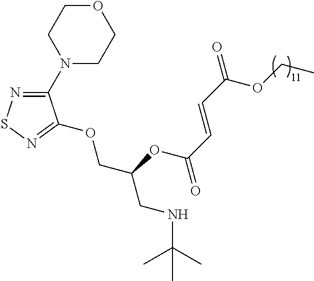 |
| | Characterization: ¹H-NMR (400 MHz, DMSO-d6) δ 6.77 (d, J = 16 Hz, 1H), 6.74 |
| | (d, J = 16 Hz, 1H), 5.30-5.22 (m, 1H), 4.67 (dd, 1H), 4.52 (dd, 1H), 4.15 (t, 2H), |
| | 3.79-3.63 (m, 4H), 3.5-3.3 (m, 4H), 2.84-2.74 (m, 2H), 1.61 (quintet, 2H), 1.37-1.22 |
| | (m, 18H), 1.01 (s, 9H), 0.85 (t, 3H). MS m/z [M + H]⁺ 583.8. |
| 13 | 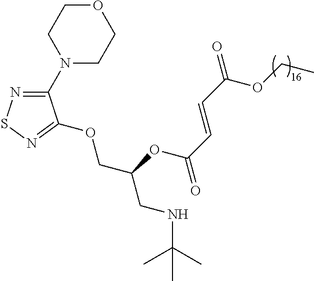 |
| 14 | 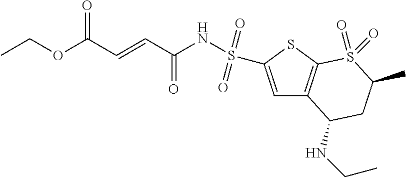 [SMILES: CCN[C@H]1C[C@H](C)S(=O)(=O)c2sc(S(=O)(=O)NC(=O)/C=C/C(=O)OCC)cc21] |
| | Characterization: ¹H NMR (400 MHz, DMSO-d6) δ 7.43 (s, 1H), 6.67 (d, 1H), 6.42 |
| | (d, 1H), 4.14 (q, 2H), 4.9-4.8 (m, 2H), 2.7-2.5 (m, 2H), 2.4-2.15 (m, 2H), 1.32 (d, |
| | 3H), 1.22 (t, 3H), 1.02 (t, 3H); m/z [M + H]⁺ 451.5. |
| 15 | 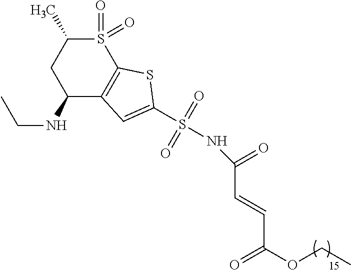 |
| | Characterization: ¹H NMR (400 MHz, DMSO-d6) δ 7.43 (s, 1H), 6.65 (d, 1H), 6.41 |
| | (d, 1H), 4.08 (t, 2H), 4.9-4.8 (m, 2H), 2.7-2.5 (m, 2H), 2.4-2.18 (m, 2H), 1.59 |
| | (quintet, 2H) 1.32 (d, 3H), 1.3-1.2 (m, 30H), 1.02 (t, 3H), 0.83 (t, 3H); m/z [M + H]⁺ |
| | 676.0. |
| 16 | 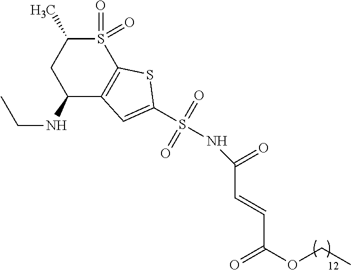 |
| 17 | 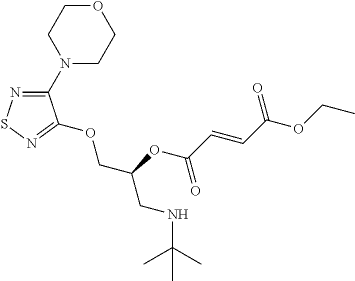 [SMILES: CCOC(=O)/C=C/C(=O)O[C@@H](CNC(C)(C)C)COc1nsnc1N1CCOCC1] |
| | Characterization: ¹H-NMR (400 MHz, DMSO-d6) δ 6.8-6.7 (m, 2H), 5.3-5.2 (m, |
| | 1H), 4.68 (dd, 1H), 4.52 (dd, 1H), 4.20 (q, 2H), 3.7-3.6 (m, 4H), 3.45-3.33 (m, 4H), |
| | 2.8-2.7 (m, 2H), 1.26, (t, 3H), 1.00 (s, 9H), MS m/z [M + H]⁺ 443.6. |
| 18 | 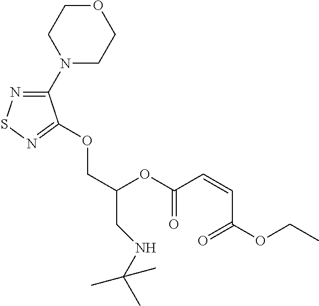 [SMILES: CCOC(=O)/C=C\C(=O)OC(CNC(C)(C)C)COc1nsnc1N1CCOCC1] |
| | Characterization: ¹H-NMR (400 MHz, DMSO-d6) δ 6.50 (d, 1H), 6.44 (d, 1H), |
| | 5.27-5.14 (m, 1H), 4.63 (dd, 1H), 4.49 (dd, 1H), 4.12 (q, 2H), 3.7-3.6 (m, 4H), 3.44- |
| | 3.2 (m, 4H), 2.8-2.7, (m, 2H), 1.19 (t, 3H), 1.00 (s, 9H),. MS m/z [M + H]⁺ 443.6. |
| 19 | 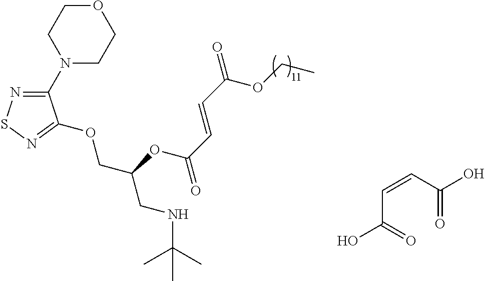 |
| | Characterization: ¹H-NMR (400 MHz, DMSO-d6) δ 8.65 (bs, 1H), 8.29 (bs, 1H), |
| | 6.87 (d, J = 15.5 Hz, 1H), 6.78 (d, J = 15.5 Hz, 1H), 6.03 (s, 2H), 5.60-5.50 (m, 1H), |
| | 4.74 (dd, 1H), 4.51 (dd, 1H), 4.17 (t, 2H), 3.68-3.62 (m, 4H), 3.5-3.2 (m, 6H), 1.62 |
| | (quintet, 2H), 1.35-1.19 (m, 27H), 0.85 (t, 3H). MS m/z [M + H]⁺ 583.7. |
| 20 | 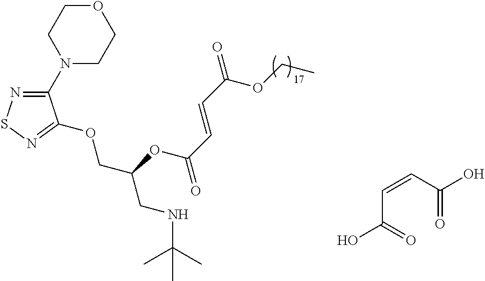 |
| | Characterization: ¹H-NMR (400 MHz, DMSO-d6) δ 8.63 (bs, 1H), 8.27 (bs, 1H), |
| | 6.87 (d, J = 16 Hz, 1H), 6.78 (d, J = 16 Hz, 1H), 6.04 (s, 2H), 5.60-5.50 (m, 1H), |
| | 4.74 (dd, 1H), 4.51 (dd, 1H), 4.17 (t, 2H), 3.70-3.61 (m, 4H), 3.5-3.2 (m, 6H), 1.62 |
| | (quintet, 2H), 1.35-1.16 (m, 39H), 0.85 (t, 3H). MS m/z [M + H]⁺ 668.0. |
| 23 | 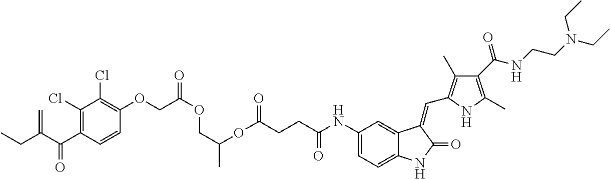 [SMILES: C=C(CC)C(=O)c1ccc(OCC(=O)OCC(C)OC(=O)CCC(=O)Nc2ccc3c(c2)/C(=C/c2[nH]c(C)c(C(=O)NCCN(CC)CC)c2C)C(=O)N3)c(Cl)c1Cl] |
| | Characterization: ¹H-NMR (400 MHz, DMSO-d6) δ 13.64 (s, 1H), 10.82 (s, 1H), |
| | 9.87 (s, 1H), 7.87 (s, 1H), 7.5-7.4 (m, 2H), 7.3-7.05 (m, 3H), 7.29-7.17 (m, 2H), 6.78 |
| | (d, 1H), 6.02 (s, 1H), 5.52 (s, 1H), 5.2-4.9 (m, 5H), 4.3-4.0 (m, 2H), 3.4-3.2 (m, 6H), |
| | 2.6-2.2 (m, 12H), 1.25 (d, 3H), 1.1-0.9 (m, 9H); MS m/z [M + H]⁺ 839.9. |
| 24 | 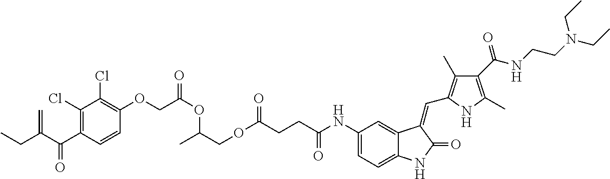 [SMILES: C=C(CC)C(=O)c1ccc(OCC(=O)OC(C)COC(=O)CCC(=O)Nc2ccc3c(c2)/C(=C/c2[nH]c(C)c(C(=O)NCCN(CC)CC)c2C)C(=O)N3)c(Cl)c1Cl] |
| 25 | 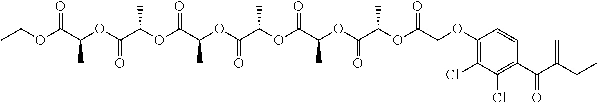 [SMILES: C=C(CC)C(=O)c1ccc(OCC(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)c(Cl)c1Cl] |
| | Characterization: ¹H-NMR (400 MHz, DMSO-d6) δ 7.32 (d, 1H, 8.8 Hz), 7.16 (d, |
| | 1H, 8.8 Hz), 6.08 (s, 1H), 5.56 (s, 1H), 5.28-5.04 (m, 8H), 4.16-4.05 (m, 2H), 2.4- |
| | 2.3 (m, 2H), 1.51-1.39 (m, 18H), 1.18 (t, 3H), 1.08 (t, 3H); MS m/z [M + H]⁺ 765.1. |
| 26 | 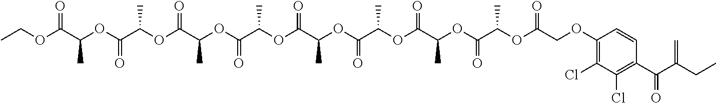 [SMILES: C=C(CC)C(=O)c1ccc(OCC(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)c(Cl)c1Cl] |
| | Characterization: ¹H-NMR (400 MHz, DMSO-d6) δ 7.32 (d, 1H, 8.8 Hz), 7.16 (d, |
| | 1H, 8.8 Hz), 6.08 (s, 1H), 5.56 (s, 1H), 5.28-5.04 (m, 10H), 4.17-4.06 (m, 2H), 2.4- |
| | 2.28 (m, 2H), 1.50-1.39 (m, 24H), 1.18 (t, 3H), 1.07 (t, 3H); MS m/z +M + H+⁺909.7. |
| 27 | 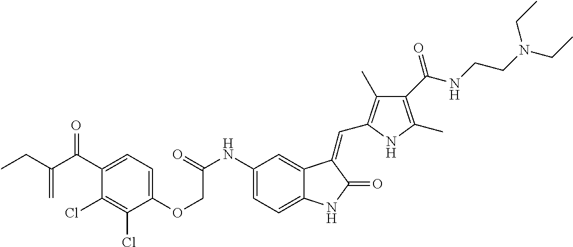 [SMILES: C=C(CC)C(=O)c1ccc(OCC(=O)Nc2ccc3c(c2)/C(=C/c2[nH]c(C)c(C(=O)NCCN(CC)CC)c2C)C(=O)N3)c(Cl)c1Cl] |
| | Characterization: ¹H-NMR (400 MHz, DMSO-d6) δ 13.73 (s, 1H), 10.92 (s, 1H), |
| | 10.10 (s, 1H), 7.98 (s, 1H), 7.73 (t, 1H), 7.50 (s, 1H), 7.37 (d, 1H), 7.29-7.17 (m, |
| | 2H), 6.86 (d, 1H), 6.06 (s, 1H), 5.59 (s, 1H), 4.97 (s, 2H), 3.6-3.4 (m, 2H), 3.3-3.0 |
| | (m, 6H), 2.5-2.25 (m, 8H), 1.17 (t, 6H), 1.07 (t, 3H); MS m/z [M + H]⁺ 681.8. |
| 28 | 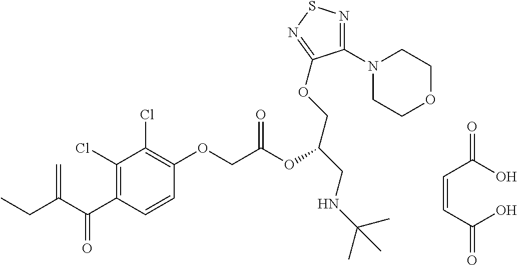 [SMILES: C=C(CC)C(=O)c1ccc(OCC(=O)O[C@@H](CNC(C)(C)C)COc2nsnc2N2CCOCC2)c(Cl)c1Cl ; O=C(O)/C=C\C(=O)O] |
| | Characterization: ¹H-NMR (400 MHz, DMSO-d6) δ 8.69 (bs, 1H), 8.36 (bs, 1H), |
| | 7.28 (d, 1H, 8.8 Hz), 7.21 (d, 1H, 8.8 Hz), 6.08 (bs, 3H), 5.6-5.5 (m, 2H) 5.13 (d, |
| | 1H, 17 Hz), 5.03 (d, 1H, 17 Hz), 4.67 (dd, 1H), 4.50 (dd, 1H), 3.7-3.58 (m, 4H), 3.5- |
| | 3.2 (m, 6H), 2.35 (q, 2H), 1.31 (s, 9H), 1.07 (t, 3H); MS m/z [M + H]⁺ 602.5. |
| 42 | 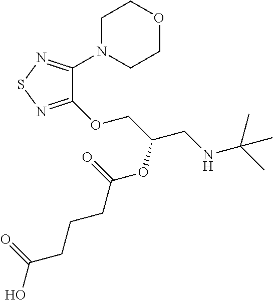 [SMILES: CC(C)(C)NC[C@@H](COc1nsnc1N1CCOCC1)OC(=O)CCCC(=O)O] |

TABLE 2A
Compounds of the Present Invention

| | |
|---|---|
| 21 | 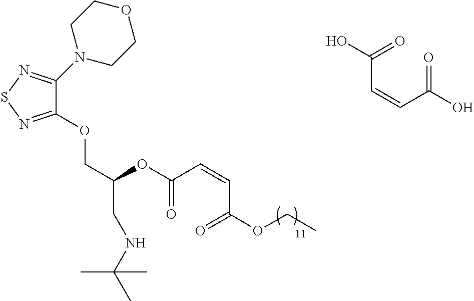 |
| 22 | 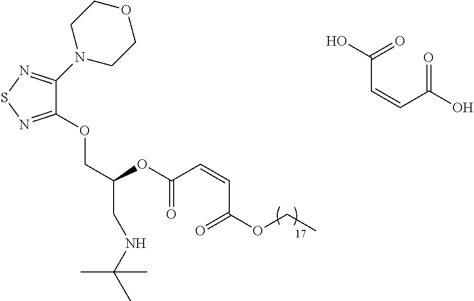 |
| 29 | 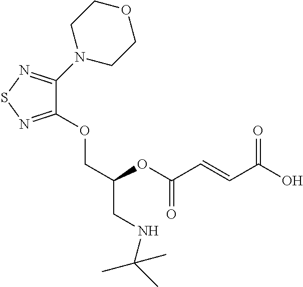 [SMILES: CC(C)(C)NC[C@@H](COc1nsnc1N1CCOCC1)OC(=O)/C=C/C(=O)O] |
| 30 | 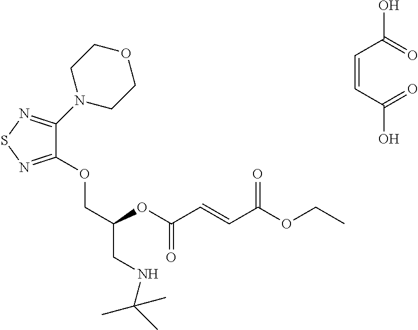 [SMILES: CCOC(=O)/C=C/C(=O)O[C@@H](CNC(C)(C)C)COc1nsnc1N1CCOCC1 ; O=C(O)/C=C\C(=O)O] |
| 31 | 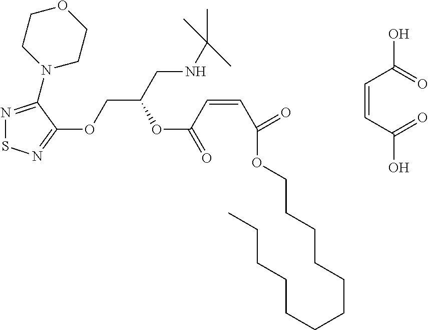 [SMILES: CCCCCCCCCCCCOC(=O)/C=C\C(=O)O[C@@H](CNC(C)(C)C)COc1nsnc1N1CCOCC1 ; O=C(O)/C=C\C(=O)O] |
| 32 | 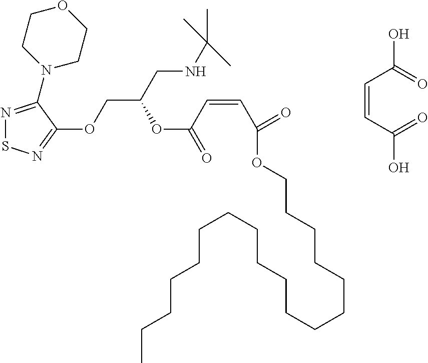 [SMILES: CCCCCCCCCCCCCCCCCCOC(=O)/C=C\C(=O)O[C@@H](CNC(C)(C)C)COc1nsnc1N1CCOCC1 ; O=C(O)/C=C\C(=O)O] |
| 33 | 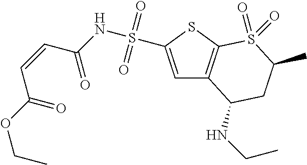 [SMILES: CCN[C@H]1C[C@H](C)S(=O)(=O)c2sc(S(=O)(=O)NC(=O)/C=C\C(=O)OCC)cc21] |
| 34 | 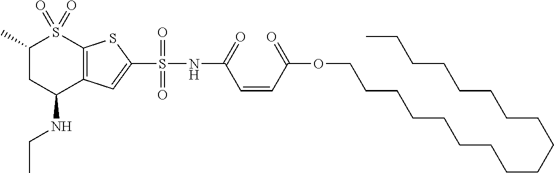 [SMILES: CCCCCCCCCCCCCCCCCCOC(=O)/C=C\C(=O)NS(=O)(=O)c1cc2c(s1)S(=O)(=O)[C@@H](C)C[C@@H]2NCC] |
| 35 | 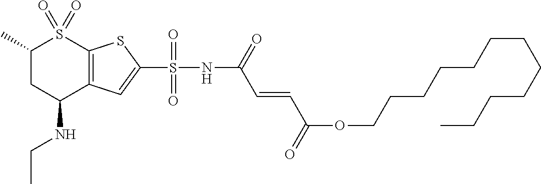 [SMILES: CCCCCCCCCCCCOC(=O)/C=C/C(=O)NS(=O)(=O)c1cc2c(s1)S(=O)(=O)[C@@H](C)C[C@@H]2NCC] |
| 36 | 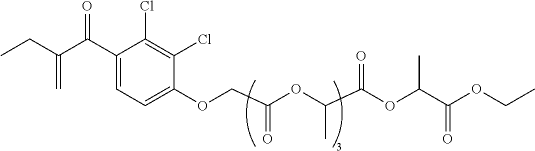 |
| 37 | 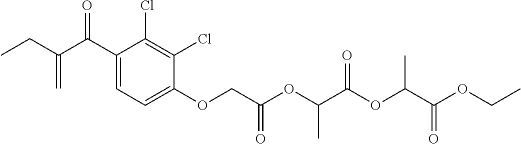 [SMILES: C=C(CC)C(=O)c1ccc(OCC(=O)OC(C)C(=O)OC(C)C(=O)OCC)c(Cl)c1Cl] |
| 38 | 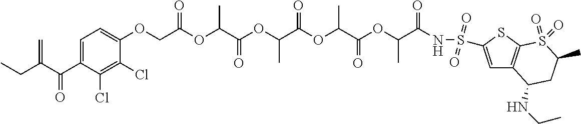 [SMILES: C=C(CC)C(=O)c1ccc(OCC(=O)OC(C)C(=O)OC(C)C(=O)OC(C)C(=O)OC(C)C(=O)NS(=O)(=O)c2cc3c(s2)S(=O)(=O)[C@@H](C)C[C@@H]3NCC)c(Cl)c1Cl] |
| 39 | 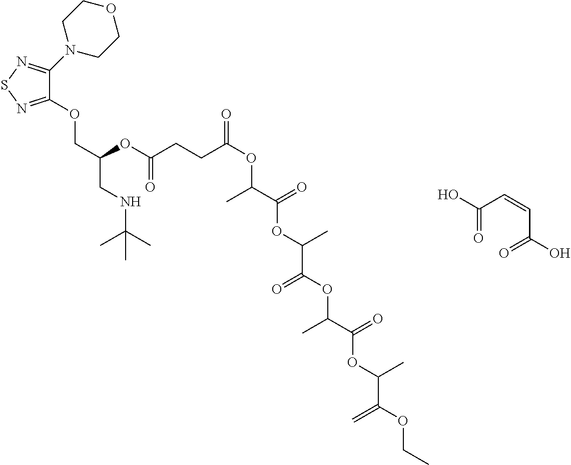 [SMILES: C=C(OCC)C(C)OC(=O)C(C)OC(=O)C(C)OC(=O)C(C)OC(=O)CCC(=O)O[C@@H](CNC(C)(C)C)COc1nsnc1N1CCOCC1 ; O=C(O)/C=C\C(=O)O] |
| 40 | 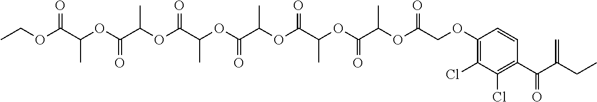 [SMILES: C=C(CC)C(=O)c1ccc(OCC(=O)OC(C)C(=O)OC(C)C(=O)OC(C)C(=O)OC(C)C(=O)OC(C)C(=O)OC(C)C(=O)OCC)c(Cl)c1Cl] |
| 41 | 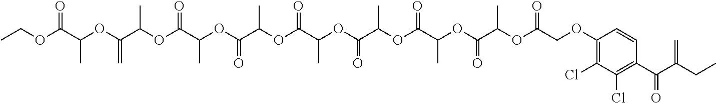 [SMILES: C=C(CC)C(=O)c1ccc(OCC(=O)OC(C)C(=O)OC(C)C(=O)OC(C)C(=O)OC(C)C(=O)OC(C)C(=O)OC(C)C(=O)OC(C)C(=C)OC(C)C(=O)OCC)c(Cl)c1Cl] |

TABLE 2B
Additional Compounds of the Present Invention

| | |
|---|---|
| 42 | 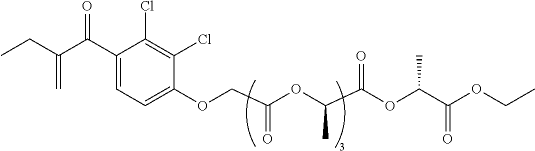 |
| 43 | 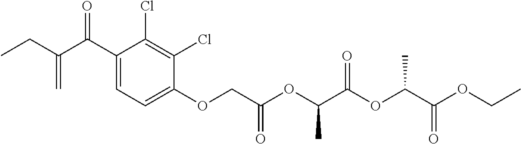 [SMILES: C=C(CC)C(=O)c1ccc(OCC(=O)O[C@H](C)C(=O)O[C@H](C)C(=O)OCC)c(Cl)c1Cl] |
| 44 | 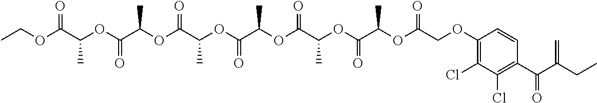 [SMILES: C=C(CC)C(=O)c1ccc(OCC(=O)O[C@H](C)C(=O)O[C@H](C)C(=O)O[C@H](C)C(=O)O[C@H](C)C(=O)O[C@H](C)C(=O)O[C@H](C)C(=O)OCC)c(Cl)c1Cl] |
| 45 | 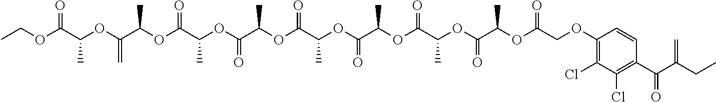 [SMILES: C=C(CC)C(=O)c1ccc(OCC(=O)O[C@H](C)C(=O)O[C@H](C)C(=O)O[C@H](C)C(=O)O[C@H](C)C(=O)O[C@H](C)C(=O)O[C@H](C)C(=O)O[C@H](C)C(=C)O[C@H](C)C(=O)OCC)c(Cl)c1Cl] |
| 46 | 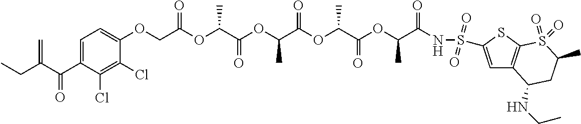 [SMILES: C=C(CC)C(=O)c1ccc(OCC(=O)O[C@H](C)C(=O)O[C@H](C)C(=O)O[C@H](C)C(=O)O[C@H](C)C(=O)NS(=O)(=O)c2cc3c(s2)S(=O)(=O)[C@@H](C)C[C@@H]3NCC)c(Cl)c1Cl] |

TABLE 3
Description of Compounds of the Present Invention

| Active Moiety | General Name | Mw | Mw w/Salt | Molecular Formula |
|---|---|---|---|---|
| Ethacrynic acid | Ethacrynic acid-PLA (n = 4)-Ethyl ester | 619.45 | NA | C₂₇H₃₂Cₗ₂O₁₂ |
| Ethacrynic acid | Ethacrynic acid-PLA (n = 2)-Ethyl ester | 475.33 | NA | C₂₁H₂₄Cₗ₂O8 |
| Timolol | Timolol-O-Ethylsuccinate-Maleate | 444.55 | 560.62 | C₁₉H₃₂N₄O₆S•C₄H₄O₄ |
| | | | (Maleate salt) | |
| Timolol | Timolol-O-Ethylsuccinate-HCl salt | 444.55 | 481.01 | C₁₉H₃₂N₄O₆S•HCl |
| | | | (HCl salt) | |
| Timolol | Timolol-O-Maleate | 414.48 | NA | C₁₇H₂₆N₄O₆S |
| Timolol | Timolol-O-Ethylmaleate-Maleic acid salt | 442.54 | 558.61 | C₁₉H₃₀N₄O₆S•C₄H₄O₄ |
| | | | (Maleate salt) | |
| Timolol | Timolol-O-Ethylmaleate-HCl salt | 442.54 | 479.0 | C₁₉H₃₀N₄O₆S•HCl |
| | | | (HCl salt) | |
| Timolol | Timolol-O-Ethylmaleate-HCl salt | 442.54 | 479.0 | C₁₉H₃₀N₄O₆S•HCl |
| | | | (HCl salt) | |
| Dorzolamide | Dorzolamaide-Ethylfumarate | 450.55 | NA | C₁₆H₂₂N₂O₇S₃ |
| Dorzolamide | Dorzolamide-Stearylfumarate | 674.99 | NA | C₃₂H₅₄N₂O₇S₃ |
| Dorzolamide | Dorzolamide-Laurylmaleate | 590.83 | NA | C₂₆H₄₂N₂O₇S₃ |
| Timolol | Timolol-O-Laurylfumarate-maleate | 582.81 | 698.88 | C₂₉H₅₀N₄O₆S•C₄H₄O₄ |
| | | | (Maleate salt) | |
| Timolol | Timolol-O-Stearylfumarate-maleate | 666.97 | 783.04 | C₃₅H₆₂N₄O₆S•C₄H₄O₄ |
| | | | (Maleate salt) | |
| Timolol | Timolol-Fumarate-Timolol-Maleate | 712.89 | 945.03 | C₃₀H₄₈N₈O₈S2•2C₄H₄O₄ |
| | | | (Maleate salt) | |
| Ethacrynic acid, | Brimonidine-PLA (n = 4)-Ethacrynic acid | 865.53 | NA | C₃₆H₃₆BrCl₂N₅O₁₁ |
| Brimonidine | | | | |
| Ethacrynic acid, | Dorzolamide-PLA (n = 4)-Ethacrynic acid | 897.83 | NA | C₃₅H₄₂Cl₂N₂O₁₅S₃ |
| Dorzolamide | | | | |
| Ethacrynic acid | Ethyl PLA (n = 6)-Ethacrynic acid | 763.58 | NA | C₃₃H₄₀Cₗ₂O₁₆ |
| Ethacrynic acid | Ethyl PLA (n = 8)-Ethacrynic acid | 907.71 | NA | C₃₉H₄₈Cₗ₂O₂₀ |
| Ethacrynic acid, | 5-Amino Sunitinib-Ethacrynic acid | 680.64 | NA | C₃₅H₃₉Cl₂N₅O₅ |
| Sunitinib | | | | |
| Timolol, | Timolol-Ethacrynic acid-maleate | 601.55 | 717.62 | C₂₆H₃₄Cₗ₂N₄O₆S•C₄H₄O₄ |
| Ethacrynic acid | | | (Maleate salt) | |
| Ethacrynic acid, | 5-Amino Sunitinib-1,2-propyleneglycol- | 838.79 | NA | C₄₂H₄₉Cₗ₂N₅O₉ |
| Sunitinib | Ethacrynic acid | | | |

### Example 7. Analytical Method Development for Compounds Containing Ethacrynic Acid

### HPLC Method for Prodrugs of Ethacrynic Acid

Prodrugs of ECA were challenged by heating at 60° C. in 50/50 water (0.1% FA)/acetonitrile (0.1% FA), and the degradants were separated using a reverse phase HPLC equipped with a C-18 bonded stationary phase. The identification of various peaks was accomplished by mass spectrometry detector and retention time comparison with available standards.

Chromatographic separation of ethacrynic acid parent compounds and their PLA conjugated derivatives was achieved using an Agilent 1260 Infinity II LCMS equipped with an Waters Symmetry C18 column (5 μm, 4.6 mm×150 mm) as the stationary phase and acetonitrile/water as the mobile phase. The gradient separation method is outlined in Table 4. The analysis was performed at an injection volume of 10 μL, a flow rate of 1.2 mL/min and a detection wavelength of 230 nm at 25° C. Retention times for ethacrynic acid and PLA-conjugated compounds are illustrated in Table 5.

TABLE 4
HPLC gradient method for separation
of Ethacrynic acid derivatives

| Time (min) | A (water + 0.1% FA) | B (CH₃CN + 0.1% FA) |
|---|---|---|
| 0 | 25 | 75.0 |
| 6.5 | 5.0 | 95.0 |

TABLE 5
Relative retention times of Ethacrynic acid and its derivatives

| PLA Repeat Units | RT (min) |
|---|---|
| Parent | 2.14 |
| n = 1 | 2.29 |
| n = 2 | 2.47 |
| n = 3 | 2.70 |
| n = 4 | 2.93 |
| n = 5 | 3.18 |
| n = 6 | 3.44 |
| ethyl ester, | 5.21 |
| n = 6 (25) | |

n is the number of LA repeat units conjugated to the parent compound

The ECA-PLA(n=6) prodrug (25) was found to hydrolyze into five intermediates to eventually releases the parent ECA compound. Table 6 illustrates the calculated mass and structure of all the individual intermediates and the parent compound. Calculated mass ions were extracted using a MS G6135B detector with positive and negative polarity acquisition between mass 100-1000, fragmentation at 250, gain at 1, threshold of 150, and speed of 2080 u/sec. Extracted ions and polarity for individual mass spectometry peak identification is outlined in Table 7.

TABLE 6
Calculated mass and structure of ECA-PLA (n = 6) (25) and its degradants

| | |
|---|---|
| 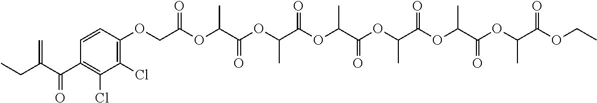 [SMILES: C=C(CC)C(=O)c1ccc(OCC(=O)OC(C)C(=O)OC(C)C(=O)OC(C)C(=O)OC(C)C(=O)OC(C)C(=O)OC(C)C(=O)OCC)c(Cl)c1Cl] | ECA-PLA6-ET (25) MF C₃₃H₄₀Cl₂O₁₆ MW 763.57 |
| 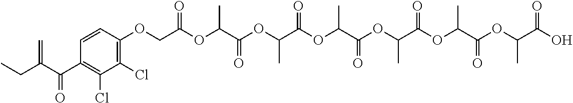 [SMILES: C=C(CC)C(=O)c1ccc(OCC(=O)OC(C)C(=O)OC(C)C(=O)OC(C)C(=O)OC(C)C(=O)OC(C)C(=O)OC(C)C(=O)O)c(Cl)c1Cl] | ECA-PLA6 MF C₃₁H₃₆Cl₂O₁₆ MW 735.51 |
| 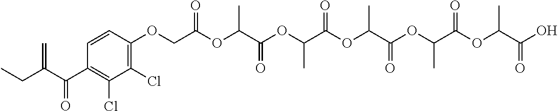 [SMILES: C=C(CC)C(=O)c1ccc(OCC(=O)OC(C)C(=O)OC(C)C(=O)OC(C)C(=O)OC(C)C(=O)OC(C)C(=O)O)c(Cl)c1Cl] | ECA-PLA5 MF C₂₈H₃₂Cl₂O₁₄ MW 663.45 |
| 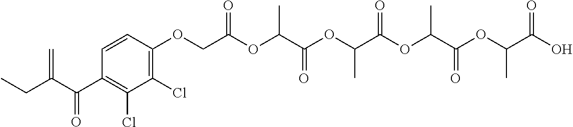 [SMILES: C=C(CC)C(=O)c1ccc(OCC(=O)OC(C)C(=O)OC(C)C(=O)OC(C)C(=O)OC(C)C(=O)O)c(Cl)c1Cl] | ECA-PLA4 MF C₂₅H₂₈Cl₂O₁₂ MW 591.39 |
| 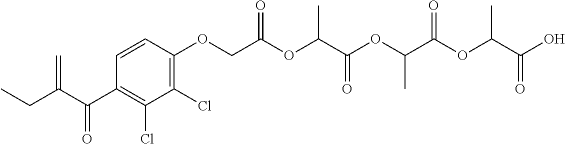 [SMILES: C=C(CC)C(=O)c1ccc(OCC(=O)OC(C)C(=O)OC(C)C(=O)OC(C)C(=O)O)c(Cl)c1Cl] | ECA-PLA3 MF C₂₂H₂₄Cl₂O₁₀ MW 519.32 |
| 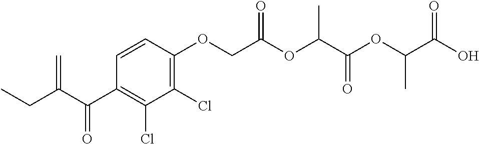 [SMILES: C=C(CC)C(=O)c1ccc(OCC(=O)OC(C)C(=O)OC(C)C(=O)O)c(Cl)c1Cl] | ECA-PLA2 MF C₁₉H₂₀Cl₂O₈ MW 447.26 |
| 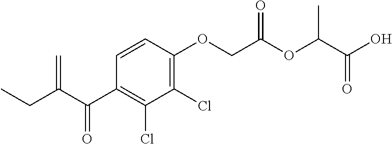 [SMILES: C=C(CC)C(=O)c1ccc(OCC(=O)OC(C)C(=O)O)c(Cl)c1Cl] | ECA-PLA1 MF C₁₆H₁₆Cl₂O₆ MW 375.20 |
| 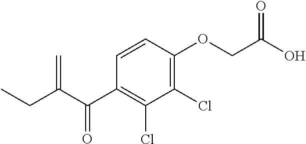 [SMILES: C=C(CC)C(=O)c1ccc(OCC(=O)O)c(Cl)c1Cl] | ECA MF C₁₃H₁₂Cl₂O₄ MW 303.14 |

TABLE 7
Extracted ions and polarity modes

| SIGNAL | ION 1 | ION 2 | SIGNAL ID |
|---|---|---|---|
| MSD2, NEG | 302 | 304 | 303 |
| MSD2, NEG | 374 | 376 | 375 |
| MSD2, NEG | 446 | 448 | 447 |
| MSD2, NEG | 518 | 520 | 519 |
| MSD2, NEG | 590 | 592 | 591 |
| MSD2, NEG | 662 | 664 | 663 |
| MSD2, NEG | 734 | 736 | 735 |
| MSD1, POS | 762 | 764 | 763 |

### Example 8. Drug Solubility of Select Compounds of the Present Invention

For each test, approximately 5-10 mg was transferred to a 10 mL glass vial. Aqueous or organic solvent was added to each vial to achieve an overall concentration of 50 mg/mL. After vortexing aggressively for 2-3 minutes and sonicating in a bath sonicator for 5 minutes, undissolved drug was spun down at 1200 rpm for 5 minutes to generate a pellet. The supernatant was collected and filtered through a 0.2 μm nylon syringe filter into HPLC vials for drug content analysis. Drug concentration was determined by comparing against a standard calibration curve. Table 4 is the solubility of select compounds in water, DMSO, and DCM.

All prodrugs of ethacrynic acid exhibited low aqueous solubility and high organic solubility (less than 1 mg/mL in aqueous solution and greater than 50 mg/mL in DMSO), respectively. Solubility of Timolol prodrugs was controlled by a number of parameters including the linker, the terminal end-group, the number of PLA repeat units and the salt form. For example, Timolol conjugated with O-ethylfumurate (17) exhibited very low aqueous solubility (<1.0 mg/mL), whereas conjugation with a O-succinate linker (4) resulted in high aqueous solubility (>50 mg/mL).

TABLE 8
Solubility of Dorzolamide Mono Prodrugs, Timolol
Mono Prodrugs, and ECA Mono Prodrugs

| | | | Solubility (mg/mL) | | |
|---|---|---|---|---|---|
| | Compound Name | Compd No. | Water | DMSO | DCM |
| Dorzolamide | Dorzolamide | | >50 | <1.0 | <1.0 |
| Parent | | | | | |
| Dorzolamide | Dorzolamide-Ethylfumurate | (14) | <1.0 | >100 | >100 |
| Mono | Dorzolamide-Stearylfumurate | (15) | >50 | >100 | — |
| Prodrugs | Dorzolamide-Laurylmaleate | (16) | <1.0 | >50 | — |
| Timolol | Timolol-O-Succinate | (4) | >50 | >50 | — |
| Mono | Timolol-O-Ethylsuccinate Maleate | (5) | 12.5 | >50 | — |
| Prodrugs | Timolol-O-Ethylsuccinate HCL | (6) | 6.25 | >50 | — |
| | Timolol-O-Maleate | (7) | >50 | >50 | — |
| | Timolol-O-Ethylmaleate-Maleic Acid | (8) | <1.0 | >50 | — |
| | Timolol-O-Ethylfumurate | (17) | <1.0 | >50 | — |
| | Timolol-O-Ethyl Maleate | (18) | >25 | >100 | — |
| | Timolol-O-Laurylfumurate-Maleate | (12) | <1.0 | >100 | — |
| | Timolol-O-Stearylfumurate-Maleate | (13) | <1.0 | <1.0 | >100 |
| Ethacrynic | Ethacrynic acid-PLA(n = 4)-Ethyl Ester | (1) | <1.0 | >40 | — |
| acid Mono | Ethacrynic acid-PLA(n = 2)-Ethyl Ester | (2) | <1.0 | >50 | — |
| Prodrugs | Ethacrynic acid-PLA(n = 6)-Ethyl Ester | (25) | <0.1 | >50 | — |
| | Ethacrynic acid-PLA(n = 8)-Ethyl Ester | (26) | <0.1 | >50 | — |

### Example 9. In Vitro Stability of Ethacrynic Acid Prodrugs

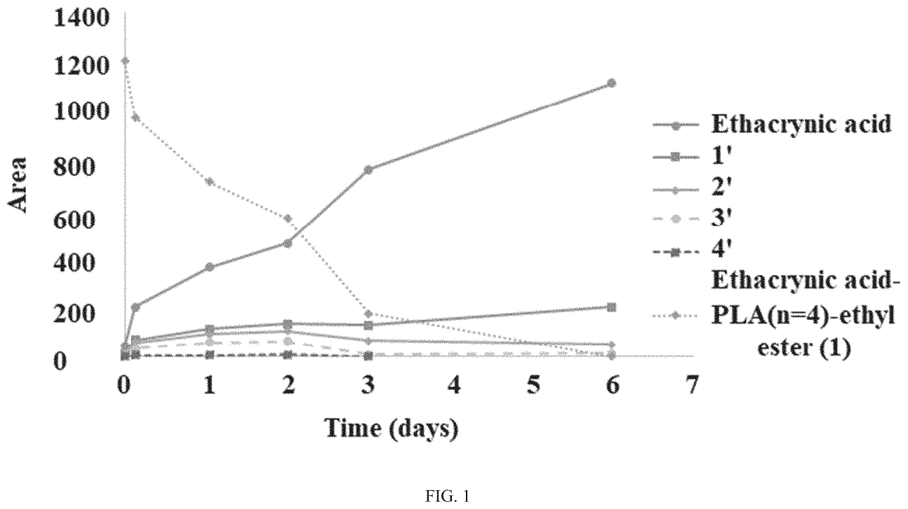

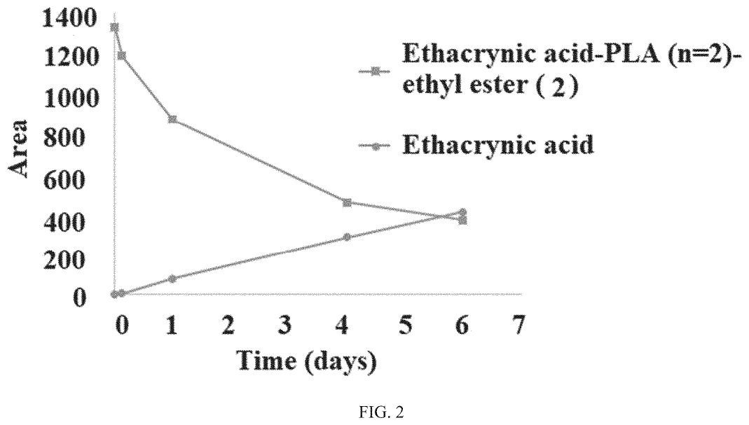

The in vitro stability of prodrugs of ethacrynic acid at 37° C. is demonstrated in FIG. 1 and FIG. 2, respectively. As evidenced, both prodrugs of ethacrynic acid degrade steadily over the 6 day period. As the ester linkages are hydrolyzed, the parent compound is generated in a linear rate. The rate of degradation to the parent compound is faster for the shorter ethacrynic acid-PLA(n=2)-ethyl ester (2) in comparison to the degradation kinetics for ethacrynic acid-PLA(n=4)-ethyl ester (1).

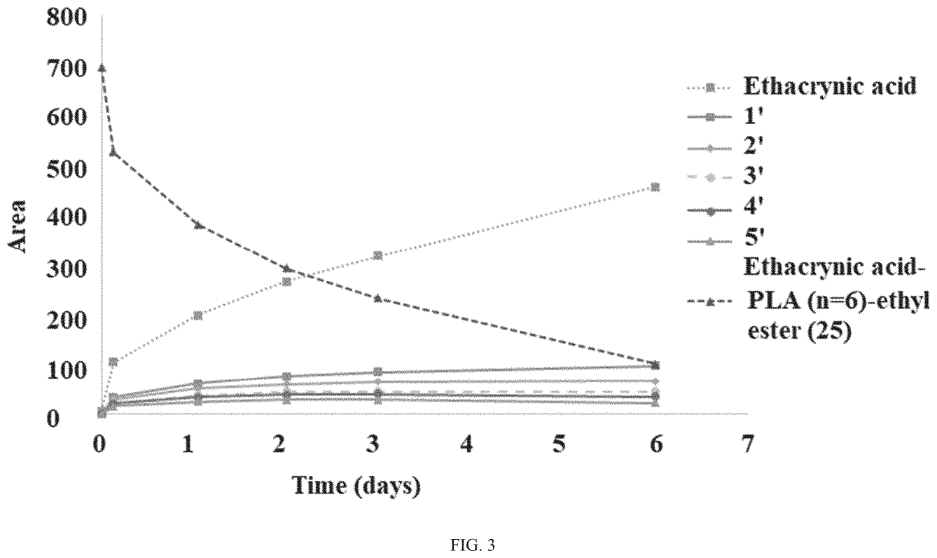

The in vitro stability of prodrugs of ethacrynic acid at 37° C. is further demonstrated in FIG. 1 and FIG. 3, respectively. The rate of degradation of the ECA-PLA(n=4)-ethyl ester prodrug (1) is slightly faster than the ECA-PLA(n=6)-ethyl ester prodrug (25). However, the rate of conversion to the free drug is significantly faster for the ECA-PLA(n=4)-ethyl ester prodrug (2) in comparison to the ECA-PLA(n=6)-ethyl ester prodrug (25). Approximately 94% of the ECA-PLA(n=4) prodrug (1) has been converted to free ECA by day 6, in comparison to only 56% for ECA-PLA(n=6) (25). Also, by day 6, all the initial prodrug has been degraded into intermediates and free drug respectively.

### Example 10. Drug Loading

To determine the percent drug loading (% DL), 10 mg of particles was weighed into a glass scintillation vial and dissolved with 10 mL of MeCN:water (1:1, v/v). The solution was filtered through a 0.2 μm nylon syringe filter and the drug content was determined by RP-HPLC referenced against a standard calibration curve. The drug loading results are presented in Table 9.

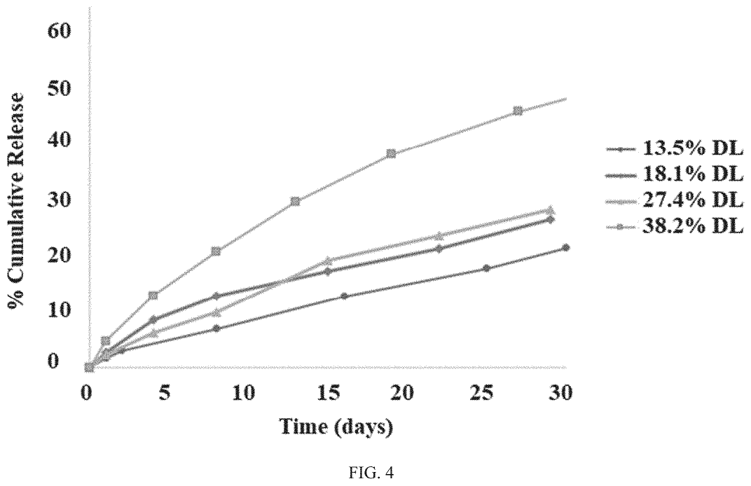

Increased theoretical loading (% drug mass/polymer mass) in the dispersed phase can increase % DL within the formed particles. ECA-PLA(n=6) particles (25) prepared with theoretical loading of 15, 20, 30, and 40% mass resulted in particles with % DL of 13.5, 18.1, 27.4 and 38.2 respectively (Table 10). The rate of drug release increased with increasing drug loading; the fastest rate of drug release was observed for the particles with 38.2% DL (FIG. 4). This increase in release rate with increasing % DL may be attributed to distribution drug near the surface of the microparticles or surface associated drug.

TABLE 9
Formulation parameters and physicochemical properties of
prodrugs of Ethacrynic acid encapsulated microparticles

| Compound | | Polymer Conc | % Drug | Mean size |
|---|---|---|---|---|
| Name | Polymer composition | (mg/mL) | loading | (um) |
| ECA-PLA(n = | PLA100 4.5A + PLGA7525 6E | 200 | 14.17 | 27.40 |
| 4)-ethyl ester (1) | (69:30) + 1% PEG-PLGA5050 | | | |
| | (99% PLA, PLGA blend + 1% PLGA-PEG) | | | |
| ECA-PLA(n = | PLA100 4.5A + PLGA8515 5A | 200 | 12.56 | 26.60 |
| 4)-ethyl ester (1) | (77:22) + 1% PEG-PLGA5050 | | | |
| | (99% PLA, PLGA blend + 1% PLGA-PEG) | | | |
| ECA-PLA(n = | PLA100 4.5A + PLGA8515 5A + | 200 | 12.68 | 29.86 |
| 4)-ethyl ester (1) | PLGA5050 4A (64:30:5) + 1% PEG-PLGA5050 | | | |
| | (99% PLA, PLGA blend + 1% PLGA-PEG) | | | |
| ECA-PLA(n = | PLA100 4.5A + PLGA8515 5A + | 200 | 13.75 | 28.34 |
| 4)-ethyl ester (1) | PLGA5050 4A (64:20:15) + 1% PEG-PLGA5050 | | | |
| | (99% PLA, PLGA blend + 1% PLGA-PEG) | | | |
| ECA-PLA(n = | PLGA7525 4A + 1% PEG-PLGA5050 | 200 | 13.09 | 29.85 |
| 4)-ethyl ester (1) | (99% PLGA blend + 1% PLGA-PEG) | | | |
| ECA-PLA(n = | PLGA8515 5A + PLGA7525 4A | 200 | 13.49 | 27.74 |
| 4)-ethyl ester (1) | (69:30) + 1% PEG-PLGA5050 | | | |
| | (99% PLGA blend + 1% PLGA-PEG) | | | |
| ECA-PLA(n = | PLA100 4.5A + 1% PEG-PLGA5050 | 200 | 13.42 | 35.06 |
| 4)-ethyl ester | (99% PLA + 1% PLGA-PEG) | | | |
| ECA-PLA(n = | PLGA7525 6A + 1% PEG-PLGA5050 | 200 | 13.15 | 25.21 |
| 4)-ethyl ester (1) | (99% PLGA blend + 1% PLGA-PEG) | | | |
| ECA-PLA(n = | PLGA8515 5A + 1% PEG-PLGA5050 | 200 | 13.59 | 28.90 |
| 4)-ethyl ester (1) | (99% PLGA blend + 1% PLGA-PEG) | | | |
| ECA-PLA(n = | PLA100 4.5A + PLGA7525 6E | 200 | 11.56 | 26.95 |
| 6)-ethyl ester (25) | (69:30) + 1% PEG-PLGA5050 | | | |
| | (99% PLA, PLGA blend + 1% PLGA-PEG) | | | |
| ECA-PLA(n = | PLA100 4.5A + PLGA8515 5A | 200 | 12.29 | 27.88 |
| 6)-ethyl ester (25) | (77:22) + 1% PEG-PLGA5050 | | | |
| | (99% PLA, PLGA blend + 1% PLGA-PEG) | | | |
| ECA-PLA(n = | PLA100 4.5A + PLGA8515 5A + | 200 | 13.53 | 26.27 |
| 6)-ethyl ester (25) | PLGA5050 4A (64:30:5) + 1% PEG-PLGA5050 | | | |
| | (99% PLA, PLGA blend + 1% PLGA-PEG) | | | |
| ECA-PLA(n = | PLA100 4.5A + PLGA8515 5A + | 200 | 14.22 | 26.59 |
| 6)-ethyl ester (25) | PLGA5050 4A (64:20:15) + 1% PEG-PLGA5050 | | | |
| | (99% PLA, PLGA blend + 1% PLGA-PEG) | | | |
| ECA-PLA(n = | PLGA7525 4A + 1% PEG-PLGA5050 | 200 | 12.43 | 26.19 |
| 6)-ethyl ester (25) | (99% PLGA blend + 1% PLGA-PEG) | | | |
| ECA-PLA(n = | PLA100 4.5A + 1% PEG-PLGA5050 | 200 | 13.83 | 27.62 |
| 6)-ethyl ester (25) | (99% PLA blend + 1% PLGA-PEG) | | | |

TABLE 10
Theoretical loading and actual loading
of ECA-PLA(n = 6) (25) microparticles

| | Theoretical | % Drug | % Encapsulation |
|---|---|---|---|
| Particle Batch | Loading | Loading | Efficiency |
| ECA-PLA(n = 6)- | 15 | 13.5 | 90.0 |
| ethyl ester (25) | | | |
| ECA-PLA(n = 6)- | 20 | 18.1 | 90.5 |
| ethyl ester (25) | | | |
| ECA-PLA(n = 6)- | 30 | 27.4 | 91.3 |
| ethyl ester (25) | | | |
| ECA-PLA(n = 6)- | 40 | 38.2 | 95.5 |
| ethyl ester (25) | | | |

### Example 11. Drug Release Kinetics of Timolol and Ethacrynic Acid Prodrugs

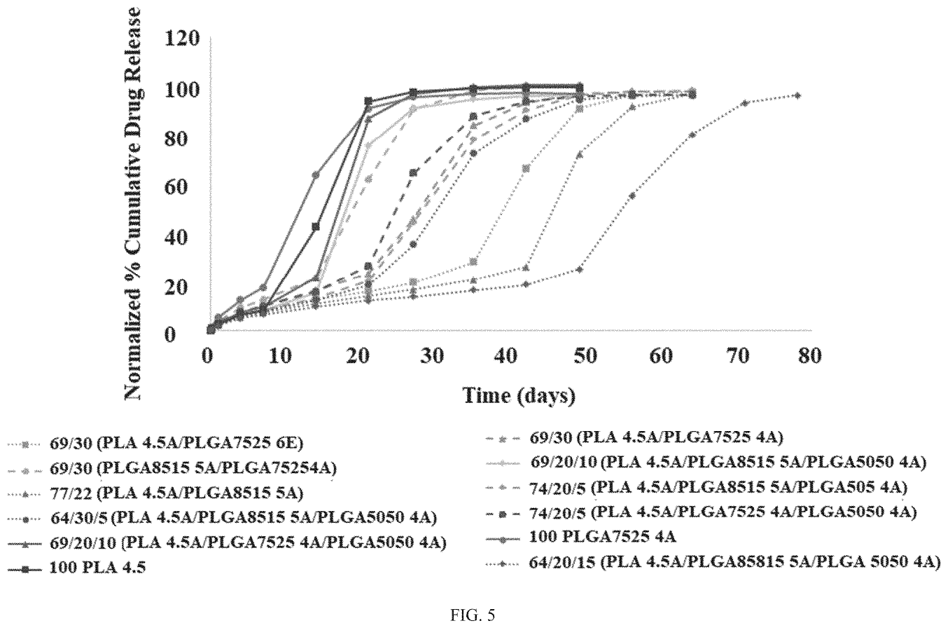

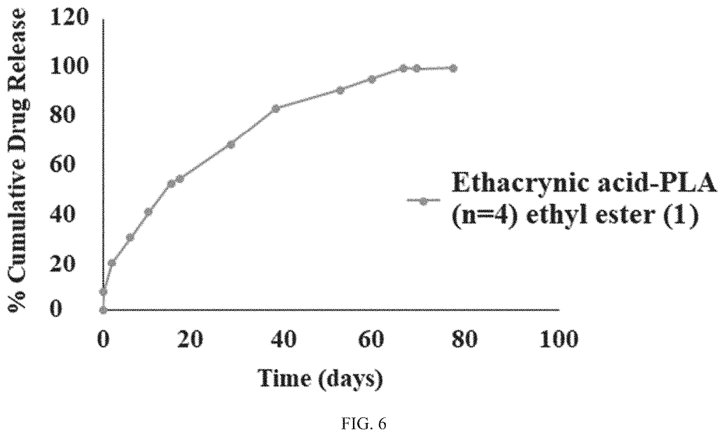

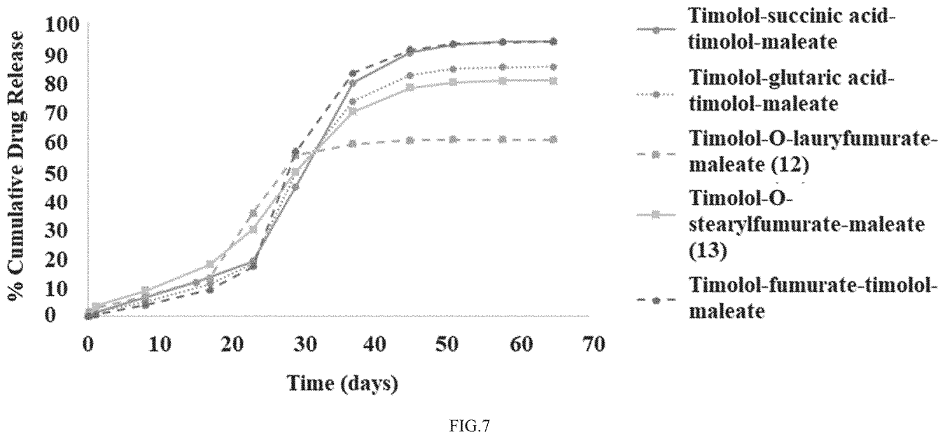

The effect of polymer composition including monomer ratio and molecular weight, polymer end-groups (ester or acid), inherent viscosity and polymer blend ratios on particle degradation and drug release kinetics of Timolol-O-ethyl fumurate (17) was evaluated and is illustrated in FIG. 5. The rate of particle degradation was slowed and drug release was prolonged when the mole % of DL-lactide over glycolide was increased. By incorporating polymers with different monomer ratios (i.e., PLA, PLGA8515, PLGA525, PLGA5050 wherein 8515 refers to 85% DL-lactide and 15% glycolide) into the particles, it is possible to fine-tune the degradation rate of the particles to achieve a linear rate of drug release from the particles to minimize burst or lag and to extend the duration of release. End-group modification of the polymer from the acid form to the ester form exhibited a similar effect at slowing particle degradation and drug release. Interestingly, blending a number of different polymers with different monomer ratios, end groups and molecular weights enabled optimization of release kinetics to achieve a linear 3-6 month release profile. The 78/22(PLA 5A/PLGA8515 4.5A) and 65/20/15(PLA 4A/PLGA6515 4.5A/PLGA5050 4A) polymer blends generated ideal particles for a 3-6 month linear release formulation. In addition, blending copolymers of PLA and PLGA resulted in a significant decrease in the rate of drug release (FIG. 6). The increased hydrophobicity due to the presence of the methyl side groups in PLA resulted in less water absorption and hydrolysis of the ester linkages within the polymer backbone resulting in slower bulk erosion of the particles. The rate of release was independent of the salt form. Comparative evaluation of blended particles with the same polymer composition encapsulating 5 different mono and bis prodrugs of Timolol (FIG. 7) revealed only minor differences between the release rates of the 5 particle formulations. Thus, release rate is not solely dictated by the physicochemical properties of the linker, but rather must be attributed to a combination of parameters that may include linker type and site of linker conjugation.

FIG. 6 illustrates the release kinetics of ethacrynic acid-PLA(n=4)-ethyl ester (1) from PLGA microparticles. The drug was released over a period of approximately 66 days.

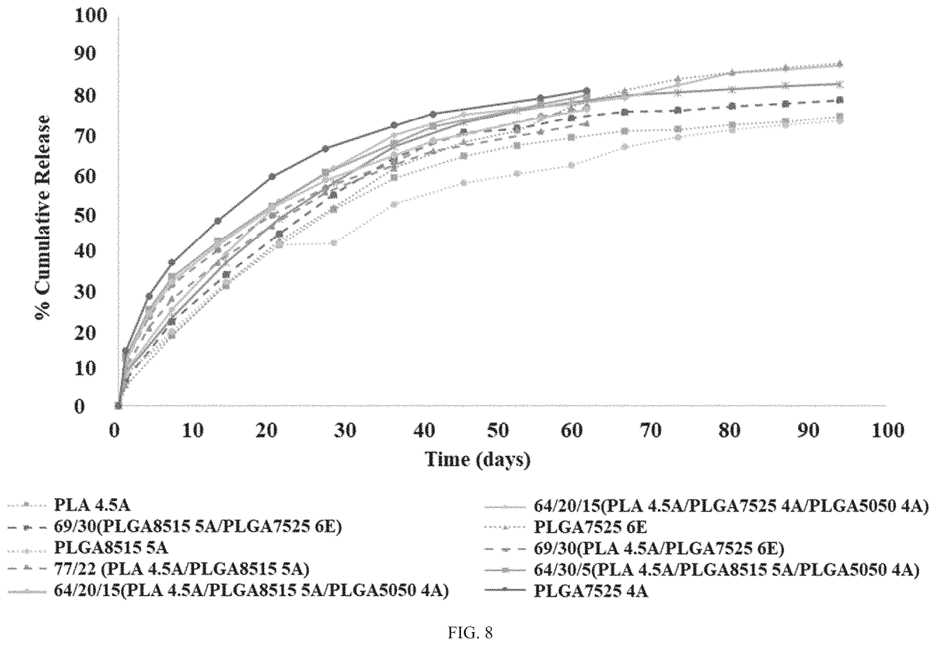

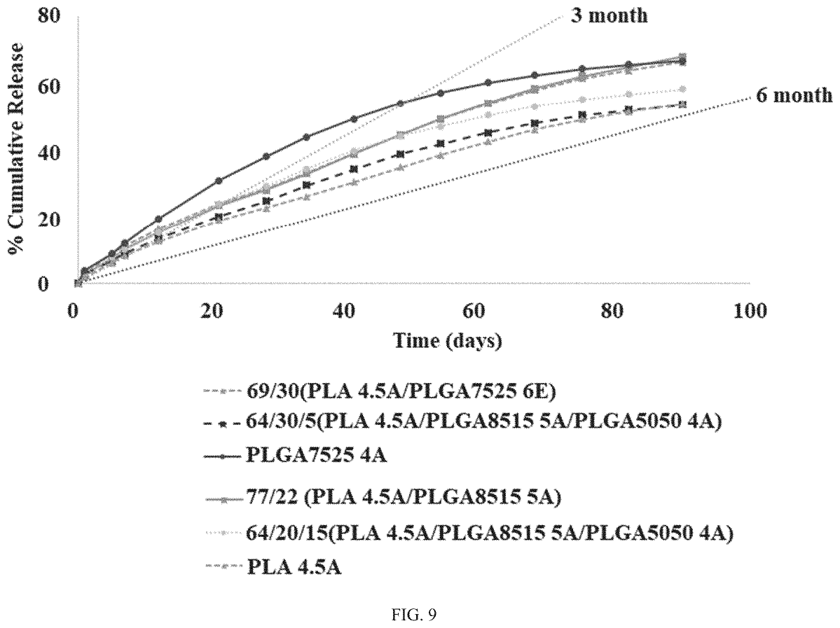

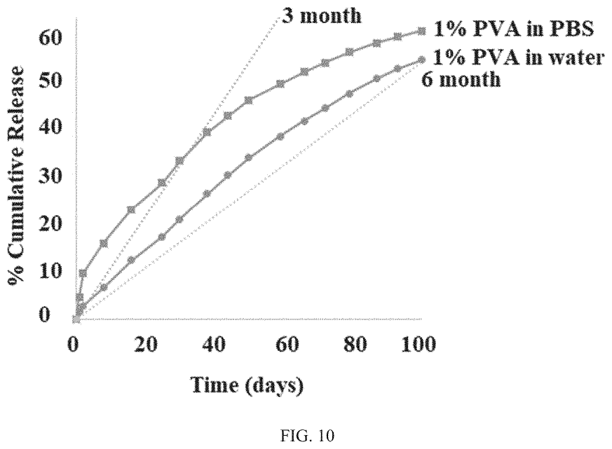

FIG. 8 and FIG. 9 illustrate the release kinetics of ethacrynic acid-PLA(n=4)-ethyl ester (1) and ethacrynic acid-PLA(n=6)-ethyl ester (25), respectively, from PLGA microparticles. The drug was released from a number of formulations over a period of greater than 90 days. The choice of continuous phase was found to also play a significant role in the physicochemical characteristics of the ECA-microparticles. Particles generated with 1% PVA in PBS was compared against particles generated in 1% PVA in water (FIG. 10). ECA-prodrug encapsulating microparticles prepared in 1% PVA water exhibited comparable size and % DL. However, the burst release was significantly higher for the particles prepared in 1% PVA in PBS than in 1% PVA in water.

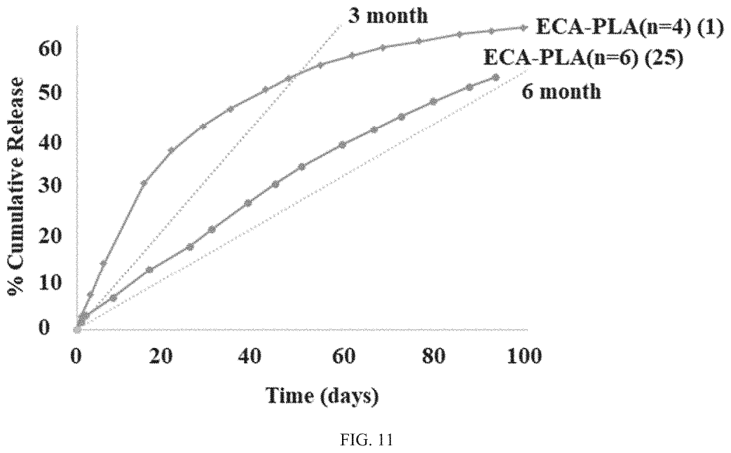

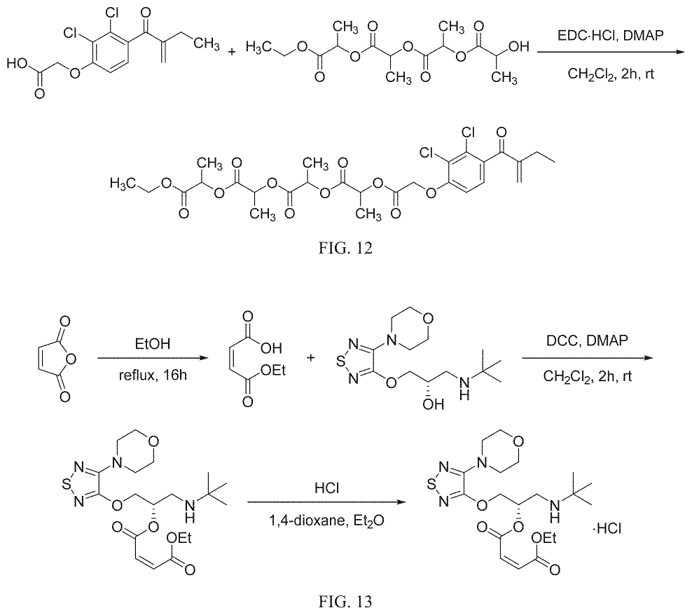

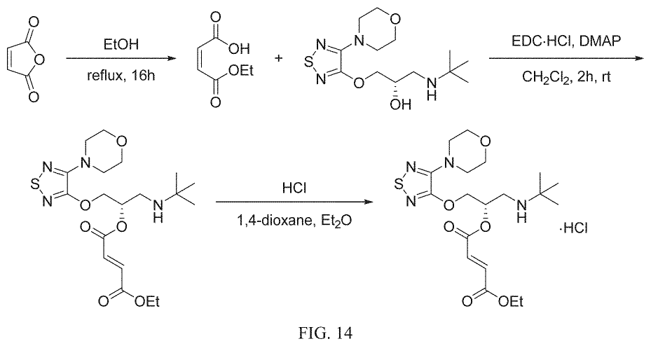

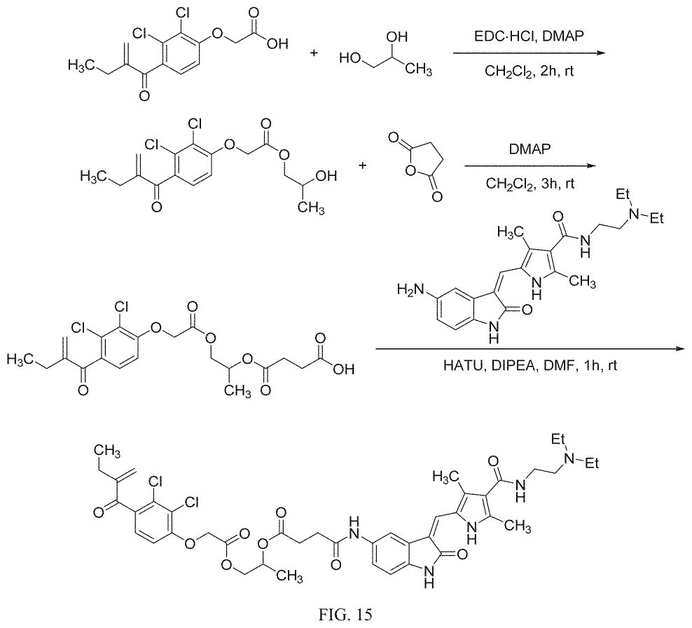

Based upon these initial formulation screens, the formulation comprising of 1% mPEG-PLGA and 99% PLA100 4.5A:PLGA8515 5A (77:22) was selected as the lead formulation for ECA-PLA(n=4) (1) and ECA-PLA(n=6) (25) prodrugs. The particles were prepared in 1% PVA in water. The release profiles for the lead ECA-PLA(n=4) (1) and ECA-PLA(=6) (25) particle formulations are illustrated in FIG. 11. Release of the ECA-PLA(n=4) (1) microparticles follows a typical biphasic release profile characterized by a rapid initial phase and a slower secondary phase. Comparison of the release curves reveals a much more linear release profile for ECA-PLA(n=6) (25) microparticles over ECA-PLA(n=4) (1) microparticles, maintaining linearity within the 3-6 month ideal release window for the duration of release, which is important in ensuring consistent daily exposure of the drug at the site of action.

### Example 12. Synthesis of Representative Linkers of the Present Invention

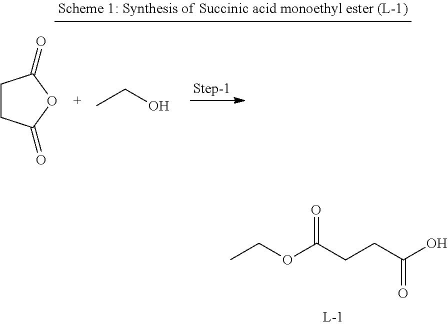

### Step-1: Preparation of Succinic Acid Monoethyl Ester (L-1)

A solution of dihydro-furan-2,5-dione (20 g, 20 mmol) in ethanol (100 mL) was allowed to stir at 80° C. over a period of 16 h. The resulting reaction mixture was directly concentrated under reduced pressure. The residue was diluted with DCM (600 mL) and washed with saturated sodium bicarbonate solution (300 mL). The aqueous layer was separated from organic, acidified with 1.5N HCl (pH=2) and extracted with DCM (300×2 mL), dried over Na₂SO₄ and concentrated under reduced pressure to obtain product L-1 as a colorless liquid 11.5 g (39.3%).

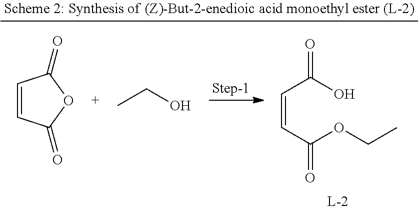

### Step-1: Preparation of (Z)-but-2-Enedioic Acid Monoethyl Ester (L-2)

A solution of furan-2,5-dione (5 g, 51.02 mmol) in ethanol (50 mL) was allowed to stir at 100° C. over a period of 16 h. The resulting reaction mixture was directly concentrated under reduced pressure. Then residue was diluted with DCM (450 mL) and washed with saturated sodium bicarbonate solution (200 mL). The aqueous layer was separated from organic, acidified with 1.5N HCl (pH=2) and extracted with DCM (150×3 mL), dried over Na₂SO₄ and concentrated under reduced pressure to obtain product L-2 as a colorless liquid 3.3 g (45.2%).

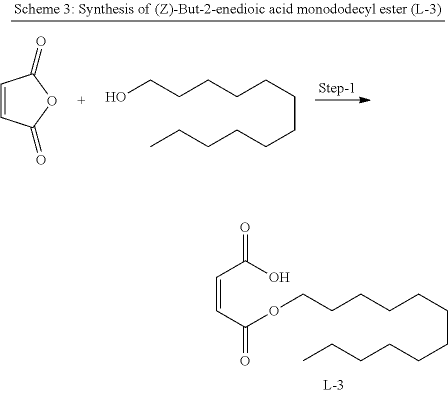 [SMILES: O=C1C=CC(=O)O1 ; CCCCCCCCCCCCO ; CCCCCCCCCCCCOC(=O)/C=C\C(=O)O]

### Step-1: Preparation of (Z)-But-2-enedioic acid monododecyl ester (L-3)

To a solution of dodecan-1-ol (1.0 g, 5.37 mmol) in toluene (10 mL) was added furan-2,5-dione (0.526 g, 5.37 mmol) at 25-30° C. The resulting mixture was allowed to stir at 100° C. over a period of 16 h. The reaction mixture was diluted with ethyl acetate (300 mL) and basified (pH=10) with sodium hydroxide solution (100 mL). The aqueous layer was separated from organic, acidified with 1.5N HCl (pH=2), extracted with ethyl acetate (100×3 mL), dried over Na₂SO₄ and concentrated under reduced pressure to obtain product L-3 as a white solid 1.1 g (73%).

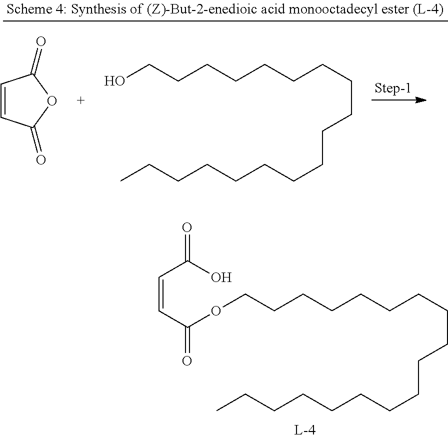 [SMILES: O=C1C=CC(=O)O1 ; CCCCCCCCCCCCCCCCCCO ; CCCCCCCCCCCCCCCCCCOC(=O)/C=C\C(=O)O]

### Step-1: Preparation of (Z)-But-2-enedioic acid monooctadecyl ester (L-4)

To a solution of octadecan-1-ol (1.0 g, 3.70 mmol) in toluene (10 mL) was added furan-2,5-dione (0.362 g, 3.70 mmol) at 25-30° C. The resulting mixture was allowed to stir at 100° C. over a period of 16 h. The reaction mixture was diluted with ethyl acetate (300 mL) and basified (pH=10) with sodium hydroxide solution (100 mL). That aqueous layer was separated from organic, acidified with 1.5N HCl (pH=2), extracted with ethyl acetate (100×3 mL), dried over Na₂SO₄ and concentrated under reduced pressure to obtained product L-4 as a white solid 0.8 g (58.8%).

### Example 13. Synthesis of Representative Compounds of the Present Invention

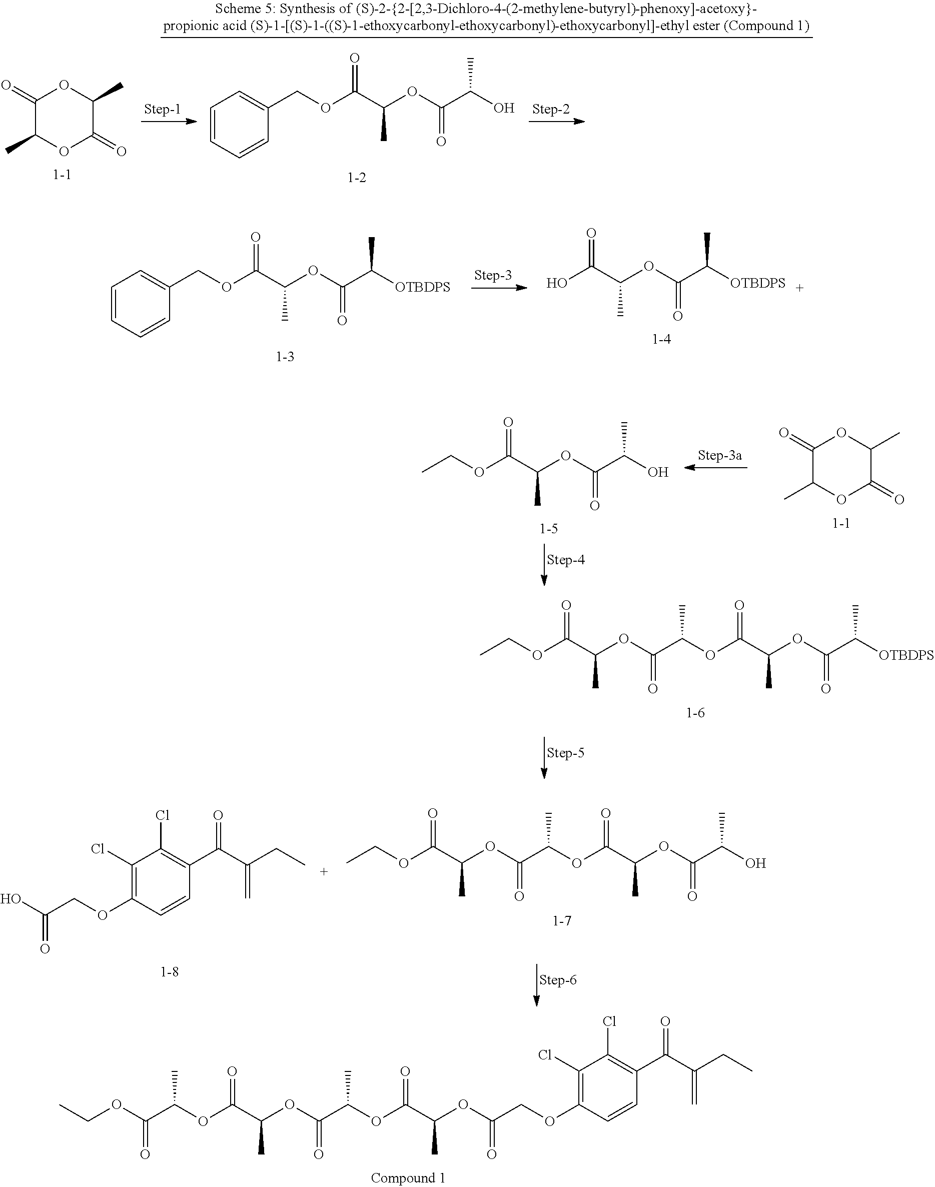

### Step 1: Preparation of (S)-2-Hydroxy-propionic acid (S)-1-benzyloxycarbonyl-ethyl ester (1-2)

To a solution of (3S,6S)-3,6-dimethyl-[1,4]dioxane-2,5-dione 1-1 (5.0 g, 34.72 mmol) in toluene (100 mL) was added benzyl alcohol (3.2 mL, 31.72 mmol) and camphor sulfonic acid (0.8 g, 3.47 mmol) at 25-30° C. The reaction mixture was allowed to stir at 80° C. over a period of 2 hours. The resulting reaction mixture was diluted with ethyl acetate (800 mL) and washed with water (2×400 mL). The crude product obtained upon evaporation of volatiles was purified through preparative HPLC to obtain product 1-2 as a pale yellow liquid 5.5 g (63%).

### Step 2: Preparation of (S)-2-(tert-butyl-Diphenyl-silanyloxy)-propionic acid (S)-1-benzyloxycarbonyl-ethyl ester (1-3)

To a solution of (S)-2-hydroxy-propionic acid (S)-1-benzyloxycarbonyl-ethyl ester 1-2 (0.1 g, 0.23 mmol) in dichloromethane (2 mL) was added triethylamine (0.23 mL, 1.61 mmol), TBDPS-Cl (0.43 mL, 1.618 mmol) and a catalytic amount of 4-dimethylaminopyridine at 0° C. The reaction mixture was stirred at room temperature over period of 8 hours. The resulting reaction mixture was quenched with water (20 mL) and extracted with ethyl acetate (2×50 mL). The volatiles were evaporated under reduced pressure to obtain product 1-3 as a colorless liquid 200 mg (74%).

### Step 3: Preparation of (S)-2-(tert-butyl-Diphenyl-silanyloxy)-propionic acid (S)-1-carboxy-ethyl ester (1-4)

(S)-2-(tert-butyl-Diphenyl-silanyloxy)-propionic acid (S)-1-benzyloxycarbonyl-ethyl ester 1-3 (1.5 g), methanol (20 mL) and 10% Pd/C (0.3 g, 50% wet) were taken in a 100 mL autoclave vessel. The reaction mixture was stirred at 25-30° C. under hydrogen pressure (5 kg/cm²) over a period of 2 hours. After completion of the reaction, the reaction mixture was filtered through celite bed and concentrated under reduced pressure. The crude product obtained upon evaporation of volatiles was purified through silica gel (60-120 mesh) column chromatography (10% methanol in dichloromethane) to afford 1-4 as a colorless liquid 700 mg (58%).

### Step 3a: Preparation of (S)-2-Hydroxy-propionic acid (S)-1-ethoxycarbonyl-ethyl ester (1-5)

To a solution of (3S,6S)-3,6-dimethyl-[1,4]dioxane-2,5-dione 1-1 (5.0 g, 34.72 mmol) in toluene (100 mL) was added ethanol (1.92 mL, 31.98 mmol) and camphor sulfonic acid (0.8 g, 3.47 mmol) at 25-30° C. The reaction mixture was allowed to stir at 80° C. over a period of 2 hours. The resulting reaction mixture was diluted with ethyl acetate (800 mL) and washed with water (2×200 mL). The crude product obtained upon evaporation of volatiles was purified through silica gel (230-400 mesh) column chromatography (13% ethyl acetate in hexane) to obtain product 1-5 as a colorless liquid 6.6 g (60%).

### Step 4: Preparation of (S)-2-(tert-Butyl-diphenyl-silanyloxy)-propionic acid (S)-1-[(S)-1-((S)-1-ethoxycarbonyl-ethoxycarbonyl)-ethoxycarbonyl]-ethyl ester (1-6)

To a solution of (S)-2-(tert-butyl-diphenyl-silanyloxy)-propionic acid (S)-1-carboxy-ethyl ester 1-4 (5.473 g, 13.68 mmol) in dichloromethane (60 mL), was added EDC.HCl (3.014 g, 15.78 mmol), (S)-2-Hydroxy-propionic acid (S)-1-ethoxycarbonyl-ethyl ester 1-5 (2 g, 10.52 mmol) and 4-dimethylaminopyridine (128 mg, 1.05 mmol) at 0° C. The reaction mixture was allowed to stir at 25-30° C. over a period of 1 hour. The resulting reaction mass was quenched with water (200 mL), extracted with dichloromethane (250×3 mL), dried over Na₂SO₄ and concentrated under reduced pressure. The crude product obtained upon evaporation of volatiles was purified through silica gel (230-400 mesh) column chromatography (3% ethyl acetate in hexane) to obtain product 1-6 as a colorless liquid 4.2 g (70%).

### Step 5: Preparation of (S)-2-Hydroxy-propionic acid (S)-1-[(S)-1-((S)-1-ethoxycarbonyl-ethoxycarbonyl)-ethoxycarbonyl]-ethyl ester (1-7)

To a solution of (S)-2-(tert-Butyl-diphenyl-silanyloxy)-propionic acid (S)-1-[(S)-1-((S)-1-ethoxycarbonyl-ethoxycarbonyl)-ethoxycarbonyl]-ethyl ester 1-6 (4 g, 6.99 mmol) in tetrahydrofuran (40 mL) were added tetra butyl ammonium fluoride (10.49 mL, 1.0M, 10.49 mmol) and acetic acid (0.63 g, 10.49 mmol) at 0° C. The reaction mixture was allowed to stir at room temperature over a period of 1 hour. The resulting reaction mixture was concentrated under reduced pressure and the crude product was obtained upon evaporation of the volatiles. The crude product was purified through silica gel (230-400 mesh) column chromatography (12% ethyl acetate in hexane) to afford product 1-7 as a colourless liquid 1.0 g (43%).

### Step 6: Preparation of (S)-2-{2-[2,3-Dichloro-4-(2-methylene-butyryl)-phenoxy]-acetoxy}-propionic acid (S)-1-[(S)-1-((S)-1-ethoxycarbonyl-ethoxycarbonyl)-ethoxycarbonyl]-ethyl ester (Compound 1)

To a solution of ethacrynic acid 1-8 (9.433 g, 31.13 mmol) in dichloromethane (80 mL) was added EDC.HCl (6.86 g, 35.92 mmol), (S)-2-Hydroxy-propionic acid (S)-1-[(S)-1-((S)-1-ethoxycarbonyl-ethoxycarbonyl)-ethoxycarbonyl]-ethyl ester 1-7 (8 g, 23.95 mmol) and 4-dimethylaminopyridine (292 mg, 2.39 mmol) at 0° C. The reaction mixture was allowed to stir at 25-30° C. over a period of 1 hour. The resulting reaction mass was quenched with water (400 mL), extracted with dichloromethane (400×2 mL), dried over Na₂SO₄ and concentrated under reduced pressure. The crude product obtained upon evaporation of volatiles was purified through silica gel (230-400 mesh) column chromatography (13% ethyl acetate in hexane) to obtain Compound 1 as a colourless wax 8 g (53.9%).

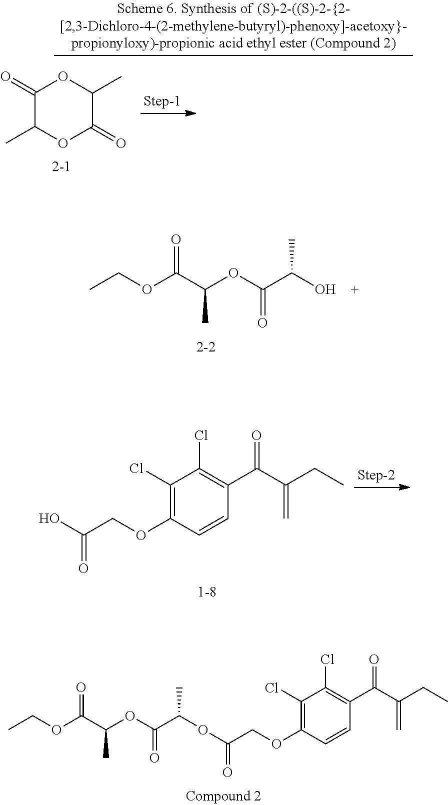

### Step 1: Preparation of (S)-2-Hydroxy-propionic acid (S)-1-ethoxycarbonyl-ethyl ester (2-2)

To a solution of (3S,6S)-3,6-dimethyl-[1,4]dioxane-2,5-dione 2-1 (5.0 g, 34.72 mmol) in toluene (100 mL) was added ethanol (1.92 mL, 31.98 mmol) and camphor sulfonic acid (0.8 g, 3.47 mmol) at 25-30° C. The reaction mixture was allowed to stir at 80° C. over a period of 2 hours. The resulting reaction mixture was diluted with ethyl acetate (800 mL) and washed with water (2×200 mL). The crude product obtained upon evaporation of volatiles was purified through silica gel (230-400 mesh) column chromatography (13% ethyl acetate in hexane) to obtain product 2-2 as a colourless liquid 6.6 g (60%).

### Step 2: Preparation of 2-{2-[2,3-Dichloro-4-(2-methylene-butyryl)-phenoxy]-acetoxy}-propionic acid 1-ethoxycarbonyl-ethyl ester (Compound 2)

To a solution of ethacrynic acid 1-8 (3.11 g, 10.2 mmol) in dichloromethane (15 mL) was added EDC.HCl (2.26 g, 11.83 mmol), (S)-2-Hydroxy-propionic acid (S)-1-ethoxycarbonyl-ethyl ester 2-2 (1.5 g, 7.89 mmol), and 4-dimethylaminopyridine (96 mg, 1.02 mmol) at 0° C. The reaction mixture was allowed to stir at 25-30° C. over a period of 1 hour. The resulting reaction mass was quenched with water (100 mL), extracted with dichloromethane (100×2 mL), dried over Na₂SO₄ and concentrated under reduced pressure. The crude product obtained upon evaporation of volatiles was purified through silica gel (230-400 mesh) column chromatography (10% ethyl acetate in hexane) to obtain Compound 2 as a colorless wax 1.4 g (37.3%).

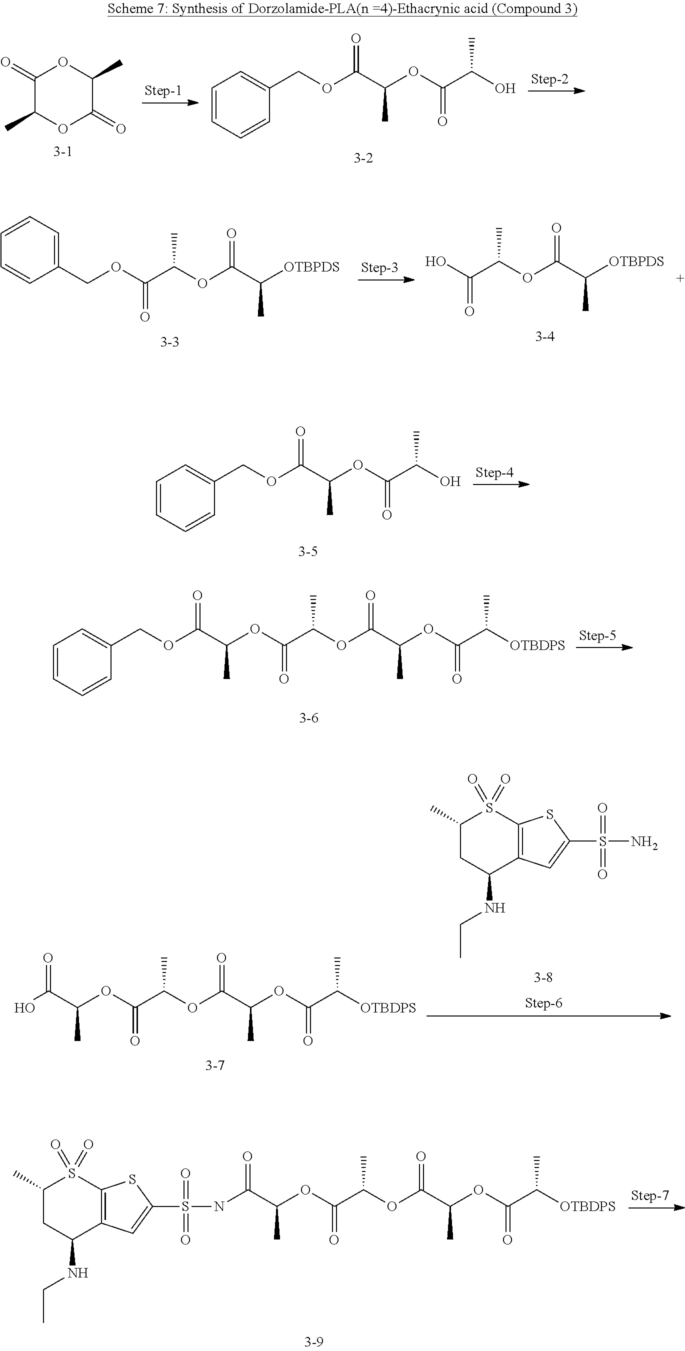

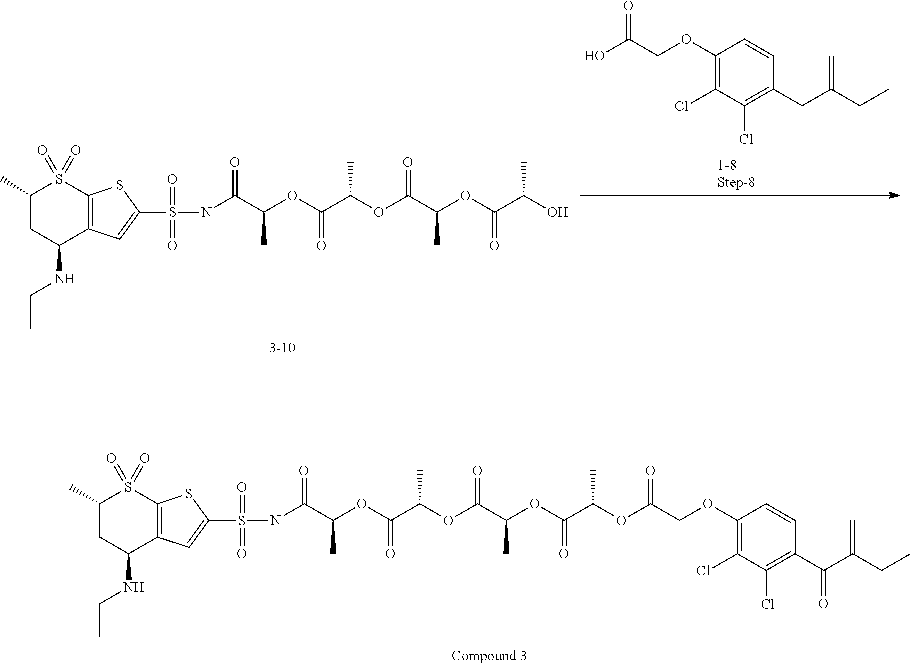 [SMILES: C=C(CC)Cc1ccc(OCC(=O)O)c(Cl)c1Cl ; CCN[C@H]1C[C@H](C)S(=O)(=O)c2sc(S(=O)(=O)NC(=O)[C@H](C)OC(=O)[C@H](C)OC(=O)[C@H](C)OC(=O)[C@H](C)O)cc21 ; C=C(CC)C(=O)c1ccc(OCC(=O)O[C@@H](C)C(=O)O[C@@H](C)C(=O)O[C@@H](C)C(=O)O[C@@H](C)C(=O)NS(=O)(=O)c2cc3c(s2)S(=O)(=O)[C@@H](C)C[C@@H]3NCC)c(Cl)c1Cl]

### Step 1: Preparation of (S)-2-Hydroxy-propionic acid (S)-1-benzyloxycarbonyl-ethyl ester (3-2)

To a solution of (3S,6S)-3,6-dimethyl-[1,4]dioxane-2,5-dione 3-1 (5.0 g, 34.72 mmol) in toluene (100 mL) was added benzyl alcohol (3.2 mL, 31.72 mmol) and camphor sulfonic acid (0.8 g, 3.47 mmol) at 25-30° C. The reaction mixture was allowed to stir at 80° C. over a period of 2 hours. The resulting reaction mixture was diluted with ethyl acetate (800 mL) and washed with water (2×400 mL). The crude product obtained upon evaporation of volatiles was purified through preparative HPLC to obtain product 3-2 as a pale yellow liquid 5.5 g (63%).

### Step 2: Preparation of (S)-2-(tert-Butyl-diphenyl-silanyloxy)-propionic acid (S)-1-benzyloxycarbonyl-ethyl ester (3-3)

To a solution of (S)-2-hydroxy-propionic acid (S)-1-benzyloxycarbonyl-ethyl ester 3-2 (0.1 g, 0.23 mmol) in dichloromethane (5 mL) was added triethylamine (0.23 mL, 1.61 mmol), TBDPS-Cl (0.43 mL, 1.61 mmol) and a catalytic amount of 4-dimethylaminopyridine at 0° C. The reaction mixture was stirred at room temperature over period of 8 hours and the resulting reaction mixture was quenched with water (20 mL) and extracted with ethyl acetate (2×50 mL). The volatiles were evaporated under reduced pressure to obtain product 3-3 as a colorless liquid 200 mg (74%).

### Step 3: Preparation of (S)-2-(tert-Butyl-diphenyl-silanyloxy)-propionic acid (S)-1-carboxy-ethyl ester (3-4)

(S)-2-(tert-butyl-diphenyl-silanyloxy)-propionic acid (S)-1-benzyloxycarbonyl-ethyl ester 3-3 (1.5 g), methanol (20 mL) and 10% Pd/C (0.3 g, 50% wet) were taken up in a 100 mL autoclave vessel. The reaction mixture was stirred at 25-30° C. under hydrogen pressure (5 kg/cm²) over a period of 2 hours. After completion of the reaction, the reaction mixture was filtered through a celite bed and concentrated under reduced pressure. The crude product obtained upon evaporation of volatiles was purified through silica gel (60-120 mesh) column chromatography (10% methanol in dichloromethane) to afford 3-4 as a colorless liquid 700 mg (58%).

### Step 4: Preparation of (S)-2-(tert-Butyl-diphenyl-silanyloxy)-propionic acid (S)-1-[(S)-1-((S)-1-benzyloxycarbonyl-ethoxycarbonyl)-ethoxycarbonyl]-ethyl ester (3-6)

To a solution of (S)-2-hydroxy-propionic acid (S)-1-benzyloxycarbonyl-ethyl ester 3-5 (6.0 g, 33.2 mmol) and (S)-2-(tert-butyl-diphenyl-silanyloxy)-propionic acid (S)-1-carboxy-ethyl ester 3-4 (17.3 g, 7.77 mmol) in dichloromethane (60 mL) was added EDC.HCl (8.2 g, 43.2 mmol) and 4-dimethylaminopyridine (405 mg, 3.3 mmol) at 0° C. The reaction mixture was allowed to stir at 25-30° C. over a period of 1 hour. The resulting reaction mass was quenched with water (200 mL), extracted with dichloromethane (250×3 mL), dried over Na₂SO₄ and concentrated under reduced pressure. The crude product obtained upon evaporation of volatiles was purified through silica gel (230-400 mesh) column chromatography (10% methanol in dichloromethane) to obtain product 3-6 as a pale yellow liquid 5.8 g (94%).

### Step 5: Preparation of (S)-2-(tert-Butyl-diphenyl-silanyloxy)-propionic acid (S)-1-[(S)-1-((S)-1-carboxy-ethoxycarbonyl)-ethoxycarbonyl]-ethyl ester (3-7)

To a 100 mL autoclave vessel was added a solution of (S)-2-(tert-butyl-diphenyl-silanyloxy)-propionic acid (S)-1-[(S)-1-((S)-1-benzyloxycarbonyl-ethoxycarbonyl)-ethoxycarbonyl]-ethyl ester 3-6 (700 mg, 1.10 mmol) in methanol (10 mL) and 10% Pd/C (140 mg, 50% wet) at 25-30° C. The reaction mixture was stirred at room temperature under hydrogen pressure (5 kg/cm²) over a period of 2 hours. After completion of the reaction, the reaction mixture was filtered through a celite bed and concentrated under reduced pressure. The crude product obtained upon evaporation of volatiles was purified through silica gel (60-120 mesh) column chromatography (10% methanol in dichloromethane) to obtain product 3-7 as a pale yellow liquid 420 mg (78%).

### Step 6: Preparation of (S)-2-(tert-Butyl-diphenyl-silanyloxy)-propionic acid (S)-1-{(S)-1-[(S)-2-((4S,6S)-4-ethylamino-6-methyl-7,7-dioxo-4,5,6,7-tetrahydro-7lambda*6*-thieno[2,3-b]thiopyran-2-sulfonylamino)-1-methyl-2-oxo-ethoxycarbonyl]-ethoxycarbonyl}-ethyl ester (3-9)

To a solution of Dorzolamide 3-8 (1.0 g, 2.7 mmol) in dichloromethane (10 mL) was added N,N-diisopropylethylamine (0.96 mL, 5.5 mmol) at 0° C. After 30 minutes, (S)-2-(tert-Butyl-diphenyl-silanyloxy)-propionic acid (S)-1-[(S)-1-((S)-1-carboxy-ethoxycarbonyl)-ethoxycarbonyl]-ethyl ester 3-7 (2.27 g, 4.1 mmol), EDC.HCl (0.79 g, 4.1 mmol) and 4-dimethylaminopyridine (33 g, 0.27 mmol) were added at 0° C. The reaction mixture was allowed to stir at 25-30° C. over a period of 1 hour. The resulting reaction mass was quenched with water (150 mL), extracted with dichloromethane (200×3 mL), dried over sodium sulphate and concentrated under reduced pressure. The crude product obtained upon evaporation of volatiles was purified through silica gel (230-400 mesh) column chromatography (4% methanol in DCM) to obtain product 3-9 as an off white solid 1.5 g (65%).

### Step 7: Preparation of (S)-2-Hydroxy-propionic acid (S)-1-{(S)-1-[(S)-2-((4S,6S)-4-ethylamino-6-methyl-7,7-dioxo-4,5,6,7-tetrahydro-7lambda*6*-thieno [23-b]thiopyran-2-sulfonylamino)-1-methyl-2-oxo-ethoxycarbonyl]-ethoxycarbonyl}-ethyl ester (3-10)

To a solution of (S)-2-(tert-Butyl-diphenyl-silanyloxy)-propionic acid (S)-1-{(S)-1-[(S)-2-((4S,6S)-4-ethylamino-6-methy-1-7,7-dioxo-4,5,6,7-tetrahydro-7lambda*6*-thieno[2,3-b]thiopyran-2-sulfonylamino)-1-methyl-2-oxo-ethoxycarb onyl]-ethoxycarbonyl}-ethyl ester (3-9) (1.8 g, 2.11 mmol) in tetrahydrofuran (20 mL) was added tetra butyl ammonium fluoride (4.23 mL, 1.0M, 4.22 mmol) and acetic acid (0.25 g, 4.22 mmol) at 0° C. The reaction mixture was allowed to stir at room temperature over a period of 12 hours. The resulting reaction mixture was concentrated under reduced pressure and crude product obtained upon evaporation of the volatiles was purified through silica gel (230-400 mesh) column chromatography (4% methanol in ethyl acetate) to give product 3-10 as an off white solid 1.0 g (77%)

### Step 8: Preparation of Dorzolamide-PLA(n=4)-Ethacrynic acid (Compound 4)

To a solution of ethacrynic acid 1-8 (2.47 g, 8.16 mmol) in dichloromethane (50 mL) was added EDC.HCl (1.87 g, 9.79 mmol), (S)-2-Hydroxy-propionic acid (S)-1-{(S)-1-[(S)-2-((4S,6S)-4-ethylamino-6-methyl-7,7-dioxo-4,5,6,7-tetrahydro-7lambda*6*-thieno[2,3-b]thiopyran-2-sulfonylamino)-1-methyl-2-oxo-ethoxycarbonyl]-ethoxycarbonyl}-ethyl ester 3-10 (5.0 g, 8.16 mmol), hydroxybenzotriazol (225 mg, 0.16 mmol), and 4-dimethylaminopyridine (100 mg, 0.82 mmol) at 0° C. The reaction mixture was allowed to stir at 25-30° C. over a period of 1 hour. The resulting reaction mass was quenched with water (200 mL), extracted with dichloromethane (200×3 mL), dried over Na₂SO₄ and concentrated under reduced pressure. The crude product obtained upon evaporation of volatiles was purified through silica gel (230-400 mesh) column chromatography (13% ethyl acetate in hexane) to obtain Compound 3 as an off white solid 2.5 g (34%).

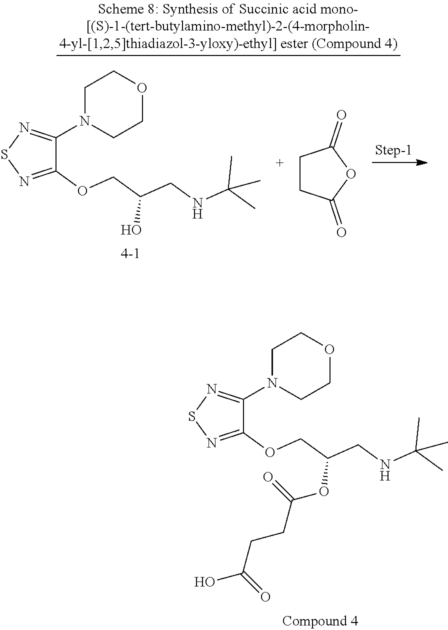

### Step 1: Preparation of Succinic acid mono-[(S)-1-(tert-butylamino-methyl)-2-(4-morpholin-4-yl-[1,2,5]thiadiazol-3-yloxy)-ethyl] ester (Compound 4)

To a solution of (S)-1-tert-butylamino-3-(4-morpholin-4-yl-[1,2,5]thiadiazol-3-yloxy)-propan-2-ol 4-1 (1.0 g, 3.16 mmol) in dichloromethane (10 mL) were added dihydro-furan-2,5-dione (0.35 g, 3.48 mmol) and 4-dimethylaminopyridine (0.039 g, 0.31 mmol) at 0° C. The reaction mixture was allowed to stir at room temperature over a period of 2 h. The resulting reaction mixture was concentrated under reduced pressure to afford Compound 4 as an off white solid 800 mg (61%).

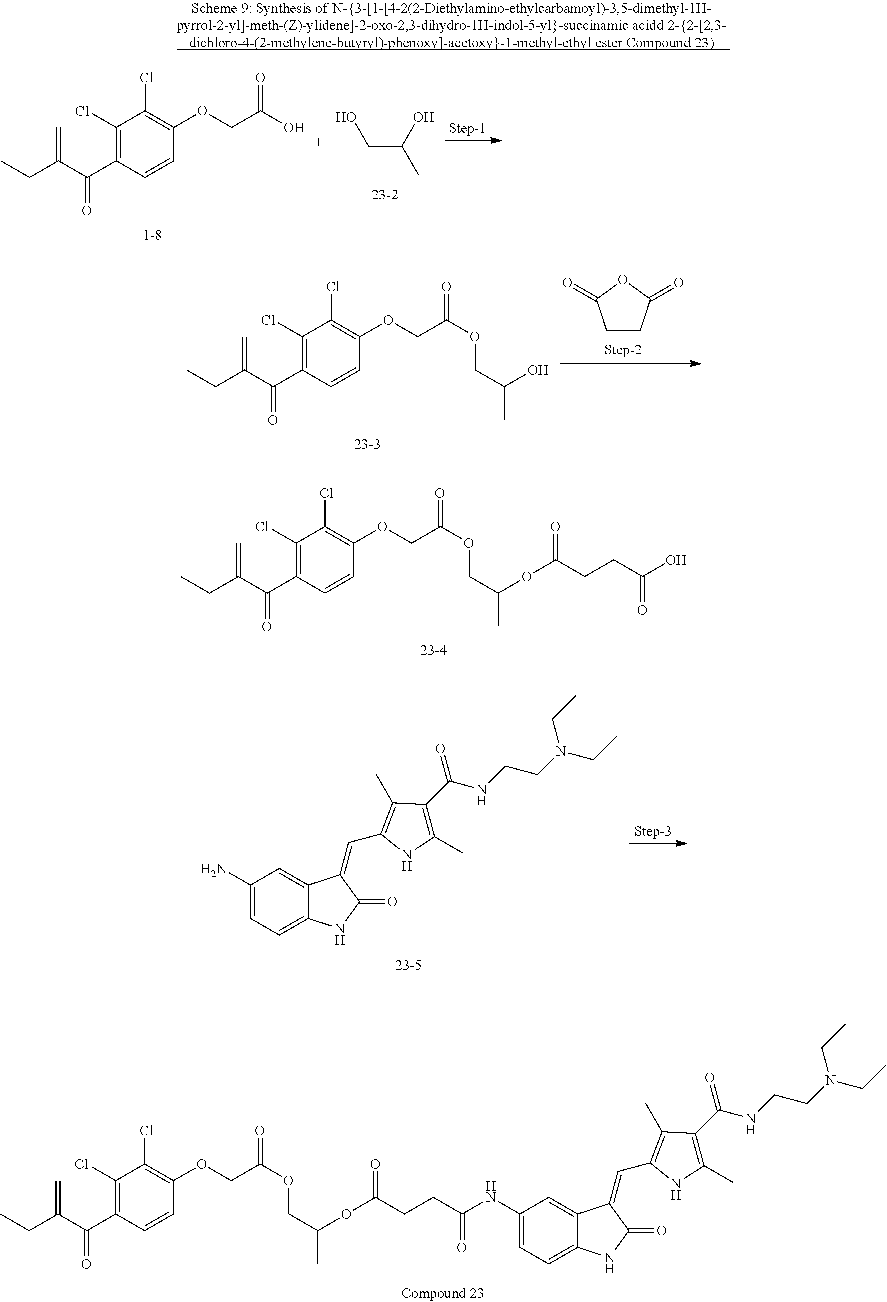

### Step 1: Preparation of [2,3-Dichloro-4-(2-methylene-butyryl)-phenoxy]-acetic acid 2-hydroxy-propyl ester (23-3)

To a solution of propane-1,2-diol 23-2 (816 mg, 10.721 mmol) in dichloromethane (6.5 mL) was added EDC.HCl (430 mg, 2.251 mmol) and 4-dimethyl amino pyridine (26 mg, 0.214 mmol) at 0° C. To the resultant reaction mixture was added ethacrynic acid 1-8 (650 mg, 2.144 mmol) portionwise at 0° C. The reaction mixture was allowed to stir at 25-30° C. over a period of 2 hours. The progress of the reaction was monitored by TLC and LCMS. The reaction mixture was diluted with water (100 mL) and extracted with dichloromethane (2×150 mL). The combined organic layer was dried over sodium sulphate and concentrated under reduced pressure. The crude product obtained upon evaporation of volatiles was purified through silica gel (60-120 mesh) column chromatography (40-50% ethyl acetate in hexane) to obtain product 23-3 as a thick colourless liquid 530 mg (68%).

### Step 2: Preparation of Succinic acid mono-(2-(2-[2,3-dichloro-4-(2-methylene-butyryl)-phenoxy]-acetoxy)-1-methyl-ethyl) ester (23-4)

To a solution of [2,3-dichloro-4-(2-methylene-butyryl)-phenoxy]-acetic acid 2-hydroxy-propyl ester 23-3 (530 mg, 1.467 mmol) in dichloromethane (5.3 mL) was added dihydro-furan-2,5-dione (190.8 mg, 1.907 mmol) and 4-dimethyl amino pyridine (18 mg, 0.146 mmol) at 25° C. The reaction mixture was allowed to stir at 25-30° C. over a period of 3 hours. The progress of the reaction was monitored by TLC and LCMS. The reaction mixture was diluted with water (100 mL) and extracted with dichloromethane (2×150 mL). The combined organic layer was dried over sodium sulphate and concentrated under reduced pressure. The crude product obtained upon evaporation of volatiles was purified through silica gel (60-120 mesh) column chromatography (1-2% methanol in dichloromethane) to obtain product 23-4 as a thick colourless liquid 510 mg (40%).

### Step 3: Preparation of N-{3-[I-[4-(2-Diethylamino-ethylcarbamoyl)-3,5-dimethyl-1H-pyrrol-2-yl]-meth-(Z)-ylidene]-2-oxo-2,3-dihydro-1H-indol-5-yl}-succinamic acid 2-(2-[2,3-dichloro-4-(2-methylene-butyryl)-phenoxy]-acetoxy)-1-methyl-ethyl ester (Compound 23)

To a solution of succinic acid mono-(2-{2-[2,3-dichloro-4-(2-methylene-butyryl)-phenoxy]-acetoxy}-1-methyl-ethyl) ester 23-4 (430 mg, 0.931 mmol) in N,N-dimethyl formamide (5 mL), were added N,N-diisopropylethylamine (0.5 mL, 2.941 mmol), HATU (373 mg, 0.980 mmol) and 5-aminoSunitinib 23-5 (500 mg, 0.980 mmol) at 0° C. The reaction mixture was allowed to stir at 25-30° C. for 3 hours. The progress of the reaction was monitored by TLC and LCMS. The reaction mixture was quenched with chilled water (50 mL). The solid precipitate was collected by filtration and dried under vacuum. The solid obtained was washed with ethyl acetate (10 mL) followed by 10% sodium bicarbonate solution, filtered and dried under vacuum to obtain Compound 23 as an orange solid 280 mg (34%).

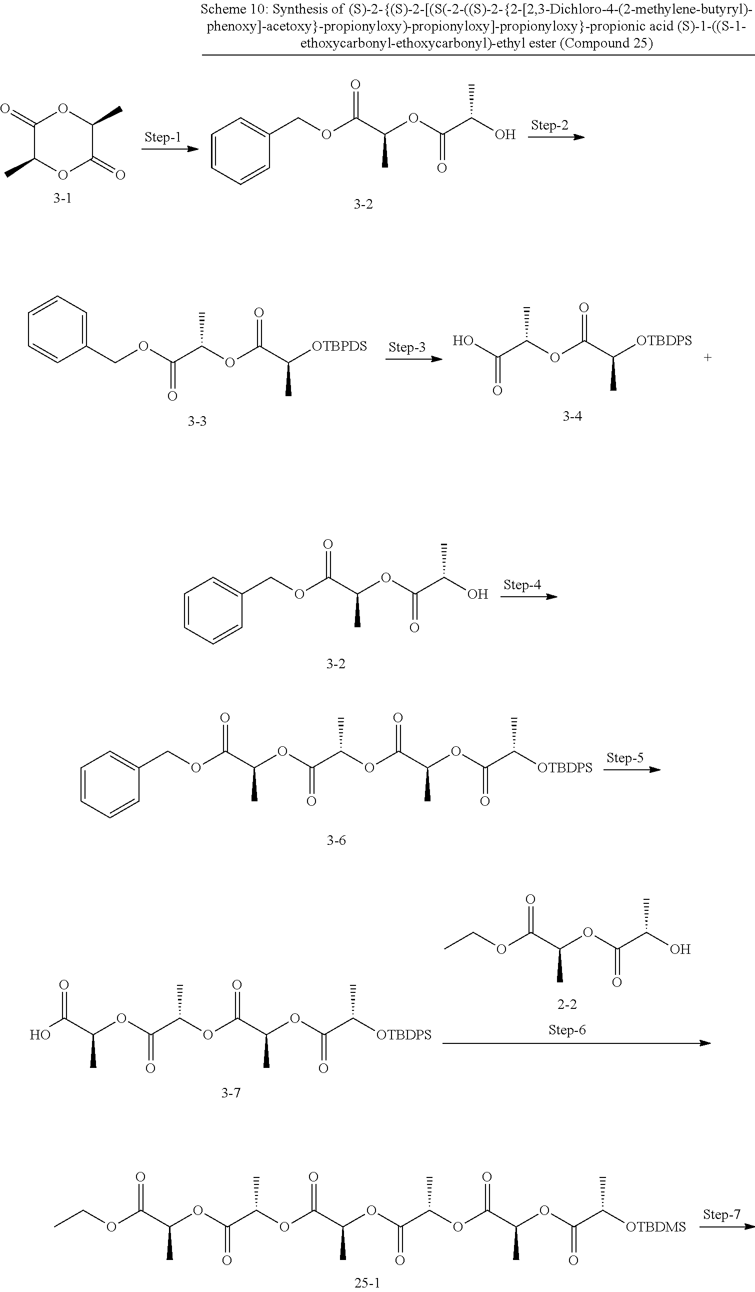

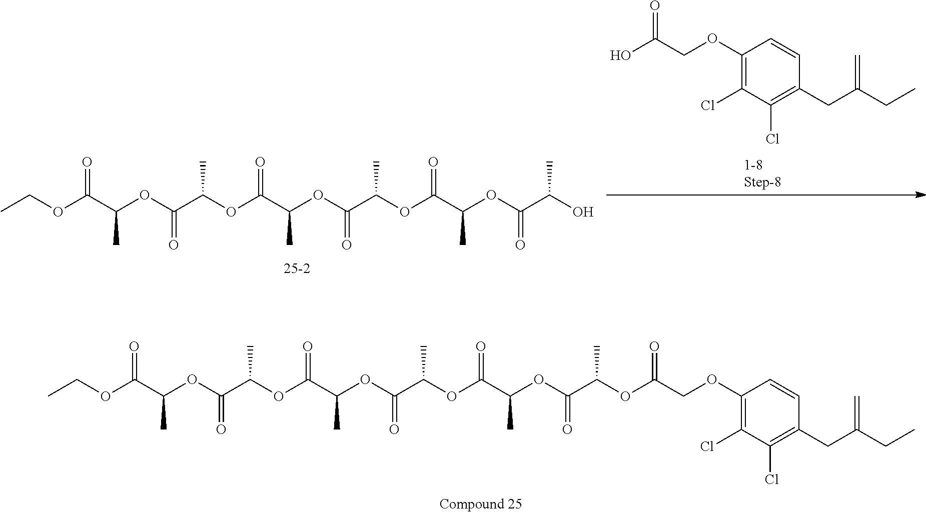 [SMILES: C=C(CC)Cc1ccc(OCC(=O)O)c(Cl)c1Cl ; CCOC(=O)[C@H](C)OC(=O)[C@H](C)OC(=O)[C@H](C)OC(=O)[C@H](C)OC(=O)[C@H](C)OC(=O)[C@H](C)O ; C=C(CC)Cc1ccc(OCC(=O)O[C@@H](C)C(=O)O[C@@H](C)C(=O)O[C@@H](C)C(=O)O[C@@H](C)C(=O)O[C@@H](C)C(=O)O[C@@H](C)C(=O)OCC)c(Cl)c1Cl]

### Step 1: Preparation of (S)-2-Hydroxy-propionic acid (S)-1-benzyloxycarbonyl-ethyl ester (3-2)

To a solution of (3S,6S)-3,6-dimethyl-[1,4]dioxane-2,5-dione 3-1 (5.0 g, 34.72 mmol) in toluene (100 mL) was added benzyl alcohol (3.2 mL, 31.72 mmol) and camphor sulfonic acid (0.8 g, 3.47 mmol) at 25-30° C. The reaction mixture was allowed to stir at 80° C. over a period of 2 h. The resulting reaction mixture was diluted with ethyl acetate (800 mL) and washed with water (2×400 mL). The crude product obtained upon evaporation of volatiles was purified through preparative HPLC to obtain product 3-2 as a pale yellow liquid 5.5 g (63%). ¹H NMR (400 MHz, DMSO-d₆) δ 7.41-7.32 (m, 5H), 5.48 (d, J=5.6 Hz, 1H), 5.15 (s, 2H), 5.10 (q, J=7 Hz, 1H), 4.20-4.18 (m, 1H), 1.42 (d, J=7 Hz, 3H), 1.16 (d, J=7 Hz, 3H). MS m/z [M+H]⁺ 253.4, [M+NH₄+]⁺ 270.3.

### Step 2: Preparation of (S)-2-(tert-Butyl-diphenyl-silanyloxy)-propionic acid (S)-1 benzyloxycarbonyl-ethyl ester (3-3)

To a solution of (S)-2-hydroxy-propionic acid (S)-1-benzyloxycarbonyl-ethyl ester 3-2 (0.1 g, 0.23 mmol) in dichloromethane (5 mL) were added triethylamine (0.23 mL, 1.61 mmol), TBDPS-Cl (0.43 mL, 1.618 mmol) and catalytic amount of 4-dimethylaminopyridine at 0° C. The reaction mixture was stirred at room temperature over period of 8 h. The resulting reaction mixture was quenched with water (20 mL) and extracted with ethyl acetate (2×50 mL). Then volatiles were evaporated under reduced pressure to obtain product 3-3 as a colorless liquid 200 mg (74%). This material was carried into the next step without further purification.

### Step 3: Preparation of (S)-2-(tert-Butyl-diphenyl-silanyloxy)-propionic acid (S)-1-carboxy-ethyl ester (3-4)

To a 100 mL autoclave vessel were added a solution of(S)-2-(tert-butyl-diphenyl-silanyloxy)-propionic acid (S)-1-benzyloxycarbonyl-ethyl ester 3-3 (1.5 g) in methanol (20 mL) and 10% Pd/C (0.3 g, 50% wet) at 25-30° C. The reaction mixture was stirred at room temperature under hydrogen pressure (5 kg/cm²) over a period of 2 h. After completion of the reaction, the reaction mixture was filtered through a celite bed and concentrated under reduced pressure. The crude product obtained upon evaporation of volatiles was purified through silica gel (60-120 mesh) column chromatography (10% methanol in dichloromethane) to obtain product 3-4 as a colorless liquid 700 mg (58%). ¹H-NMR (400 MHz, DMSO-d₆) δ 13.1 (bs, 1H), 7.63-7.62 (m, 4H), 7.62-7.37 (m, 6H), 4.77 (q, J=7.6 Hz, 1H), 4.26 (q, J=8.0.0 Hz, 1H), 1.31 (d, J=6.8 Hz, 3H), 1.23 (d, J=7.2 Hz, 3H), 1.02 (s, 9H); MS m/z [M−H]⁺ 399.1.

### Step 4: Preparation of (S)-2-(tert-Butyl-diphenyl-silanyloxy)-propionic acid (S)-1-[(S)-1-((S)-1-benzyloxycarbonyl-ethoxycarbonyl)-ethoxycarbonyl]-ethyl ester (3-6)

To a solution of (S)-2-hydroxy-propionic acid (S)-1-benzyloxycarbonyl-ethyl ester 3-2 (6.0 g, 33.2 mmol) and (S)-2-(tert-butyl-diphenyl-silanyloxy)-propionic acid (S)-1-carboxy-ethyl ester 3-4 (17.3 g, 7.77 mmol) in dichloromethane (60 mL) were added EDC.HCl (8.2 g, 43.2 mmol), 4-dimethylaminopyridine (405 mg, 3.3 mmol) at 0° C. The reaction mixture was allowed to stir at 25-30° C. over a period of 1 h. The resulting reaction mass was quenched with water (200 mL), extracted with dichloromethane (3×250 mL), dried over Na₂SO₄ and concentrated under reduced pressure. The crude product obtained upon evaporation of volatiles was purified through silica gel (230-400 mesh) column chromatography (10% methanol in dichloromethane) to obtain product 3-6 as a pale yellow liquid 5.8 g (94%). ¹H NMR (400 MHz, DMSO-d₆) δ 7.60 (d, J=8 Hz, 4H), 7.49-7.33 (m, 11H), 5.20-5.15 (m, 4H), 4.95 (q, J=7.2 Hz, 1H), 4.29 (q, J=6.4 Hz, 1H), 1.43 (d, J=7.2 Hz, 3H), 1.39 (d, J=7.2 Hz, 3H), 1.31 (d, J=6.8 Hz, 3H), 1.28 (d, J=1.28 Hz, 3H), 1.02 (s, 9H); MS m/z [M+NH₄]⁺ 652.8.

### Step 5: Preparation of (S)-2-(tert-Butyl-diphenyl-silanyloxy)-propionic acid (S)-1-[(S)-1-((S)-1-carboxy-ethoxycarbonyl)-ethoxycarbonyl]-ethyl ester (3-7)

To a 100 mL autoclave vessel were added a solution of(S)-2-(tert-Butyl-diphenyl-silanyloxy)-propionic acid (S)-1-[(S)-1-((S)-1-benzyloxycarbonyl-ethoxycarbonyl)-ethoxycarbonyl]-ethyl ester 3-6 (700 mg, 1.10 mmol) in methanol (10 mL) and 10% Pd/C (140 mg, 50% wet) at 25-30° C. The reaction mixture was stirred at room temperature under hydrogen pressure (5 kg/cm²) over a period of 2 h. After completion of the reaction, the reaction mixture was filtered through a celite bed and concentrated under reduced pressure. The crude product obtained upon evaporation of volatiles was purified through silica gel (60-120 mesh) column chromatography (10% methanol in dichloromethane) to obtain product 3-7 as a pale yellow liquid 420 mg (78%). ¹H NMR (400 MHz, DMSO-d₆) δ 13.2 (bs, 1H), 7.62-7.60 (m, 4H), 7.59-7.40 (m, 6H), 5.16 (q, J=7.2 Hz 1H), 4.98-4.93 (m, 2H), 4.29 (q, J=6.8, 1H), 1.44 (d, J=7.2 Hz, 3H), 1.40 (d, J=7.2 Hz, 3H), 1.31-1.30 (m, 6H), 1.01 (s, 9H); MS m/z [M+NH₄]⁺ 562.3; MS m/z [M−H]⁺ 543.1.

### Step 6: Preparation of (S)-2-(tert-Butyl-diphenyl-silanyloxy)-propionic acid (S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethyl ester (25-1)

To a solution of (S)-2-(tert-Butyl-diphenyl-silanyloxy)-propionic acid (S)-1-[(S)-1-((S)-1-carboxy-ethoxycarbonyl)-ethoxycarbonyl]-ethyl ester 3-7 (7.44 g, 13.68 mmol) in dichloromethane (20 mL) were added EDC.HCl (2.411 g, 12.62 mmol), (S)-2-Hydroxy-propionic acid (S)-1-ethoxycarbonyl-ethyl ester (2 g, 10.52 mmol) and 4-dimethylaminopyridine (128 mg, 1.05 mmol) at 0° C. The reaction mixture was allowed to stir at 25-30° C. over a period of 1 h. The resulting reaction mass was quenched with water (200 mL), extracted with dichloromethane (2×250 mL), dried over Na₂SO₄ and concentrated under reduced pressure. The crude product obtained upon evaporation of volatiles was purified through silica gel (230-400 mesh) column chromatography (5% ethyl acetate in hexane) to obtain product 25-1 as a colorless liquid 6.0 g (79%). ¹H NMR (400 MHz, DMSO-d₆) δ 7.63-7.57 (m, 4H), 7.51-7.36 (m, 6H), 5.23-5.15 (m, 3H), 5.08 (q, J=7 Hz, 1H), 4.95 (q, J=7 Hz, 1H), 4.28 (q, J=7 Hz, 1H), 4.16-4.06 (m, 2H), 1.50-1.39 (m, 12H), 1.34-1.25 (m, 6H), 1.18 (t, 3H), 1.02 (s, 9H); MS m/z [M+NH₄]⁺ 735.0.

### Step 7: Preparation of (S)-2-Hydroxy-propionic acid (S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethyl ester (25-2)

To a solution of (S)-2-(tert-Butyl-diphenyl-silanyloxy)-propionic acid (S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethyl ester 25-1 (7 g, 9.78 mmol) in tetrahydrofuran (70 mL) were added tetra butyl ammonium fluoride (14.64 mL, 1.0M, 14.66 mmol) and acetic acid (0.88 g, 14.66 mmol) at 0° C. The reaction mixture was allowed to stir at room temperature over a period of 1 h. The resulting reaction mixture was concentrated under reduced pressure and crude product obtained upon evaporation of the volatiles was purified through silica gel (230-400 mesh) column chromatography (14% ethyl acetate in hexane) to afford product 25-2 as a colorless liquid 3.0 g (64%). ¹H NMR (400 MHz, DMSO-d₆) δ 5.49 (d, 1H), 5.24-5.15 (m, 3H), 5.15-5.04 (m, 2H), 4.20 (quintet, 1H), 4.16-4.06 (m, 2H), 1.50-1.39 (m, 15H), 1.28 (d, 3H), 1.18 (t, 3H); MS m/z [M+NH₄]⁺ 496.7.

### Step 8: Preparation of (S)-2-((S)-2-[(S)-2-((S)-2-(2-[2,3-Dichloro-4-(2-methylene-butyryl)-phenoxy]-acetoxy)-propionyloxy)-propionyloxy]-propionyloxy)-propionic acid (S)-1-((S)-1-ethoxycarbonyl-ethoxycarbonyl)-ethyl ester (Compound 25)

To a solution of etacrynic acid 1-8 (1.95 g, 6.40 mmol) in dichloromethane (20 mL) were added EDC.HCl (1.18 g, 7.61 mmol), 25-2 (2.8 g, 5.85 mmol) and 4-dimethyl amino pyridine (71 mg, 0.58 mmol) at 0° C. The reaction mixture was allowed to stir at 25-30° C. over a period of 2 h. The reaction mixture was diluted with water (300 mL) and extracted with dichloromethane (2×300 mL). The combined organic layer was dried over sodium sulfate and concentrated under reduced pressure. The crude product obtained upon evaporation of volatiles was purified through silica gel (60-120 mesh) column (13% ethyl acetate in hexane) to obtain product Compound 25 as a colorless wax 2.2 g (49%). ¹H NMR (400 MHz, DMSO-d₆) δ 7.32 (d, 8.6 Hz, 1H), 7.16 (d, 8.6 Hz, 1H), 6.08 (s, 1H), 5.56 (s, 1H), 5.27-5.13 (m, 7H), 5.09 (q, 1H), 4.16-4.06 (m, 2H), 2.40-2.29 (m, 2H), 1.50-1.39 (m, 18H), 1.18 (t, 3H), 1.08 (t, 3H); MS m/z [M+H]⁺ 765.1, [M+NH₄]⁺ 781.1.

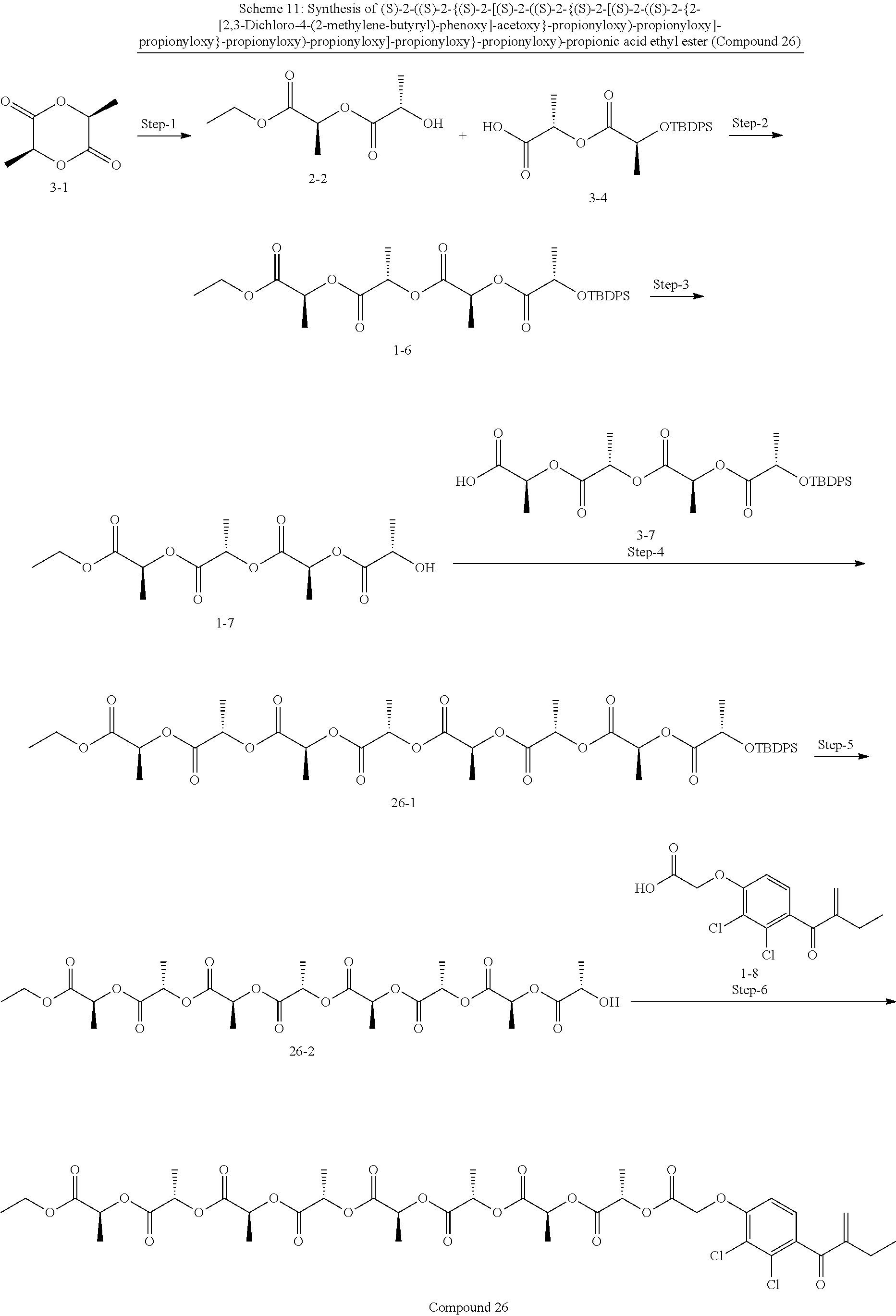

### Step 1: Preparation of (S)-2-Hydroxy-propionic acid (S)-1-ethoxycarbonyl-ethyl ester (2-2)

To a solution of (3 S,6S)-3,6-dimethyl-[1,4]-dioxane-2,5-dione 3-1 (5.0 g, 34.72 mmol) in toluene (100 mL) was added ethanol (1.92 mL, 31.98 mmol) and camphor sulfonic acid (0.8 g, 3.47 mmol) at 25-30° C. The reaction mixture was allowed to stir at 80° C. over a period of 2 h. The resulting reaction mixture was diluted with ethyl acetate (800 mL) and washed with water (2×200 mL). The crude product obtained upon evaporation of volatiles was purified through silica gel (230-400 mesh) column chromatography (13% ethyl acetate in hexane) to obtain product 2-2 as a colorless liquid 6.6 g (60%). ¹H-NMR (400 MHz, DMSO-d₆) δ 5.45 (d, 1H), 5.03 (q, 1H), 4.24-4.06 (m, 3H), 1.41 (d, J=7 Hz, 3H), 1.29 (d, J=7 Hz, 3H), 1.18 (t, 3H); MS m/z, [M+Na]⁺ 213.7.

### Step 2: Preparation of (S)-2-(tert-Butyl-diphenyl-silanyloxy)-propionic acid (S)-1-[(S)-1-((S)-1-ethoxycarbonyl-ethoxycarbonyl)-ethoxycarbonyl]-ethyl ester (1-6)

To a solution of (S)-2-(tert-Butyl-diphenyl-silanyloxy)-propionic acid (S)-1-carboxy-ethyl ester 3-4 (5.4 g, 13.68 mmol) in dichloromethane (60 mL) were added EDC.HCl (3.0 g, 15.78 mmol), (S)-2-Hydroxy-propionic acid (S)-1-ethoxycarbonyl-ethyl ester (2.0 g, 10.52 mmol) and 4-dimethylaminopyridine (0.12 g, 1.05 mmol) at 0° C. The reaction mixture was allowed to stir at 25-30° C. over a period of 1 h. The resulting reaction mass was quenched with water (200 mL), extracted with dichloromethane (3×250 mL), dried over Na₂SO₄ and concentrated under reduced pressure. The crude product obtained upon evaporation of volatiles was purified through silica gel (230-400 mesh) column chromatography (3% ethyl acetate in hexane) to obtain product 1-6 as a colorless liquid 4.2 g (70%). ¹H-NMR (400 MHz, DMSO-d₆) δ 7.64-7.67 (m, 4H), 7.61-7.36 (m, 6H), 5.17 (q, 1H), 5.08 (q, 1H), 4.95 (q, 1H), 4.29 (q, 1H), 4.15-4.06 (m, 2H), 1.45 (d, J=7 Hz, 3H), 1.41 (d, J=7 Hz, 3H), 1.34-1.26 (m, 6H), 1.7 (t, 3H), 1.02 (s, 9H).

### Step 3: Preparation of (S)-2-Hydroxy-propionic acid (S)-1-[(S)-1-((S)-1-ethoxycarbonyl-ethoxycarbonyl)-ethoxycarbonyl]-ethyl ester (1-7)

To a solution of (S)-2-(tert-Butyl-diphenyl-silanyloxy)-propionic acid (S)-1-[(S)-1-((S)-1-ethoxycarbonyl-ethoxycarbonyl)-ethoxycarbonyl]-ethyl ester 1-6 (4 g, 6.99 mmol) in tetrahydrofuran (40 mL) were added tetra butyl ammonium fluoride (10.49 mL, 1.0M, 10.49 mmol) and acetic acid (0.63 g, 10.49 mmol) at 0° C. The reaction mixture was allowed to stir at room temperature over a period of 1 h. The resulting reaction mixture was concentrated under reduced pressure and crude product obtained upon evaporation of the volatiles was purified through silica gel (230-400 mesh) column chromatography (12% ethyl acetate in hexane) to give product 1-7 as a colorless liquid 1.0 g (43%). ¹H-NMR (400 MHz, DMSO-d₆) δ 5.50 (d, 1H), 5.21-5.03 (m, 3H), 4.23-4.05 (m, 3H), 1.51-1.38 (m, 9H), 1.28 (d, 3H), 1.71 (t, 3H).

### Step 4: Preparation of (S)-2-(tert-Butyl-diphenyl-silanyloxy)-propionic acid (S)-1-{(S)-1-[(S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethoxycarbonyl]-ethoxycarbonyl}-ethyl ester (26-1)

To a solution of (S)-2-(tert-Butyl-diphenyl-silanyloxy)-propionic acid (S)-1-[(S)-1-((S)-1-carboxy-ethoxycarbonyl)-ethoxycarbonyl]-ethyl ester 3-7 (17.78 g, 32.69 mmol) in dichloromethane (84 mL) were added EDC.HCl (7.2 g, 37.72 mmol), (S)-2-Hydroxy-propionic acid (S)-1-[(S)-1-((S)-1-ethoxycarbonyl-ethoxycarbonyl)-ethoxycarbonyl]-ethyl ester (8.4 g, 25.15 mmol) and 4-dimethylaminopyridine (0.30 g, 2.51 mmol) at 0° C. The reaction mixture was allowed to stir at 25-30° C. over a period of 1 h. The resulting reaction mass was quenched with water (500 mL), extracted with dichloromethane (4×250 mL), dried over Na₂SO₄ and concentrated under reduced pressure. The crude product obtained upon evaporation of volatiles was purified through silica gel (230-400 mesh) column chromatography (8% ethyl acetate in hexane) to obtain product 26-1 as a colorless liquid 10.0 g (47.6%). ¹H NMR (400 MHz, DMSO-d₆) δ 7.64-7.57 (m, 4H), 7.52-7.36 (m, 6H), 5.25-5.15 (m, 5H), 5.11 (q, 1H), 4.93 (q, 1H), 4.29 (q, 1H), 4.15-4.04 (m, 2H), 1.50-1.39 (m, 18H), 1.35-1.26 (m, 6H), 1.18 (t, 3H), 1.02 (s, 9H).

### Step 5: Preparation of (S)-2-Hydroxy-propionic acid (S)-1-{(S)-1-[(S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethoxycarbonyl]-ethoxycarbonyl}-ethyl ester (26-2)

To a solution of (S)-2-(tert-Butyl-diphenyl-silanyloxy)-propionic acid (S)-1-{(S)-1-[(S)-1-((S)-1-{(S)-1-[(S)-1-((S)-1-ethoxycarbonyl-ethoxycarbonyl)-ethoxycarbonyl]-ethoxycarbonyl}-ethoxycarbonyl)-ethoxycarbonyl]-ethoxycarbonyl}-ethyl ester 26-1 (10.0 g, 11.63 mmol) in tetrahydrofuran (100 mL) were added tetra butyl ammonium fluoride (17.44 mL, 1.0M, 17.44 mmol) and acetic acid (0.88 g, 17.44 mmol) at 0° C. The reaction mixture was allowed to stir at room temperature over a period of 1 h. The resulting reaction mixture was concentrated under reduced pressure and crude product obtained upon evaporation of the volatiles was purified through silica gel (230-400 mesh) column chromatography (14% ethyl acetate in hexane) to give product 26-2 as a colorless liquid 4.5 g (62%). ¹H NMR (400 MHz, DMSO-d₆) δ 5.49 (d, 1H), 5.24-5.04 (m, 7H), 4.21 (quintet, 1H), 4.16-4.06 (m, 2H), 1.50-1.39 (m, 21H), 1.28 (d, 3H), 1.18 (t, 3H); MS m/z [M+NH₄]⁺ 640.8.

### Step 6: Preparation of (S)-2-((S)-2-{(S)-2-[(S)-2-((S)-2-{(S)-2-[(S)-2-((S)-2-{2-[2,3-Dichloro-4-(2-methylene-butyryl)-phenoxy]-acetoxy}-propionyloxy)-propionyloxy]-propionyloxy}-propionyloxy]-propionyloxy}-propionyloxy)-propionic acid ethyl ester (Compound 26)

To a solution of etacrynic acid 18-1 (2.85 g, 9.40 mmol) in dichloromethane (30 mL) were added EDC.HCl (1.68 g, 10.85 mmol), (S)-2-Hydroxy-propionic acid (S)-1-{(S)-1-[(S)-1-((S)-1-{(S)-1-[(S)-1-((S)-1-ethoxycarbonyl-ethoxycarbonyl)-ethoxycarbonyl]-ethoxycarbonyl)}-ethoxycarbonyl)-ethoxycarbonyl]-ethoxycarbonyl)}-ethyl ester 26-2 (4.5 g, 7.23 mmol) and 4-dimethyl amino pyridine (88 mg, 0.72 mmol) at 0° C. The reaction mixture was allowed to stir at 25-30° C. over a period of 2 h. The reaction mixture was diluted with water (300 mL) and extracted with dichloromethane (2×300 mL). The combined organic layer was dried over sodium sulfate and concentrated under reduced pressure. The crude product obtained upon evaporation of volatiles was purified through silica gel (60-120 mesh) column (13% ethyl acetate in hexane) to obtain product Compound 26 as a colorless wax 3.0 g (45%). ¹H NMR (400 MHz, DMSO-d₆) δ 7.32 (d, 8.6 Hz, 1H), 7.16 (d, 8.6 Hz, 1H), 6.08 (s, 1H), 5.56 (s, 1H), 5.27-5.13 (m, 9H), 5.09 (q, 1H), 4.17-4.06 (m, 2H), 2.40-2.29 (m, 2H), 1.51-1.39 (m, 24H), 1.18 (t, 3H), 1.07 (t, 3H); MS m/z [M+H]⁺ 909.7.

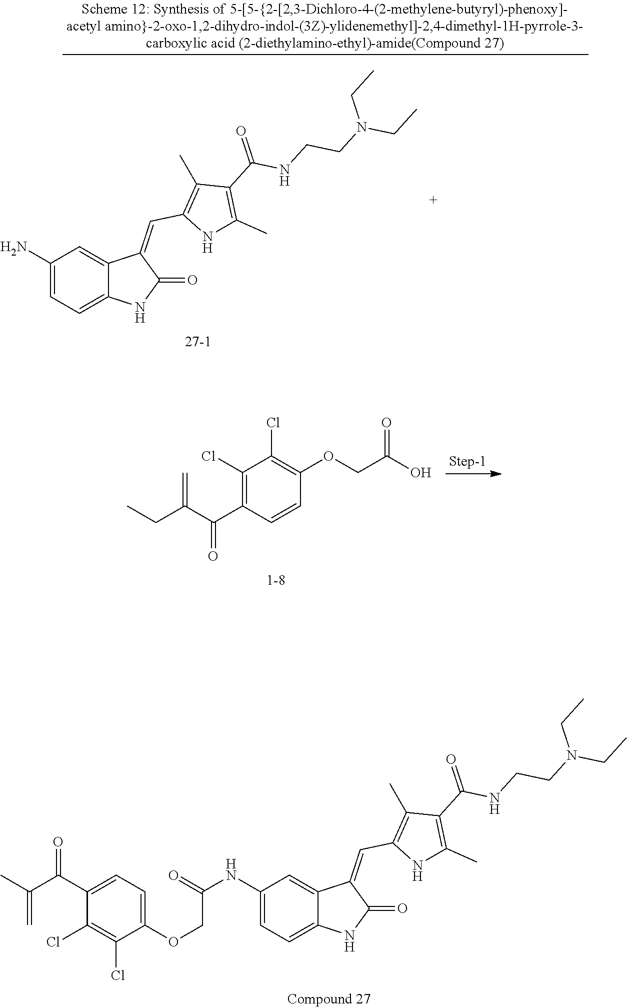

### Step 1: Preparation of 5-[5-{2-[2,3-Dichloro-4-(2-methylene-butyryl)-phenoxy]-acetyl amino}-2-oxo-1,2-dihydro-indol-(3Z)-ylidenemethyl]-2,4-dimethyl-1H-pyrrole-3-carboxylic acid (2-diethylamino-ethyl)-amide (Compound 27)

To a solution of ethacrynic acid 1-8 (1.48 g, 4.901 mmol) in N,N-dimethyl formamide (25 mL) was added N,N-diisopropylethylamine (2.5 mL, 14.705 mmol), HATU (1.86 g, 4.901 mmol) and 5-aminoSunitinib 27-1 (2.5 g, 4.901 mmol) at 0° C. The reaction mixture was allowed to stir at 25-30° C. over a period of 3 hours. The progress of the reaction was monitored by TLC and LCMS. The reaction mixture was quenched with chilled water. The solid precipitate was collected by filtration and dried under vacuum. The solid obtained was washed with ethyl acetate (10 mL) followed by 10% sodium bicarbonate solution, filtered and dried under vacuum to obtain Compound 27 as an orange solid 2.0 g (60%).

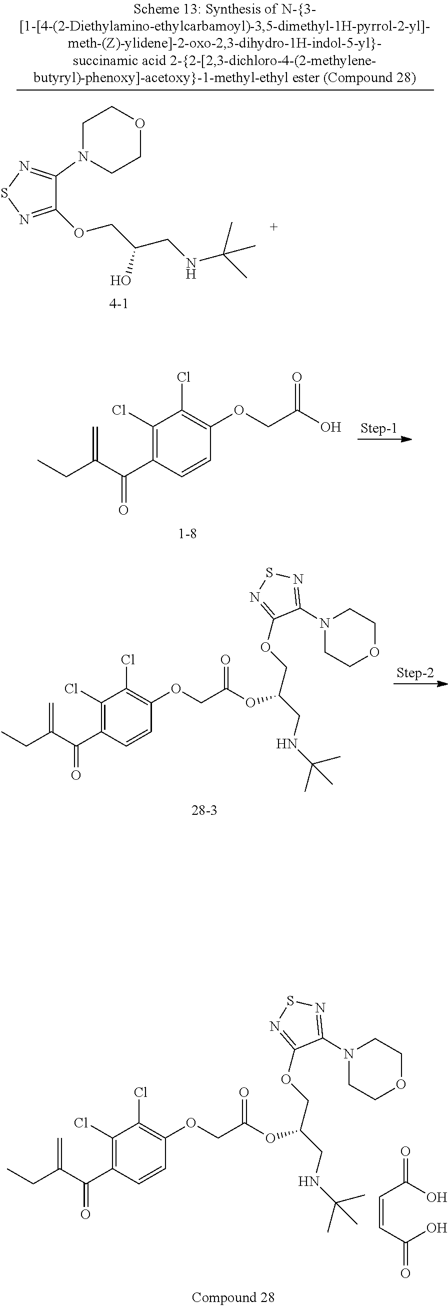

### Step 1: Preparation of [2,3-Dichloro-4-(2-methylene-butyryl)-phenoxy]-acetic acid (S)-1-(tert-butylamino-methyl)-2-(4-morpholin-4-yl-[1,2,5] thiadiazol-3-yloxy)-ethyl ester (28-3)

To a solution of ethacrynic acid 1-8 (2.63 g, 8.691 mmol) in dichloromethane (25 mL) was added N,N-diisopropylethylamine (1.8 mL, 11.061 mmol), HATU (3.6 g, 9.481 mmol) and Timolol 4-1 (2.5 g, 7.901 mmol) at 0° C. The reaction mixture was allowed to stir at 25-30° C. over a period of 3 hours. The progress of the reaction was monitored by TLC and LCMS. The reaction mixture was diluted with water (250 mL) and extracted with dichloromethane (2×400 mL), dried over Na₂SO₄ and concentrated under reduced pressure. The crude product obtained upon evaporation of volatiles was purified through silica gel (60-120 mesh) column chromatography (80% ethyl acetate in hexane) to obtain product 28-3 as an off white solid 2.5 g (52%).

### Step 2: Preparation of [2,3-Dichloro-4-(2-methylene-butyryl)-phenoxy]-acetic acid (S)-1-(tert-butylamino-methyl)-2-(4-morpholin-4-yl-[1,2,5]thiadiazol-3-yloxy)-ethyl ester maleate salt (Compound 28)

To a solution of [2,3-Dichloro-4-(2-methylene-butyryl)-phenoxy]-acetic acid(S)-1-(tert-butylamino-methyl)-2-(4-morpholin-4-yl-[1,2,5]thiadiazol-3-yloxy)-ethyl ester 28-3 (2.5 g, 4.155 mmol) in acetone (7.5 mL) was added maleic acid (0.434 g, 3.740 mmol) and the reaction mixture stirred for 10 minutes at 25° C. The reaction mixture was concentrated under reduced pressure to obtain Compound 28 as an off white solid 2.3 g (77%).

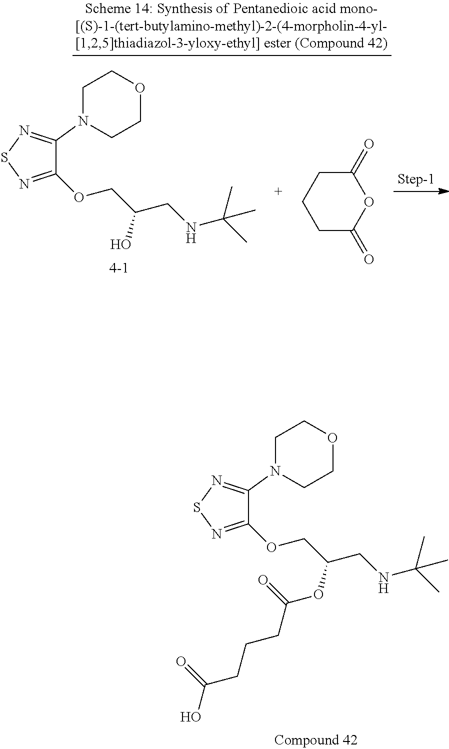 [SMILES: CC(C)(C)NC[C@H](O)COc1nsnc1N1CCOCC1 ; O=C1CCCC(=O)O1 ; CC(C)(C)NC[C@@H](COc1nsnc1N1CCOCC1)OC(=O)CCCC(=O)O]

### Step 1: Preparation of Pentanedioic acid mono-[(S)-1-(tert-butylamino-methyl)-2-(4-morpholin-4-yl-[1,2,5]thiadiazol-3-yloxy)-ethyl] ester (Compound 42)

To a solution of (S)-1-tert-butylamino-3-(4-morpholin-4-yl-[1,2,5]thiadiazol-3-yloxy)-propan-2-ol 4-1 (2.0 g, 6.32 mmol) in dichloromethane (20 mL) were added dihydro-pyran-5,6-dione (0.86 g, 7.59 mmol) and 4-dimethylaminopyridine (0.079 g, 0.62 mmol) at 0° C. The reaction mixture was allowed to stir at room temperature over a period of 2 h. The resulting reaction mixture was concentrated under reduced pressure to give Compound 42 as an off white solid 2.0 g (73%).

### Example 14. Drug Release of Timolol Compounds

A series of Timolol prodrugs (Compound 50, Compound 51, Compound 52, Compound 53, Compound 54, Compound 55, and Compound 56) were encapsulated in polymeric microparticles.

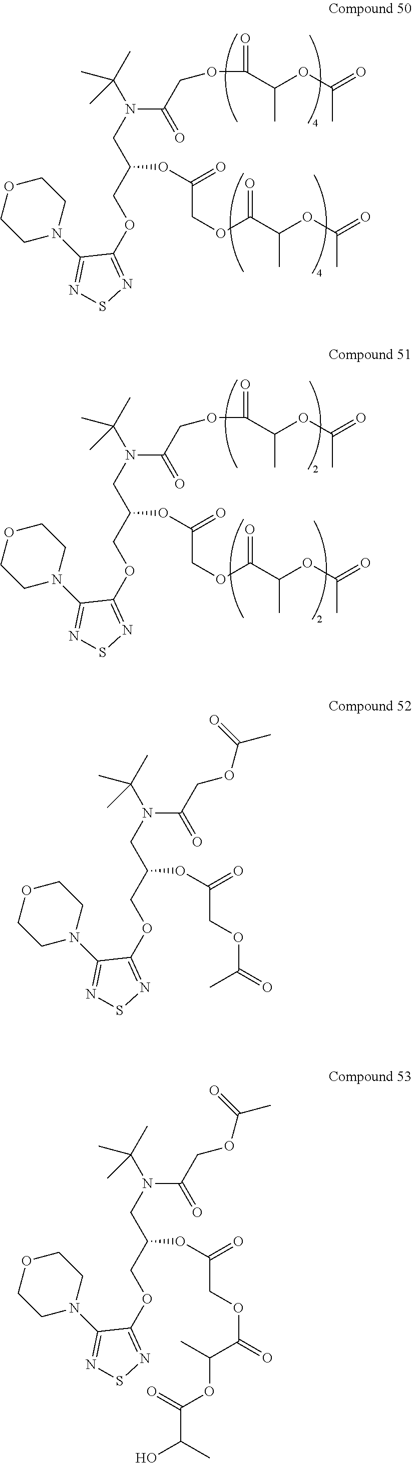

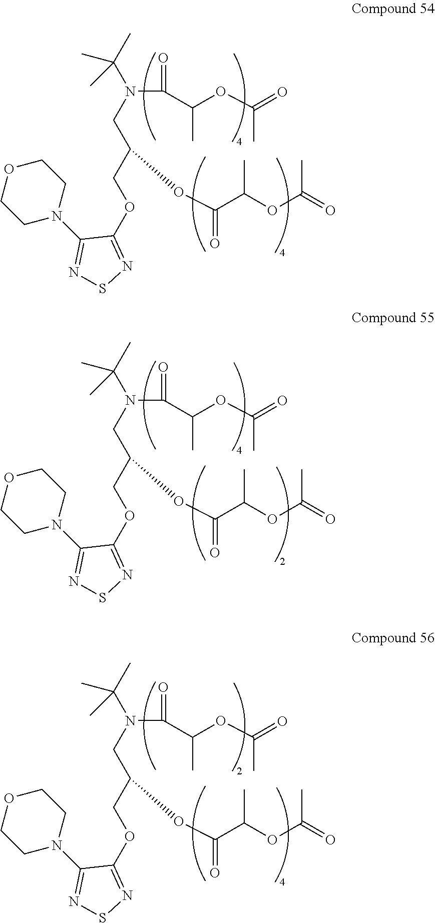

The microparticles containing the prodrugs of Timolol were formulated using an oil-in-water solvent evaporation microencapsulation method. The polymer was initially dissolved in a water immiscible organic solvent to which dissolved drug was added. Briefly, PLGA (140-200 mg), PLA (140-200 mg/mL) or a blend of PLGA and PLA with varying lactide to glycolide compositions, and PLGA50/50-PEG5k (1.4-2 mg/mL) was dissolved in 2 mL of methylene chloride. The prodrug (13.8-50% theoretical loading) was dissolved in 1 mL of DMSO or ethyl acetate after vigorous vortexing and ultrasonication in a bath sonicator and added to the polymer solution. The aqueous phase consisted of 200 mL of PBS or water with 1% PVA as a surfactant to stabilize the emulsification. The aqueous phase was mixed at 5000 rpms using a Silverson L5A-M benchtop mixer. The dispersed phase was rapidly added to the aqueous phase and allowed to mix at 5000 rpms for 1 minute to generate an oil-in-water emulsion and disperse the materials as droplets. The organic solution was allowed to evaporate under constant stirring at 500 rpms for 2 hours under ambient temperatures. The particle suspension was allowed to settle for 30 min, after which the solution was decanted and remaining particles were collected, suspended in distilled deionized water, and washed 3 times using water via centrifugation at 1000 rpms for 5 minutes to remove any residual solvent. The pellet was collected and lyophilized overnight.

Particle size and size distribution was determined using a Beckman Coulter Multsizer IV with a 100 μm diameter aperture based on a sample size of at least 50,000 counts. Particle size is expressed as volume-weighted mean diameters. Briefly, 2-5 mg of particles were suspended in 1 mL of double distilled water and added to a beaker containing 100 mL of ISOTON II solution. Measurements were obtained once the coincidence of particles reached 6-10%. Table 11 outlines the size of the microparticles generated for each test compound. The volume-weighted mean diameters for all Timolol bis-prodrug loaded microparticles ranged from approximately 26 μm to 29 μm depending on the formulation parameters.

To determine the % drug loading (DL), 10 mg of particles was weighed into a glass scintillation vial and dissolved with 10 mL of MeCN:water (1:1, v/v). The solution was filtered through a 0.2 μm nylon syringe filter and the drug content was determined by RP-HPLC referenced against a standard calibration curve. The drug loading results are presented in Table 11. Overall, the bis-prodrugs of Timolol were amenable to microparticle encapsulation with high loading efficiency even at the 30% theoretical load level.

TABLE 11
Formulation Parameters of Microparticles encapsulating Timolol Prodrugs

| | | | Mean | % | |
|---|---|---|---|---|---|
| | Batch | Polymer | Particle Size | Theoretical | % Drug |
| Compound | ID | Composition | (μm) | Loading | Loading |
| Compound 50 | 50-1 | 77% PLA 4A | 28.50 | 30 | 28.18 |
| (Timolol-Bis-N,O-Glycolic | | 22% PLGA8515 5A | | | |
| acid-Acetyl-PLA (n = 4)) | | 1% PEG-PLGA5050 | | | |
| Compound 52 | 52-1 | 77% PLA 4A | 29.24 | 15 | 9.81 |
| (Timolol-N,O-bis-Glycolic | | 22% PLGA8515 5A | | | |
| acid-OAc) | | 1% PEG-PLGA5050 | | | |
| Compound 53 | 53-1 | 77% PLA 4A | 26.82 | 15 | 5.65 |
| (Timolol-N-Glycolic | | 22% PLGA8515 5A | | | |
| acid-OAc-O-Glycolic | | 1% PEG-PLGA5050 | | | |
| acid-PLA (n = 2)-OH) | | | | | |
| Compound 54 | 54-1 | 64% PLA 4A | 27.73 | 15 | 14.31 |
| (Timolol-Bis-Acetyl | | 20% PLGA8515 5A | | | |
| PLA (n = 4)) | | 15% PLGA5050 4A | | | |
| | | 1% PEG-PLGA5050 | | | |
| | 54-2 | 77% PLA 4A | 28.51 | 15 | 14.23 |
| | | 22% PLGA8515 5A | | | |
| | | 1% PEG-PLGA5050 | | | |
| Compound 55 | 55-1 | 99% PLA 4A | 28.17 | 15 | 13.36 |
| (Timolol-Bis-N-Acetyl-PLA | | 1% PEG-PLGA5050 | | | |
| (n = 4)-O-Acetyl | 55-2 | 77% PLA 4A | 27.29 | 15 | 14.06 |
| PLA (n = 2)) | | 22% PLGA8515 5A | | | |
| | | 1% PEG-PLGA5050 | | | |
| | 55-3 | 64% PLA 4A | 26.87 | 15 | 13.40 |
| | | 20% PLGA8515 5A | | | |
| | | 15% PLGA5050 4A | | | |
| | | 1% PEG-PLGA5050 | | | |
| Compound 56 | 56-1 | 99% PLA 4A | 28.24 | 15 | 12.65 |
| (Timolol-Bis-N-Acetyl-PLA | | 1% PEG-PLGA5050 | | | |
| (n = 2)-O-Acetyl-PLA | 56-2 | 77% PLA 4A | 27.31 | 15 | 16.33 |
| (n = 4)) | | 22% PLGA8515 5A | | | |
| | | 1% PEG-PLGA5050 | | | |
| | 56-3 | 64% PLA 4A | 27.6 | 15 | 12.60 |
| | | 20% PLGA8515 5A | | | |
| | | 15% PLGA5050 4A | | | |
| | | 1% PEG-PLGA5050 | | | |

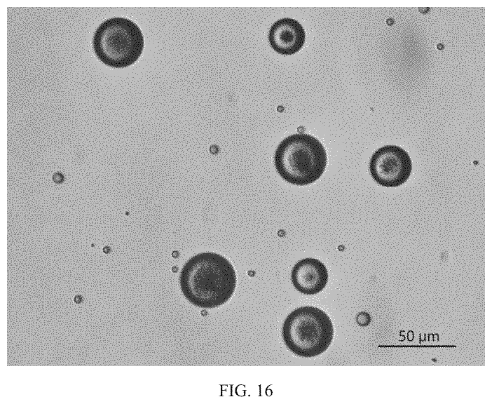

Particle morphology was assessed using a Nikon Eclipse TS-100 light microscope. Briefly, 3-5 mg of particles were suspended in 1 mL of water. A volume of 10 uL of the particle suspension was transferred onto a glass slide and imaged directly. All microparticle formulations of bis-prodrugs of Timolol were found to be spherical in morphology (FIG. 16).

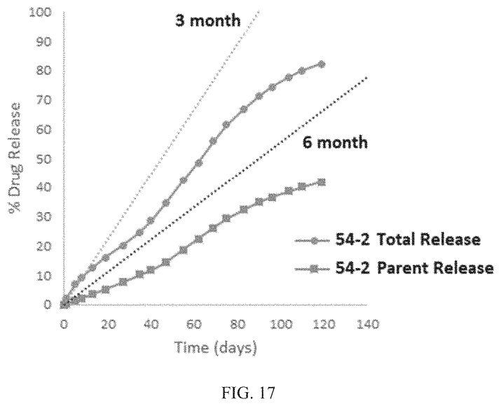

In vitro drug release kinetics were evaluated in a release medium composed of PBS and 1% Tween 20 (pH 7.4). Briefly, 10 mg of particles was suspended in 4 mL of the release medium The particle suspension was incubated on an orbital shaker at 150 rpm at 37° C. At various time points, 3 mL of release media was collected, and the suspension was replenished with 3 mL of fresh release media. Collected release samples were frozen and stored at −80° C. until analysis for drug content. The collected samples were filtered through a 0.2 μm syringe filter and analyzed by RP-HPLC. FIG. 17-FIG. 22 illustrate the total and parent Timolol release profile for the microparticles encapsulating bis-prodrugs of Timolol of Table 11.

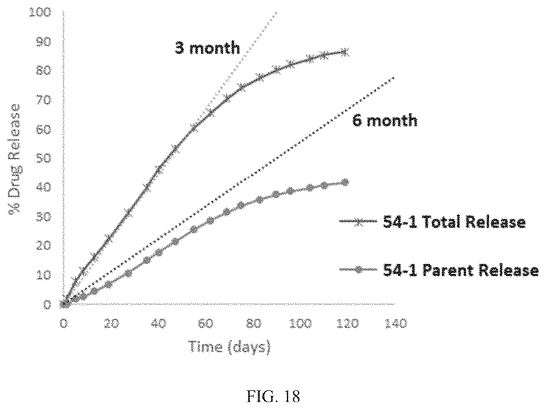

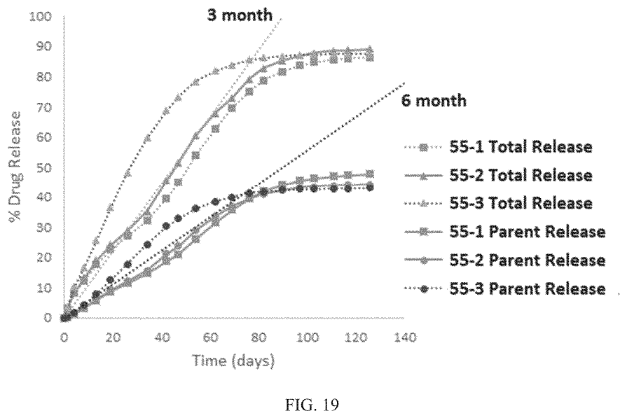

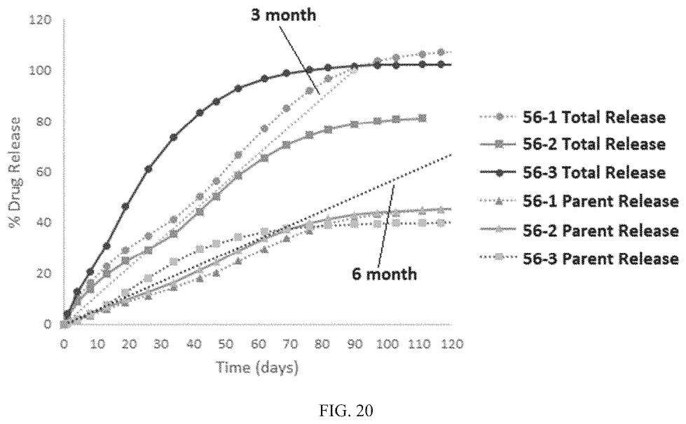

Microparticle formulations encapsulating Timolol-bis-acetyl PLA(n=4) (Compound 54), Timolol-bis-N-acetyl-PLA (n=4)-O-acetyl PLA (n=2) (Compound 55), and Timolol-bis-N-acetyl-PLA (n=2)-O-acetyl PLA (n=4) (Compound 56) were all able to release the total prodrug, intermediates and parent for upwards of 120 days (FIG. 17, FIG. 18, FIG. 19, and FIG. 20). However, the amount of parent Timolol drug released is significantly less than the total release of all the prodrugs and intermediates combined. As the beta-adrenoceptor blocking or antagonist activity of Timolol is highest in its native form, this could affect its bioactivity in vivo.

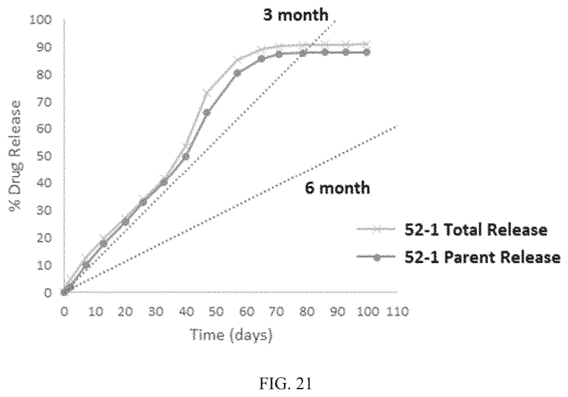

In contrast, two bis-prodrugs of Timolol, Timolol-N,O-bis-glycolic acid-OAc (Compound 52) and Timolol-bis-N,O-glycolic acid-acetyl-PLA (n=4) (Compound 50) were found to degrade and release primarily as native parent Timolol (FIG. 21 and FIG. 22) and not other breakdown products. As shown in FIG. 22, the correlation between total drug release and parent Timolol is high. However, Timolol-N,O-bis-glycolic acid-OAc (Compound 52) only had a maximum release duration of approximately 70 days in vitro (FIG. 21). In contrast, Timolol-bis-N, O-glycolic acid-acetyl-PLA (n=4) (Compound 50) had a linear release profile that extended beyond 140 days with parent Timolol as the primary compound released from the microparticles (FIG. 22).

The drug release of additional batches of polymeric microparticles encapsulating Timolol prodrugs were studied. The microparticles were prepared as described above and the formulation parameters are given in Table 12.

TABLE 12
Formulation Parameters of Additional Microparticles encapsulating Timolol Prodrugs

| Batch | | Polymer | Polymer | Drug Loading | Mean size | |
|---|---|---|---|---|---|---|
| ID | Compd | composition | concentration | (Theoretical 30%) | (um) | Yield |
| 50-A | 50 | 78% PLA1004.5A | 260 mg/mL | Prodrug DL: 28.18% | 28.5 | 34.53% |
| | | 22% PLGA8515 5A | | Parent DL: 8.16% | | |
| | | 1% PEG-PLGA5050 | | | | |
| 50-B | 50 | 78% PLA1004.5A | 260 mg/mL | Prodrug DL: 27.78% | 28.94 | 54.76% |
| | | 22% PLGA8515 5A | | Parent DL: 8.04% | | |
| | | 1% PEG-PLGA5051 | | | | |
| 51-A | 51 | 78% PLA1004.5A | 260 mg/mL | Prodrug DL: 19.9% | 26.73 | — |
| | | 22% PLGA8515 5A | | Parent DL: 7.82% | | |
| | | 1% PEG-PLGA5050 | | | | |
| 52-A | 52 | 78% PLA1004.5A | 260 mg/mL | Prodrug DL: 9.81% | 29.24 | — |
| | | 22% PLGA8515 5A | | Parent DL: 6.01% | | |
| | | 1% PEG-PLGA5050 | | | | |
| 53-A | 53 | 99% PLA1004.5A | 260 mg/mL | Prodrug DL: 5.93% | 26.63 | — |
| | | 1% PEG-PLGA5050 | | Parent DL: 3.03% | | |
| 53-B | 53 | 78% PLA1004.5A | 260 mg/mL | Prodrug DL: 5.65% | 26.82 | — |
| | | 22% PLGA8515 5A | | Parent DL: 2.89% | | |
| | | 1% PEG-PLGA5051 | | | | |

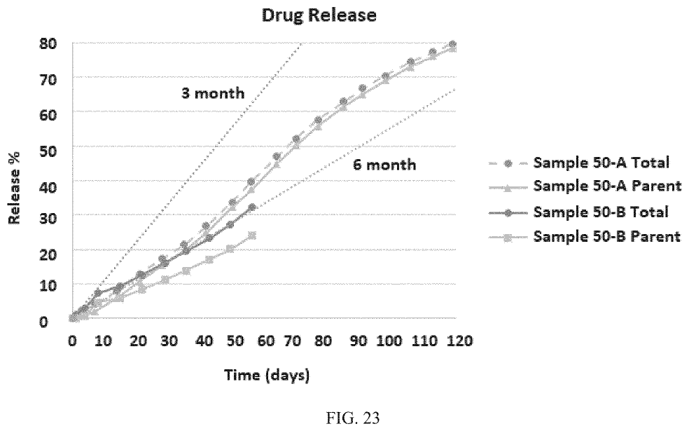

As shown in FIG. 23, both batches of Compound 50 (Batch 50-A and Batch 50-B) achieved a 4-month linear drug release. As also shown in FIG. 23, total drug release (defined as Compound 50+other breakdown products+Timolol) correlates well with Timolol release. The total drug release of Compound 50 is primarily the Timolol parent drug.

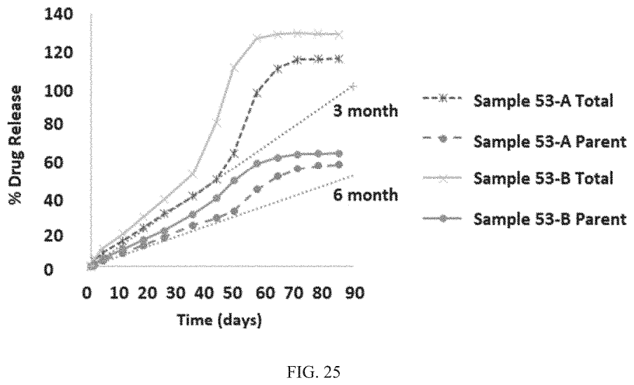

As shown in FIG. 24, the drug release of Compound 51 did not meet the 4-month criteria and the correlation between the total drug release and the parent drug release was poor. FIG. 25 shows that Compound 53 also did not consistently meet the 4-month criteria for linear drug release. Linear 4-month drug release was only achieved with Compound 50. Compound 50 exhibited the best correlation between total drug release and parent Timolol drug release compared to the other Timolol prodrugs of Table 11 and Table 12.

### Example 15. Stability of Timolol Prodrugs

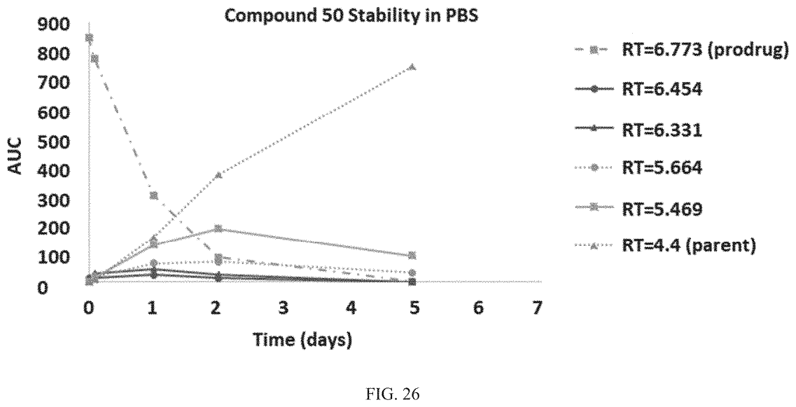

The stability of Compound 50 in PBS over 5 days is shown in FIG. 26. Each point on the graph is the retention time of Compound 50, parent Timolol, and 4 breakdown products at day 0, 1, 2, 3, 4, or 5 as measured via HPLC. The prodrug (retention time=6.773) break downs over the course of 5 days into the parent Timolol drug (retention time=4.4) and the other breakdown products, but as shown in FIG. 26, Compound 50 primarily breaks downs into the Timolol parent drug and the Timolol concentration as measured via absorbance is high compared to the other breakdown products.

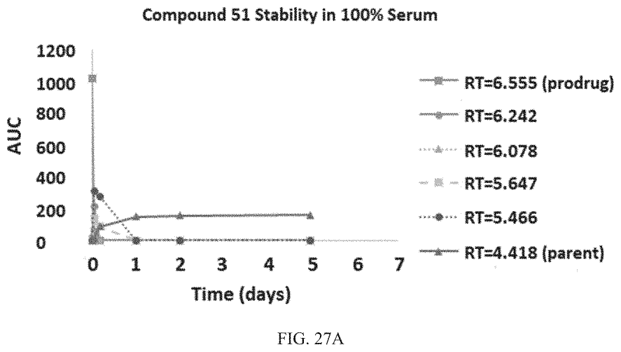

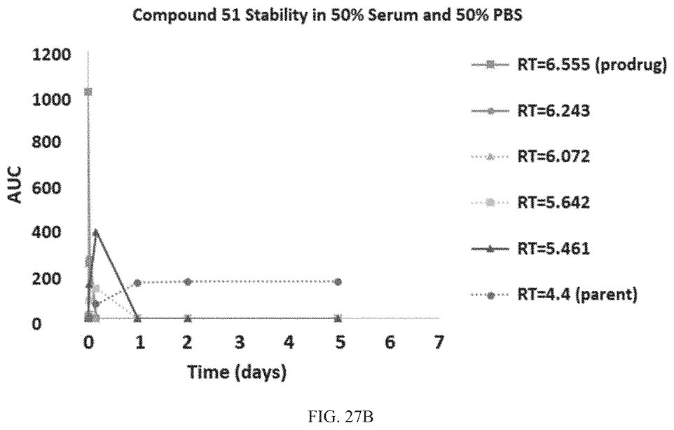

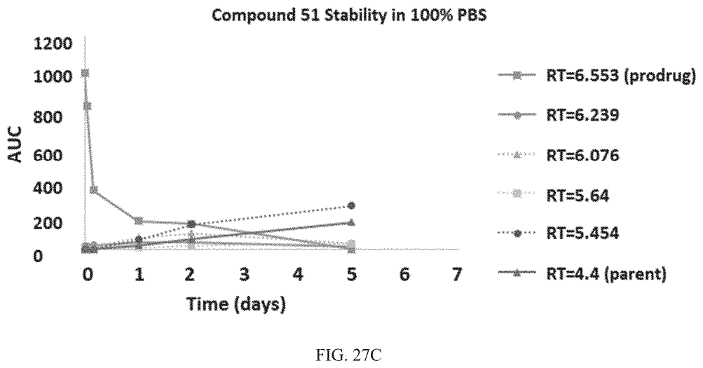

The stability of Compound 51 was measured in 100% serum (FIG. 27A), 50% serum and 50% PBS (FIG. 27B), and 100% PBS (FIG. 27C) via HPLC. Each point on the graph is the retention time of Compound 51, parent Timolol, and 4 breakdown products at day 0, 1, 2, 3, 4, or 5 as measured. In 100% serum (FIG. 27A), Compound 51 (retention time=6.555) broke down over the course of five days into Timolol parent and the four other breakdown products, but the concentration of Timolol parent at day 5 is similar to the concentration of the other breakdown products. Similar results were achieved when the stability was measured in 50% serum and 50% PBS (FIG. 27B). In 100% PBS (FIG. 27C), parent Timolol was not even the most prominent breakdown product at the end of the study on day 5. The concentration of the breakdown product with a retention time of 5.454 minutes was higher than the concentration of parent Timolol (retention time=4.4 minutes).

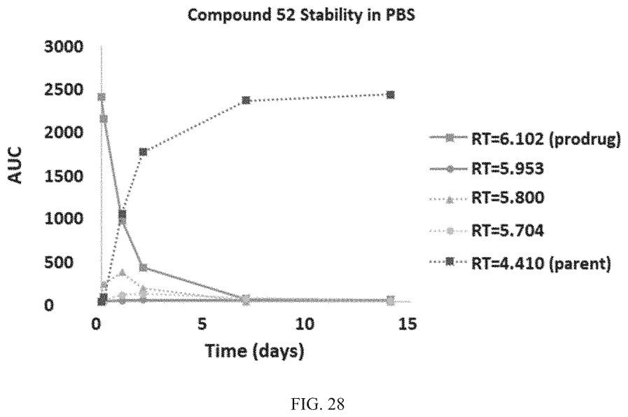

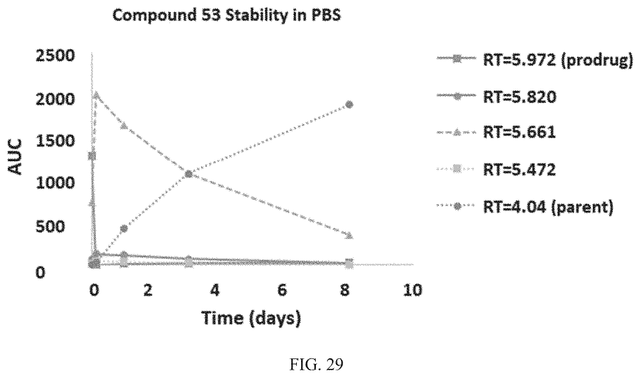

The stability of Compound 52 was measured in PBS (FIG. 28) and the stability of Compound 53 was measured in PBS (FIG. 29). Compound 52 breaks down into parent Timolol over the course of the study. Compound 53 breaks down into parent Timolol (retention time=4.04 minutes), but a significant amount of an additional breakdown product with a retention time of 5.661 was also observed over the course of the eight day study.

These results highlight the surprising drug release characteristics of Compound 50. Compound 50 was the only compound to consistently achieve linear 4-month drug release and during the stability study, Compound 50 also produced high concentrations of Timolol during the breakdown process.

This specification has been described with reference to embodiments of the invention. However, one of ordinary skill in the art appreciates that various modifications and changes can be made without departing from the scope of the invention as set forth herein. Accordingly, the specification is to be regarded in an illustrative rather than a restrictive sense, and all such modifications are intended to be included within the scope of invention.

What is claimed is:

1. A compound of Formula 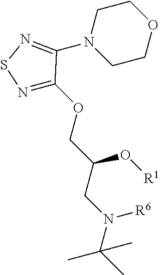
or a pharmaceutically acceptable salt thereof;
wherein
R¹ is selected from 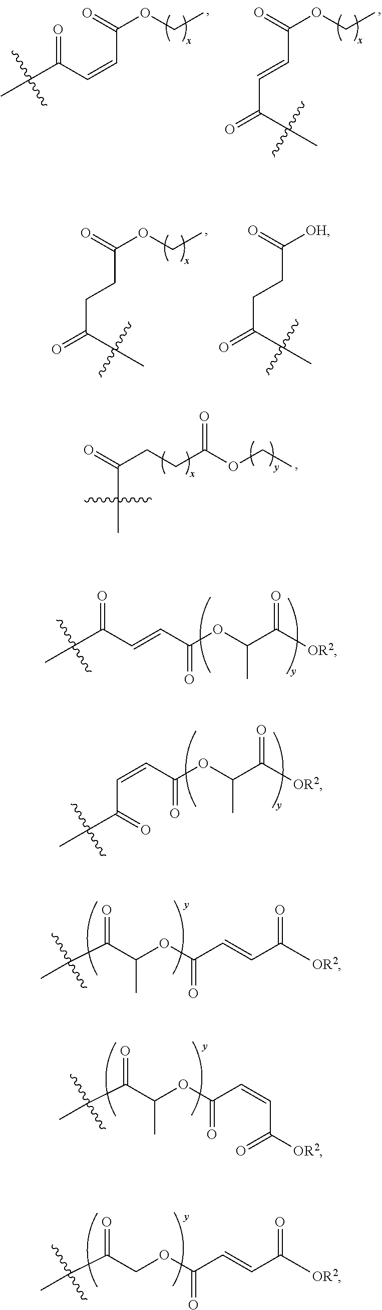 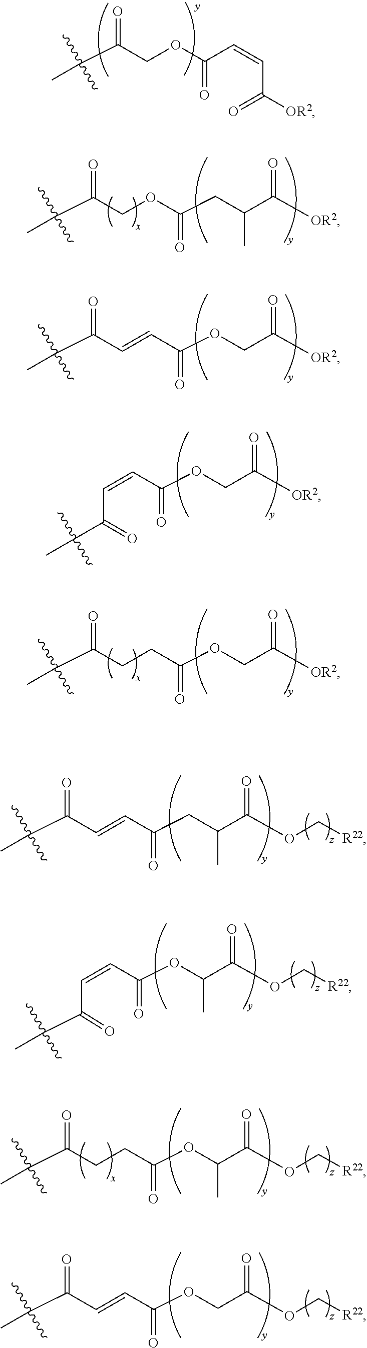 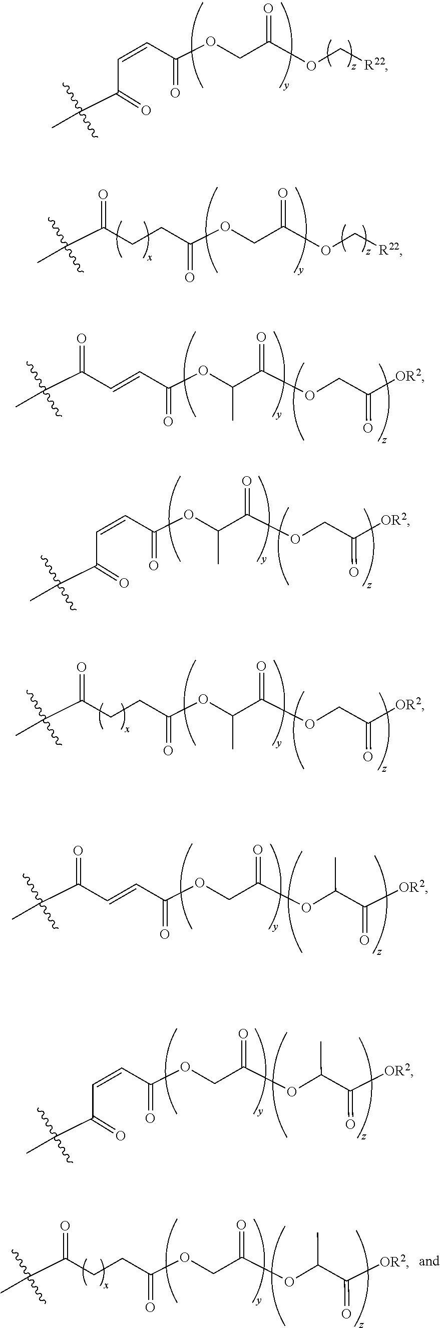 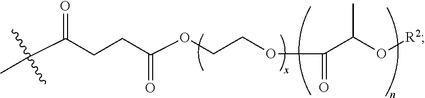
R² is hydrogen, alkyl, alkenyl, alkynyl cycloalkyl, cycloalkylalkyl, heterocyclyl, heterocycloalkyl, aryl, arylalkyl, heteroaryl, or heteroarylalkyl;
R²² is hydrogen, hydroxy, amino, A, alkyl, alkoxy, alkenyl, alkynyl cycloalkyl, cycloalkylalkyl, heterocyclyl, heterocycloalkyl, aryl, arylalkyl, heteroaryl, heteroarylalkyl, aryloxy, or stearoyl;
A is selected from H, alkyl, cycloalkyl, cycloalkylalkyl, heterocycle, heterocycloalkyl, aryl, arylalkyl, heteroaryl, heteroarylalkyl, aryloxy, and alkyloxy;
R⁶ is selected from C(O)A, hydrogen, and R³⁶;
R³⁶ is selected from C(O)A, 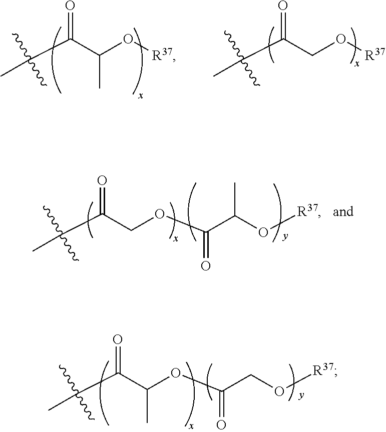
R³⁷ is selected from hydrogen, —C(O)A, —C(O)alkyl, aryl, alkyl, cycloalkyl, cycloalkylalkyl, heterocyclyl, heterocycloalkyl, arylalkyl, heteroaryl, and heteroarylalkyl; and
x, y, and z are independently selected from 1, 2, 3, 4, 5, 6, 7, 8, 9, or 10.

2. The compound of claim 1, wherein R¹ is selected from

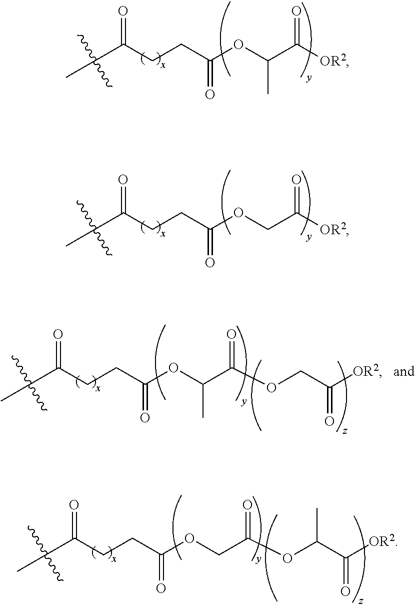

3. The compound of claim 2, wherein R² is alkyl.

4. The compound of claim 2, wherein x is an integer selected from 1, 2, 3, 4, 5, or 6.

5. The compound of claim 2, wherein y is an integer selected from 1, 2, 3, 4, 5, or 6.

6. The compound of claim 2, wherein z is an integer selected from 1, 2, 3, 4, 5, or 6.

7. The compound of claim 1, wherein R¹ is selected from

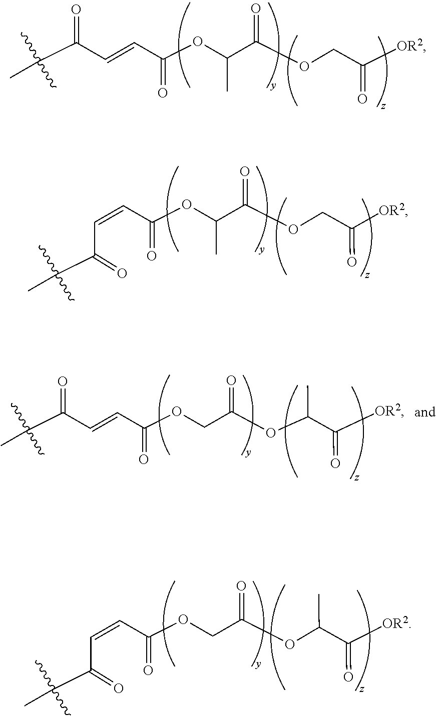

8. The compound of claim 7, wherein R² is alkyl.

9. The compound of claim 7, wherein x is an integer selected from 1, 2, 3, 4, 5, or 6.

10. The compound of claim 7, wherein y is an integer selected from 1, 2, 3, 4, 5, or 6.

11. The compound of claim 7, wherein z is an integer selected from 1, 2, 3, 4, 5, or 6.

12. The compound of claim 1, wherein R⁶ is hydrogen.

13. The compound of claim 1, wherein R⁶ is R³⁶ and R³⁶ is selected from

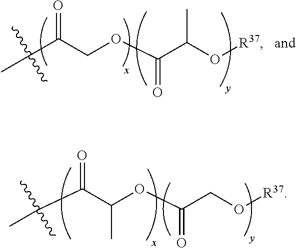

14. The compound of claim 13, wherein R³⁶ is 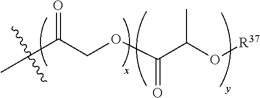
and R³⁷ is —C(O)alkyl.

15. The compound of claim 13, wherein R³⁶ is 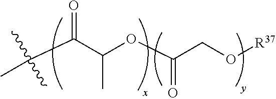
and R³⁷ is —C(O)alkyl.

16. The compound of claim 1 of the formula 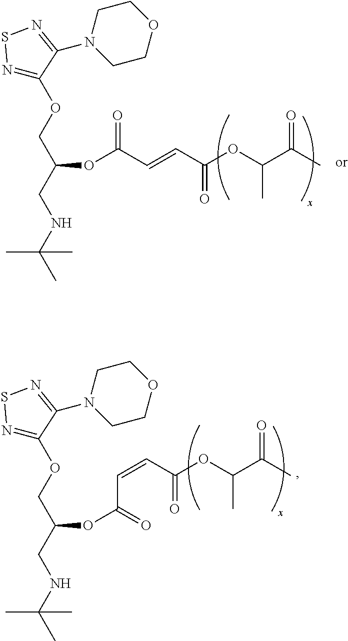
or a pharmaceutically acceptable salt thereof.

17. The compound of claim 16 wherein x is selected from 1, 2, 3, 4, 5, or 6.

18. The compound of claim 16 of the formula 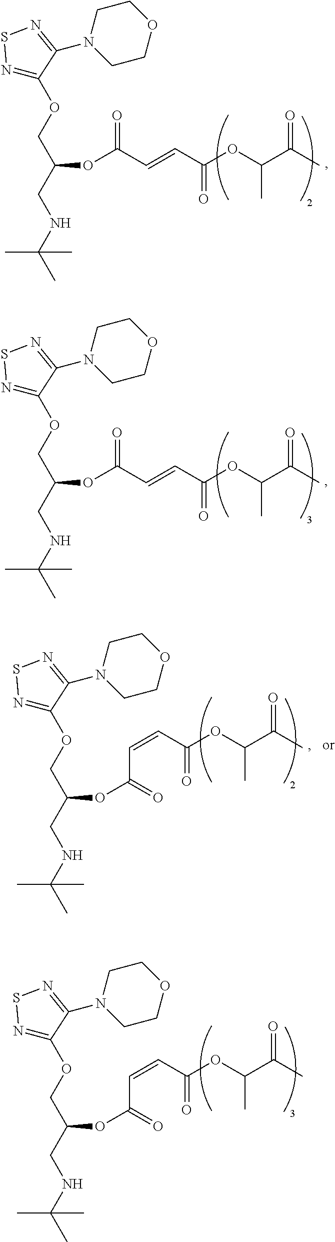
or a pharmaceutically acceptable salt thereof.

19. A pharmaceutical composition comprising a compound of claim 1 or a pharmaceutically acceptable salt thereof in a pharmaceutically acceptable carrier.

20. A method for the treatment of an ocular disorder comprising administering a compound of claim 1 or a pharmaceutically acceptable salt thereof to a host in need thereof wherein the disorder is selected from glaucoma, wet age-related macular degeneration, and a disorder related to an increase in intraocular pressure (IOP).

21. The method of claim 20, wherein the compound is administered via intravitreal injection.

22. The method of claim 20, wherein the compound is administered via subconjunctival injection.

23. The method of claim 20, wherein the compound is administered via suprachoroidal injection.
